(19) United States
(12) Patent Application Publication
(10) Pub. No.: US 2023/0312684 A1
(43) Pub. Date: Oct. 5, 2023

(54) NEUROPILIN AND ANGIOTENSIN CONVERTING ENZYME 2 FUSION PEPTIDES FOR TREATING VIRAL INFECTIONS

(71) Applicant: PINETREE THERAPEUTICS, INC., Cambridge, MA (US)

(72) Inventors: Ho Juhn SONG, Cambridge, MA (US); Euijoon JEONG, Cambridge, MA (US); Anthony John ROSSOMANDO, Cambridge, MA (US); Sung Hugh CHOI, Cambridge, MA (US); Clemens REINSHAGEN, Cambridge, MA (US); Yongbin TAK, Cambridge, MA (US); DeLuna XAVIER, Cambridge, MA (US); Chikwamba KUDZAI, Cambridge, MA (US)

(21) Appl. No.: 18/040,083

(22) PCT Filed: Aug. 2, 2021
(86) PCT No.: PCT/US2021/044135

§ 371 Date: Jan. 31, 2023

Related U.S. Application Data
(60) Provisional application No. 63/059,915, filed on Jul. 31, 2020.

(51) Int. Cl. C07K 14/71; A61P 31/14; C12N 9/48

(52) U.S. Cl. CPC C07K 14/71; A61P 31/14; C12N 9/485; C12Y 304/17023; C07K 2319/30; A61K 38/00

(57) ABSTRACT

The present disclosure relates to fusion protein compositions and methods of reducing and treating viral infections. The fusion proteins include a polypeptide comprising a b1 domain, or a derivative or fragment thereof, of a neuropilin; an ACE2 domain, or a derivative or a fragment thereof, of an angiotensin converting enzyme 2; and or an immunoglobulin domain. Both the b1 and ACE2 domains are capable of binding to a coat protein of a virus selected from the group consisting of herpesviridae, papillomaviridae, coronaviridae, flaviviridae, togaviridae, bomaviridae, bunyaviridae, filoviridae, orthomyxoviridae, paramyxoviridae, pneumoviridae, and retro viridae. In some embodiments, the b1 domain, or a derivative or fragment thereof, and/or the (ACE2) domain, can be used to specifically bind S proteins of COVID-19 particles.

### CROSS REFERENCE TO RELATED APPLICATIONS

This application claims the benefit of, and priority to, U.S. Provisional Application Ser. No. 63/059,915, filed on Jul. 31, 2020, the disclosure of which is incorporated by reference herein in its entirety.

### SEQUENCE

This application incorporates by reference in its entirety the Sequence Listing entitled “268824-494885_ST25.txt” (980 KB), which was created on Jul. 30, 2021 at 12:21 PM, and filed electronically herewith.

### TECHNICAL FIELD

The present disclosure relates to fusion protein compositions and methods of reducing and treating viral infections, and more specifically, polypeptides comprising a combination of neuropilin-1 (NRP1) domain, neuropilin-2 (NRP2) domain, angiotensin converting enzyme 2 (ACE2) domain, and/or an immunoglobulin domain that can be used to specifically bind a coat protein of a virus particle such as a S protein of a COVID-19 virus.

### BACKGROUND

Coronavirus disease 2019 (COVID-19) is an infectious disease caused by severe acute respiratory syndrome coronavirus 2 (SARS-CoV-2). As of Jul. 27, 2020, more than 16.1 million cases have been reported across 188 countries and territories, resulting in more than 647,000 deaths.

Common symptoms include fever, cough, fatigue, shortness of breath, and loss of smell and taste. While the majority of cases result in mild symptoms, some progress to acute respiratory distress syndrome (ARDS) possibly precipitated by a cytokine storm, multi-organ failure, septic shock, and blood clots. The time from exposure to onset of symptoms is typically around five days, but may range from two to fourteen days.

The virus is primarily spread between people during close contact, most often via small droplets produced by coughing, sneezing, and talking. The droplets usually fall to the ground or onto surfaces rather than travelling through air over long distances. Transmission may also occur through smaller droplets that are able to stay suspended in the air for longer periods of time. Less commonly, people may become infected by touching a contaminated surface and then touching their face. It is most contagious during the first three days after the onset of symptoms, although spread is possible before symptoms appear, and from people who do not show symptoms. The standard method of diagnosis is by real-time reverse transcription polymerase chain reaction (rRT-PCR) from a nasopharyngeal swab.

There are currently no vaccines nor specific antiviral treatments for COVID-19. Management involves the treatment of symptoms, supportive care, isolation, and experimental measures. The World Health Organization (WHO) declared the COVID-19 outbreak a public health emergency of international concern (PHEIC) on Jan. 30, 2020 and a pandemic on Mar. 11, 2020.

### SUMMARY

In some aspects, the present disclosure provides a polypeptide comprising: a b1 domain, or a derivative or fragment thereof, of a neuropilin; and an immunoglobulin domain, wherein the b1 domain is capable of binding to a coat protein of a virus selected from the group consisting of herpesviridae, papillomaviridae, coronaviridae, flaviviridae, togaviridae, bornaviridae, bunyaviridae, filoviridae, orthomyxoviridae, paramyxoviridae, pneumoviridae, and retroviridae.

In other aspects, the present disclosure provides a polypeptide comprising: an ACE2 domain, or a derivative or a fragment thereof, of an angiotensin converting enzyme 2; and an immunoglobulin domain, wherein the ACE2 domain is capable of binding to a coat protein of a virus selected from the group consisting of herpesviridae, papillomaviridae, coronaviridae, flaviviridae, togaviridae, bornaviridae, bunyaviridae, filoviridae, orthomyxoviridae, paramyxoviridae, pneumoviridae, and retroviridae.

In still other aspects, the present disclosure provides a polypeptide comprising: a b1 domain, or a derivative or fragment thereof, of a neuropilin; and an ACE2 domain, or a derivative or fragment thereof, of angiotensin converting enzyme 2, wherein the b1 domain and ACE2 domain are each capable of binding to a coat protein of a virus selected from the group consisting of herpesviridae, papillomaviridae, coronaviridae, flaviviridae, togaviridae, bornaviridae, bunyaviridae, filoviridae, orthomyxoviridae, paramyxoviridae, pneumoviridae, and retroviridae.

In yet other aspects, the present disclosure provides a polypeptide comprising: a b1 domain, or a derivative or fragment thereof, of a neuropilin; an ACE2 domain, or a derivative or fragment thereof, of angiotensin converting enzyme 2; and an immunoglobulin domain, wherein the b1 domain and ACE2 domain are each capable of binding to a coat protein of a virus selected from the group consisting of herpesviridae, papillomaviridae, coronaviridae, flaviviridae, togaviridae, bornaviridae, bunyaviridae, filoviridae, orthomyxoviridae, paramyxoviridae, pneumoviridae, and retroviridae.

Embodiments of these aspects of the invention directed to toward polypeptides comprising a combination of two or more domains including a b1 domain, or a derivative or fragment thereof, of a neuropilin; an ACE2 domain, or a derivative or fragment thereof, of angiotensin converting enzyme 2; and an immunoglobulin domain, may include one or more of the following optional features. In some embodiments, the b1 domain, or derivative or fragment thereof, comprises the amino acid sequence of SEQ ID NOs: SEQ ID NO: 3 (NRP1 b1) or SEQ ID NO: 11 (NRP2 b1). In some embodiments, the b1 domain, or derivative or fragment thereof, comprises the amino acid sequence of SEQ ID NO: 3. In some embodiments, the polypeptide is capable of binding to a coat protein of a coronaviridae virus. In some embodiments, the polypeptide is capable of binding to a coat protein of COVID-19. In some embodiments, the coat protein is an S protein of COVID-19. In some embodiments, the b1 domain, or derivative or fragment thereof, comprises a mutation that enhances the affinity for an S protein of COVID-19 when compared with the unmutated b1 domain. In some embodiments, the b1 domain, or derivative or fragment thereof, comprises a mutation at a position selected from the group consisting of E319 and K351. In some embodiments, the b1 domain comprises the amino acid sequence of any of SEQ ID. NOS: SEQ ID NO: 4 (NRP1 b1 E319A) and SEQ ID NO: 5 (NRP1 b1 K351A). In some embodiments, the polypeptide contains a plurality of b1 domains, or derivatives or fragments thereof. In some embodiments, the b1 domain, or derivative or fragment thereof, further comprises a linker, a b2 domain of neuropilin, or a combination thereof. In some embodiments, the b1 domain, or derivative or fragment thereof, is selected from the group consisting of SEQ ID NOS: SEQ ID NO: 7-SEQ ID NO: 14. In some embodiments, the ACE2 domain, or derivative or fragment thereof comprises a sequence selected from the group consisting of SEQ ID NOS: SEQ ID NO: 38-SEQ ID NO: 39. In some embodiments, the polypeptide comprises a plurality of ACE2 domains, or derivatives or fragments thereof. In some embodiments, the ACE2 domain contains a mutation at a position selected from the group consisting of F28, D30, and L79. In some embodiments, the ACE2 domain, derivative or fragment thereof comprises the amino acid sequence of SEQ ID. NOs: SEQ ID NO: 40-SEQ ID NO: 43. In some embodiments, the polypeptide further comprises a linker between the b1 domain and ACE2 domain. In some embodiments, the linker is selected from the group consisting of SEQ ID NOs: 44-50. In some embodiments, the immunoglobulin domain comprises a Fc domain. In some embodiments, the immunoglobulin domain consists essentially of a Fc domain. In some embodiments, the Fc domain contains a mutation that reduces ADCC when compared with a wildtype Fc domain. In some embodiments, the mutation is at position N297 as determined by KABAT numbering. In some embodiments, the Fc domain contains one or more mutations that enhances affinity for a FcRn when compared with a wildtype Fc domain. In some embodiments, the mutation is at a position selected from the group consisting of T307, E380, and N434 as determined by KABAT numbering, or combinations thereof. In some embodiments, the Fc domain contains a mutation that reduces affinity for Fcγ receptor subtypes when compared with a wildtype Fc domain. In some embodiments, the mutation is at a position selected from the group consisting of L324 and L325 as determined by KABAT numbering, or combinations thereof. In some embodiments, the Fc domain is selected from the group consisting of human IgG1, human IgG2, human IgG3, human IgG4, and human IgA. In some embodiments, the Fc domain comprises the amino acid sequence selected from the group consisting of SEQ ID. NOs: 23-31. In some embodiments, the Fc domain sequence comprises the amino acid sequence of SEQ ID. NOS: 23, 30, or 31. In some embodiments, the polypeptide has a configuration selected from the group consisting of: a (b1), IgG1 WT, ACE2-1 polypeptide; a (b1b2), IgG1 (T307A/E380A/N434A), ACE2-2 polypeptide; a (b1b1)-(G4S)*2-(b1b1), IgG1 (N297A), ACE2-3 polypeptide; a (b1b2)-(G4S)*2-(b1b2), IgG1 (L324A/L325A), ACE2-4 polypeptide; a (b1b2)-(G4S)*2-(b1b2) with b1 (E319A), IgG1 (N297A/T307A/E380A/N434A), ACE2-5 polypeptide; and a (b1b2)-(G4S)*2-(b1b2) with b1 (K351A), IgG1 (L324A/L325A/T307A/E380A/N434A), ACE2-6 polypeptide. In some embodiments, the polypeptide comprises the amino acid sequence selected from the group consisting of SEQ ID. NOS: 88-110. In some embodiments, the b1 domain is attached to the C-terminus of the Fc domain. In some embodiments, the b1 domain is attached to the N-terminus of the Fc domain. In some embodiments, the ACE2 domain is attached to the C-terminus of the Fc domain. In some embodiments, the ACE2 domain is attached to the N-terminus of the Fc domain. In some embodiments, the polypeptide further comprises a signal peptide. In some embodiments, the signal peptide comprises the SEQ ID NO: 51.

In some aspects, the present disclosure provides a method of producing the polypeptides disclosed herein, the method comprising recombinantly expressing a nucleic acid vector encoding the polypeptide in a host cell.

In some aspects, the present disclosure provides a pharmaceutical composition comprising the polypeptides disclosed herein and a pharmaceutically acceptable excipient.

In some aspects, the present disclosure provides a method of reducing COVID infection, the method comprising the administration of the polypeptides disclosed herein to a subject in need thereof.

In some aspects, the present disclosure provides a method of treating a subject suffering from COVID infection, the method comprising the administration of the polypeptides disclosed herein to a subject in need thereof.

In some aspects, the present disclosure provides a method of preventing COVID infection, the method comprising the administration of the polypeptides disclosed herein to a subject in need thereof.

In some aspects, the present disclosure provides a method of reducing symptoms of a COVID infection, the method comprising the administration of the polypeptides disclosed herein to a subject in need thereof.

In some aspects, the present disclosure provides a method of reducing transmission of a COVID infection, the method comprising the administration of the polypeptides disclosed herein to a subject in need thereof.

In addition, the present disclosure describes a recombinant polypeptide comprising: (a) one or more mutant neuropilin (NRP) domains, or fragments thereof, and (b) an immunoglobulin domain; wherein the one or more mutant NRP domains result in reduced binding of the recombinant polypeptide to heparin or heparan sulfate relative to a wild-type NRP domain.

In addition, the present disclosure describes a recombinant polypeptide comprising: (a) one or more mutant neuropilin (NRP) b1 domains, NRP b2 domains, or fragments thereof, and (b) an Fc domain; wherein the one or more mutant NRP b1 domains, NRP b2 domains, or fragments thereof are derived from an NRP1 or an NRP2 protein; wherein the one or more mutant NRP b1 domains, NRP b2 domains, or fragments thereof have one or more amino substitutions selected from groups consisting of: K373E, K351A, E319A, K358E, R513E, K514E, K516E, R513A, K514A, K516A, Y297A, S345A, and Y353A, relative to the wild-type amino acid sequence set forth in SEQ ID NO: 1; and wherein the one or more one or more amino substitutions result in reduced binding of the recombinant polypeptide to heparin or heparan sulfate.

In addition, the present disclosure describes a recombinant polypeptide comprising: (a) one or more mutant neuropilin (NRP) b1 domains (b1), NRP b2 domains (b2), or fragments thereof, and (b) an Fe domain; wherein (a) and (b) comprise a construct having an orientation of: b1-Fc; b1b1-Fc; b1b1b1-Fc; b1-Fc; b1b1b1-Fc; b1b2-Fc; b1b2-Fc; b1b2-Fc; b1b2-Fc; Fc-b1b2; Fc-b1b2; b1-Fc-b1; b1b1-Fc-b1; b1-Fc; b1b1-Fc; b1b1b1-Fc; b1-Fc; b1b1b1-Fc; b1-Fc; b1b1b1-Fc; b1b2-Fc; b1b2-Fc; Fc-b1b2; Fc-b1b2; b1-Fc-b1; b1b1-Fc-b1; wherein the one or more b1, b2, or fragments thereof, are derived from an NRP1 or an NRP2 protein; wherein the one or more b1, b2, or fragments thereof have one or more amino substitutions selected from groups consisting of: K373E, K351A, E319A, K358E, R513E, K514E, K516E, R513A, K514A, K516A, Y297A, S345A, and Y353A, relative to the wild-type amino acid sequence set forth in SEQ ID NO: 1; and wherein the one or more one or more amino substitutions result in reduced binding of the recombinant polypeptide to heparin or heparan sulfate.

In addition, the present disclosure describes a recombinant polypeptide comprising: (a) one or more mutant neuropilin (NRP) domains, or fragments thereof, and (b) an immunoglobulin domain; wherein the recombinant polypeptide is operable to bind to virus having an —Z₁-X₁-X₂—Z₂- (CendR) motif, wherein Z₁ and Z₂ are arginine or lysine, and X₁ and X₂ are any amino acid.

In addition, the present disclosure describes a recombinant polypeptide comprising: (a) one or more mutant neuropilin (NRP) b1 domains (b1), NRP b2 domains (b2), or fragments thereof, and (b) an Fc domain; wherein the one or more b1, b2, or fragments thereof, are derived from an NRP1 or an NRP2 protein; wherein the recombinant polypeptide is operable to bind to virus having an —Z₁-X₁-X₂—Z₂- (CendR) motif, wherein Z₁ and Z₂ are arginine or lysine, and X₁ and X₂ are any amino acid; and wherein the virus is selected from the group consisting of: Dengue; respiratory syncytial virus (RSV); Hantavirus; Epstein-Barr virus (EBV); EBV (uncleaved); SARS-CoV-2 Wuhan; SARS-CoV-2 Wuhan (uncleaved); SARS-CoV-2 UK; SARS-CoV-2 India; SARS-CoV-2 India (uncleaved); HCoV-OC43; MERS-CoV; MERS-CoV (uncleaved); Herpes simplex virus (HSV) 1; HSV 1 (uncleaved); influenza A H5N1 virus (IAV H5N1); human papillomavirus (HPV); Human Metapneumovirus; and human immunodeficiency virus (HIV).

In addition, the present disclosure describes a recombinant polypeptide comprising: (a) one or more mutant neuropilin (NRP) b1 domains (b1), NRP b2 domains (b2), or fragments thereof, and (b) an Fc domain; wherein the one or more b1, b2, or fragments thereof, are derived from an NRP1 or an NRP2 protein; wherein the recombinant polypeptide is operable to bind to virus having an —Z₁-X₁-X₂—Z₂- (CendR) motif, wherein Z₁ and Z₂ are arginine or lysine, and X₁ and X₂ are any amino acid; and wherein the virus has a CendR motif selected from the group consisting of:

GTCTQSGERRREKR;
KNTNVTLSKKRKRR;
LTHKMIEESHRLRR;
VSFKPPPPPSRRRR;
VSFKPPPPPSRRRRGACVVY;
CASYQTQTNSPRRAR;
CASYQTQTNSPRRARSVASQSIIAYTMSLG;
ASYQTQTNSHRRAR;
ASYQTQTNSRRRAR;
ASYQTQTNSRRRARSVASQSIIAY;
GSGYCVDYSKNRRSR;
LLEPVSISTGSRSAR;
LLEPVSISTGSRSARSAIEDLLFDK;
ERPRAPARSASRPRR;
ERPRAPARSASRPRRPV;
VLATGLRNVPQRKKR;
PTTSSTSTTAKRKKR;
IDMLKARVKNRVAR;
AKRRVVQREKR;
and
AKRRVVQREKRAVGIGALFLG.

In addition, the present disclosure describes a recombinant polypeptide comprising an amino acid sequence that is at least 90% identical to an amino acid sequence set forth in any one of SEQ ID NOs: 113-116, 121-122, 133-137, 148-149, 154, 162, and 193-201, or a pharmaceutically acceptable salt thereof.

In addition, the present disclosure describes a recombinant polypeptide consisting of an amino acid sequence that is at least 90% identical to an amino acid sequence according to set forth in any one of SEQ ID NOs: 113-116, 121-122, 133-137, 148-149, 154, 162, and 193-201, or a pharmaceutically acceptable salt thereof.

In addition, the present disclosure describes a recombinant polypeptide consisting of an amino acid sequence set forth in any one of SEQ ID NOs: 113-116, 121-122, 133-137, 148-149, 154, 162, and 193-201, or a pharmaceutically acceptable salt thereof.

In addition, the present disclosure describes a method of limiting the occurrence of, reducing the risk of, reducing the severity of, or treating a viral infection, in a subject in need thereof, said method comprising administering to the subject a composition comprising a therapeutically effective amount of a recombinant polypeptide comprising: (a) one or more mutant neuropilin (NRP) domains, or fragments thereof, and (b) an immunoglobulin domain; wherein the recombinant polypeptide is operable to bind to virus having an —Z₁-X₁-X₂—Z₂- (CendR) motif, wherein Z₁ and Z₂ are arginine or lysine, and X₁ and X₂ are any amino acid.

In addition, the present disclosure describes a method of limiting the occurrence of, reducing the risk of, reducing the severity of, or treating a viral infection, in a subject in need thereof, said method comprising administering to the subject a composition comprising a therapeutically effective amount of a recombinant polypeptide comprising: (a) one or more mutant neuropilin (NRP) b1 domains (b1), NRP b2 domains (b2), or fragments thereof, and (b) an Fc domain; wherein the one or more b1, b2, or fragments thereof, are derived from an NRP1 or an NRP2 protein; wherein the recombinant polypeptide is operable to bind to virus having an —Z₁-X₁-X₂—Z₂- (CendR) motif, wherein Z₁ and Z₂ are arginine or lysine, and X₁ and X₂ are any amino acid; and wherein the virus is selected from the group consisting of: Dengue; respiratory syncytial virus (RSV); Hantavirus; Epstein-Barr virus (EBV); EBV (uncleaved); SARS-CoV-2 Wuhan; SARS-CoV-2 Wuhan (uncleaved); SARS-CoV-2 UK; SARS-CoV-2 India; SARS-CoV-2 India (uncleaved); HCoV-OC43; MERS-CoV; MERS-CoV (uncleaved); Herpes simplex virus (HSV) 1; HSV 1 (uncleaved); influenza A H5N1 virus (IAV H5N1); human papillomavirus (HPV); Human Metapneumovirus; and human immunodeficiency virus (HIV).

In addition, the present disclosure describes a method of limiting the occurrence of, reducing the risk of, reducing the severity of, or treating a viral infection, in a subject in need thereof, said method comprising administering to the subject a composition comprising a therapeutically effective amount of a recombinant polypeptide comprising: (a) one or more mutant neuropilin (NRP) b1 domains (b1), NRP b2 domains (b2), or fragments thereof, and (b) an Fc domain; wherein the one or more b1, b2, or fragments thereof, are derived from an NRP1 or an NRP2 protein; wherein the recombinant polypeptide is operable to bind to virus having an —Z₁-X₁-X₂—Z₂- (CendR) motif, wherein Z₁ and Z₂ are arginine or lysine, and X₁ and X₂ are any amino acid; and wherein the virus has a CendR motif selected from the group consisting of:

GTCTQSGERRREKR;
KNTNVTLSKKRKRR;
LTHKMIEESHRLRR;
VSFKPPPPPSRRRR;
VSFKPPPPPSRRRRGACVVY;
CASYQTQTNSPRRAR;
CASYQTQTNSPRRARSVASQSIIAYTMSLG;
ASYQTQTNSHRRAR;
ASYQTQTNSRRRAR;
ASYQTQTNSRRRARSVASQSIIAY;
GSGYCVDYSKNRRSR;
LLEPVSISTGSRSAR;
LLEPVSISTGSRSARSAIEDLLFDK;
ERPRAPARSASRPRR;
ERPRAPARSASRPRRPV;
VLATGLRNVPQRKKR;
PTTSSTSTTAKRKKR;
IDMLKARVKNRVAR;
AKRRVVQREKR;
and
AKRRVVQREKRAVGIGALFLG.

In addition, the present disclosure describes a method of limiting the occurrence of, reducing the risk of, reducing the severity of, or treating a viral infection, in a subject in need thereof, said method comprising administering to the subject a composition comprising a therapeutically effective amount of a recombinant polypeptide comprising an amino acid sequence that is at least 90% identical to an amino acid sequence set forth in any one of SEQ ID NOs: 113-116, 121-122, 133-137, 148-149, 154, 162, and 193-201, or a pharmaceutically acceptable salt thereof.

In addition, the present disclosure describes a method of limiting the occurrence of, reducing the risk of, reducing the severity of, or treating a viral infection, in a subject in need thereof, said method comprising administering to the subject a composition comprising a therapeutically effective amount of a recombinant polypeptide consisting of an amino acid sequence that is at least 90% identical to an amino acid sequence according to set forth in any one of SEQ ID NOs: 113-116, 121-122, 133-137, 148-149, 154, 162, and 193-201, or a pharmaceutically acceptable salt thereof.

In addition, the present disclosure describes a method of limiting the occurrence of, reducing the risk of, reducing the severity of, or treating a viral infection, in a subject in need thereof, said method comprising administering to the subject a composition comprising a therapeutically effective amount of a recombinant polypeptide consisting of an amino acid sequence set forth in any one of SEQ ID NOs: 113-116, 121-122, 133-137, 148-149, 154, 162, and 193-201, or a pharmaceutically acceptable salt thereof.

In addition, the present disclosure describes a recombinant polypeptide comprising an amino acid sequence that is at least 90% identical to an amino acid sequence set forth in any one of SEQ ID NOs: 113-116, 121-122, 133-137, 148-149, 154, 162, and 193-201, or a pharmaceutically acceptable salt thereof, and further comprising an excipient.

In addition, the present disclosure describes a method of limiting the occurrence of, reducing the risk of, reducing the severity of, or treating a viral infection, in a subject in need thereof, said method comprising administering to the subject a composition comprising a therapeutically effective amount of a recombinant polypeptide of the present invention, wherein the virus is a virus belonging to the Family: Astroviridae; Bunyaviridae; Bornaviridae; Chuviridae; Flaviviridae; Filoviridae; Hantaviridae; Hepeviridae; Herpesviridae; Nairoviridae; Orthomyxoviridae; Papillomaviridae; Paramyxoviridae; Peribunyaviridae; Phenuiviridae; Pneumoviridae; Poxviridae; Retroviridae; Rhabdoviridae; or Togaviridae.

In addition, the present disclosure describes a method of limiting the occurrence of, reducing the risk of, reducing the severity of, or treating a viral infection, in a subject in need thereof, said method comprising administering to the subject a composition comprising a therapeutically effective amount of a recombinant polypeptide of the present invention, wherein the virus is selected from the group consisting of: Dengue; respiratory syncytial virus (RSV); Hantavirus; Epstein-Barr virus (EBV); EBV (uncleaved); HCoV-OC43; MERS-CoV; MERS-CoV (uncleaved); Herpes simplex virus (HSV) 1; HSV 1 (uncleaved); influenza A H5N1 virus (IAV H5N1); human papillomavirus (HPV); Human Metapneumovirus; and human immunodeficiency virus (HIV).

In addition, the present disclosure describes a method of limiting the occurrence of, reducing the risk of, reducing the severity of, or treating a viral infection, in a subject in need thereof, said method comprising administering to the subject a composition comprising a therapeutically effective amount of a recombinant polypeptide of the present invention, wherein the virus has a CendR motif selected from the group consisting of:

GTCTQSGERRREKR;
KNTNVTLSKKRKRR;
LTHKMIEESHRLRR;
VSFKPPPPPSRRRR;
VSFKPPPPPSRRRRGACVVY;
GSGYCVDYSKNRRSR;
LLEPVSISTGSRSAR;
LLEPVSISTGSRSARSAIEDLLFDK;
ERPRAPARSASRPRR;
ERPRAPARSASRPRRPV;
VLATGLRNVPQRKKR;
PTTSSTSTTAKRKKR;
IDMLKARVKNRVAR;
AKRRVVQREKR;
and
AKRRVVQREKRAVGIGALFLG.

In addition, the present disclosure describes a method of limiting the occurrence of, reducing the risk of, reducing the severity of, or treating a viral infection, in a subject in need thereof, said method comprising administering to the subject a composition comprising a therapeutically effective amount of a recombinant polypeptide, or a pharmaceutically acceptable salt thereof, comprising: (a) one or more mutant neuropilin (NRP) b1 domains (b1), NRP b2 domains (b2), or fragments thereof; and (b) an Fc domain; wherein the one or more b1, b2, or fragments thereof, are derived from an NRP1 or an NRP2 protein; wherein the recombinant polypeptide is operable to bind to virus having an —Z₁-X₁-X₂-Z₂- (CendR) motif, wherein Z₁ and Z₂ are arginine or lysine, and X₁ and X₂ are any amino acid, wherein Z is arginine or lysine, and X is any amino acid; and wherein the virus is selected from the group consisting of: Dengue; respiratory syncytial virus (RSV); Hantavirus; Epstein-Barr virus (EBV); EBV (uncleaved); HCoV-OC43; MERS-CoV; MERS-CoV (uncleaved); Herpes simplex virus (HSV) 1; HSV 1 (uncleaved); influenza A H5N1 virus (IAV H5N1); human papillomavirus (HPV); Human Metapneumovirus; and human immunodeficiency virus (HIV).

In addition, the present disclosure describes a method of limiting the occurrence of, reducing the risk of, reducing the severity of, or treating a viral infection, in a subject in need thereof, said method comprising administering to the subject a composition comprising a therapeutically effective amount of a recombinant polypeptide, or a pharmaceutically acceptable salt thereof, comprising: (a) one or more mutant neuropilin (NRP) b1 domains (b1), NRP b2 domains (b2), or fragments thereof; and (b) an Fc domain; wherein the one or more b1, b2, or fragments thereof, are derived from an NRP1 or an NRP2 protein; wherein the recombinant polypeptide is operable to bind to virus having an —Z₁-X₁-X₂-Z₂- (CendR) motif, wherein Z₁ and Z₂ are arginine or lysine, and X₁ and X₂ are any amino acid; and wherein the virus has a CendR motif selected from the group consisting of:

GTCTQSGERRREKR;
KNTNVTLSKKRKRR;
LTHKMIEESHRLRR;
VSFKPPPPPSRRRR;
VSFKPPPPPSRRRRGACVVY;
GSGYCVDYSKNRRSR;
LLEPVSISTGSRSAR;
LLEPVSISTGSRSARSAIEDLLFDK;
ERPRAPARSASRPRR;
ERPRAPARSASRPRRPV;
VLATGLRNVPQRKKR;
PTTSSTSTTAKRKKR;
IDMLKARVKNRVAR;
AKRRVVQREKR;
and
AKRRVVQREKRAVGIGALFLG.

In addition, the present disclosure describes a method of limiting the occurrence of, reducing the risk of, reducing the severity of, or treating a viral infection, in a subject in need thereof, said method comprising administering to the subject a composition comprising a therapeutically effective amount of a recombinant polypeptide of the present invention, or a pharmaceutically acceptable salt thereof, wherein the virus is not SARS-CoV-2.

In addition, the present disclosure describes a method of limiting the occurrence of, reducing the risk of, reducing the severity of, or treating a viral infection, in a subject in need thereof, said method comprising administering to the subject a composition comprising a therapeutically effective amount of a recombinant polypeptide of the present invention, or a pharmaceutically acceptable salt thereof, wherein the virus is not SARS-CoV-2 Wuhan; SARS-CoV-2 Wuhan (uncleaved); SARS-CoV-2 UK; SARS-CoV-2 India; or SARS-CoV-2 India (uncleaved).

In addition, the present disclosure describes a method of limiting the occurrence of, reducing the risk of, reducing the severity of, or treating a viral infection, in a subject in need thereof, said method comprising administering to the subject a composition comprising a therapeutically effective amount of a recombinant polypeptide of the present invention, or a pharmaceutically acceptable salt thereof, wherein the virus is a virus that does not belong to the Coronaviridae family.

In addition, the present disclosure describes a method of limiting the occurrence of, reducing the risk of, reducing the severity of, or treating a viral infection, in a subject in need thereof, said method comprising administering to the subject a composition comprising a therapeutically effective amount of a recombinant polypeptide of the present invention, or a pharmaceutically acceptable salt thereof, wherein the virus is a virus that does not belong to the Betacoronavirus genus.

In addition, the present disclosure describes a method of limiting the occurrence of, reducing the risk of, reducing the severity of, or treating a viral infection, in a subject in need thereof, said method comprising administering to the subject a composition comprising a therapeutically effective amount of a recombinant polypeptide of the present invention, or a pharmaceutically acceptable salt thereof, wherein the virus is a virus that does not belong to the Sarbecovirus subgenus.

### BRIEF DESCRIPTION OF FIGURES

The following figures are provided by way of example and are not intended to limit the scope of the invention.

FIG. 1A is schematic drawing of the ACE2 receptor and function in addition to the mechanism that a SARS-CoV-2 spike protein binds and allows for viral entry.

FIG. 1B is a schematic drawing of antibody neutralization of SARS-CoV-2 virus particles.

FIG. 1C is a comparative chart for the SARS-CoV-1 and SARS-CoV-2 entry sites and corresponding symptoms and infected tissues.

FIG. 2A is an illustration of the polypeptide used to neutralize the SARS-CoV-2 virus.

FIG. 2B is a schematic representation of the polypeptide having sNRPT (b1), sACE2 (ACE2), linker, and immunoglobulin (Fc) domains that can effectively bind one or more SARS-CoV-2 virus particles.

FIG. 2C represents the Furin cleavage site on the spike protein of the SARS-CoV-2 virus that correspondingly binds the b1 NRP1 binding region.

FIG. 3A is a schematic representation of the SARS-CoV-2's pinocytosis infection using the NRP1 and/or the ACE2 receptors.

FIG. 3B illustrates how an antibody construct can neutralize and opsonize the SARS-CoV-2 virus.

FIG. 3C is the proposed mechanism for current vaccine and/or therapeutic polypeptide therapies for SARS-CoV-2 treatment.

FIG. 3D is the proposed mechanism for the disclosed polypeptide therapeutics in SARS-CoV-2 treatment.

FIG. 4 provides a schematic representation of exemplary N1-Fc polypeptide constructs disclosed in the current invention.

FIG. 5 provides a schematic representation of exemplary N1-Fc-ACE2 polypeptide constructs disclosed in the current invention.

FIG. 6 is a plot of the Relative Fluorescence Units (RFU) of the wells corresponding to ACE2 peptide binding to the S1/S2 spike protein (A) and S protein CendR binding to hu-b1b2-His (B).

FIG. 7 is a plot of the HuN1abHuIgG binding to the SARS-CoV-2 spike protein as determined using Relative Fluorescence Units (RFU).

FIG. 8 depicts a graph showing average daily weight of hamsters inoculated with 51 plaque forming units (PFU) of SARS-CoV-2. The three groups include the no virus control; hamsters inoculated with 51 PFU; and hamsters inoculated with 51 PFU and treated with a 15 mg/kg intraperitoneal injection of Compound 1 (SEQ ID NO: 122). Error bars show the standard deviation (SD).

FIG. 9 depicts a graph showing average daily weight of hamsters inoculated with 510 plaque forming units PFU of SARS-CoV-2. The three groups include the no virus control; hamsters inoculated with 510 PFU; and hamsters inoculated with 510 PFU and treated with a 15 mg/kg intraperitoneal injection of Compound 1 (SEQ ID NO: 122). or SAD-S35. Error bars show the standard deviation (SD).

FIG. 10 depicts a is a graphical representation of the respiratory cycle, showing various measurements that are used for calculation of the respiratory parameters for comparison between the groups in this study. A single respiratory cycle is depicted, showing the various measurements that are used to calculate the (enhanced pause) Penh respiratory parameter. Here, PEF is peak expiratory flow of breath; PIF is peak inspiratory flow of breath; Te is time of expiratory portion of breath; and Tr is time required to exhale 65% of breath volume.

FIG. 11 depicts a single expiratory portion of the respiratory cycle, showing the time to peak expiratory flow relative to the total time of expiration, Te.

FIG. 12 depicts a single expiratory portion of the respiratory cycle, showing the time to expelling 50% of the total expiratory volume.

FIG. 13 depicts the Log Penh results of the plethysmography data from hamsters in the 51 PFU-treated group. Data are presented as averages+one standard error.

FIG. 14 depicts the Log Penh results of the plethysmography data from hamsters in the 510 PFU-treated group. Data are presented as averages+one standard error.

FIG. 15 depicts the natural logarithm (ln) Penh results of the plethysmography data from hamsters in the 51 PFU-treated group. Data are presented as averages+one standard error.

FIG. 16 depicts the In Penh results of the plethysmography data from hamsters in the 510 PFU-treated group. Data are presented as averages+one standard error.

FIG. 17 depicts the square root EF50 results of the plethysmography data from hamsters in the 51 PFU-treated group. Data are presented as averages+one standard error.

FIG. 18 depicts the square root EF50 results of the plethysmography data from hamsters in the 510 PFU-treated group. Data are presented as averages+one standard error.

FIG. 19 is a photomicrograph at 4× magnification of a paraffin histology slice stained with H&E that was obtained from a hamster belonging to the media inoculated control group. Inflammation is absent. Red size bar=150 μM.

FIG. 20 is a photomicrograph at 4× magnification of a paraffin histology slice stained with H&E that was obtained from a hamster in the 51 PFU-treated arm. Several areas of chronic-active inflammation consisting of macrophages, lymphocytes, plasma cells and neutrophils are present; see, e.g., insert (A) at 40× magnification. Red size bar=150 μM, 40× and green size bar is 50 μM.

FIG. 21 is a photomicrograph at 4× magnification of a paraffin histology slice stained with H&E that was obtained from a hamster belonging to the 510 PFU SARS-CoV-2 inoculated control group. Several areas of chronic-active inflammation consisting of macrophages, lymphocytes, plasma cells and neutrophils are present; see, e.g., insert (A) at 40× magnification. Red size bar=150 μM, 40× and green size bar is 50 μM.

FIG. 22 is a photomicrograph at 4× magnification of a paraffin histology slice stained with H&E that was obtained from a hamster treated with 51 PFU and SEQ ID NO: 122 (15 mg/kg) via intraperitoneal injection. Red size bar=150 μM, 40× and green size bar is 50 μM.

FIG. 23 is a photomicrograph at 4× magnification of a paraffin histology slice stained with H&E that was obtained from a hamster treated with 510 PFU and SEQ ID NO: 122 (15 mg/kg) via intraperitoneal injection. Red size bar=150 μM, 40× and green size bar is 50 μM.

FIG. 24 is a photomicrograph at 4× magnification of a paraffin histology slice stained with H&E that was obtained from a hamster treated with 510 PFU and SAD35 (15 mg/kg) via intraperitoneal injection. Red size bar=150 μM, 40× and green size bar is 50 μM.

FIG. 25 depicts a graph summarizing body weight in grams over time for hamsters treated with constructs and challenged with 1500 PFU of SARS CoV-2. Here, 1=SEQ ID NO: 122; 2=SEQ ID NO: 154; and 3=SEQ ID NO: 192. Error bars show the standard deviation (SD).

FIG. 26 depicts a graph showing viral titer detected as nucleocapsid gene copies/μL RNA extracted from throat swabs or BAL. Here, 1=SEQ ID NO: 122; 2=SEQ ID NO: 154; and 3=SEQ ID NO: 192. Error bars show the standard deviation (SD).

FIG. 27 depicts a graph showing SARS CoV-2 copy/μL of RNA extracted from olfactory bulb. Here, 1=SEQ ID NO: 122; 2=SEQ ID NO: 154; and 3=SEQ ID NO: 192. Error bars show the standard deviation (SD).

FIG. 28 depicts a graph showing EF50 (mL/sec) in groups over time. Here, 1=SEQ ID NO: 122; 2=SEQ ID NO: 154; and 3=SEQ ID NO: 192. Error bars show the standard deviation (SD).

FIG. 29 depicts a graph showing Penh in groups over time. Here, 1=SEQ ID NO: 122; 2=SEQ ID NO: 154; and 3=SEQ ID NO: 192. Error bars show the standard deviation (SD).

FIG. 30 depicts a graph showing Rpef in groups over time. Here, 1=SEQ ID NO: 122; 2=SEQ ID NO: 154; and 3=SEQ ID NO: 192. Error bars show the standard deviation (SD).

FIG. 31 depicts a graph showing Interferon-gamma (IFNγ) levels (pg/mL) in groups over time. Here, 1=SEQ ID NO: 122; 2=SEQ ID NO: 154; and 3=SEQ ID NO: 192. Error bars show the standard deviation (SD).

FIG. 32 depicts a graph showing the results of the cytokine analysis for angiotensin 1-7 (Ang 1-7) levels during SARS CoV-2 infection in hamsters over time. Here, 1=SEQ ID NO: 122; 2=SEQ ID NO: 154; and 3=SEQ ID NO: 192. Error bars show the standard deviation (SD).

FIG. 33 depicts a graph showing the results of the cytokine analysis for Angiotensin II levels during SARS CoV-2 infection in hamsters over time. Here, 1=SEQ ID NO: 122; 2=SEQ ID NO: 154; and 3=SEQ ID NO: 192. Error bars show the standard deviation (SD).

FIG. 34 depicts a graph showing ratio of Angiotensin II to Ang 1-7 during SARS CoV-2 infection in hamsters over time. Here, 1=SEQ ID NO: 122; 2=SEQ ID NO: 154; and 3=SEQ ID NO: 192. Error bars show the standard deviation (SD).

FIG. 35 shows photomicrographs of formalin fixed H&E stained hamster lungs at 4× magnification. Evidence of bronchopneumonia is shown on day 7 at experiment end. Panel A=media control; Panel B=Virus control; Panel C=SEQ ID NO: 122; Panel D=SEQ ID NO: 154; and Panel E=SEQ ID NO: 192. Note the areas of intense blue inflammation, which comprises macrophages, neutrophils and lymphocytes. Size bar=1 mm.

FIG. 36 depicts a histogram showing the scoring of inflammation in the histology sections provided in FIG. 35 were scored using the following scheme: (a) Lesion distribution: none=0, focal=1, multifocal=2, diffuse=3; (b) Inflammation intensity: none=0, mild (2-3 inflammatory cells thick)=1, moderate (inflammatory cells=3-20 cells thick), severe (inflammatory cells>20 cells thick); (c) Small vessel thrombosis: absent=0, present=1. There were not significant differences between the treatment groups and control (Wilcoxon Rank Sum test).

FIG. 37 depicts the body weight of all K18ACE2 mice taken daily during the progression of B1.351. strain SARS CoV-2 infection. Body weights of K18-ACE2 mice challenged with SARS-CoV-2 B1.351 in the following four arms: Arm (1): Virus inoculation with SEQ ID NO: 113 (15 mg/kg) administration group n=13 each; Arm (2): Virus inoculation with 1.2 mg/kg of an antibody that binds a SARS-CoV-2 spike protein, said antibody having a heavy chain comprising the amino acid sequence set forth in SEQ ID NO: 189 and a light chain comprising the amino acid sequence set forth in SEQ ID NO: 190 (anti-SARS-CoV-2 spike protein antibody), administration group n=13 each; Arm (3): Cell culture medium intranasal control with IP saline administration n=6; Arm (4): Virus inoculated with IP saline administration n=6. Viral inoculated mice in arms 1 and 4 began to show signs clinical signs of illness by day 4, a few succumbed to the viral infection, and the remainder were all ill by day 7 at study termination. Here, 1=SEQ ID NO: 113; 2=anti-SARS-CoV-2 spike protein antibody.

FIG. 38 shows the clinical scores of all K18ACE2 mice taken daily during the progression of B1.351. strain SARS CoV-2 infection. Here, 1=SEQ ID NO: 113; 2=anti-SARS-CoV-2 spike protein antibody.

FIG. 39 depicts a graph showing serum fibrin degradation products of all K18ACE2 mice taken daily during the progression of B1.351. strain SARS CoV-2 infection. Here, 1=SEQ ID NO: 113; 2=anti-SARS-CoV-2 spike protein antibody.

FIG. 40 depicts a graph showing D-dimer levels in K18ACE2 mice taken at the end of the study after B1.351. strain SARS CoV-2 infection. Although there appears to be a lowering of the average D-dimer serum level in the SEQ ID NO: 113 and anti-SARS-CoV-2 spike protein antibody, treated groups, the averages did not differ statistically from the placebo and viral controls (one-way ANOVA using Tukey's multiple comparisons). Here, 1=SEQ ID NO: 113; 2=anti-SARS-CoV-2 spike protein antibody (a heavy chain comprising the amino acid sequence set forth in SEQ ID NO: 189 and a light chain comprising the amino acid sequence set forth in SEQ ID NO: 190).

FIG. 41 shows the Heparin binding affinity profiles for construct molecules of the present invention. (A) is SEQ ID NO: 113; (B) is SEQ ID NO: 121; (C) is SEQ ID NO: 122; (N) is SEQ ID NO: 191; (O) is SEQ ID NO: 121; (G) is SEQ ID NO: 128; (L) is SEQ ID NO: 129; (M) is SEQ ID NO: 154; (D) is SEQ ID NO: 135; (E) is SEQ ID NO: 136; (F) is SEQ ID NO: 137; (H) is SEQ ID NO: 114; (I) is SEQ ID NO: 115; (J) is SEQ ID NO: 116; (K) is SEQ ID NO: 133.

FIG. 42 shows a representation of the constructs of the present invention. The top grey portion represents the neuropilin-b1 domain linked by a short peptide sequence known as a G4S linker to additional (double tandem shown) b1 domains and the IgG Fc stem in black consisting of the constant heavy chains (CH2 and CH3).

FIG. 43 shows the cumulative distribution of PiTou scores in human peptides.

FIG. 44 shows the cumulative distribution of PiTou scores in viral peptides.

FIG. 45 shows the cumulative distribution of PiTou scores in bacterial peptides.

FIG. 46 shows the PiTou scores at known viral cleavage sites.

FIG. 47 shows a prioritized PiTou score distribution.

### DETAILED DESCRIPTION

### Definitions

Unless defined otherwise, all technical and scientific terms used herein have the same meaning as commonly understood by one of ordinary skill in the art to which this disclosure belongs. Accordingly, the following terms are intended to have the following meanings:

As used in the specification and claims, the singular form “a”, “an” and “the” includes plural references unless the context clearly dictates otherwise.

As used herein, “administration” of a disclosed polypeptide encompasses the delivery to a subject of a polypeptide or composition of the present invention, as described herein, or a prodrug or other pharmaceutically acceptable derivative thereof, using any suitable formulation or route of administration, e.g., as described herein.

As used herein, the term “and/or,” when used in a list of two or more items, means that any one of the listed items can be employed by itself, or any combination of two or more of the listed items can be employed. For example, if a composition is described as comprising components A, B, and/or C, the composition can contain A alone; B alone; C alone; A and B in combination; A and C in combination; B and C in combination; or A, B, and C in combination.

As used herein, “treatment” and “treating”, are used interchangeably herein, and refer to an approach for obtaining beneficial or desired results including, but not limited to, therapeutic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient can still be afflicted with the underlying disorder. The term “treat”, in all its verb forms, is used herein to mean to relieve, alleviate, prevent, and/or manage at least one symptom of a disorder in a subject.

A “subject,” as used herein, can refer to any animal which is subject to a viral infection, e.g., a mammal, such as an experimental animal, a farm animal, pet, or the like. In some embodiments, the animal is a primate, preferably a human. As used herein, the terms “subject” and “patient” are used interchangeably. The terms “subject” and “patient” refer to an animal (e.g., a bird such as a chicken, quail or turkey, or a mammal), specifically a “mammal” including a non-primate (e.g., a cow, pig, horse, sheep, rabbit, guinea pig, rat, cat, dog, and mouse) and a primate (e.g., a monkey, chimpanzee and a human), and more specifically a human. In one embodiment, the subject is a non-human animal such as a farm animal (e.g., a horse, cow, pig or sheep), or a pet (e.g., a dog, cat, guinea pig or rabbit). In a preferred embodiment, the subject is a “human”.

As used herein, the term “fusion” refers to unifying two molecules having the same or different function or structure, and the methods of fusing may include any physical, chemical or biological method capable of binding the peptide to the protein, the small-molecule drug, the nanoparticle or the liposome. Preferably, the fusion may be mediated by a linker peptide, and for example, the linker peptide may be fused to the C-terminus of a fragment of an antibody light-chain variable region (Fc).

The terms, “disease”, “disorder”, and “condition” may be used interchangeably here to refer to a virus mediated medical or pathological condition.

The term “biological sample”, as used herein, includes, without limitation, cell cultures or extracts thereof, biopsied material obtained from a mammal or extracts thereof, blood, saliva, urine, feces, semen, tears, or other body fluids or extracts thereof.

As used herein, “multiplicity of infection” or “MOI” is the ratio of infectious agents (e.g. phage or virus) to infection targets (e.g. cell). For example, when referring to a group of cells inoculated with infectious virus particles, the multiplicity of infection or MOI is the ratio defined by the number of infectious virus particles deposited in a well divided by the number of target cells present in that well.

As used herein the term “inhibition of the replication of SARS-CoV-2 virus” includes both the reduction in the amount of virus replication (e.g. the reduction by at least 10%) and the complete arrest of virus replication (i.e., 100% reduction in the amount of virus replication). In some embodiments, the replication of SARS-CoV-2 is inhibited by at least 50%, at least 65%, at least 75%, at least 85%, at least 90%, or at least 95%.

As used herein a “viral titer (or titre)” is a measure of virus concentration. Titer testing can employ serial dilution to obtain approximate quantitative information from an analytical procedure that inherently only evaluates as positive or negative. The titer corresponds to the highest dilution factor that still yields a positive reading; for example, positive readings in the first 8 serial twofold dilutions translate into a titer of 1:256. A specific example is viral titer. To determine the titer, several dilutions can be prepared, such as 10⁻¹, 10⁻², 10⁻³, . . . , 10⁻⁸. The lowest concentration of virus that still infects cells is the viral titer.

As used herein, the terms “treat” and “treatment” and “treating” and the like are used herein to generally refer to obtaining a desired pharmacologic and/or physiologic effect. The effect can be prophylactic in terms of completely or partially preventing a disease or symptom thereof and/or may be therapeutic in terms of a partial or complete stabilization or cure for a disease and/or adverse effect attributable to the disease. “Treatment” as used herein covers any treatment of a disease in a subject, particularly a human, and includes: (a) preventing the disease or symptom from occurring in a subject which may be predisposed to the disease or symptom but has not yet been diagnosed as having it; (b) inhibiting the disease symptom, i.e., arresting its development; or (c) relieving the disease symptom, i.e., causing regression of the disease or symptom. Those in need of treatment include individuals already diagnosed with a disease, e.g., a viral infection, as well as those in which the disease is to be prevented. Thus, the terms “treat” or “treatment” or “treating” refer to both therapeutic and prophylactic treatments. For example, therapeutic treatments includes the reduction or amelioration of the progression, severity and/or duration of a disease's (e.g., a virus's) mediated conditions, or the amelioration of one or more symptoms (specifically, one or more discernible symptoms) of the disease's mediated conditions, resulting from the administration of one or more therapies (e.g., one or more therapeutic agents such as a polypeptide or composition of the invention).

In specific embodiments, the therapeutic treatment includes the amelioration of at least one measurable physical parameter of a virus mediated condition. In other embodiments the therapeutic treatment includes the inhibition of the progression of the virus's mediated condition, either physically by, e.g., stabilization of a discernible symptom, physiologically by, e.g., stabilization of a physical parameter, or both. In other embodiments the therapeutic treatment includes the reduction or stabilization of the virus's mediated infections. Antiviral drugs can be used in the community setting to treat people who already have COVID-19 to reduce the severity of symptoms and reduce the number of days that they are sick.

The terms “prophylaxis” or “prophylactic use” and “prophylactic treatment” as used herein, refer to any medical or public health procedure whose purpose is to prevent, rather than treat or cure a disease. As used herein, the terms “prevent”, “prevention” and “preventing” refer to the reduction in the risk of acquiring or developing a given condition, or the reduction or inhibition of the recurrence or said condition in a subject who is not ill, but who has been or may be near a person with the disease. The term “chemoprophylaxis” refers to the use of medications, e.g. small molecule drugs (rather than “vaccines”) for the prevention of a disorder or disease.

As used herein, prophylactic use includes the use in situations in which an outbreak has been detected, to prevent contagion or spread of the infection in places where a lot of people that are at high risk of serious viral (e.g., COVID-19) complications live in close contact with each other (e.g. in a hospital ward, day-care center, prison, nursing home, etc.). It also includes the use among populations who require protection from the SARS-CoV-2 but who either do not get protection after vaccination (e.g. due to weak immune system), or when the vaccine is unavailable to them, or when they cannot get the vaccine because of side effects. It also includes use during the two weeks following vaccination, since during that time the vaccine is still ineffective. Prophylactic use may also include treating a person who is not ill with the SARS-CoV-2 or not considered at high risk for complications, in order to reduce the chances of getting infected with the SARS-CoV-2 and passing it on to a high-risk person in close contact with him (for instance, healthcare workers, nursing home workers, etc.).

As used herein, an “effective amount” refers to an amount sufficient to elicit the desired biological response. In the present invention the desired biological response is to inhibit the replication of the virus (e.g., SARS-CoV-2), to reduce the amount of viruses or to reduce or ameliorate the severity, duration, progression, or onset of a viral infection, prevent the advancement of a viral infection, prevent the recurrence, development, onset or progression of a symptom associated with the viral infection, or enhance or improve the prophylactic or therapeutic effect(s) of another therapy used against viral infections. The precise amount of compound administered to a subject will depend on the mode of administration, the type and severity of the infection and on the characteristics of the subject, such as general health, age, sex, body weight and tolerance to drugs. The skilled artisan will be able to determine appropriate dosages depending on these and other factors. When co-administered with other anti-viral agents, e.g., when co-administered with an anti-viral medication, an “effective amount” of the second agent will depend on the type of drug used. Suitable dosages are known for approved agents and can be adjusted by the skilled artisan according to the condition of the subject, the type of condition(s) being treated and the amount of a polypeptide described herein being used. In cases where no amount is expressly noted, an effective amount should be assumed. For example, compounds described herein can be administered to a subject in a dosage range from between approximately 0.01 to 100 mg/kg body weight/day for therapeutic or prophylactic treatment.

The terms “polypeptide,” “peptide” and “protein” are used interchangeably herein to refer to a polymer of amino acid residues.

The term “reduce” or other forms of the word, such as “reducing” or “reduction,” generally refers to the lowering of an event or characteristic (e.g., one or more symptoms, or the binding of one protein to another). It is understood that this is typically in relation to some standard or expected value, in other words it is relative, but that it is not always necessary for the standard or relative value to be referred to. In some embodiments, the term “reducing,” as used in the context of “reducing the risk” or “reducing the severity” means decreasing risk of being infected with a given disease or virus; or decreasing the severity and/or frequency of the symptom(s) and/or elimination of the symptom(s) of a given disease or virus, relative to a subject that has not been treated pursuant to the compositions and/or methods of the present invention. Here

The term “reduced,” as used in the context of “reduced binding” refers to a decrease in the affinity of one molecule to another molecule. For example, in some embodiments, a protein, domain, or motif can specifically bind to a particular target, e.g., a peptide, polypeptide, protein, carbohydrate, saccharide, polysaccharide, glycosaminoglycan, or any epitope thereof, with a given affinity; and, a reduction said binding refers to a decrease in the affinity of said protein, domain, or motif to the target. Measuring the affinity of binding is well known in the art. In some embodiments, the affinity of one molecule for another molecule to which it specifically binds is characterized by a dissociation constant (K_{D} or K_{d})

“Affinity” refers to the strength of the sum total of non-covalent interactions between a single binding site of a molecule and its binding target or partner (e.g., an antigen). The affinity of a molecule for its target can generally be represented by the dissociation constant (K_{D}), which is the ratio of dissociation and association rate constants (k_{off} and kₒₙ, respectively). Briefly, the strength, or affinity of binding interactions can be expressed in terms of the dissociation constant (K_{D}) of the interaction, wherein a smaller K_{D} represents a greater affinity. The binding properties of selected polypeptides can be quantified using methods well known in the art. One such method entails measuring the rates of antigen-binding site/antigen complex formation and dissociation, wherein those rates depend on the concentrations of the complex partners, the affinity of the interaction, and geometric parameters that equally influence the rate in both directions. Thus, both the “on rate constant” (Kₒₙ) and the “off rate constant” (K_{off}) can be determined by calculation of the concentrations and the actual rates of association and dissociation. (See Nature 361:186-87 (1993)). The ratio of K_{off}/Kₒₙ enables the cancellation of all parameters not related to affinity, and is equal to the dissociation constant K_{D}. (See, generally, Davies et al. (1990) Annual Rev Biochem 59:439-473).

Thus, equivalent affinities may comprise different rate constants, as long as the ratio of the rate constants remains the same. Affinity can be measured by well-established methods known in the art, including those described herein. Thus, in some embodiments, “reduced binding” refers to a decrease in affinity for the respective interaction. Conversely, “increased binding” refers to an increase in binding affinity for the respective interaction.

In some embodiments, a recombinant polypeptide of the present invention can specifically bind to an epitope when the equilibrium binding constant (K_{D}) is ≤1 μM. In some embodiments, a recombinant polypeptide of the present invention can specifically bind to an epitope when the equilibrium binding constant (K_{D}) is 100 nM. In some embodiments, a recombinant polypeptide of the present invention can specifically bind to an epitope when the equilibrium binding constant (K_{D}) is 10 nM. In some embodiments, a recombinant polypeptide of the present invention can specifically bind to an epitope when the equilibrium binding constant (K_{D}) is 100 μM to about 1 μM, as measured by assays such as Surface Plasmon Resonance (SPR), Octet assays, or similar assays known to those skilled in the art. In some embodiments, a K_{D} can be 10⁻⁵ M or less (e.g., 10⁻⁶ M or less, 10⁻⁷ M or less, 10⁻⁸ M or less, 10⁻⁸ M or less, 10⁻¹⁰ M or less, 10⁻¹¹ M or less, 10⁻¹² M or less, 10⁻¹³ M or less, 10⁻¹⁴ M or less, 10⁻¹⁵ M or less, or 10⁻¹⁶ M or less).

In some embodiments, there can be a reduction of binding of 1%, 2%, 3%, 4%, 5%, 6%, 7%, 8%, 9%, 10%, 20%, 30%, 40%, 50%, 60%, 70%, 80%, 90% or 100% in a recombinant polypeptide compared to a control. For example, in recombinant polypeptides having reduced heparin or heparan sulfate binding, there can be there can be a reduction of binding of 1%, 2%, 3%, 4%, 5%, 6%, 7%, 8%, 9%, 10%, 20%, 30%, 40%, 50%, 60%, 70%, 80%, 90% or 100% in a recombinant polypeptide compared to a control.

“Derived” or “derived from” refers to obtaining a peptide, polypeptide, protein or polynucleotide from a known and/or originating peptide, polypeptide, protein or polynucleotide. Thus, as used herein, the term “derived from” encompasses, without limitation: a protein or polynucleotide that is isolated or obtained directly from an originating source (e.g. an organism); a synthetic or recombinantly generated protein or polynucleotide that is identical, substantially related to, or modified from, a protein or polynucleotide from an known/originating source (e.g., an NRP1 or NRP2); or protein or polynucleotide that is made from a protein or polynucleotide of an known/originating source or a fragment thereof. The term “substantially related”, as used herein, means that the protein may have been modified by chemical, physical or other means (e.g. sequence modification).

Accordingly, “derived” can refer to either directly or indirectly obtaining a protein or polynucleotide from a known and/or originating protein or polynucleotide. For example, in some embodiments, “derived” can refer to obtaining a protein or polynucleotide from a known and/or originating protein or polynucleotide by looking at the sequence of a known/originating protein or polynucleotide and preparing a protein or polynucleotide having a sequence similar, at least in part, to the sequence of the known and/or originating protein or polynucleotide. In yet other embodiments, “derived” can refer to obtaining a protein or polynucleotide from a known and/or originating protein or polynucleotide by isolating a protein or polynucleotide from an organism that is related to a known protein or polynucleotide. Other methods of “deriving” a protein or polynucleotide from a known protein or polynucleotide are known to one of skill in the art.

In some embodiments, “derived” in the context of a protein (e.g., “a protein derived from an organism”) describes a condition wherein said protein was originally identified in an organism, and has been reproduced therefrom via isolation from the organism, or through synthetic or recombinant means.

“Excipient” refers to any pharmacologically inactive, natural, or synthetic, component or substance that is formulated alongside (e.g., concomitantly), or subsequent to, the active ingredient of the present invention. In some embodiments, an excipient can be any additive, adjuvant, binder, bulking agent, carrier, coating, diluent, disintegrant, filler, glidant, lubricant, preservative, vehicle, or combination thereof, with which a recombinant polypeptide of the present invention can be administered, and or which is useful in preparing a composition of the present invention. Excipients, include any such materials known in the art that are nontoxic and do not interact with other components of a composition. In some embodiments, excipients can be formulated alongside a recombinant polypeptide when preparing a composition for the purpose of bulking up compositions (thus often referred to as bulking agents, fillers or diluents). In other embodiments, an excipient can be used to confer an enhancement on the active ingredient in the final dosage form, such as facilitating absorption and/or solubility. In yet other embodiments, an excipient can be used to provide stability, or prevent contamination (e.g., microbial contamination). In other embodiments, an excipient can be used to confer a physical property to a composition (e.g., a composition that is a dry granular, or dry flowable powder physical form). Reference to an excipient includes both one and more than one such excipients. Suitable pharmaceutical excipients are described in Remington's Pharmaceutical Sciences, by E. W. Martin, the disclosure of which is incorporated herein by reference in its entirety.

“Homologous” refers to the sequence similarity or sequence identity between two polypeptides or between two nucleic acid molecules. When a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared ×100. Thus, in some embodiments, the term “homologous” refers to the sequence similarity between two polypeptide molecules, or between two nucleic acid molecules. When a position in both of the two compared sequences is occupied by the same base or amino acid monomeric subunit, e.g., if a position in each of two DNA molecules is occupied by adenine, then the molecules are homologous at that position. The homology between two sequences is a function of the number of matching or homologous positions shared by the two sequences. For example, if 6 of 10 of the positions in two sequences are matched or homologous then the two sequences are 60% homologous. By way of example, the DNA sequences ATTGCC and TATGGC share 50% homology.

The term “homology,” when used in relation to nucleic acids, refers to a degree of complementarity. There may be partial homology, or complete homology and thus identical. “Sequence identity” refers to a measure of relatedness between two or more nucleic acids, and is given as a percentage with reference to the total comparison length. The identity calculation takes into account those nucleotide residues that are identical and in the same relative positions in their respective larger sequences.

“Identity” refers to a relationship between two or more polypeptide sequences or two or more polynucleotide sequences, as determined by comparing said sequences. The term “identity” also means the degree of sequence relatedness between polypeptide or polynucleotide sequences, as the case may be, as determined by the match between strings of such sequences. “Identity” and “similarity” can be readily calculated by any one of the myriad methods known to those having ordinary skill in the art, including but not limited to those described in: Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48: 1073 (1988), the disclosures of which are incorporated herein by reference in their entireties. Furthermore, methods to determine identity and similarity are codified in publicly available computer programs. For example in some embodiments, methods to determine identity and similarity between two sequences include, but are not limited to, the GCG program package (Devereux, J., et al., Nucleic Acids Research 12(1): 387 (1984)), BLASTP, BLASTN, and FASTA (Altschul, S. F. et al., J. Molec. Biol. 215: 403-410 (1990). The BLAST X program is publicly available from NCBI and other sources (BLAST Manual, Altschul, S., et al., NCBI NLM NIH Bethesda, Md. 20894; Altschul, S., et al., J. Mol. Biol. 215: 403-410 (1990), the disclosures of which are incorporated herein by reference in their entireties.

“Mutant” refers to an organism, DNA sequence, polynucleotide, amino acid sequence, peptide, polypeptide, or protein, that has an alteration, variation, or modification (for example, in the nucleotide sequence or the amino acid sequence), which causes said organism and/or sequence to be different from the naturally occurring or wild-type organism, wild-type sequence, and/or reference sequence with which the mutant is being compared. In some embodiments, this alteration, variation, or modification can be one or more nucleotide and/or amino acid substitutions or modifications (e.g., deletion or addition). In some embodiments, the one or more amino acid substitutions or modifications can be conservative; here, such a conservative amino acid substitution and/or modification in a “mutant” does not substantially diminish the activity of the mutant in relation to its non-mutant form. For example, in some embodiments, a “mutant” possesses one or more conservative amino acid substitutions when compared to a peptide with a disclosed and/or claimed sequence, as indicated by a SEQ ID NO.

“Operable” refers to the ability to be used, the ability to do something, and/or the ability to accomplish some function or result. For example, in some embodiments, “operable” refers to the ability of a polynucleotide, DNA sequence, RNA sequence, or other nucleotide sequence or gene to encode a peptide, polypeptide, and/or protein. For example, in some embodiments, a polynucleotide may be operable to encode a protein, which means that the polynucleotide contains information that imbues it with the ability to create a protein (e.g., by transcribing mRNA, which is in turn translated to protein).

“Wild type” or “WT” refers to the phenotype and/or genotype (i.e., the appearance or sequence) of an organism, polynucleotide sequence, and/or polypeptide sequence, as it is found and/or observed in its naturally occurring state or condition.

Throughout this specification, unless the context requires otherwise, the word “comprise,” or variations such as “comprises” or “comprising,” will be understood to imply the inclusion of a stated step or element or integer or group of steps or elements or integers but not the exclusion of any other step or element or integer or group of elements or integers.

All patent applications, patents, and printed publications referred to herein are incorporated by reference in their entirety to the same extent as if each individual publication, patent, or patent application was specifically and individually indicated to be incorporated by reference in its entirety. And, all patent applications, patents, and printed publications cited herein are incorporated herein by reference in the entireties, except for any definitions, subject matter disclaimers, or disavowals, and except to the extent that the incorporated material is inconsistent with the express disclosure herein, in which case the language in this disclosure controls.

SARS-CoV-2 Virion Particle Model Background and Mechanism

Severe acute respiratory syndrome coronavirus 2 (SARS-CoV-2) incites an immune response in the body leading to increased vascular permeability, increased endothelial inflammatory response, reduced levels of nitric oxide (NO) and impaired angiogenesis. The endothelial dysfunction cause by the cytokine storm from SARS-CoV-2 can lead to multiorgan failure, including the heart and kidney. Comorbidity factors such as age, hypertension, and/or obesity can exacerbate the effects of SARS-CoV-2. In many severe cases of SARS-CoV-2, the severe acute respiratory syndrome occurs in the alveolus and endothelium in the lungs where the COVID-19 includes vascular leakage, clotting, and inflammation. SARS-CoV-2 is a respiratory virus that has the ability to infect blood vessel cells and circulate through the body unlike the original SARS virus, H1N1, or other types of viruses such as Ebola or Dengue that can damage endothelial cells but do not infect the lungs.

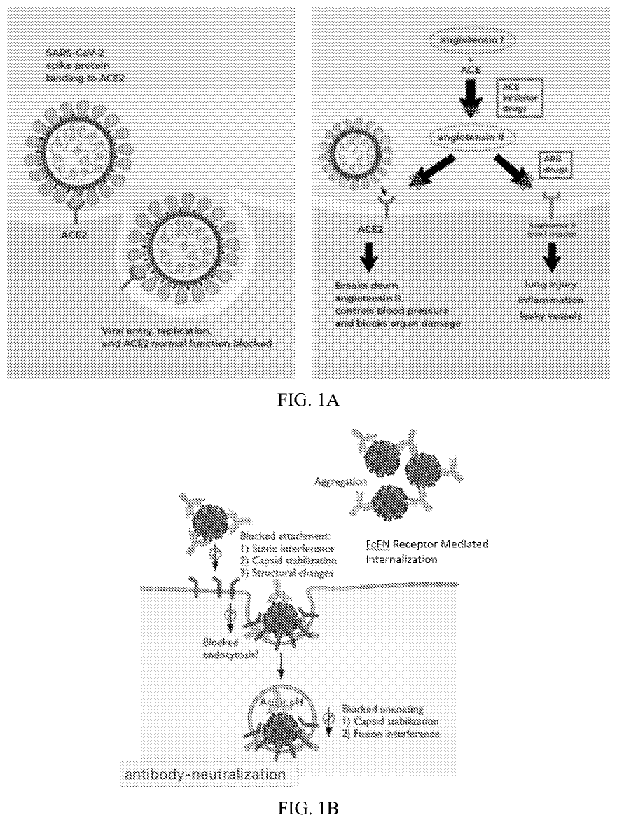

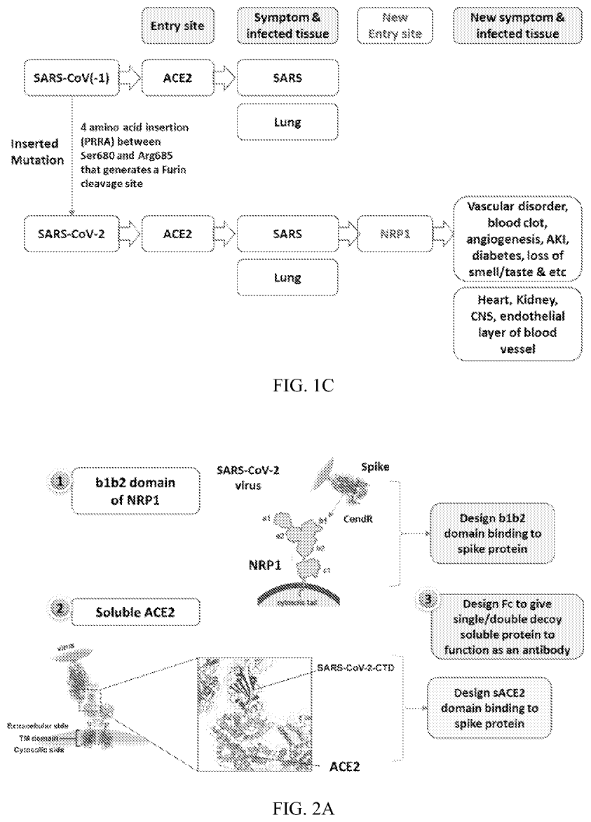

As illustrated in FIG. 1A, a SARS-CoV-2 virus can enter and infect a human cell by attaching its spike protein (SARS-S) to an angiotensin converting enzyme 2 (ACE2) cellular receptor that resides on the surface of the cell. Once the spike protein is bound to the ACE2 receptor, the SARS-CoV-2 virus can enter into the cell where the virus shell is broken apart, releasing RNA into the host cell where it replicates and generates more viral particles. The ability of the ACE2 receptor to break down angiotensin II, control blood pressure, and block organ damage can be severely deterred during the SARS-CoV-2 infection.

Referring now to FIG. 1B, one of the known mechanisms that can be used to help neutralize the spread of SARS-CoV-2 infection is antibody neutralization. As illustrated in FIG. 1B, the antibody or an immunoglobulin construct can bind to the SARS-CoV-2 particle to block its attachment to the ACE2 cellular receptors of the cell by imparting steric interference, capsid stabilization, and or structural changes. In some cases, the antibody, polypeptide, or immunoglobulin construct can aggregate to more than one SARS-CoV-2 particle to further prevent the internalization of the virus into the cell. In these cases where the SARS-CoV-2 particle is coupled to one or more antibodies, the corresponding large particle may enter the cell through phagocytosis where the conjugated SARS-CoV-2 particle is neutralized.

Based on the pathology of the coronavirus where blood clots can be found in almost every organ during autopsies on COVID-19 patients, scientists are beginning to consider the coronavirus as a blood vessel disease. If COVID-19 is in fact a vascular disease, it is believed that the best antiviral therapy might not actually be a traditional antiviral therapy. As explained above, it is understood that both SARS-CoV-1 and SARS-CoV-2 enter the cell through the ACE2 cellular receptor. Based on the additional symptoms and increase in infected tissues in patients infected with SARS-CoV-2, it is believed that one or more additional entry sites may explain the prevalence of microthrombosis and amount of angiogenesis in the lung as compared Influenzas A and SARS-CoV-1. Referring to FIG. 1C, a flowchart is provided that outlines the use of neuropilin-1 (NRP-1) as a means for the SARS-CoV-2 to enter the cell in addition to the ACE2 receptor could explain the additional symptoms observed such as vascular disorders, blood clots, angiogenesis, AKI and diabetes and additionally infected tissues such as the heart, kidney, and endothelial layer of the blood vessels where the loss of taste/smell is also frequently observed.

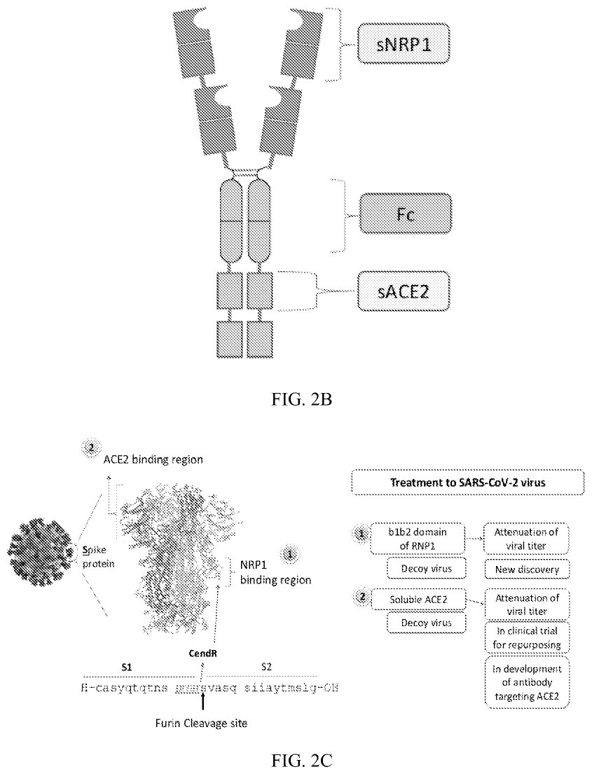

Recent research has identified that neuropilin-1 facilitates SARS-CoV-2 cell entry and provides a possible pathway into the central nervous system. It has additionally been noted that neuropilin-1 is a host factor for SARS-CoV-2 infection. Referring now to FIG. 2A, the soluble domains of the SARS-CoV-2 virus are shown to utilize the binding ability of the b1b2 domain of a neuropilin-1 receptor and the soluble ACE2 receptor. By designing the Fc domain of the immunoglobulin to provide a double decoy soluble protein, this corresponding fusion polypeptide can effectively bind at least 1, at least 2, at least 3, at least 4, at least 5, or at least 6 different spike proteins of one or more SARS-CoV-2 virus particles. As illustrated in FIG. 2B and explained in more detail herein, an exemplary fusion polypeptide can include sNRP1 (b1), sACE2 (ACE2), linker, and immunoglobulin (Fc) domains that can effectively bind at least 1, at least 2, at least 3, at least 4, at least 5, or at least 6 different spike proteins of one or more SARS-CoV-2 virus particles.

Referring now to FIG. 2C, SARS-CoV-2 has a four (4) amino acid insertion (PRRA) between Ser680 and ARG685 that generates a Furin cleavage site. The Furin cleavage of SARS-CoV-2 exposes a C-terminal motif RXXR-OH (C-end R rule) that is known to bind to the neuropilin-1 (NRP1) and/or neuropilin-2 (NRP2) b1 binding sites.

Accordingly, in designing a therapeutic that would be effective at reducing and treating viral infections such as the SARS-CoV-2 virus, fusion peptides comprising a combination of a neuropilin-1 (NRP1) domain, a neuropilin-2 (NRP2) domain, an angiotensin converting enzyme 2 (ACE2) domain, or a combination thereof are designed herein to specifically bind a coat protein of a virus particle, in particular, a S protein of COVID-19 particles. In some embodiments, these fusion peptides further comprise an immunoglobulin domain, for example, and Fc domain derived from a human immunoglobulin.

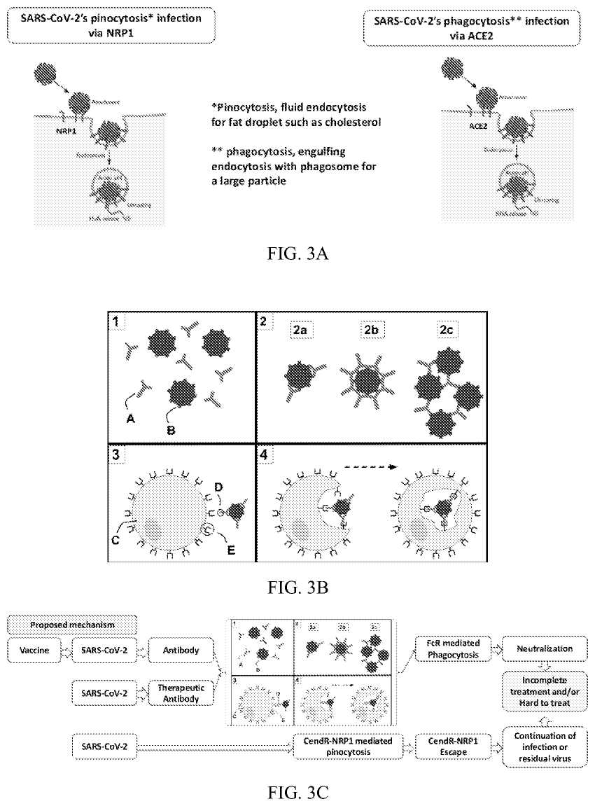

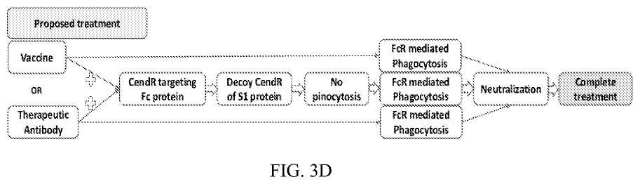

Referring to FIG. 3A, a SARS-CoV-2 virus's pinocytosis infection is illustrated using a NRP1 receptor and or an ACE2 receptor. In each case, the SARS-CoV-2 virus is introduced into the cell by the budding of a small vesicle from the cell membrane where the virus shell can be broken apart in an acidic environment, releasing RNA into the host cell where it replicates and generates more viral particles.

Referring now to FIG. 3B, an illustrated schematic is provided that demonstrates how the disclosed polypeptides can neutralize and opsonize a SAR-CoV-2 virus particle. In step 1, the polypeptides (A) and pathogens (B) freely circulate and roam in the blood. In step 2, the disclosed polypeptides can bind to the pathogens, and can do so in different formations such as: opsonization (2a), neutralization (2b), and agglutination (2c). In step 3 a phagocyte (C) approaches the pathogen where the b1 and/or ACE2 domains, optionally coupled to the Fc region (D) of the disclosed polypeptides binds to one of the receptors (E) on the phagocyte. Lastly, in step 4, phagocytosis occurs as the pathogen is ingested.

The currently proposed mechanism for reducing and treating a SAR-CoV-2 viral infection is illustrated in FIG. 3C. In this mechanism, therapeutic antibodies and/or a vaccine would be provided that would produce antibodies that could bind to the SAR-CoV-2 virus and trigger phagocytosis. The problem with this approach is the SAR-CoV-2 virus particles that could alternatively bind to the NRP1 receptor and infect the cell through CendR-NRP1 mediated pinocytosis. The SAR-CoV-2 virus that may remain active in the body through the CendR-NRP1 mediated pinocytosis mechanism could lead to incomplete treatment and/or difficulty in effectively treating the patient.

Alternatively, as illustrated in FIG. 3D, the proposed treatment using the polypeptides as disclosed herein could remedy the deficiencies of the mechanism outlined in FIG. 3C. For example, a polypeptide comprising both a b1 domain, or a derivative or fragment thereof, of a neuropilin; and an ACE2 domain, or a derivative or fragment thereof, of angiotensin converting enzyme 2 would target both the CendR-NRP1 and ACE2 mediated pinocytosis mechanisms in which the SAR-CoV-2 virus enters the cell. By eliminating the mechanisms that the SAR-CoV-2 virus uses to enter the cell and effectively neutralizing the virus's effects through phagocytosis, a complete and effective treatment can be provided to the patient.

Neuropilin Neutralization of SARS-CoV-2

Neuropilin broadly consists of five domains, and from the N-terminus, a1 and a2 domains are classified as CUB domains, and an Ig-like C2 type of semaphorin binds thereto. Particularly, this site forms a complex with plexin, and plays a role of increasing the binding force with semaphorin-plexin. The b1 and b2 domains are classified as FV/VIII domains, and the C-terminus of VEGF family ligand or class 3 semaphorin ligand binds thereto. Particularly, in this portion, a site to which heparin is capable of binding is present and this facilitates the binding of ligands with many (+) charged residues. Further, MAM induces oligomerization, trans-membrane domain (TM) enables neuropilin to be fixed onto cell surface, and in a cytosolic domain, a site capable of binding to a Postsynaptic density 95, Disk large, Zona occludens 1 (PDZ) domain is present.1

The upper four extracellular domains (a1, a2, b1, and b2) determine the binding specificity of multiple ligands to NRP1/2 and the last extracellular c domain, along with the transmembrane domain, is implicated in the dimerization or oligomerization between NRPs and their co-receptors. Both VEGF and Sema3 family ligands specifically bind to the VEGF-binding region in the b1 domain of NRP1/2 through the C-terminal R/K-x-x-R/K sequence motif, where x stands for any amino acid. In fact, all known proteins, and peptides binding to the ligand-binding pocket in the NRP1-b1 domain share the sequence motif. This basic sequence motif in the NRP1-binding ligands and peptides must be exposed at the C-terminus for binding to NRP1, with a stringent requirement for Arg (or rarely Lys) at the last C-terminal residue; this requirement is called the “C-end rule” (CendR)

Multiple viruses possess CendR motifs within their capsid proteins and may undergo proteolytic cleavage to expose the CendR motif to be infective, most viral envelope glycoproteins need to be proteolytically cleaved before they can mediate viral entry into host cells. In many cases, viruses exploit cellular trypsin- or subtilisin-like endoproteases for this purpose. While subtilisin-like proteases such as furin require polybasic cleavage sites, trypsin-like proteases also recognize monobasic motifs and cleave after single arginine or lysine residues. Furin-mediated cleavage has been described for envelope glycoproteins encoded by numerous evolutionarily diverse virus families, including Herpes-, Corona-, Flavi-, Toga-, Boma-, Bunya-, Filo-, Orthomyxo-, Paramyxo-, Pneumo- and Retroviridae.

Recently, researchers observed that NRP1 facilitated the ability of SARS-CoV-2 to infect cells during cell culture experiments. Their findings also showed that it was the S1 polypeptide that binds to NRP1. The S1 polypeptide is one to two polypeptides formed when the spike protein of SARS-CoV-2 is cleaved and activated. It contains a sequence that conforms to the C-end rule (CendR).

In addition, the b1b2 domain of Nrp1 and Nrp2 contain structural determinants capable of C-terminal Sema3F and VEGF binding. An intact b1b2 domain serves as the VEGF165-, PlGF-2-, and heparin-binding sites in NRP1, and that heparin is a critical component for regulating VEGF165 and PlGF-2 interactions with NRP1 by physically interacting with both receptor and ligands.

The Mutations of VEGF binding pocket within b1 domain changed the binding affinity to VEGF165A. A Y297A/S346A/Y353A mutation of b1 domain cannot bind to VEGF. Also an E319A mutation of b1 domain has a stronger binding affinity to VEGFA with heparin while T349A and K351A mutation of b1 domain has a weaker binding affinity to VEGFA w/ or w/o heparin.

Neuropilin, a transmembrane glycoprotein, is divided into two types: neuropilin-1 (NRP1; the human NRP1 amino acid sequence is provided as SEQ ID NO:1) and neuropilin-1 (NRP2; the human NRP2 amino acid sequence is provided as SEQ ID NO:1) (Kolodkin et al. 1997). Neuropilin-1 and -2 consist of 923 and 931 amino acids, respectively, and show an amino acid sequence homology of about 44%, and share several structural aspects and biological activities. Neuropilin-1 and -2 consist commonly of extracellular a1, a2, b1, b2 and MAM domains and an intracellular PDZ-binding domain (Appleton et al. 2007). Neuropilin is very weakly expressed in normal cells, but is overexpressed in most tumor-associated endothelial cells, solid tumor cells and blood tumor cells (Grandclement, C. and C. Borg 2011). Neuropilin acts as a co-receptor of VEGF receptors (VEGFRs) by binding to VEGF 25 family ligands. Particularly, NRP1 acts as a co-receptor of VEGFR1, VEGFR2 and VEGFR3 to bind to various VEGF ligands, thereby contributing to angiogenesis, cell migration & adhesion and invasion. On the other hand, NRP2 acts as a co-receptor of VEGFR2 and VEGFR3, thereby contributing lymphangiogenesis and cell adhesion. Furthermore, neuropilin 1 and 2 act as a co-receptor of plexin family receptors to bind to secreted class-3 semaphorin ligands (Sema3A, Sema3B, Sema3C, Sema3D, Sema3E, Sema3F, Sema3G). Since neuropilin has no domain in functional cells, it has no activity by itself, even if a ligand is binding thereto. It is known that neuropilin signal transduction occurs through VEGF receptor, which is a co-receptor, or through plexin co-receptor. Sema3 binds to neuropilin and plexin receptor at a ratio of 2:2:2 and acts. However, many study results show that neuropilin protein alone can perform signal transduction without its interaction with the VEGF receptor or plexin co-receptor. However, an exact molecular mechanism for this signal transduction is still unclear.

Cases have been reported in which the activities of neuropilin and co-receptor are inhibited even when only neuropilin is targeted. For example, it has been reported that anti-neuropilin-1 antibody binds to only neuropilin-1 competitively with VEGF-A known to bind to VEGFR2 and neuropilin-1, and functions to inhibit angiogenesis, cell survival, migration & adhesion and invasion, which are the actions of VEGFR2 (Pan Q et al. 2007). It has been reported that anti-neuropilin-2 antibody binds to neuropilin-2 competitively with VEGF-C known to binds to both VEGFR3 and neuropilin-2, and functions to inhibit lymphangiogenesis and cell adhesion, which are the operations of VEGFR3 (Caunt M et al. 2008).

The C-terminal region of each of the VEGF ligand family and Sema3 ligands, which bind to neuropilin 1 and 2, binds to the VEGF-binding sites (so-called arginine-binding pocket) in the b1 domain present commonly in neuropilin 1 and 2 (MW Parker et al. 2012). Herein, binding to the arginine-binding pocket occurs by a motif of R/K-x-x-R/K (R=arginine, K=lysine, and x=any amino acids), which is present commonly in the C-terminal region of neuropilin binding ligands. When mutation is induced with an amino acid sequence deviating from the motif, the ligands have a reduced binding affinity for neuropilin or do not bind to neuropilin, and thus lose their biological activity. Particularly, cationic arginine (Arg) or lysine (Lys) in the C-terminal region is essential for binding, and thus when it is substituted with another amino acid residue, the ligand loses its binding affinity for neuropilin, and loses its biological activity. Accordingly, the necessity of the R/K-x-x-R/K motif in the C-terminal region of such neuropilin binding ligands is called “C-end rule” (CendR) (Teesalu et al. 2009). A protein or peptide containing a C-end rule sequence is capable of binding to neuropilin by the C-terminal arginine (Arg) or lysine (Lys) residue (Zanuy et al, 2013).

The C-terminal regions of VEGF ligands and Sema3 ligands commonly have the R/K-x-x-R/K motif, and thus most of the ligands have the property of binding to both neuropilin 1 and 2 rather than binding selectively to any one of neuropilin 1 and 2.

In addition to ligands that bind to neuropilin 1 and 2, many peptides that bind to neuropilin have been selected or designed and reported. These peptides all have the R/K-x-x-R/K motif, and thus appear to bind to the arginine-binding pocket in the b1 domain of neuropilin 1 and 2. Furthermore, an iRGD peptide (Sugahara et al. 2010) that binds to neuropilin 1 and 2 to increase tumor tissue penetration of a co-administered drug, and an A22p peptide (Shin et al. 2014) that is fused to the heavy-chain end of an antibody to increase tumor tissue penetration of the antibody, also have amino acid sequences, following the CendR rule.

The neuropilin domain, or a derivative or fragment thereof, as used herein includes a b1 domain, or a derivative or fragment thereof. Table 1 provides several exemplary neuropilin domains, but not limited to the full-length NRP1 and NRP2 portions in addition to several smaller domains.

TABLE 1
Neuropilin Domains (SEQ ID NOs: 1-22).

| Combined | SEQ ID | | |
|---|---|---|---|
| Name | NO: | Length | Sequence |
| NRP1 Full- | 1 | 923 | MERGLPLLCAVLALVLAPAGAFRNDKCGDTIKIESPGYL |
| length | | | TSPGYPHSYHPSEKCEWLIQAPDPYQRIMINFNPHFDLE |
| | | | DRDCKYDYVEVFDGENENGHFRGKFCGKIAPPPVVSSGP |
| | | | FLFIKFVSDYETHGAGFSIRYEIFKRGPECSQNYTTPSG |
| | | | VIKSPGFPEKYPNSLECTYIVFVPKMSEIILEFESFDLE |
| | | | PDSNPPGGMFCRYDRLEIWDGFPDVGPHIGRYCGQKTPG |
| | | | RIRSSSGILSMVFYTDSAIAKEGFSANYSVLQSSVSEDF |
| | | | KCMEALGMESGEIHSDQITASSQYSTNWSAERSRLNYPE |
| | | | NGWTPGEDSYREWIQVDLGLLRFVTAVGTQGAISKETKK |
| | | | KYYVKTYKIDVSSNGEDWITIKEGNKPVLFQGNTNPTDV |
| | | | VVAVFPKPLITRFVRIKPATWETGISMRFEVYGCKITDY |
| | | | PCSGMLGMVSGLISDSQITSSNQGDRNWMPENIRLVTSR |
| | | | SGWALPPAPHSYINEWLQIDLGEEKIVRGIIIQGGKHRE |
| | | | NKVFMRKFKIGYSNNGSDWKMIMDDSKRKAKSFEGNNNY |
| | | | DTPELRTFPALSTRFIRIYPERATHGGLGLRMELLGCEV |
| | | | EAPTAGPTTPNGNLVDECDDDQANCHSGTGDDFQLTGGT |
| | | | TVLATEKPTVIDSTIQSEFPTYGFNCEFGWGSHKTFCHW |
| | | | EHDNHVQLKWSVLTSKTGPIQDHTGDGNFIYSQADENQK |
| | | | GKVARLVSPVVYSQNSAHCMTFWYHMSGSHVGTLRVKLR |
| | | | YQKPEEYDQLVWMAIGHQGDHWKEGRVLLHKSLKLYQVI |
| | | | FEGEIGKGNLGGIAVDDISINNHISQEDCAKPADLDKKN |
| | | | PEIKIDETGSTPGYEGEGEGDKNISRKPGNVLKTLDPIL |
| | | | ITIIAMSALGVLLGAVCGVVLYCACWHNGMSERNLSALE |
| | | | NYNFELVDGVKLKKDKLNTQSTYSEA |
| NRP2 Full- | 2 | 926 | MDMFPLTWVFLALYFSRHQVRGQPDPPCGGRLNSKDAGY |
| length | | | ITSPGYPQDYPSHQNCEWIVYAPEPNQKIVLNFNPHFEI |
| | | | EKHDCKYDFIEIRDGDSESADLLGKHCGNIAPPTIISSG |
| | | | SMLYIKFTSDYARQGAGFSLRYEIFKTGSEDCSKNFTSP |
| | | | NGTIESPGFPEKYPHNLDCTFTILAKPKMEIILQFLIFD |
| | | | LEHDPLQVGEGDCKYDWLDIWDGIPHVGPLIGKYCGTKT |
| | | | PSELRSSTGILSLTFHTDMAVAKDGFSARYYLVHQEPLE |
| | | | NFQCNVPLGMESGRIANEQISASSTYSDGRWTPQQSRLH |
| | | | GDDNGWTPNLDSNKEYLQVDLRFLTMLTAIATQGAISRE |
| | | | TONGYYVKSYKLEVSTNGEDWMVYRHGKNHKVFQANNDA |
| | | | TEVVLNKLHAPLLTRFVRIRPQTWHSGIALRLELFGCRV |
| | | | TDAPCSNMLGMLSGLIADSQISASSTQEYLWSPSAARLV |
| | | | SSRSGWFPRIPQAQPGEEWLQVDLGTPKTVKGVIIQGAR |
| | | | GGDSITAVEARAFVRKFKVSYSLNGKDWEYIQDPRTQQP |
| | | | KLFEGNMHYDTPDIRRFDPIPAQYVRVYPERWSPAGIGM |
| | | | RLEVLGCDWTDSKPTVETLGPTVKSEETTTPYPTEEEAT |
| | | | ECGENCSFEDDKDLQLPSGFNCNFDFLEEPCGWMYDHAK |
| | | | WLRTTWASSSSPNDRTFPDDRNFLRLQSDSQREGQYARL |
| | | | ISPPVHLPRSPVCMEFQYQATGGRGVALQVVREASQESK |
| | | | LLWVIREDQGGEWKHGRIILPSYDMEYQIVFEGVIGKGR |
| | | | SGEIAIDDIRISTDVPLENCMEPISAFAVDIPEIHEREG |
| | | | YEDEIDDEYEVDWSNSSSATSGSGAPSTDKEKSWLYTLD |
| | | | PILITIIAMSSLGVLLGATCAGLLLYCTCSYSGLSSRSC |
| | | | TTLENYNFELYDGLKHKVKMNHQKCCSEA |
| NRP1 (b1) | 3 | 150 | CMEALGMESGEIHSDQITASSQYSTNWSAERSRLNYPEN |
| | | | GWTPGEDSYREWIQVDLGLLRFVTAVGTQGAISKETKKK |
| | | | YYVKTYKIDVSSNGEDWITIKEGNKPVLFQGNTNPTDVV |
| | | | VAVFPKPLITRFVRIKPATWETGISMRFEVYGC |
| NRP1 (b1)- | 4 | 150 | CMEALGMESGEIHSDQITASSQYSTNWSAERSRLNYPEN |
| E319A | | | GWTPGADSYREWIQVDLGLLRFVTAVGTQGAISKETKKK |
| | | | YYVKTYKIDVSSNGEDWITIKEGNKPVLFQGNTNPTDVV |
| | | | VAVFPKPLITRFVRIKPATWETGISMRFEVYGC |
| NRP1 (b1)- | 5 | 150 | CMEALGMESGEIHSDQITASSQYSTNWSAERSRLNYPEN |
| K351A | | | GWTPGEDSYREWIQVDLGLLRFVTAVGTQGAISKETKAK |
| | | | YYVKTYKIDVSSNGEDWITIKEGNKPVLFQGNTNPTDVV |
| | | | VAVFPKPLITRFVRIKPATWETGISMRFEVYGC |
| NRP1 (b1)- | 6 | 150 | CMEALGMESGEIHSDQITASSQASTNWSAERSRLNYPEN |
| Y297A/S346A/ | | | GWTPGEDSYREWIQVDLGLLRFVTAVGTQGAIAKETKKK |
| Y353A | | | AYVKTYKIDVSSNGEDWITIKEGNKPVLFQGNTNPTDVV |
| | | | VAVFPKPLITRFVRIKPATWETGISMRFEVYGC |
| NRP1 (b1b2) | 7 | 309 | CMEALGMESGEIHSDQITASSQYSTNWSAERSRLNYPEN |
| | | | GWTPGEDSYREWIQVDLGLLRFVTAVGTQGAISKETKKK |
| | | | YYVKTYKIDVSSNGEDWITIKEGNKPVLFQGNTNPTDVV |
| | | | VAVFPKPLITRFVRIKPATWETGISMRFEVYGCKITDYP |
| | | | CSGMLGMVSGLISDSQITSSNQGDRNWMPENIRLVTSRS |
| | | | GWALPPAPHSYINEWLQIDLGEEKIVRGIIIQGGKHREN |
| | | | KVFMRKFKIGYSNNGSDWKMIMDDSKRKAKSFEGNNNYD |
| | | | TPELRTFPALSTRFIRIYPERATHGGLGLRMELLGC |
| NRP1 (b1b2)- | 8 | 309 | CMEALGMESGEIHSDQITASSQYSTNWSAERSRLNYPEN |
| E319A | | | GWTPGADSYREWIQVDLGLLRFVTAVGTQGAISKETKKK |
| | | | YYVKTYKIDVSSNGEDWITIKEGNKPVLFQGNTNPTDVV |
| | | | VAVFPKPLITRFVRIKPATWETGISMRFEVYGCKITDYP |
| | | | CSGMLGMVSGLISDSQITSSNQGDRNWMPENIRLVTSRS |
| | | | GWALPPAPHSYINEWLQIDLGEEKIVRGIIIQGGKHREN |
| | | | KVFMRKFKIGYSNNGSDWKMIMDDSKRKAKSFEGNNNYD |
| | | | TPELRTFPALSTRFIRIYPERATHGGLGLRMELLGC |
| NRP1 (b1b2)- | 9 | 309 | CMEALGMESGEIHSDQITASSQYSTNWSAERSRLNYPEN |
| K351A | | | GWTPGEDSYREWIQVDLGLLRFVTAVGTQGAISKETKAK |
| | | | YYVKTYKIDVSSNGEDWITIKEGNKPVLFQGNTNPTDVV |
| | | | VAVFPKPLITRFVRIKPATWETGISMRFEVYGCKITDYP |
| | | | CSGMLGMVSGLISDSQITSSNQGDRNWMPENIRLVTSRS |
| | | | GWALPPAPHSYINEWLQIDLGEEKIVRGIIIQGGKHREN |
| | | | KVFMRKFKIGYSNNGSDWKMIMDDSKRKAKSFEGNNNYD |
| | | | TPELRTFPALSTRFIRIYPERATHGGLGLRMELLGC |
| NRP1 (b1b2)- | 10 | 309 | CMEALGMESGEIHSDQITASSQASTNWSAERSRLNYPEN |
| Y297A/S346A/ | | | GWTPGEDSYREWIQVDLGLLRFVTAVGTQGAIAKETKKK |
| Y353A | | | AYVKTYKIDVSSNGEDWITIKEGNKPVLFQGNTNPTDVV |
| | | | VAVFPKPLITRFVRIKPATWETGISMRFEVYGCKITDYP |
| | | | CSGMLGMVSGLISDSQITSSNQGDRNWMPENIRLVTSRS |
| | | | GWALPPAPHSYINEWLQIDLGEEKIVRGIIIQGGKHREN |
| | | | KVFMRKFKIGYSNNGSDWKMIMDDSKRKAKSFEGNNNYD |
| | | | TPELRTFPALSTRFIRIYPERATHGGLGLRMELLGC |
| NRP2 (b1) | 11 | 152 | CNVPLGMESGRIANEQISASSTYSDGRWTPQQSRLHGDD |
| | | | NGWTPNLDSNKEYLQVDLRFLTMLTAIATQGAISRETQN |
| | | | GYYVKSYKLEVSTNGEDWMVYRHGKNHKVFQANNDATEV |
| | | | VLNKLHAPLLTRFVRIRPQTWHSGIALRLELFGC |
| NRP2 (b1)- | 12 | 152 | CNVPLGMESGRIANEQISASSTASDGRWTPQQSRLHGDD |
| Y297A/S346A/ | | | NGWTPNLDSNKEYLQVDLRFLTMLTAIATQGAIARETQN |
| Y353A | | | GAYVKSYKLEVSTNGEDWMVYRHGKNHKVFQANNDATEV |
| | | | VLNKLHAPLLTRFVRIRPQTWHSGIALRLELFGC |
| NRP2 (b1b2) | 13 | 316 | CNVPLGMESGRIANEQISASSTYSDGRWTPQQSRLHGDD |
| | | | NGWTPNLDSNKEYLQVDLRFLTMLTAIATQGAISRETQN |
| | | | GYYVKSYKLEVSTNGEDWMVYRHGKNHKVFQANNDATEV |
| | | | VLNKLHAPLLTRFVRIRPQTWHSGIALRLELFGCRVTDA |
| | | | PCSNMLGMLSGLIADSQISASSTQEYLWSPSAARLVSSR |
| | | | SGWFPRIPQAQPGEEWLQVDLGTPKTVKGVIIQGARGGD |
| | | | SITAVEARAFVRKFKVSYSLNGKDWEYIQDPRTQQPKLF |
| | | | EGNMHYDTPDIRRFDPIPAQYVRVYPERWSPAGIGMRLE |
| | | | VLGC |
| NRP2 (b1b2)- | 14 | 316 | CNVPLGMESGRIANEQISASSTASDGRWTPQQSRLHGDD |
| Y297A/S346A/ | | | NGWTPNLDSNKEYLQVDLRFLTMLTAIATQGAIARETQN |
| Y353A | | | GAYVKSYKLEVSTNGEDWMVYRHGKNHKVFQANNDATEV |
| | | | VLNKLHAPLLTRFVRIRPQTWHSGIALRLELFGCRVTDA |
| | | | PCSNMLGMLSGLIADSQISASSTQEYLWSPSAARLVSSR |
| | | | SGWFPRIPQAQPGEEWLQVDLGTPKTVKGVIIQGARGGD |
| | | | SITAVEARAFVRKFKVSYSLNGKDWEYIQDPRTQQPKLF |
| | | | EGNMHYDTPDIRRFDPIPAQYVRVYPERWSPAGIGMRLE |
| | | | VLGC |
| NRP1 (b1b1)- | 15 | 622 | CMEALGMESGEIHSDQITASSQYSTNWSAERSRLNYPEN |
| (G4S)₂-(b1b1) | | | GWTPGEDSYREWIQVDLGLLRFVTAVGTQGAISKETKKK |
| | | | YYVKTYKIDVSSNGEDWITIKEGNKPVLFQGNTNPTDVV |
| | | | VAVFPKPLITRFVRIKPATWETGISMRFEVYGCKITDYP |
| | | | CMEALGMESGEIHSDQITASSQYSTNWSAERSRLNYPEN |
| | | | GWTPGEDSYREWIQVDLGLLRFVTAVGTQGAISKETKKK |
| | | | YYVKTYKIDVSSNGEDWITIKEGNKPVLFQGNTNPTDVV |
| | | | VAVFPKPLITRFVRIKPATWETGISMRFEVYGCGGGGSG |
| | | | GGGSCMEALGMESGEIHSDQITASSQYSTNWSAERSRLN |
| | | | YPENGWTPGEDSYREWIQVDLGLLRFVTAVGTQGAISKE |
| | | | TKKKYYVKTYKIDVSSNGEDWITIKEGNKPVLFQGNTNP |
| | | | TDVVVAVFPKPLITRFVRIKPATWETGISMRFEVYGCKI |
| | | | TDYPCMEALGMESGEIHSDQITASSQYSTNWSAERSRLN |
| | | | YPENGWTPGEDSYREWIQVDLGLLRFVTAVGTQGAISKE |
| | | | TKKKYYVKTYKIDVSSNGEDWITIKEGNKPVLFQGNTNP |
| | | | TDVVVAVFPKPLITRFVRIKPATWETGISMRFEVYGC |
| NRP1 (b1b2)- | 16 | 628 | CMEALGMESGEIHSDQITASSQYSTNWSAERSRLNYPEN |
| (G4S)₂-(b1b2) | | | GWTPGEDSYREWIQVDLGLLRFVTAVGTQGAISKETKKK |
| | | | YYVKTYKIDVSSNGEDWITIKEGNKPVLFQGNTNPTDVV |
| | | | VAVFPKPLITRFVRIKPATWETGISMRFEVYGCKITDYP |
| | | | CSGMLGMVSGLISDSQITSSNQGDRNWMPENIRLVTSRS |
| | | | GWALPPAPHSYINEWLQIDLGEEKIVRGIIIQGGKHREN |
| | | | KVFMRKFKIGYSNNGSDWKMIMDDSKRKAKSFEGNNNYD |
| | | | TPELRTFPALSTRFIRIYPERATHGGLGLRMELLGCGGG |
| | | | GSGGGGSCMEALGMESGEIHSDQITASSQYSTNWSAERS |
| | | | RLNYPENGWTPGEDSYREWIQVDLGLLRFVTAVGTQGAI |
| | | | SKETKKKYYVKTYKIDVSSNGEDWITIKEGNKPVLFQGN |
| | | | TNPTDVVVAVFPKPLITRFVRIKPATWETGISMRFEVYG |
| | | | CKITDYPCSGMLGMVSGLISDSQITSSNQGDRNWMPENI |
| | | | RLVTSRSGWALPPAPHSYINEWLQIDLGEEKIVRGIIIQ |
| | | | GGKHRENKVFMRKFKIGYSNNGSDWKMIMDDSKRKAKSF |
| | | | EGNNNYDTPELRTFPALSTRFIRIYPERATHGGLGLRME |
| | | | LLGC |
| NRP1 (b1b2)- | 17 | 628 | CMEALGMESGEIHSDQITASSQYSTNWSAERSRLNYPEN |
| (G4S)₂-(b1b2) | | | GWTPGADSYREWIQVDLGLLRFVTAVGTQGAISKETKKK |
| with | | | YYVKTYKIDVSSNGEDWITIKEGNKPVLFQGNTNPTDVV |
| b1(E319A) | | | VAVFPKPLITRFVRIKPATWETGISMRFEVYGCKITDYP |
| | | | CSGMLGMVSGLISDSQITSSNQGDRNWMPENIRLVTSRS |
| | | | GWALPPAPHSYINEWLQIDLGEEKIVRGIIIQGGKHREN |
| | | | KVFMRKFKIGYSNNGSDWKMIMDDSKRKAKSFEGNNNYD |
| | | | TPELRTFPALSTRFIRIYPERATHGGLGLRMELLGCGGG |
| | | | GSGGGGSCMEALGMESGEIHSDQITASSQYSTNWSAERS |
| | | | RLNYPENGWTPGADSYREWIQVDLGLLRFVTAVGTQGAI |
| | | | SKETKKKYYVKTYKIDVSSNGEDWITIKEGNKPVLFQGN |
| | | | TNPTDVVVAVFPKPLITRFVRIKPATWETGISMRFEVYG |
| | | | CKITDYPCSGMLGMVSGLISDSQITSSNQGDRNWMPENI |
| | | | RLVTSRSGWALPPAPHSYINEWLQIDLGEEKIVRGIIIQ |
| | | | GGKHRENKVFMRKFKIGYSNNGSDWKMIMDDSKRKAKSF |
| | | | EGNNNYDTPELRTFPALSTRFIRIYPERATHGGLGLRME |
| | | | LLGC |
| NRP1 (b1b2)- | 18 | 628 | CMEALGMESGEIHSDQITASSQYSTNWSAERSRLNYPEN |
| (G4S)₂-(b1b2) | | | GWTPGEDSYREWIQVDLGLLRFVTAVGTQGAISKETKAK |
| with | | | YYVKTYKIDVSSNGEDWITIKEGNKPVLFQGNTNPTDVV |
| b1(K351A) | | | VAVFPKPLITRFVRIKPATWETGISMRFEVYGCKITDYP |
| | | | CSGMLGMVSGLISDSQITSSNQGDRNWMPENIRLVTSRS |
| | | | GWALPPAPHSYINEWLQIDLGEEKIVRGIIIQGGKHREN |
| | | | KVFMRKFKIGYSNNGSDWKMIMDDSKRKAKSFEGNNNYD |
| | | | TPELRTFPALSTRFIRIYPERATHGGLGLRMELLGCGGG |
| | | | GSGGGGSCMEALGMESGEIHSDQITASSQYSTNWSAERS |
| | | | RLNYPENGWTPGEDSYREWIQVDLGLLRFVTAVGTQGAI |
| | | | SKETKAKYYVKTYKIDVSSNGEDWITIKEGNKPVLFQGN |
| | | | TNPTDVVVAVFPKPLITRFVRIKPATWETGISMRFEVYG |
| | | | CKITDYPCSGMLGMVSGLISDSQITSSNQGDRNWMPENI |
| | | | RLVTSRSGWALPPAPHSYINEWLQIDLGEEKIVRGIIIQ |
| | | | GGKHRENKVFMRKFKIGYSNNGSDWKMIMDDSKRKAKSF |
| | | | EGNNNYDTPELRTFPALSTRFIRIYPERATHGGLGLRME |
| | | | LLGC |
| NRP1 (b1b2)- | 19 | 628 | CMEALGMESGEIHSDQITASSQASTNWSAERSRLNYPEN |
| (G4S)₂-(b1b2) | | | GWTPGEDSYREWIQVDLGLLRFVTAVGTQGAIAKETKKK |
| with | | | AYVKTYKIDVSSNGEDWITIKEGNKPVLFQGNTNPTDVV |
| (Y297A/S346A/ | | | VAVFPKPLITRFVRIKPATWETGISMRFEVYGCKITDYP |
| Y353A) | | | CSGMLGMVSGLISDSQITSSNQGDRNWMPENIRLVTSRS |
| | | | GWALPPAPHSYINEWLQIDLGEEKIVRGIIIQGGKHREN |
| | | | KVFMRKFKIGYSNNGSDWKMIMDDSKRKAKSFEGNNNYD |
| | | | TPELRTFPALSTRFIRIYPERATHGGLGLRMELLGCGGG |
| | | | GSGGGGSCMEALGMESGEIHSDQITASSQASTNWSAERS |
| | | | RLNYPENGWTPGEDSYREWIQVDLGLLRFVTAVGTQGAI |
| | | | AKETKKKAYVKTYKIDVSSNGEDWITIKEGNKPVLFQGN |
| | | | TNPTDVVVAVFPKPLITRFVRIKPATWETGISMRFEVYG |
| | | | CKITDYPCSGMLGMVSGLISDSQITSSNQGDRNWMPENI |
| | | | RLVTSRSGWALPPAPHSYINEWLQIDLGEEKIVRGIIIQ |
| | | | GGKHRENKVFMRKFKIGYSNNGSDWKMIMDDSKRKAKSF |
| | | | EGNNNYDTPELRTFPALSTRFIRIYPERATHGGLGLRME |
| | | | LLGC |
| NRP2 (b1b1)- | 20 | 624 | CNVPLGMESGRIANEQISASSTYSDGRWTPQQSRLHGDD |
| (G4S)₂-(b1b1) | | | NGWTPNLDSNKEYLQVDLRFLTMLTAIATQGAISRETQN |
| | | | GYYVKSYKLEVSTNGEDWMVYRHGKNHKVFQANNDATEV |
| | | | VLNKLHAPLLTRFVRIRPQTWHSGIALRLELFGCRVTDA |
| | | | CNVPLGMESGRIANEQISASSTYSDGRWTPQQSRLHGDD |
| | | | NGWTPNLDSNKEYLQVDLRFLTMLTAIATQGAISRETQN |
| | | | GYYVKSYKLEVSTNGEDWMVYRHGKNHKVFQANNDATEV |
| | | | VLNKLHAPLLTRFVRIRPQTWHSGIALRLELFGCGGGGS |
| | | | GGGGSCNVPLGMESGRIANEQISASSTYSDGRWTPQQSR |
| | | | LHGDDNGWTPNLDSNKEYLQVDLRFLTMLTAIATQGAIS |
| | | | RETQNGYYVKSYKLEVSTNGEDWMVYRHGKNHKVFQANN |
| | | | DATEVVLNKLHAPLLTRFVRIRPQTWHSGIALRLELFGC |
| | | | RVTDACNVPLGMESGRIANEQISASSTYSDGRWTPQQSR |
| | | | LHGDDNGWTPNLDSNKEYLQVDLRFLTMLTAIATQGAIS |
| | | | RETQNGYYVKSYKLEVSTNGEDWMVYRHGKNHKVFQANN |
| | | | DATEVVLNKLHAPLLTRFVRIRPQTWHSGIALRLELFGC |
| NRP2 (b1b2)- | 21 | 642 | CNVPLGMESGRIANEQISASSTYSDGRWTPQQSRLHGDD |
| (G4S)₂-(b1b2) | | | NGWTPNLDSNKEYLQVDLRFLTMLTAIATQGAISRETQN |
| | | | GYYVKSYKLEVSTNGEDWMVYRHGKNHKVFQANNDATEV |
| | | | VLNKLHAPLLTRFVRIRPQTWHSGIALRLELFGCRVTDA |
| | | | PCSNMLGMLSGLIADSQISASSTQEYLWSPSAARLVSSR |
| | | | SGWFPRIPQAQPGEEWLQVDLGTPKTVKGVIIQGARGGD |
| | | | SITAVEARAFVRKFKVSYSLNGKDWEYIQDPRTQQPKLF |
| | | | EGNMHYDTPDIRRFDPIPAQYVRVYPERWSPAGIGMRLE |
| | | | VLGCGGGGSGGGGSCNVPLGMESGRIANEQISASSTYSD |
| | | | GRWTPQQSRLHGDDNGWTPNLDSNKEYLQVDLRFLTMLT |
| | | | AIATQGAISRETQNGYYVKSYKLEVSTNGEDWMVYRHGK |
| | | | NHKVFQANNDATEVVLNKLHAPLLTRFVRIRPQTWHSGI |
| | | | ALRLELFGCRVTDAPCSNMLGMLSGLIADSQISASSTQE |
| | | | YLWSPSAARLVSSRSGWFPRIPQAQPGEEWLQVDLGTPK |
| | | | TVKGVIIQGARGGDSITAVEARAFVRKFKVSYSLNGKDW |
| | | | EYIQDPRTQQPKLFEGNMHYDTPDIRRFDPIPAQYVRVY |
| | | | PERWSPAGIGMRLEVLGC |
| NRP2 (b1b2)- | 22 | 642 | CNVPLGMESGRIANEQISASSTASDGRWTPQQSRLHGDD |
| (G4S)₂-(b1b2) | | | NGWTPNLDSNKEYLQVDLRFLTMLTAIATQGAIARETQN |
| with | | | GAYVKSYKLEVSTNGEDWMVYRHGKNHKVFQANNDATEV |
| (Y297A/S346A/ | | | VLNKLHAPLLTRFVRIRPQTWHSGIALRLELFGCRVTDA |
| Y353A) | | | PCSNMLGMLSGLIADSQISASSTQEYLWSPSAARLVSSR |
| | | | SGWFPRIPQAQPGEEWLQVDLGTPKTVKGVIIQGARGGD |
| | | | SITAVEARAFVRKFKVSYSLNGKDWEYIQDPRTQQPKLF |
| | | | EGNMHYDTPDIRRFDPIPAQYVRVYPERWSPAGIGMRLE |
| | | | VLGCGGGGSGGGGSCNVPLGMESGRIANEQISASSTASD |
| | | | GRWTPQQSRLHGDDNGWTPNLDSNKEYLQVDLRFLTMLT |
| | | | AIATQGAIARETQNGAYVKSYKLEVSTNGEDWMVYRHGK |
| | | | NHKVFQANNDATEVVLNKLHAPLLTRFVRIRPQTWHSGI |
| | | | ALRLELFGCRVTDAPCSNMLGMLSGLIADSQISASSTQE |
| | | | YLWSPSAARLVSSRSGWFPRIPQAQPGEEWLQVDLGTPK |
| | | | TVKGVIIQGARGGDSITAVEARAFVRKFKVSYSLNGKDW |
| | | | EYIQDPRTQQPKLFEGNMHYDTPDIRRFDPIPAQYVRVY |
| | | | PERWSPAGIGMRLEVLGC |

Immunoglobulins

In some embodiments, the polypeptides of the present invention comprise an immunoglobulin domain. The immunoglobulin domain can be derived from an immunoglobulin molecule from any mammal. In one embodiment, the immunoglobulin domain is derived from a human immunoglobulin, and can be derived from an immunoglobulin isotype selected from the group consisting of IgG, IgA, and IgD antibody isotypes. In a particular embodiment, the immunoglobulin domain is derived from an IgG isotype. In still another embodiment, the immunoglobulin domain is derived from a subclass of IgG selected from the group consisting of IgG1, IgG2, and IgG4.

As the biocompatible material capable of increasing the half-life of a physiologically active polypeptide linked thereto, IgG Fc regions including a hinge region were selected.

In order to reduce this unintended effector function, human IgG4 variant L235E or F234A/L235A, and the human IgG1 variant L234A/L235A were generated, all of which reduced inflammatory cytokine release. Another early approach intended to reduce effector function was to mutate the glycosylation site at N297 with mutations such as N297A, N297Q, and N297G. This glycosylation approach has proven successful in abrogating Fc interactions with the low affinity FcγRs and effector functions such as CDC and ADCC. Among the four IgG subclasses, each has a different ability to elicit immune effector functions. For instance, IgG1 and IgG3 have been recognized to recruit complement more effectively than IgG2 and IgG4. Additionally, IgG2 and IgG4 have very limited ability to elicit ADCC. Therefore, several investigators have employed a cross-subclass approach to reduce effector function.

FcRn is known to prolong the half-life of IgG, the obvious strategy has been to modulate FcRn-IgG interaction to either extend or shorten the antibody half-life. Half-life extension of therapeutic antibodies would help maintain drug therapeutic levels and reduce the frequency of administration, while half-life reduction would be ideal for diagnostic tests or toxicity control.

Attempts to prolong antibody half-life by mutations of the Fc region critical for FcRn binding have been relatively successful. However, increasing Fc to FcRn binding does not necessarily prolong serum half-life. In fact, IgG1 mutants created to significantly increase binding at pH 6.0 as well as pH 7.4 did not contribute to increase serum half-life, but instead offset the benefit of enhanced binding at pH 6.0 alone. It is believed that FcRn-IgG binding at pH 7.4 prevents IgG release into the circulation and instead diverts it to the degradation pathway. Furthermore, it has been suggested that the rate of dissociation at pH 7.4 is equally or perhaps more important in determining serum half-life.

Among a previous comprehensive screen of Fc mutations in FcRn binding study, N434A and T307A/E380A/N434A (AAA), were shown to have 3.4-fold and 11.8-fold increases in binding to FcRn (human). Substitutions in trastuzumab resulted in 1.3- and 3.3-fold increases in binding to FcRn (human) using a cell based assay and 2.2- and 2.5-fold increases in the serum half-life in mice that expressed the human FcRn transgene and deficient in the endogenous FcRn (hFcRn-Tg).

The fusion proteins to bind spike protein of virus comprises the IgG Fc region which includes a hinge region, ACE2 (angiotensin-converting enzyme 2) fragment which includes a spike protein binding region and NRP (Neuropilin) fragment which includes a CendR binding region using a linker or by a method of direct fusion.

In some embodiments, the polypeptides of the present invention comprise an immunoglobulin domain which includes a portion of the heavy chain. In one embodiment, the immunoglobulin domain comprises the fragment crystallizable (Fc) domain, which comprises hinge-CH2-CH3 of the antibody. In a particular embodiment, the polypeptide comprises an immunoglobulin domain that comprises an Fc domain selected from the IgG1 and IgG2 subclass.

In some embodiments, the immunoglobulin domain comprises a modified Fc domain.

For example, in certain embodiments, it may be preferable to have increased affinity for the Fc Fragment of IgG Receptor and Transporter (FcRn). In adults, FcRn is expressed in epithelial tissue. FcRn is able to carry out diverse roles through the transport and recycling of bound IgG within and across cells. Antibodies and other Fc containing peptides are internalized via binding with the FcRn and are targeted to the acidic endosomes and lysosomes of the cells for degradation. Therefore, in certain embodiments, the polypeptides comprise an immunoglobulin domain containing an Fc domain with increased binding affinity for FcRn. In one embodiment, the immunoglobulin domain comprises an amino acid sequence selected from the group consisting of SEQ ID NOs: 24, 27 and 29.

In other embodiments, it may be desirable to have reduced affinity for one or more Fcγ receptors. In some instances, such as HIV and dengue virus, the virus-antibody complex that binds to Fcγ receptors (FcγR) results in the virus coming into close proximity to the virus-specific receptor, resulting in infection. As such, a virus complexed with an antibody may result in a phenomenon referred to as Antibody-Dependent Enhancement (ADE) of infection. Therefore, in certain embodiments, the polypeptide comprises an immunoglobulin domain comprising an Fc domain with reduced affinity for one or more Fcγ receptors. In one embodiment, the immunoglobulin domain comprises an amino acid sequence selected from the group consisting of SEQ ID NOs: 26 and 28.

In still other embodiments, it may be desirable to reduce the antibody-dependent cellular cytotoxicity (ADCC) elicited by the immunoglobulin domain. In some instances, hyperactivation of ADCC may result in an uncontrolled, high release of cytokines, precipitating in a “cytokine storm”. In other instances, ADCC may result in internalization of the virus particle into susceptible target cells. As such, in one embodiment, the polypeptide comprises an immunoglobulin domain that is deficient in eliciting ADCC. In one embodiment, the polypeptide comprises an immunoglobulin domain comprising an Fc domain with reduced ADCC, for example, containing an N297A mutation. In one embodiment, the immunoglobulin domain comprises an amino acid sequence selected from the group consisting of SEQ ID NOs: 25 and 27.

The term “heavy chain” as used herein may be interpreted to include a full-length heavy chain including heavy chain variable region domain VH including an amino acid sequence having a variable region sequence sufficient to confer antigen-specificity and three heavy chain constant region domains CH1, CH2 and CH3, and a fragment thereof. Also, the term “light chain” as used herein may be interpreted to include a full-length light chain including a light chain variable region domain VL including an amino acid sequence having a variable region sequence sufficient to confer antigen-specificity and a light chain constant region domain CL, and a fragment thereof.

Moreover, the antibody fragment may be a monomer, a dimer, or a multimer.

The antibody includes monoclonal antibodies, non-specific antibodies, non-human antibodies, human antibodies, humanized antibodies, chimeric antibodies, single-chain Fvs (scFV), single chain antibodies, Fab fragments, F(ab′) fragments, disulfide-linked Fvs (sdFV) and anti-idiotype (anti-Id) antibodies, and epitope-binding fragments of these antibodies, but is not limited thereto.

The monoclonal antibody may be IgG, IgM, IgA, IgD, or IgE. For example, the monoclonal antibody may be IgG1, IgG2, IgG3, IgG4, IgM, IgE, IgAl, IgA5, or IgD type, and may be IgG1 type. In addition, the light-chain constant region of the antibody may be of k or K type.

The peptide may bind to a heavy chain constant region (Fc) fragment of an antibody, preferably to the C-terminus of a heavy chain constant region (Fc) fragment of an antibody. The binding may be performed by a linker peptide.

In some embodiments, the polypeptides of the present invention comprise an Fc domain.

Table 2 provides several exemplary immunoglobulin domains used in the therapeutic polypeptides described herein.

```
TABLE 2
Immunoglobulin Domains (SEQ ID NOs: 23-31). Amino acid mutations are shown
  in parenthesis where applicable. The column labeled effect describes
  the characteristics possessed by an IgG as bestowed by a given mutation.
```

| | | | SEQ ID | | |
|---|---|---|---|---|---|
| IgG Name | Effect | Source | NO: | Length | Sequence |
| IgG1 WT | NA | IgG1 | 23 | 232 | EPKSCDKTHTCPPCPAPELLGGPSVFLFPPKPKDTLMISR |
| | | | | | TPEVTCVVVDVSHEDPEVKFNWYVDGVEVHNAKTKPREEQ |
| | | | | | YNSTYRVVSVLTVLHQDWLNGKEYKCKVSNKALPAPIEKT |
| | | | | | ISKAKGQPREPQVYTLPPSRDELTKNQVSLTCLVKGFYPS |
| | | | | | DIAVEWESNGQPENNYKTTPPVLDSDGSFFLYSKLTVDKS |
| | | | | | RWQQGNVFSCSVMHEALHNHYTQKSLSLSPGK |
| IgG1 | Increased FcRn | IgG1 | 24 | 232 | EPKSCDKTHTCPPCPAPELLGGPSVFLFPPKPKDTLMISR |
| (T307A/E380A/ | binding | | | | TPEVTCVVVDVSHEDPEVKFNWYVDGVEVHNAKTKPREEQ |
| N434A) | | | | | YNSTYRVVSVLAVLHQDWLNGKEYKCKVSNKALPAPIEKT |
| | | | | | ISKAKGQPREPQVYTLPPSRDELTKNQVSLTCLVKGFYPS |
| | | | | | DIAVAWESNGQPENNYKTTPPVLDSDGSFFLYSKLTVDKS |
| | | | | | RWQQGNVFSCSVMHEALHAHYTQKSLSLSPGK |
| IgG1 (N297A) | No ADCC | IgG1 | 25 | 232 | EPKSCDKTHTCPPCPAPELLGGPSVFLFPPKPKDTLMISR |
| | | | | | TPEVTCVVVDVSHEDPEVKFNWYVDGVEVHNAKTKPREEQ |
| | | | | | YNSTYRVVSVLAVLHQDWLNGKEYKCKVSNKALPAPIEKT |
| | | | | | ISKAKGQPREPQVYTLPPSRDELTKNQVSLTCLVKGFYPS |
| | | | | | DIAVAWESNGQPENNYKTTPPVLDSDGSFFLYSKLTVDKS |
| | | | | | RWQQGNVFSCSVMHEALHAHYTQKSLSLSPGK |
| IgG1 | LALA mutation | IgG1 | 26 | 232 | EPKSCDKTHTCPPCPAPEAAGGPSVFLFPPKPKDTLMISR |
| (L324A/L325A) | (removes Fc | | | | TPEVTCVVVDVSHEDPEVKFNWYVDGVEVHNAKTKPREEQ |
| | gamma receptor | | | | YNSTYRVVSVLTVLHQDWLNGKEYKCKVSNKALPAPIEKT |
| | binding) | | | | ISKAKGQPREPQVYTLPPSRDELTKNQVSLTCLVKGFYPS |
| | | | | | DIAVEWESNGQPENNYKTTPPVLDSDGSFFLYSKLTVDKS |
| | | | | | RWQQGNVFSCSVMHEALHNHYTQKSLSLSPGK |
| IgG1 | Increased FcRn | IgG1 | 27 | 232 | EPKSCDKTHTCPPCPAPELLGGPSVFLFPPKPKDTLMISR |
| (N297A/T307A/ | binding with No | | | | TPEVTCVVVDVSHEDPEVKFNWYVDGVEVHNAKTKPREEQ |
| E380A/N434A) | ADCC | | | | YASTYRVVSVLAVLHQDWLNGKEYKCKVSNKALPAPIEKT |
| | | | | | ISKAKGQPREPQVYTLPPSRDELTKNQVSLTCLVKGFYPS |
| | | | | | DIAVAWESNGQPENNYKTTPPVLDSDGSFFLYSKLTVDKS |
| | | | | | RWQQGNVFSCSVMHEALHAHYTQKSLSLSPGK |
| IgG1 | Increased FcRn | IgG1 | 28 | 232 | EPKSCDKTHTCPPCPAPEAAGGPSVFLFPPKPKDTLMISR |
| (L324A/L325A/ | binding with | | | | TPEVTCVVVDVSHEDPEVKFNWYVDGVEVHNAKTKPREEQ |
| T307A/E380A/ | LALA mutation | | | | YNSTYRVVSVLAVLHQDWLNGKEYKCKVSNKALPAPIEKT |
| N434A) | | | | | ISKAKGQPREPQVYTLPPSRDELTKNQVSLTCLVKGFYPS |
| | | | | | DIAVAWESNGQPENNYKTTPPVLDSDGSFFLYSKLTVDKS |
| | | | | | RWQQGNVFSCSVMHEALHAHYTQKSLSLSPGK |
| IgG2 (T250Q, | Increased FcRn | IgG2 | 29 | 228 | ERKCCVECPPCPAPPVAGPSVFLFPPKPKDQLMISRTPEV |
| M428L) | binding | | | | TCVVVDVSHEDPEVQFNWYVDGVEVHNAKTKPREEQFNST |
| | | | | | FRVVSVLTVVHQDWLNGKEYKCKVSNKGLPAPIEKTISKT |
| | | | | | KGQPREPQVYTLPPSREEMTKNQVSLTCLVKGFYPSDIAV |
| | | | | | EWESNGQPENNYKTTPPMLDSDGSFFLYSKLTVDKSRWQQ |
| | | | | | GNVFSCSVLHEALHNHYTQKSLSLSPGK |
| IgG2 WT | NA | IgG2 | 30 | 228 | ERKCCVECPPCPAPPVAGPSVFLFPPKPKDTLMISRTPEV |
| | | | | | TCVVVDVSHEDPEVQFNWYVDGVEVHNAKTKPREEQFNST |
| | | | | | FRVVSVLTVVHQDWLNGKEYKCKVSNKGLPAPIEKTISKT |
| | | | | | KGQPREPQVYTLPPSREEMTKNQVSLTCLVKGFYPSDIAV |
| | | | | | EWESNGQPENNYKTTPPMLDSDGSFFLYSKLTVDKSRWQQ |
| | | | | | GNVFSCSVMHEALHNHYTQKSLSLSPGK |
| IgG4 WT | NA | lgG4 | 31 | 229 | ESKYGPPCPSCPAPEFLGGPSVFLFPPKPKDTLMISRTPE |
| | | | | | VTCVVVDVSQEDPEVQFNWYVDGVEVHNAKTKPREEQFNS |
| | | | | | TYRVVSVLTVLHQDWLNGKEYKCKVSNKGLPSSIEKTISK |
| | | | | | AKGQPREPQVYTLPPSQEEMTKNQVSLTCLVKGFYPSDIA |
| | | | | | VEWESNGQPENNYKTTPPVLDSDGSFFLYSRLTVDKSRWQ |
| | | | | | EGNVFSCSVMHEALHNHYTQKSLSLSLGK |

“WT” means wild-type.
“ADCC” refers to Antibody-dependent cellular cytotoxicity (ADCC).
“NA” means not applicable.

Angiotensin Converting Enzyme 2 Neutralization of SARS-CoV-2

The angiotensin-converting enzyme (ACE)-related carboxypeptidase, ACE2, is a type I integral membrane protein of about 805 amino acids that contains one HEXXH+E zinc-binding consensus sequence. ACE2 is a close homolog of the somatic angiotensin-converting enzyme (ACE; EC 3.4.15.1), a peptidyl dipeptidase that plays an important role in the renin-angiotensin system. ACE2 sequence includes an N-terminal signal sequence (amino acids 1 to 18), a potential transmembrane domain (amino acids 740 to 763), and a potential metalloprotease zinc-binding site (amino acids 374 to 378, HEMGH).

A crystal structure of ACE2 bound to an S protein fragment containing the RBD (residues 306 to 527) was published. ACE2 residues that made direct contact with the RBD included Q24, T27, K31, H34, E37, D38, Y41, Q42, L45, L79, M82, Y83, N90, Q325, E329, N330, K353 and G354. The comparative structural analyses suggest that most ACE2 key residues involved in S-protein binding are found on the N-terminal back-to-back alpha-helices 1 and 2. To determine whether the two helices do in fact remain stable in complex with the S-protein.

In some embodiments of the invention, polypeptides comprising an ACE2 domain are disclosed. In some embodiments, the ACE2 domain is derived from a mammalian ACE2 sequence. In one embodiment, the ACE2 domain is the human ACE2 sequence (the full-length amino acid sequence, including the signal sequence, is provided in SEQ ID NO: 32), a derivative or fragment thereof that comprises amino acids 22-44 of SEQ ID NO: 32. For example, in one particular embodiment, the ACE2 domain comprises the ACE2 domain comprises the amino acid sequence selected from:

(SEQ ID NO: 34)
EEQAKTFLDKFNHEAEDLFYQSS
and
(SEQ ID NO: 35)
IEEQAKTFLDKFNHEAEDLFYQSSLASWNYNTNITEENVQNMNNAGDKWS
AFLKEQSTLAQMYPLQEI

In another embodiment, the ACE2 domain further comprises amino acids 351-357 of SEQ ID NO: 32. For example, in another embodiment, the ACE2 domain is selected from the group consisting of:

(SEQ ID NO: 36)
EEQAKTFLDKFNHEAEDLFYQSS(X)ₙLGKGDFR
and
(SEQ ID NO: 37)
IEEQAKTFLDKFNHEAEDLFYQSSLASWNYNTNITEENVQNMNNAGDKWS
AFLKEQSTLAQMYPLQEI(X)nWDLGKGDFR

wherein:
n=0-30
X=any amino acid selected from Gly, Ala, Ser, Ile, Leu and Val, or any combination thereof.

In certain embodiments, the ACE2 domain can be modified, for example, to contain amino acid substitutions to alter the affinity for virus particles. In one embodiment, the ACE2 domain comprises an amino acid substitution at a position selected from the group consisting of F28, D30, and L79 (amino acid numbering based on the full-length human ACE2 sequence). In one embodiment, the ACE2 domain comprises an F28W substitution. In another embodiment, the ACE2 domain comprises a D30A substitution. In still another embodiment, the ACE2 domain comprises a L79T substitution. Non-limiting examples of the various ACE2 domains include, but are not limited to: ACE2-1, ACE2-2, ACE2-3, ACE2-4, ACE2-5, and ACE2-6. The ACE2-1 domain includes an α-helix 1+β-sheet)-(G4S)*2-(α-helix 1+β-sheet. The ACE2-2 domain includes an α-helix 1+α-helix 2+β-sheet)-(G4S)*2-(α-helix 1+α-helix 2+β-sheet. The ACE2-3 includes ACE2-1 with F28W. The ACE2-4 includes ACE2-2 with F28W. The ACE2-5 includes ACE2-2 with D30A. The ACE2-6 includes ACE2-2 with L79T.

In order to induce decoy proteins specifically binding to spike proteins, sequences of angiotensin-converting enzyme 2 (ACE2), neuropilin-1 (NRP-1) and neuropilin-2 (NRP-2) were analyzed. Representatively, the whole sequences of angiotensin-converting enzyme 2, neuropilin-1 and neuropilin-2 were selected from the PubMed Entrez Protein Database.

Table 3 provides several exemplary ACE2 domains used in the therapeutic polypeptides described herein.

TABLE 3
ACE2 Domains (SEQ ID NOs: 32-45). Here, “(X)ₙ” means X
can be any amino acid selected from Gly, Ala, Ser, Ile,
Leu and Val, or any combination thereof; and n = 0-30.

| | Multimer, | | | |
|---|---|---|---|---|
| | F28W, D30A, | | | |
| | or L79T | | | |
| | Mutant | SEQ ID | | |
| Name | Content | NO: | Length | Sequence |
| Full length | None | 32 | 555 | MSSSSWLLLSLVAVTAAQSTIEEQAKTFLDK |
| ACE2 | | | | FNHEAEDLFYQSSLASWNYNTNITEENVQNM |
| | | | | NNAGDKWSAFLKEQSTLAQMYPLQEIQNLTV |
| | | | | KLQLQALQQNGSSVLSEDKSKRLNTILNTMS |
| | | | | TIYSTGKVCNPDNPQECLLLEPGLNEIMANS |
| | | | | LDYNERLWAWESWRSEVGKQLRPLYEEYVVL |
| | | | | KNEMARANHYEDYGDYWRGDYEVNGVDGYDY |
| | | | | SRGQLIEDVEHTFEEIKPLYEHLHAYVRAKL |
| | | | | MNAYPSYISPIGCLPAHLLGDMWGRFWTNLY |
| | | | | SLTVPFGQKPNIDVTDAMVDQAWDAQRIFKE |
| | | | | AEKFFVSVGLPNMTQGFWENSMLTDPGNVQK |
| | | | | AVCHPTAWDLGKGDFRILMCTKVTMDDFLTA |
| | | | | HHEMGHIQYDMAYAAQPFLLRNGANEGFHEA |
| | | | | VGEIMSLSAATPKHLKSIGLLSPDFQEDNET |
| | | | | EINFLLKQALTIVGTLPFTYMLEKWRWMVFK |
| | | | | GEIPKDQWMKKWWEMKREIVGVVEPVPHDET |
| | | | | YCDPASLFHVSDDYSFIRYYTRTLYQFQFQE |
| | | | | ALCQAAKHEGPLHKCDISNSTEAGQKLL |
| Soluble | None | 33 | 598 | QSTIEEQAKTFLDKFNHEAEDLFYQSSLASW |
| ACE-2 | | | | NYNTNITEENVQNMNNAGDKWSAFLKEQSTL |
| | | | | AQMYPLQEIQNLTVKLQLQALQQNGSSVLSE |
| | | | | DKSKRLNTILNTMSTIYSTGKVCNPDNPQEC |
| | | | | LLLEPGLNEIMANSLDYNERLWAWESWRSEV |
| | | | | GKQLRPLYEEYVVLKNEMARANHYEDYGDYW |
| | | | | RGDYEVNGVDGYDYSRGQLIEDVEHTFEEIK |
| | | | | PLYEHLHAYVRAKLMNAYPSYISPIGCLPAH |
| | | | | LLGDMWGRFWTNLYSLTVPFGQKPNIDVTDA |
| | | | | MVDQAWDAQRIFKEAEKFFVSVGLPNMTQGF |
| | | | | WENSMLTDPGNVQKAVCHPTAWDLGKGDFRI |
| | | | | LMCTKVTMDDFLTAHHEMGHIQYDMAYAAQP |
| | | | | FLLRNGANEGFHEAVGEIMSLSAATPKHLKS |
| | | | | IGLLSPDFQEDNETEINFLLKQALTIVGTLP |
| | | | | FTYMLEKWRWMVFKGEIPKDQWMKKWWEMKR |
| | | | | EIVGVVEPVPHDETYCDPASLFHVSNDYSFI |
| | | | | RYYTRTLYQFQFQEALCQAAKHEGPLHKCDI |
| | | | | SNSTEAGQKLFNMLRLGKSEPWTLALENVVG |
| | | | | AKNMNVRPLLNYFEPLFTWLKDQNKNSFVGW |
| | | | | STDWSPYAD |
| Monomeric | | 34 | 23 | EEQAKTFLDKFNHEAEDLFYQSS |
| ACE2-1 | | | | |
| minimal | | | | |
| Monomeric | | 35 | 68 | IEEQAKTFLDKFNHEAEDLFYQSSLASWNYN |
| ACE2-1 | | | | TNITEENVQNMNNAGDKWSAFLKEQSTLAQM |
| minimal | | | | YPLQEI |
| Monomeric | | 36 | 31 | EEQAKTFLDKFNHEAEDLFYQSS(X)ₙLGKG |
| ACE2-1 | | | | DFR |
| Motif | | | | |
| Monomeric | | 37 | 78 | IEEQAKTFLDKFNHEAEDLFYQSSLASWNYN |
| ACE2-1 | | | | TNITEENVQNMNNAGDKWSAFLKEQSTLAQM |
| Motif | | | | YPLQEI(X)ₙWDLGKGDFR |
| Monomeric | | 38 | 31 | EEQAKTFLDKFNHEAEDLFYQSSGLGKGDFR |
| ACE2-1 | | | | |
| Monomeric | | 39 | 77 | IEEQAKTFLDKFNHEAEDLFYQSSLASWNYN |
| ACE2-2 | | | | TNITEENVQNMNNAGDKWSAFLKEQSTLAQM |
| | | | | YPLQEIWDLGKGDFR |
| ACE2-1 | Dimer of SEQ | 40 | 72 | EEQAKTFLDKFNHEAEDLFYQSSGLGKGDFR |
| | ID NO: 36 | | | GGGGSGGGGSEEQAKTFLDKFNHEAEDLFYQ |
| | | | | SSGLGKGDFR |
| ACE2-2 | Dimer of SEQ | | 164 | IEEQAKTFLDKFNHEAEDLFYQSSLASWNYN |
| | ID NO: 37 | | | TNITEENVQNMNNAGDKWSAFLKEQSTLAQM |
| | | | | YPLQEIWDLGKGDFRGGGGSGGGGSIEEQAK |
| | | | | TFLDKFNHEAEDLFYQSSLASWNYNTNITEE |
| | | | | NVQNMNNAGDKWSAFLKEQSTLAQMYPLQEI |
| | | | | WDLGKGDFR |
| ACE2-3 | SEQ ID NO:38 | 42 | 72 | EEQAKTWLDKFNHEAEDLFYQSSGLGKGDFR |
| | with F28W | | | GGGGSGGGGSEEQAKTWLDKFNHEAEDLFYQ |
| | Substitution | | | SSGLGKGDFR |
| ACE2-4 | SEQ ID NO: | 43 | 164 | IEEQAKTWLDKFNHEAEDLFYQSSLASWNYN |
| | 39 with F28W | | | TNITEENVQNMNNAGDKWSAFLKEQSTLAQM |
| | Substitution | | | YPLQEIWDLGKGDFRGGGGSGGGGSIEEQAK |
| | | | | TWLDKFNHEAEDLFYQSSLASWNYNTNITEE |
| | | | | NVQNMNNAGDKWSAFLKEQSTLAQMYPLQEI |
| | | | | WDLGKGDFR |
| ACE2-5 | SEQ ID NO: | 44 | 164 | IEEQAKTFLAKFNHEAEDLFYQSSLASWNYN |
| | 39 with D30A | | | TNITEENVQNMNNAGDKWSAFLKEQSTLAQM |
| | Substitution | | | YPLQEIWDLGKGDFRGGGGSGGGGSIEEQAK |
| | | | | TFLAKFNHEAEDLFYQSSLASWNYNTNITEE |
| | | | | NVQNMNNAGDKWSAFLKEQSTLAQMYPLQEI |
| | | | | WDLGKGDFR |
| ACE2-6 | SEQ ID NO: | 45 | 164 | IEEQAKTFLDKFNHEAEDLFYQSSLASWNYN |
| | 39 with L79T | | | TNITEENVQNMNNAGDKWSAFLKEQSTTAQM |
| | Substitution | | | YPLQEIWDLGKGDFRGGGGSGGGGSIEEQAK |
| | | | | TFLDKFNHEAEDLFYQSSLASWNYNTNITEE |
| | | | | NVQNMNNAGDKWSAFLKEQSTTAQMYPLQEI |
| | | | | WDLGKGDFR |

Linker Portions

The peptide binding specifically to NRP1 of an aspect of the present disclosure may further comprise a linker peptide. The linker peptide may comprise or consist of 1 to 50 amino acids, 4 to 20 amino acids, or 4 to 10 amino acids. In addition, the linker peptide may comprise or consist of glycine or serine, and may comprise or consist of an amino acid sequence of (GGGGS)n (wherein n is each independently an integer between 1 and 20), or may comprise or consist of an amino acid sequence of (GGGGS)₂.

In some embodiments of the present disclosure, the peptide having the linker peptide bound thereto may comprise the amino acid sequence of any one of SEQ ID NOs: 46 to 52 as provided below in Table 4.

TABLE 4
Linker Domains (SEQ ID NOs: 46-52).

| SEQ ID | | | |
|---|---|---|---|
| NO: | NAME | LENGTH | SEQUENCE |
| 46 | (G4S)2 | 10 | GGGGSGGGGS |
| | Linker | | |
| 47 | NRP1 B1 | 6 | KITDYP |
| | linker | | |
| 48 | NRP2 B1 | 6 | RVTDAP |
| | linker | | |
| 49 | NRP1 B1 | 4 | EDFK |
| | linker | | |
| 50 | NRP2 B1 | 4 | ENFQ |
| | linker | | |
| 51 | (G4A)2 | 10 | GGGGAGGGGA |
| | Linker | | |
| 52 | (GA)5 | 10 | GAGAGAGAGA |
| | Linker | | |

Signal Sequence

Signal sequences can be located on the N-terminus of peptides and can enable those proteins to find their correct location outside the cell membrane. The signal sequence can tag the protein for transport through the cell membrane to be removed from the cell. Signal peptides can function to prompt a cell to translocate the protein, usually to the cellular membrane. In prokaryotes, signal peptides can direct the newly synthesized protein to the SecYEG protein-conducting channel, which is present in the plasma membrane.

In some embodiments of the present disclosure, the signal sequence may comprise the amino acid sequence of SEQ ID NO: 53 as provided below in Table 5.

TABLE 5
Signal Sequence Domain (SEQ ID NO: 53).

| SEQ ID NO: | LENGTH | SEQUENCE |
|---|---|---|
| 53 | 19 | MGWSCIILFLVATATGVHS |

Immunoglobulin-ACE2 Constructs

In one aspect of the current invention, a polypeptide may comprise an ACE2 domain, or a derivative or a fragment thereof, of an angiotensin converting enzyme 2; and an immunoglobulin domain. The ACE2 domain of these polypeptides is capable of binding to a coat protein of a virus selected from the group consisting of herpesviridae, papillomaviridae, coronaviridae, flaviviridae, togaviridae, bornaviridae, bunyaviridae, filoviridae, orthomyxoviridae, paramyxoviridae, pneumoviridae, and retroviridae. Table 6 provides a list of exemplary, but not limited to, immunoglobulin-ACE2 constructs.

TABLE 6
Immunoglobulin-ACE2 constructs (SEQ ID NOs: 54-56).

| Construct | | SEQ ID | | |
|---|---|---|---|---|
| No. | IgG Subtype | NO: | Length | Sequence |
| 1 | IgG1 Wt Fc - | 54 | 831 | TCPPCPAPELLGGPSVFLFPPKPKDTLMISRTP |
| | (G4S)₂-Full | | | EVTCVVVDVSHEDPEVKFNWYVDGVEVHNAKTK |
| | length ACE2 | | | PREEQYNSTYRVVSVLTVLHQDWLNGKEYKCKV |
| | | | | SNKALPAPIEKTISKAKGQPREPQVYTLPPSRD |
| | | | | ELTKNQVSLTCLVKGFYPSDIAVEWESNGQPEN |
| | | | | NYKTTPPVLDSDGSFFLYSKLTVDKSRWQQGNV |
| | | | | FSCSVMHEALHNHYTQKSLSLSPGKGGGGSGGG |
| | | | | GSQSTIEEQAKTFLDKFNHEAEDLFYQSSLASW |
| | | | | NYNTNITEENVQNMNNAGDKWSAFLKEQSTLAQ |
| | | | | MYPLQEIQNLTVKLQLQALQQNGSSVLSEDKSK |
| | | | | RLNTILNTMSTIYSTGKVCNPDNPQECLLLEPG |
| | | | | LNEIMANSLDYNERLWAWESWRSEVGKQLRPLY |
| | | | | EEYVVLKNEMARANHYEDYGDYWRGDYEVNGVD |
| | | | | GYDYSRGQLIEDVEHTFEEIKPLYEHLHAYVRA |
| | | | | KLMNAYPSYISPIGCLPAHLLGDMWGRFWTNLY |
| | | | | SLTVPFGQKPNIDVTDAMVDQAWDAQRIFKEAE |
| | | | | KFFVSVGLPNMTQGFWENSMLTDPGNVQKAVCH |
| | | | | PTAWDLGKGDFRILMCTKVTMDDFLTAHHEMGH |
| | | | | IQYDMAYAAQPFLLRNGANEGFHEAVGEIMSLS |
| | | | | AATPKHLKSIGLLSPDFQEDNETEINFLLKQAL |
| | | | | TIVGTLPFTYMLEKWRWMVFKGEIPKDQWMKKW |
| | | | | WEMKREIVGVVEPVPHDETYCDPASLFHVSNDY |
| | | | | SFIRYYTRTLYQFQFQEALCQAAKHEGPLHKCD |
| | | | | ISNSTEAGQKLFNMLRLGKSEPWTLALENVVGA |
| | | | | KNMNVRPLLNYFEPLFTWLKDQNKNSFVGWSTD |
| | | | | WSPYAD |
| 2 | IgG1 Wt Fc - | 55 | 305 | TCPPCPAPELLGGPSVFLFPPKPKDTLMISRTP |
| | (G4S)₂-Ace2- | | | EVTCVVVDVSHEDPEVKFNWYVDGVEVHNAKTK |
| | 1 | | | PREEQYNSTYRVVSVLTVLHQDWLNGKEYKCKV |
| | | | | SNKALPAPIEKTISKAKGQPREPQVYTLPPSRD |
| | | | | ELTKNQVSLTCLVKGFYPSDIAVEWESNGQPEN |
| | | | | NYKTTPPVLDSDGSFFLYSKLTVDKSRWQQGNV |
| | | | | FSCSVMHEALHNHYTQKSLSLSPGKGGGGSGGG |
| | | | | GSEEQAKTFLDKFNHEAEDLFYQSSGLGKGDFR |
| | | | | GGGGSGGGGSEEQAKTFLDKFNHEAEDLFYQSS |
| | | | | GLGKGDFR |
| 3 | IgG1 Wt Fc - | 56 | 397 | TCPPCPAPELLGGPSVFLFPPKPKDTLMISRTP |
| | (G4S)₂-Ace2- | | | EVTCVVVDVSHEDPEVKFNWYVDGVEVHNAKTK |
| | 1 | | | PREEQYNSTYRVVSVLTVLHQDWLNGKEYKCKV |
| | | | | SNKALPAPIEKTISKAKGQPREPQVYTLPPSRD |
| | | | | ELTKNQVSLTCLVKGFYPSDIAVEWESNGQPEN |
| | | | | NYKTTPPVLDSDGSFFLYSKLTVDKSRWQQGNV |
| | | | | FSCSVMHEALHNHYTQKSLSLSPGKGGGGSGGG |
| | | | | GSIEEQAKTFLDKFNHEAEDLFYQSSLASWNYN |
| | | | | TNITEENVONMNNAGDKWSAFLKEQSTLAQMYP |
| | | | | LQEIWDLGKGDFRGGGGSGGGGSIEEQAKTFLD |
| | | | | KFNHEAEDLFYQSSLASWNYNTNITEENVQNMN |
| | | | | NAGDKWSAFLKEQSTLAQMYPLQEIWDLGKGDF |
| | | | | R |

Immunoglobulin-Neuropilin Constructs

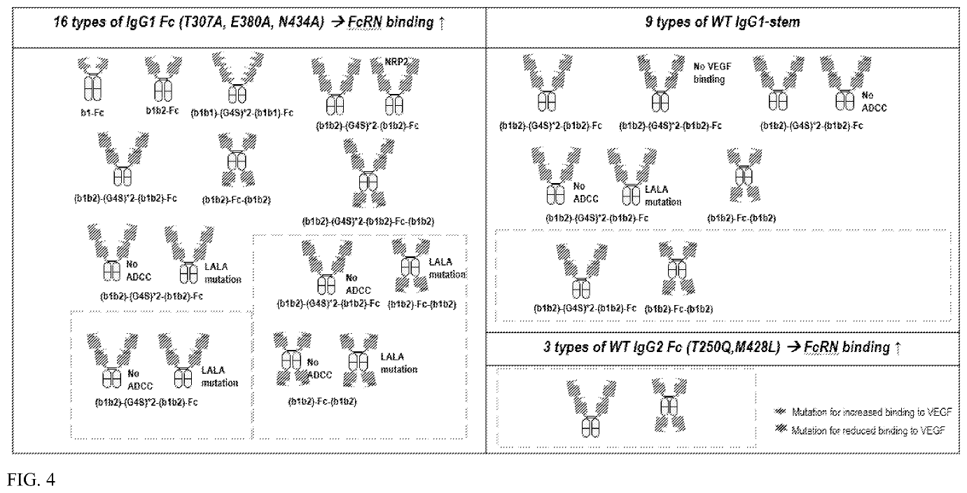

In another aspect of the current invention, a polypeptide may comprise a b1 domain, or a derivative or fragment thereof, of a neuropilin; and an immunoglobulin domain. The b1 domain of these polypeptides is capable of binding to a coat protein of a virus selected from the group consisting of herpesviridae, papillomaviridae, coronaviridae, flaviviridae, togaviridae, bornaviridae, bunyaviridae, filoviridae, orthomyxoviridae, paramyxoviridae, pneumoviridae, and retroviridae. In some embodiments, the b1 domain may include the full-length NRP1 or NRP2 peptide. In other embodiments, the b1 domain, or derivative or fragment thereof, further comprises one or more additional b1 domains, or derivative or fragment thereof; one or more b2 domains, or derivative or fragment thereof, of neuropilin, or a combination thereof. Referring to FIG. 4, a variety of exemplary schematic designs are provided for these polypeptides where the b1 domain, or a derivative or fragment thereof, is coupled to an immunoglobulin domain. Table 7A outlines the general structure and connectivity of several exemplary constructs, but not intended to be limited to, neuropilin-immunoglobulin constructs (Construct Nos. 12-36) or polypeptides including b1, linker, and immunoglobulin domains. In some embodiments, the polypeptide has a configuration selected from the group of Construct Nos. 12-36. Table 7B provides a list of these same exemplary constructs or neuropilin-immunoglobulin polypeptides including b1, linker, and immunoglobulin domains and their corresponding peptide sequences.

TABLE 7A
Immunoglobulin-Neuropilin constructs.

| Construct | | | | | C-term | | NRP | IgG |
|---|---|---|---|---|---|---|---|---|
| No. | N-term (NRP1) | Linker | Stem | Linker4 | (ACE2) | Note | type | Subtype |
| 12 | b1 | (G4S)2 | IgG1 Fc | — | — | | NRP1 | IgG1 |
| 13 | (b1b2) | (G4S)2 | | — | | | NRP1 | IgG1 |
| 14 | (b1b1)-(G4S)2-(b1b1) | (G4S)2 | | — | | | NRP1 | IgG1 |
| 15 | (b1b2)-(G4S)2-(b1b2) | (G4S)2 | | — | | | NRP1 | IgG1 |
| 16 | (b1b2)-(G4S)2-(b1b2) | (G4S)2 | | — | — | b1(E319A) | NRP1 | IgG1 |
| 17 | (b1b2) | (G4S)2 | | (G4S)2 | (b1b2) | b1(E319A) | NRP1 | IgG1 |
| 18 | (b1b2)-(G4S)2-(b1b2) | (G4S)2 | | (G4S)2 | (b1b2) | b1(E319A) | NRP1 | IgG1 |
| 19 | (b1b2)-(G4S)2-(b1b2) | (G4S)2 | | — | — | b1b2 of NRP2 | NRP2 | IgG1 |
| 20 | (b1b2)-(G4S)2-(b1b2) | (G4S)2 | | — | — | b1(E319A)-No ADCC | NRP1 | IgG1 |
| 21 | (b1b2) | (G4S)2 | | (G4S)2 | (b1b2) | b1(E319A)-No ADCC | NRP1 | IgG1 |
| 22 | (b1b2)-(G4S)2-(b1b2) | (G4S)2 | IgG1 Fc | — | — | WT Fc | NRP1 | IgG1 |
| 23 | (b1b2)-(G4S)2-(b1b2) | (G4S)2 | | — | — | b1(Y297A, S346A, Y353A)- | NRP1 | IgG1 |
| | | | | | | No binding to VEGF165A | | |
| 24 | (b1b2)-(G4S)2-(b1b2) | (G4S)2 | | — | — | No ADCC | NRP1 | IgG1 |
| 25 | (b1b2)-(G4S)2-(b1b2) | (G4S)2 | | — | — | LALA mutation | NRP1 | IgG1 |
| 26 | (b1b2)-(G4S)2-(b1b2) | (G4S)2 | | — | — | b1(E319A) | NRP1 | IgG1 |
| 27 | (b1b2)-(G4S)2-(b1b2) | (G4S)2 | | — | — | b1(E319A)-No ADCC | NRP1 | IgG1 |
| 28 | (b1b2) | (G4S)2 | | (G4S)2 | (b1b2) | | NRP1 | IgG1 |
| 29 | (b1b2)-(G4S)2-(b1b2) | (G4S)2 | | — | — | b1(K351A)-No ADCC | NRP1 | IgG1 |
| 30 | (b1b2)-(G4S)2-(b1b2) | (G4S)2 | | — | — | b1(K351A)-LALA | NRP1 | IgG1 |
| | | | | | | mutation | | |
| 31 | (b1b2)-(G4S)2-(b1b2) | (G4S)2 | | — | — | No ADCC | NRP1 | IgG1 |
| 32 | (b1b2)-(G4S)2-(b1b2) | (G4S)2 | | — | — | LALA mutation | NRP1 | IgG1 |
| 33 | (b1b2)-(G4S)2-(b1b2) | (G4S)2 | | — | — | b1(E319A)-LALA | NRP1 | IgG1 |
| | | | | | | mutation | | |
| 34 | (b1b2) | (G4S)2 | | (G4S)2 | (b1b2) | b1(E319A)-LALA | NRP1 | IgG1 |
| | | | | | | mutation | | |
| 35 | (b1b2)-(G4S)2-(b1b2) | (G4S)2 | IgG2 Fc | — | — | | NRP1 | IgG2 |
| 36 | (b1b2) | (G4S)2 | IgG2 Fc | (G4S)2 | (b1b2) | | NRP1 | IgG2 |

“ADCC” refers to Antibody-dependent cellular cytotoxicity (ADCC).
“NA” means not applicable.

TABLE 7B
Immunoglobulin-Neuropilin constructs (SEQ ID NOs: 65-89).

| Con- | SEQ | | |
|---|---|---|---|
| struct | ID | | |
| No. | NO: | Length | Sequence |
| 12 | 65 | 392 | CMEALGMESGEIHSDQITASSQYSTNWSAERSRLNYPENGWTPGEDSYREWIQVDLGLLRFVTAVGTQGAISKETKKKYYVK |
| | | | TYKIDVSSNGEDWITIKEGNKPVLFQGNTNPTDVVVAVFPKPLITRFVRIKPATWETGISMRFEVYGCGGGGSGGGGSEPKS |
| | | | CDKTHTCPPCPAPELLGGPSVFLFPPKPKDTLMISRTPEVTCVVVDVSHEDPEVKFNWYVDGVEVHNAKTKPREEQYNSTYR |
| | | | VVSVLAVLHQDWLNGKEYKCKVSNKALPAPIEKTISKAKGQPREPQVYTLPPSRDELTKNQVSLTCLVKGFYPSDIAVAWES |
| | | | NGQPENNYKTTPPVLDSDGSFFLYSKLTVDKSRWQQGNVFSCSVMHEALHAHYTQKSLSLSPGK |
| 13 | 66 | 551 | CMEALGMESGEIHSDQITASSQYSTNWSAERSRLNYPENGWTPGEDSYREWIQVDLGLLRFVTAVGTQGAISKETKKKYYVK |
| | | | TYKIDVSSNGEDWITIKEGNKPVLFQGNTNPTDVVVAVFPKPLITRFVRIKPATWETGISMRFEVYGCKITDYPCSGMLGMV |
| | | | SGLISDSQITSSNQGDRNWMPENIRLVTSRSGWALPPAPHSYINEWLQIDLGEEKIVRGIIIQGGKHRENKVFMRKFKIGYS |
| | | | NNGSDWKMIMDDSKRKAKSFEGNNNYDTPELRTFPALSTRFIRIYPERATHGGLGLRMELLGCGGGGSGGGGSEPKSCDKTH |
| | | | TCPPCPAPELLGGPSVFLFPPKPKDTLMISRTPEVTCVVVDVSHEDPEVKFNWYVDGVEVHNAKTKPREEQYNSTYRVVSVL |
| | | | AVLHQDWLNGKEYKCKVSNKALPAPIEKTISKAKGQPREPQVYTLPPSRDELTKNQVSLTCLVKGFYPSDIAVAWESNGQPE |
| | | | NNYKTTPPVLDSDGSFFLYSKLTVDKSRWQQGNVFSCSVMHEALHAHYTQKSLSLSPGK |
| 14 | 67 | 864 | CMEALGMESGEIHSDQITASSQYSTNWSAERSRLNYPENGWTPGEDSYREWIQVDLGLLRFVTAVGTQGAISKETKKKYYVK |
| | | | TYKIDVSSNGEDWITIKEGNKPVLFQGNTNPTDVVVAVFPKPLITRFVRIKPATWETGISMRFEVYGCKITDYPCMEALGME |
| | | | SGEIHSDOITASSQYSTNWSAERSRLNYPENGWTPGEDSYREWIQVDLGLLRFVTAVGTQGAISKETKKKYYVKTYKIDVSS |
| | | | HSDQITASSQYSTNWSAERSRLNYPENGWTPGEDSYREWIQVDLGLLRFVTAVGTQGAISKETKKKYYVKTYKIDVSSNGED |
| | | | WITIKEGNKPVLFQGNTNPTDVVVAVFPKPLITRFVRIKPATWETGISMRFEVYGCKITDYPCMEALGMESGEIHSDQITAS |
| | | | SQYSTNWSAERSRLNYPENGWTPGEDSYREWIQVDLGLLRFVTAVGTQGAISKETKKKYYVKTYKIDVSSNGEDWITIKEGN |
| | | | KPVLFQGNTNPTDVVVAVFPKPLITRFVRIKPATWETGISMRFEVYGCGGGGSGGGGSEPKSCDKTHTCPPCPAPELLGGPS |
| | | | VFLFPPKPKDTLMISRTPEVTCVVVDVSHEDPEVKFNWYVDGVEVHNAKTKPREEQYNSTYRVVSVLAVLHQDWLNGKEYKC |
| | | | KVSNKALPAPIEKTISKAKGQPREPQVYTLPPSRDELTKNQVSLTCLVKGFYPSDIAVAWESNGQPENNYKTTPPVLDSDGS |
| | | | FFLYSKLTVDKSRWQQGNVFSCSVMHEALHAHYTQKSLSLSPGK |
| 15 | 68 | 870 | CMEALGMESGEIHSDOITASSQYSTNWSAERSRLNYPENGWTPGEDSYREWIQVDLGLLRFVTAVGTQGAISKETKKKYYVK |
| | | | TYKIDVSSNGEDWITIKEGNKPVLFQGNTNPTDVVVAVFPKPLITRFVRIKPATWETGISMRFEVYGCKITDYPCSGMLGMV |
| | | | SGLISDSQITSSNQGDRNWMPENIRLVTSRSGWALPPAPHSYINEWLQIDLGEEKIVRGIIIQGGKHRENKVFMRKFKIGYS |
| | | | NNGSDWKMIMDDSKRKAKSFEGNNNYDTPELRTFPALSTRFIRIYPERATHGGLGLRMELLGCGGGGSGGGGSCMEALGMES |
| | | | GEIHSDQITASSQYSTNWSAERSRLNYPENGWTPGEDSYREWIQVDLGLLRFVTAVGTQGAISKETKKKYYVKTYKIDVSSN |
| | | | TSSNQGDRNWMPENIRLVTSRSGWALPPAPHSYINEWLQIDLGEEKIVRGIIIQGGKHRENKVFMRKFKIGYSNNGSDWKMI |
| | | | MDDSKRKAKSFEGNNNYDTPELRTFPALSTRFIRIYPERATHGGLGLRMELLGCGGGGSGGGGSEPKSCDKTHTCPPCPAPE |
| | | | LLGGPSVFLFPPKPKDTLMISRTPEVTCVVVDVSHEDPEVKFNWYVDGVEVHNAKTKPREEQYNSTYRVVSVLAVLHQDWLN |
| | | | GKEYKCKVSNKALPAPIEKTISKAKGQPREPQVYTLPPSRDELTKNQVSLTCLVKGFYPSDIAVAWESNGQPENNYKTTPPV |
| | | | LDSDGSFFLYSKLTVDKSRWQQGNVFSCSVMHEALHAHYTQKSLSLSPGK |
| 16 | 69 | 870 | CMEALGMESGEIHSDQITASSQYSTNWSAERSRLNYPENGWTPGADSYREWIQVDLGLLRFVTAVGTQGAISKETKKKYYVK |
| | | | TYKIDVSSNGEDWITIKEGNKPVLFQGNTNPTDVVVAVFPKPLITRFVRIKPATWETGISMRFEVYGCKITDYPCSGMLGMV |
| | | | SGLISDSQITSSNQGDRNWMPENIRLVTSRSGWALPPAPHSYINEWLQIDLGEEKIVRGIIIQGGKHRENKVFMRKFKIGYS |
| | | | NNGSDWKMIMDDSKRKAKSFEGNNNYDTPELRTFPALSTRFIRIYPERATHGGLGLRMELLGCGGGGSGGGGSCMEALGMES |
| | | | GEIHSDQITASSQYSTNWSAERSRLNYPENGWTPGADSYREWIQVDLGLLRFVTAVGTQGAISKETKKKYYVKTYKIDVSSN |
| | | | TSSNQGDRNWMPENIRLVTSRSGWALPPAPHSYINEWLQIDLGEEKIVRGIIIQGGKHRENKVFMRKFKIGYSNNGSDWKMI |
| | | | MDDSKRKAKSFEGNNNYDTPELRTFPALSTRFIRIYPERATHGGLGLRMELLGCGGGGSGGGGSEPKSCDKTHTCPPCPAPE |
| | | | LLGGPSVFLFPPKPKDTLMISRTPEVTCVVVDVSHEDPEVKFNWYVDGVEVHNAKTKPREEQYNSTYRVVSVLAVLHQDWLN |
| | | | GKEYKCKVSNKALPAPIEKTISKAKGQPREPQVYTLPPSRDELTKNQVSLTCLVKGFYPSDIAVAWESNGQPENNYKTTPPV |
| | | | LDSDGSFFLYSKLTVDKSRWQQGNVFSCSVMHEALHAHYTQKSLSLSPGK |
| 17 | 70 | 874 | CMEALGMESGEIHSDQITASSQYSTNWSAERSRLNYPENGWTPGADSYREWIQVDLGLLRFVTAVGTQGAISKETKKKYYVK |
| | | | TYKIDVSSNGEDWITIKEGNKPVLFQGNTNPTDVVVAVFPKPLITRFVRIKPATWETGISMRFEVYGCKITDYPCSGMLGMV |
| | | | SGLISDSQITSSNQGDRNWMPENIRLVTSRSGWALPPAPHSYINEWLQIDLGEEKIVRGIIIQGGKHRENKVFMRKFKIGYS |
| | | | NNGSDWKMIMDDSKRKAKSFEGNNNYDTPELRTFPALSTRFIRIYPERATHGGLGLRMELLGCGGGGSGGGGSEPKSCDKTH |
| | | | TCPPCPAPELLGGPSVFLFPPKPKDTLMISRTPEVTCVVVDVSHEDPEVKFNWYVDGVEVHNAKTKPREEQYNSTYRVVSVL |
| | | | AVLHQDWLNGKEYKCKVSNKALPAPIEKTISKAKGQPREPQVYTLPPSRDELTKNQVSLTCLVKGFYPSDIAVAWESNGQPE |
| | | | NNYKTTPPVLDSDGSFFLYSKLTVDKSRWQQGNVFSCSVMHEALHAHYTQKSLSLSPGKGGGGSGGGGSCMEALGMESGEIH |
| | | | SDQITASSQYSTNWSAERSRLNYPENGWTPGADSYREWIQVDLGLLRFVTAVGTQGAISKETKKKYYVKTYKIDVSSNGEDW |
| | | | ITIKEGNKPVLFQGNTNPTDVVVAVFPKPLITRFVRIKPATWETGISMRFEVYGCKITDYPCSGMLGMVSGLISDSQITSSN |
| | | | QGDRNWMPENIRLVTSRSGWALPPAPHSYINEWLQIDLGEEKIVRGIIIQGGKHRENKVFMRKFKIGYSNNGSDWKMIMDDS |
| | | | KRKAKSFEGNNNYDTPELRTFPALSTRFIRIYPERATHGGLGLRMELLGCEVEA |
| 18 | 71 | 1193 | CMEALGMESGEIHSDQITASSQYSTNWSAERSRLNYPENGWTPGADSYREWIQVDLGLLRFVTAVGTQGAISKETKKKYYVK |
| | | | TYKIDVSSNGEDWITIKEGNKPVLFQGNTNPTDVVVAVFPKPLITRFVRIKPATWETGISMRFEVYGCKITDYPCSGMLGMV |
| | | | SGLISDSQITSSNQGDRNWMPENIRLVTSRSGWALPPAPHSYINEWLQIDLGEEKIVRGIIIQGGKHRENKVFMRKFKIGYS |
| | | | NNGSDWKMIMDDSKRKAKSFEGNNNYDTPELRTFPALSTRFIRIYPERATHGGLGLRMELLGCGGGGSGGGGSCMEALGMES |
| | | | GEIHSDQITASSQYSTNWSAERSRLNYPENGWTPGADSYREWIQVDLGLLRFVTAVGTQGAISKETKKKYYVKTYKIDVSSN |
| | | | GEDWITIKEGNKPVLFQGNTNPTDVVVAVFPKPLITRFVRIKPATWETGISMRFEVYGCKITDYPCSGMLGMVSGLISDSQI |
| | | | TSSNQGDRNWMPENIRLVTSRSGWALPPAPHSYINEWLQIDLGEEKIVRGIIIQGGKHRENKVFMRKFKIGYSNNGSDWKMI |
| | | | MDDSKRKAKSFEGNNNYDTPELRTFPALSTRFIRIYPERATHGGLGLRMELLGCGGGGSGGGGSEPKSCDKTHTCPPCPAPE |
| | | | LLGGPSVFLFPPKPKDTLMISRTPEVTCVVVDVSHEDPEVKFNWYVDGVEVHNAKTKPREEQYNSTYRVVSVLAVLHQDWLN |
| | | | GKEYKCKVSNKALPAPIEKTISKAKGQPREPQVYTLPPSRDELTKNQVSLTCLVKGFYPSDIAVAWESNGQPENNYKTTPPV |
| | | | LDSDGSFFLYSKLTVDKSRWQQGNVFSCSVMHEALHAHYTQKSLSLSPGKGGGGSGGGGSCMEALGMESGEIHSDQITASSQ |
| | | | VLFQGNTNPTDVVVAVFPKPLITRFVRIKPATWETGISMRFEVYGCKITDYPCSGMLGMVSGLISDSQITSSNQGDRNWMPE |
| | | | NIRLVTSRSGWALPPAPHSYINEWLQIDLGEEKIVRGIIIQGGKHRENKVFMRKFKIGYSNNGSDWKMIMDDSKRKAKSFEG |
| | | | NNNYDTPELRTFPALSTRFIRIYPERATHGGLGLRMELLGCEVEA |
| 19 | 72 | 884 | CNVPLGMESGRIANEQISASSTYSDGRWTPQQSRLHGDDNGWTPNLDSNKEYLQVDLRFLTMLTAIATQGAISRETQNGYYV |
| | | | KSYKLEVSTNGEDWMVYRHGKNHKVFQANNDATEVVLNKLHAPLLTRFVRIRPQTWHSGIALRLELFGCRVTDAPCSNMLGM |
| | | | LSGLIADSQISASSTQEYLWSPSAARLVSSRSGWFPRIPQAQPGEEWLQVDLGTPKTVKGVIIQGARGGDSITAVEARAFVR |
| | | | KFKVSYSLNGKDWEYIQDPRTQQPKLFEGNMHYDTPDIRRFDPIPAQYVRVYPERWSPAGIGMRLEVLGCGGGGSGGGGSCN |
| | | | VPLGMESGRIANEQISASSTYSDGRWTPQQSRLHGDDNGWTPNLDSNKEYLQVDLRFLTMLTAIATQGAISRETQNGYYVKS |
| | | | YKLEVSTNGEDWMVYRHGKNHKVFQANNDATEVVLNKLHAPLLTRFVRIRPQTWHSGIALRLELFGCRVTDAPCSNMLGMLS |
| | | | GLIADSQISASSTQEYLWSPSAARLVSSRSGWFPRIPQAQPGEEWLQVDLGTPKTVKGVIIQGARGGDSITAVEARAFVRKF |
| | | | KVSYSLNGKDWEYIQDPRTQQPKLFEGNMHYDTPDIRRFDPIPAQYVRVYPERWSPAGIGMRLEVLGCGGGGSGGGGSEPKS |
| | | | CDKTHTCPPCPAPELLGGPSVFLFPPKPKDTLMISRTPEVTCVVVDVSHEDPEVKFNWYVDGVEVHNAKTKPREEQYNSTYR |
| | | | VVSVLAVLHQDWLNGKEYKCKVSNKALPAPIEKTISKAKGQPREPQVYTLPPSRDELTKNQVSLTCLVKGFYPSDIAVAWES |
| | | | NGQPENNYKTTPPVLDSDGSFFLYSKLTVDKSRWQQGNVFSCSVMHEALHAHYTQKSLSLSPGK |
| 20 | 73 | 870 | CMEALGMESGEIHSDQITASSQYSTNWSAERSRLNYPENGWTPGADSYREWIQVDLGLLRFVTAVGTQGAISKETKKKYYVK |
| | | | TYKIDVSSNGEDWITIKEGNKPVLFQGNTNPTDVVVAVFPKPLITRFVRIKPATWETGISMRFEVYGCKITDYPCSGMLGMV |
| | | | SGLISDSQITSSNQGDRNWMPENIRLVTSRSGWALPPAPHSYINEWLQIDLGEEKIVRGIIIQGGKHRENKVFMRKFKIGYS |
| | | | NNGSDWKMIMDDSKRKAKSFEGNNNYDTPELRTFPALSTRFIRIYPERATHGGLGLRMELLGCGGGGSGGGGSCMEALGMES |
| | | | GEIHSDOITASSQYSTNWSAERSRLNYPENGWTPGADSYREWIQVDLGLLRFVTAVGTQGAISKETKKKYYVKTYKIDVSSN |
| | | | TSSNQGDRNWMPENIRLVTSRSGWALPPAPHSYINEWLQIDLGEEKIVRGIIIQGGKHRENKVFMRKFKIGYSNNGSDWKMI |
| | | | MDDSKRKAKSFEGNNNYDTPELRTFPALSTRFIRIYPERATHGGLGLRMELLGCGGGGSGGGGSEPKSCDKTHTCPPCPAPE |
| | | | LLGGPSVFLFPPKPKDTLMISRTPEVTCVVVDVSHEDPEVKFNWYVDGVEVHNAKTKPREEQYASTYRVVSVLAVLHQDWLN |
| | | | GKEYKCKVSNKALPAPIEKTISKAKGQPREPQVYTLPPSRDELTKNQVSLTCLVKGFYPSDIAVAWESNGQPENNYKTTPPV |
| | | | LDSDGSFFLYSKLTVDKSRWQQGNVFSCSVMHEALHAHYTQKSLSLSPGK |
| 21 | 74 | 874 | CMEALGMESGEIHSDOITASSQYSTNWSAERSRLNYPENGWTPGADSYREWIQVDLGLLRFVTAVGTQGAISKETKKKYYVK |
| | | | TYKIDVSSNGEDWITIKEGNKPVLFQGNTNPTDVVVAVFPKPLITRFVRIKPATWETGISMRFEVYGCKITDYPCSGMLGMV |
| | | | SGLISDSQITSSNQGDRNWMPENIRLVTSRSGWALPPAPHSYINEWLQIDLGEEKIVRGIIIQGGKHRENKVFMRKFKIGYS |
| | | | NNGSDWKMIMDDSKRKAKSFEGNNNYDTPELRTFPALSTRFIRIYPERATHGGLGLRMELLGCGGGGSGGGGSEPKSCDKTH |
| | | | TCPPCPAPELLGGPSVFLFPPKPKDTLMISRTPEVTCVVVDVSHEDPEVKFNWYVDGVEVHNAKTKPREEQYASTYRVVSVL |
| | | | AVLHQDWLNGKEYKCKVSNKALPAPIEKTISKAKGQPREPQVYTLPPSRDELTKNQVSLTCLVKGFYPSDIAVAWESNGQPE |
| | | | NNYKTTPPVLDSDGSFFLYSKLTVDKSRWQQGNVFSCSVMHEALHAHYTQKSLSLSPGKGGGGSGGGGSCMEALGMESGEIH |
| | | | SDQITASSQYSTNWSAERSRLNYPENGWTPGADSYREWIQVDLGLLRFVTAVGTQGAISKETKKKYYVKTYKIDVSSNGEDW |
| | | | ITIKEGNKPVLFQGNTNPTDVVVAVFPKPLITRFVRIKPATWETGISMRFEVYGCKITDYPCSGMLGMVSGLISDSQITSSN |
| | | | QGDRNWMPENIRLVTSRSGWALPPAPHSYINEWLQIDLGEEKIVRGIIIQGGKHRENKVFMRKFKIGYSNNGSDWKMIMDDS |
| | | | KRKAKSFEGNNNYDTPELRTFPALSTRFIRIYPERATHGGLGLRMELLGCEVEA |
| 22 | | 870 | CMEALGMESGEIHSDQITASSQYSTNWSAERSRLNYPENGWTPGEDSYREWIQVDLGLLRFVTAVGTQGAISKETKKKYYVK |
| | | | TYKIDVSSNGEDWITIKEGNKPVLFQGNTNPTDVVVAVFPKPLITRFVRIKPATWETGISMRFEVYGCKITDYPCSGMLGMV |
| | | | SGLISDSQITSSNQGDRNWMPENIRLVTSRSGWALPPAPHSYINEWLQIDLGEEKIVRGIIIQGGKHRENKVFMRKFKIGYS |
| | | | NNGSDWKMIMDDSKRKAKSFEGNNNYDTPELRTFPALSTRFIRIYPERATHGGLGLRMELLGCGGGGSGGGGSCMEALGMES |
| | | | GEIHSDQITASSQYSTNWSAERSRLNYPENGWTPGEDSYREWIQVDLGLLRFVTAVGTQGAISKETKKKYYVKTYKIDVSSN |
| | | | GEDWITIKEGNKPVLFQGNTNPTDVVVAVFPKPLITRFVRIKPATWETGISMRFEVYGCKITDYPCSGMLGMVSGLISDSQI |
| | | | TSSNQGDRNWMPENIRLVTSRSGWALPPAPHSYINEWLQIDLGEEKIVRGIIIQGGKHRENKVFMRKFKIGYSNNGSDWKMI |
| | | | MDDSKRKAKSFEGNNNYDTPELRTFPALSTRFIRIYPERATHGGLGLRMELLGCGGGGSGGGGSEPKSCDKTHTCPPCPAPE |
| | | | LLGGPSVFLFPPKPKDTLMISRTPEVTCVVVDVSHEDPEVKFNWYVDGVEVHNAKTKPREEQYNSTYRVVSVLTVLHQDWLN |
| | | | GKEYKCKVSNKALPAPIEKTISKAKGQPREPQVYTLPPSRDELTKNQVSLTCLVKGFYPSDIAVEWESNGQPENNYKTTPPV |
| | | | LDSDGSFFLYSKLTVDKSRWQQGNVFSCSVMHEALHNHYTQKSLSLSPGK |
| 23 | 76 | 870 | CMEALGMESGEIHSDQITASSQASTNWSAERSRLNYPENGWTPGEDSYREWIQVDLGLLRFVTAVGTQGAIAKETKKKAYVK |
| | | | TYKIDVSSNGEDWITIKEGNKPVLFQGNTNPTDVVVAVFPKPLITRFVRIKPATWETGISMRFEVYGCKITDYPCSGMLGMV |
| | | | SGLISDSQITSSNQGDRNWMPENIRLVTSRSGWALPPAPHSYINEWLQIDLGEEKIVRGIIIQGGKHRENKVFMRKFKIGYS |
| | | | NNGSDWKMIMDDSKRKAKSFEGNNNYDTPELRTFPALSTRFIRIYPERATHGGLGLRMELLGCGGGGSGGGGSCMEALGMES |
| | | | GEIHSDQITASSQASTNWSAERSRLNYPENGWTPGEDSYREWIQVDLGLLRFVTAVGTQGAIAKETKKKAYVKTYKIDVSSN |
| | | | GEDWITIKEGNKPVLFQGNTNPTDVVVAVFPKPLITRFVRIKPATWETGISMRFEVYGCKITDYPCSGMLGMVSGLISDSQI |
| | | | TSSNQGDRNWMPENIRLVTSRSGWALPPAPHSYINEWLQIDLGEEKIVRGIIIQGGKHRENKVFMRKFKIGYSNNGSDWKMI |
| | | | MDDSKRKAKSFEGNNNYDTPELRTFPALSTRFIRIYPERATHGGLGLRMELLGCGGGGSGGGGSEPKSCDKTHTCPPCPAPE |
| | | | LLGGPSVFLFPPKPKDTLMISRTPEVTCVVVDVSHEDPEVKFNWYVDGVEVHNAKTKPREEQYNSTYRVVSVLTVLHQDWLN |
| | | | GKEYKCKVSNKALPAPIEKTISKAKGQPREPQVYTLPPSRDELTKNQVSLTCLVKGFYPSDIAVEWESNGQPENNYKTTPPV |
| | | | LDSDGSFFLYSKLTVDKSRWQQGNVFSCSVMHEALHNHYTQKSLSLSPGK |
| 24 | 77 | 870 | CMEALGMESGEIHSDQITASSQYSTNWSAERSRLNYPENGWTPGEDSYREWIQVDLGLLRFVTAVGTQGAISKETKKKYYVK |
| | | | TYKIDVSSNGEDWITIKEGNKPVLFQGNTNPTDVVVAVFPKPLITRFVRIKPATWETGISMRFEVYGCKITDYPCSGMLGMV |
| | | | SGLISDSQITSSNQGDRNWMPENIRLVTSRSGWALPPAPHSYINEWLQIDLGEEKIVRGIIIQGGKHRENKVFMRKFKIGYS |
| | | | NNGSDWKMIMDDSKRKAKSFEGNNNYDTPELRTFPALSTRFIRIYPERATHGGLGLRMELLGCGGGGSGGGGSCMEALGMES |
| | | | GEIHSDQITASSQYSTNWSAERSRLNYPENGWTPGEDSYREWIQVDLGLLRFVTAVGTQGAISKETKKKYYVKTYKIDVSSN |
| | | | TSSNQGDRNWMPENIRLVTSRSGWALPPAPHSYINEWLQIDLGEEKIVRGIIIQGGKHRENKVFMRKFKIGYSNNGSDWKMI |
| | | | MDDSKRKAKSFEGNNNYDTPELRTFPALSTRFIRIYPERATHGGLGLRMELLGCGGGGSGGGGSEPKSCDKTHTCPPCPAPE |
| | | | LLGGPSVFLFPPKPKDTLMISRTPEVTCVVVDVSHEDPEVKFNWYVDGVEVHNAKTKPREEQYASTYRVVSVLTVLHQDWLN |
| | | | GKEYKCKVSNKALPAPIEKTISKAKGQPREPQVYTLPPSRDELTKNQVSLTCLVKGFYPSDIAVEWESNGQPENNYKTTPPV |
| | | | LDSDGSFFLYSKLTVDKSRWQQGNVFSCSVMHEALHNHYTQKSLSLSPGK |
| 25 | 78 | 870 | CMEALGMESGEIHSDQITASSQYSTNWSAERSRLNYPENGWTPGEDSYREWIQVDLGLLRFVTAVGTQGAISKETKKKYYVK |
| | | | TYKIDVSSNGEDWITIKEGNKPVLFQGNTNPTDVVVAVFPKPLITRFVRIKPATWETGISMRFEVYGCKITDYPCSGMLGMV |
| | | | SGLISDSQITSSNQGDRNWMPENIRLVTSRSGWALPPAPHSYINEWLQIDLGEEKIVRGIIIQGGKHRENKVFMRKFKIGYS |
| | | | NNGSDWKMIMDDSKRKAKSFEGNNNYDTPELRTFPALSTRFIRIYPERATHGGLGLRMELLGCGGGGSGGGGSCMEALGMES |
| | | | GEIHSDQITASSQYSTNWSAERSRLNYPENGWTPGEDSYREWIQVDLGLLRFVTAVGTQGAISKETKKKYYVKTYKIDVSSN |
| | | | TSSNQGDRNWMPENIRLVTSRSGWALPPAPHSYINEWLQIDLGEEKIVRGIIIQGGKHRENKVFMRKFKIGYSNNGSDWKMI |
| | | | MDDSKRKAKSFEGNNNYDTPELRTFPALSTRFIRIYPERATHGGLGLRMELLGCGGGGSGGGGSEPKSCDKTHTCPPCPAPE |
| | | | AAGGPSVFLFPPKPKDTLMISRTPEVTCVVVDVSHEDPEVKFNWYVDGVEVHNAKTKPREEQYNSTYRVVSVLTVLHQDWLN |
| | | | GKEYKCKVSNKALPAPIEKTISKAKGQPREPQVYTLPPSRDELTKNQVSLTCLVKGFYPSDIAVEWESNGQPENNYKTTPPV |
| | | | LDSDGSFFLYSKLTVDKSRWQQGNVFSCSVMHEALHNHYTQKSLSLSPGK |
| 26 | 79 | 870 | CMEALGMESGEIHSDQITASSQYSTNWSAERSRLNYPENGWTPGADSYREWIQVDLGLLRFVTAVGTQGAISKETKKKYYVK |
| | | | TYKIDVSSNGEDWITIKEGNKPVLFQGNTNPTDVVVAVFPKPLITRFVRIKPATWETGISMRFEVYGCKITDYPCSGMLGMV |
| | | | SGLISDSQITSSNQGDRNWMPENIRLVTSRSGWALPPAPHSYINEWLQIDLGEEKIVRGIIIQGGKHRENKVFMRKFKIGYS |
| | | | NNGSDWKMIMDDSKRKAKSFEGNNNYDTPELRTFPALSTRFIRIYPERATHGGLGLRMELLGCGGGGSGGGGSCMEALGMES |
| | | | GEIHSDQITASSQYSTNWSAERSRLNYPENGWTPGADSYREWIQVDLGLLRFVTAVGTQGAISKETKKKYYVKTYKIDVSSN |
| | | | GEDWITIKEGNKPVLFQGNTNPTDVVVAVFPKPLITRFVRIKPATWETGISMRFEVYGCKITDYPCSGMLGMVSGLISDSQI |
| | | | TSSNQGDRNWMPENIRLVTSRSGWALPPAPHSYINEWLQIDLGEEKIVRGIIIQGGKHRENKVFMRKFKIGYSNNGSDWKMI |
| | | | MDDSKRKAKSFEGNNNYDTPELRTFPALSTRFIRIYPERATHGGLGLRMELLGCGGGGSGGGGSEPKSCDKTHTCPPCPAPE |
| | | | LLGGPSVFLFPPKPKDTLMISRTPEVTCVVVDVSHEDPEVKFNWYVDGVEVHNAKTKPREEQYNSTYRVVSVLTVLHQDWLN |
| | | | GKEYKCKVSNKALPAPIEKTISKAKGQPREPQVYTLPPSRDELTKNQVSLTCLVKGFYPSDIAVEWESNGQPENNYKTTPPV |
| | | | LDSDGSFFLYSKLTVDKSRWQQGNVFSCSVMHEALHNHYTQKSLSLSPGK |
| 27 | 80 | 870 | CMEALGMESGEIHSDQITASSQYSTNWSAERSRLNYPENGWTPGADSYREWIQVDLGLLRFVTAVGTQGAISKETKKKYYVK |
| | | | TYKIDVSSNGEDWITIKEGNKPVLFQGNTNPTDVVVAVFPKPLITRFVRIKPATWETGISMRFEVYGCKITDYPCSGMLGMV |
| | | | SGLISDSQITSSNQGDRNWMPENIRLVTSRSGWALPPAPHSYINEWLQIDLGEEKIVRGIIIQGGKHRENKVFMRKFKIGYS |
| | | | NNGSDWKMIMDDSKRKAKSFEGNNNYDTPELRTFPALSTRFIRIYPERATHGGLGLRMELLGCGGGGSGGGGSCMEALGMES |
| | | | GEIHSDQITASSQYSTNWSAERSRLNYPENGWTPGADSYREWIQVDLGLLRFVTAVGTQGAISKETKKKYYVKTYKIDVSSN |
| | | | GEDWITIKEGNKPVLFQGNTNPTDVVVAVFPKPLITRFVRIKPATWETGISMRFEVYGCKITDYPCSGMLGMVSGLISDSQI |
| | | | TSSNQGDRNWMPENIRLVTSRSGWALPPAPHSYINEWLQIDLGEEKIVRGIIIQGGKHRENKVFMRKFKIGYSNNGSDWKMI |
| | | | MDDSKRKAKSFEGNNNYDTPELRTFPALSTRFIRIYPERATHGGLGLRMELLGCGGGGSGGGGSEPKSCDKTHTCPPCPAPE |
| | | | LLGGPSVFLFPPKPKDTLMISRTPEVTCVVVDVSHEDPEVKFNWYVDGVEVHNAKTKPREEQYASTYRVVSVLTVLHQDWLN |
| | | | GKEYKCKVSNKALPAPIEKTISKAKGQPREPQVYTLPPSRDELTKNQVSLTCLVKGFYPSDIAVEWESNGQPENNYKTTPPV |
| | | | LDSDGSFFLYSKLTVDKSRWQQGNVFSCSVMHEALHNHYTQKSLSLSPGK |
| 28 | 81 | 874 | CMEALGMESGEIHSDQITASSQYSTNWSAERSRLNYPENGWTPGEDSYREWIQVDLGLLRFVTAVGTQGAISKETKKKYYVK |
| | | | SGLISDSQITSSNQGDRNWMPENIRLVTSRSGWALPPAPHSYINEWLQIDLGEEKIVRGIIIQGGKHRENKVFMRKFKIGYS |
| | | | NNGSDWKMIMDDSKRKAKSFEGNNNYDTPELRTFPALSTRFIRIYPERATHGGLGLRMELLGCGGGGSGGGGSEPKSCDKTH |
| | | | TCPPCPAPELLGGPSVFLFPPKPKDTLMISRTPEVTCVVVDVSHEDPEVKFNWYVDGVEVHNAKTKPREEQYNSTYRVVSVL |
| | | | TVLHQDWLNGKEYKCKVSNKALPAPIEKTISKAKGQPREPQVYTLPPSRDELTKNQVSLTCLVKGFYPSDIAVEWESNGQPE |
| | | | NNYKTTPPVLDSDGSFFLYSKLTVDKSRWQQGNVFSCSVMHEALHNHYTQKSLSLSPGKGGGGSGGGGSCMEALGMESGEIH |
| | | | SDQITASSQYSTNWSAERSRLNYPENGWTPGEDSYREWIQVDLGLLRFVTAVGTQGAISKETKKKYYVKTYKIDVSSNGEDW |
| | | | QGDRNWMPENIRLVTSRSGWALPPAPHSYINEWLQIDLGEEKIVRGIIIQGGKHRENKVFMRKFKIGYSNNGSDWKMIMDDS |
| | | | KRKAKSFEGNNNYDTPELRTFPALSTRFIRIYPERATHGGLGLRMELLGCEVEA |
| 29 | 82 | 870 | CMEALGMESGEIHSDQITASSQYSTNWSAERSRLNYPENGWTPGEDSYREWIQVDLGLLRFVTAVGTQGAISKETKAKYYVK |
| | | | TYKIDVSSNGEDWITIKEGNKPVLFQGNTNPTDVVVAVFPKPLITRFVRIKPATWETGISMRFEVYGCKITDYPCSGMLGMV |
| | | | SGLISDSQITSSNQGDRNWMPENIRLVTSRSGWALPPAPHSYINEWLQIDLGEEKIVRGIIIQGGKHRENKVFMRKFKIGYS |
| | | | NNGSDWKMIMDDSKRKAKSFEGNNNYDTPELRTFPALSTRFIRIYPERATHGGLGLRMELLGCGGGGSGGGGSCMEALGMES |
| | | | GEIHSDQITASSQYSTNWSAERSRLNYPENGWTPGEDSYREWIQVDLGLLRFVTAVGTQGAISKETKAKYYVKTYKIDVSSN |
| | | | TSSNQGDRNWMPENIRLVTSRSGWALPPAPHSYINEWLQIDLGEEKIVRGIIIQGGKHRENKVFMRKFKIGYSNNGSDWKMI |
| | | | MDDSKRKAKSFEGNNNYDTPELRTFPALSTRFIRIYPERATHGGLGLRMELLGCGGGGSGGGGSEPKSCDKTHTCPPCPAPE |
| | | | LLGGPSVFLFPPKPKDTLMISRTPEVTCVVVDVSHEDPEVKFNWYVDGVEVHNAKTKPREEQYASTYRVVSVLAVLHQDWLN |
| | | | GKEYKCKVSNKALPAPIEKTISKAKGQPREPQVYTLPPSRDELTKNQVSLTCLVKGFYPSDIAVAWESNGQPENNYKTTPPV |
| | | | LDSDGSFFLYSKLTVDKSRWQQGNVFSCSVMHEALHAHYTQKSLSLSPGK |
| 30 | 83 | 870 | CMEALGMESGEIHSDQITASSQYSTNWSAERSRLNYPENGWTPGEDSYREWIQVDLGLLRFVTAVGTQGAISKETKAKYYVK |
| | | | TYKIDVSSNGEDWITIKEGNKPVLFQGNTNPTDVVVAVFPKPLITRFVRIKPATWETGISMRFEVYGCKITDYPCSGMLGMV |
| | | | SGLISDSQITSSNQGDRNWMPENIRLVTSRSGWALPPAPHSYINEWLQIDLGEEKIVRGIIIQGGKHRENKVFMRKFKIGYS |
| | | | NNGSDWKMIMDDSKRKAKSFEGNNNYDTPELRTFPALSTRFIRIYPERATHGGLGLRMELLGCGGGGSGGGGSCMEALGMES |
| | | | GEIHSDQITASSQYSTNWSAERSRLNYPENGWTPGEDSYREWIQVDLGLLRFVTAVGTQGAISKETKAKYYVKTYKIDVSSN |
| | | | TSSNQGDRNWMPENIRLVTSRSGWALPPAPHSYINEWLQIDLGEEKIVRGIIIQGGKHRENKVFMRKFKIGYSNNGSDWKMI |
| | | | MDDSKRKAKSFEGNNNYDTPELRTFPALSTRFIRIYPERATHGGLGLRMELLGCGGGGSGGGGSEPKSCDKTHTCPPCPAPE |
| | | | AAGGPSVFLFPPKPKDTLMISRTPEVTCVVVDVSHEDPEVKFNWYVDGVEVHNAKTKPREEQYNSTYRVVSVLAVLHQDWLN |
| | | | GKEYKCKVSNKALPAPIEKTISKAKGQPREPQVYTLPPSRDELTKNQVSLTCLVKGFYPSDIAVAWESNGQPENNYKTTPPV |
| | | | LDSDGSFFLYSKLTVDKSRWQQGNVFSCSVMHEALHAHYTQKSLSLSPGK |
| 31 | 84 | 870 | CMEALGMESGEIHSDQITASSQYSTNWSAERSRLNYPENGWTPGEDSYREWIQVDLGLLRFVTAVGTQGAISKETKKKYYVK |
| | | | TYKIDVSSNGEDWITIKEGNKPVLFQGNTNPTDVVVAVFPKPLITRFVRIKPATWETGISMRFEVYGCKITDYPCSGMLGMV |
| | | | SGLISDSQITSSNQGDRNWMPENIRLVTSRSGWALPPAPHSYINEWLQIDLGEEKIVRGIIIQGGKHRENKVFMRKFKIGYS |
| | | | NNGSDWKMIMDDSKRKAKSFEGNNNYDTPELRTFPALSTRFIRIYPERATHGGLGLRMELLGCGGGGSGGGGSCMEALGMES |
| | | | GEIHSDQITASSQYSTNWSAERSRLNYPENGWTPGEDSYREWIQVDLGLLRFVTAVGTQGAISKETKKKYYVKTYKIDVSSN |
| | | | TSSNQGDRNWMPENIRLVTSRSGWALPPAPHSYINEWLQIDLGEEKIVRGIIIQGGKHRENKVFMRKFKIGYSNNGSDWKMI |
| | | | MDDSKRKAKSFEGNNNYDTPELRTFPALSTRFIRIYPERATHGGLGLRMELLGCGGGGSGGGGSEPKSCDKTHTCPPCPAPE |
| | | | LLGGPSVFLFPPKPKDTLMISRTPEVTCVVVDVSHEDPEVKFNWYVDGVEVHNAKTKPREEQYASTYRVVSVLAVLHQDWLN |
| | | | GKEYKCKVSNKALPAPIEKTISKAKGQPREPQVYTLPPSRDELTKNQVSLTCLVKGFYPSDIAVAWESNGQPENNYKTTPPV |
| | | | LDSDGSFFLYSKLTVDKSRWQQGNVFSCSVMHEALHAHYTQKSLSLSPGK |
| 32 | 85 | 870 | CMEALGMESGEIHSDQITASSQYSTNWSAERSRLNYPENGWTPGEDSYREWIQVDLGLLRFVTAVGTQGAISKETKKKYYVK |
| | | | TYKIDVSSNGEDWITIKEGNKPVLFQGNTNPTDVVVAVFPKPLITRFVRIKPATWETGISMRFEVYGCKITDYPCSGMLGMV |
| | | | SGLISDSQITSSNQGDRNWMPENIRLVTSRSGWALPPAPHSYINEWLQIDLGEEKIVRGIIIQGGKHRENKVFMRKFKIGYS |
| | | | NNGSDWKMIMDDSKRKAKSFEGNNNYDTPELRTFPALSTRFIRIYPERATHGGLGLRMELLGCGGGGSGGGGSCMEALGMES |
| | | | GEIHSDOITASSQYSTNWSAERSRLNYPENGWTPGEDSYREWIQVDLGLLRFVTAVGTQGAISKETKKKYYVKTYKIDVSSN |
| | | | TSSNQGDRNWMPENIRLVTSRSGWALPPAPHSYINEWLQIDLGEEKIVRGIIIQGGKHRENKVFMRKFKIGYSNNGSDWKMI |
| | | | MDDSKRKAKSFEGNNNYDTPELRTFPALSTRFIRIYPERATHGGLGLRMELLGCGGGGSGGGGSEPKSCDKTHTCPPCPAPE |
| | | | AAGGPSVFLFPPKPKDTLMISRTPEVTCVVVDVSHEDPEVKFNWYVDGVEVHNAKTKPREEQYNSTYRVVSVLAVLHQDWLN |
| | | | GKEYKCKVSNKALPAPIEKTISKAKGQPREPQVYTLPPSRDELTKNQVSLTCLVKGFYPSDIAVAWESNGQPENNYKTTPPV |
| | | | LDSDGSFFLYSKLTVDKSRWQQGNVFSCSVMHEALHAHYTQKSLSLSPGK |
| 33 | 86 | 870 | CMEALGMESGEIHSDQITASSQYSTNWSAERSRLNYPENGWTPGADSYREWIQVDLGLLRFVTAVGTQGAISKETKKKYYVK |
| | | | TYKIDVSSNGEDWITIKEGNKPVLFQGNTNPTDVVVAVFPKPLITRFVRIKPATWETGISMRFEVYGCKITDYPCSGMLGMV |
| | | | SGLISDSQITSSNQGDRNWMPENIRLVTSRSGWALPPAPHSYINEWLQIDLGEEKIVRGIIIQGGKHRENKVFMRKFKIGYS |
| | | | NNGSDWKMIMDDSKRKAKSFEGNNNYDTPELRTFPALSTRFIRIYPERATHGGLGLRMELLGCGGGGSGGGGSCMEALGMES |
| | | | GEIHSDQITASSQYSTNWSAERSRLNYPENGWTPGADSYREWIQVDLGLLRFVTAVGTQGAISKETKKKYYVKTYKIDVSSN |
| | | | GEDWITIKEGNKPVLFQGNTNPTDVVVAVFPKPLITRFVRIKPATWETGISMRFEVYGCKITDYPCSGMLGMVSGLISDSQI |
| | | | TSSNQGDRNWMPENIRLVTSRSGWALPPAPHSYINEWLQIDLGEEKIVRGIIIQGGKHRENKVFMRKFKIGYSNNGSDWKMI |
| | | | MDDSKRKAKSFEGNNNYDTPELRTFPALSTRFIRIYPERATHGGLGLRMELLGCGGGGSGGGGSEPKSCDKTHTCPPCPAPE |
| | | | AAGGPSVFLFPPKPKDTLMISRTPEVTCVVVDVSHEDPEVKFNWYVDGVEVHNAKTKPREEQYNSTYRVVSVLAVLHQDWLN |
| | | | GKEYKCKVSNKALPAPIEKTISKAKGQPREPQVYTLPPSRDELTKNQVSLTCLVKGFYPSDIAVAWESNGQPENNYKTTPPV |
| | | | LDSDGSFFLYSKLTVDKSRWQQGNVFSCSVMHEALHAHYTQKSLSLSPGK |
| 34 | 87 | 874 | CMEALGMESGEIHSDQITASSQYSTNWSAERSRLNYPENGWTPGADSYREWIQVDLGLLRFVTAVGTQGAISKETKKKYYVK |
| | | | TYKIDVSSNGEDWITIKEGNKPVLFQGNTNPTDVVVAVFPKPLITRFVRIKPATWETGISMRFEVYGCKITDYPCSGMLGMV |
| | | | SGLISDSQITSSNQGDRNWMPENIRLVTSRSGWALPPAPHSYINEWLQIDLGEEKIVRGIIIQGGKHRENKVFMRKFKIGYS |
| | | | NNGSDWKMIMDDSKRKAKSFEGNNNYDTPELRTFPALSTRFIRIYPERATHGGLGLRMELLGCGGGGSGGGGSEPKSCDKTH |
| | | | TCPPCPAPEAAGGPSVFLFPPKPKDTLMISRTPEVTCVVVDVSHEDPEVKFNWYVDGVEVHNAKTKPREEQYNSTYRVVSVL |
| | | | AVLHQDWLNGKEYKCKVSNKALPAPIEKTISKAKGQPREPQVYTLPPSRDELTKNQVSLTCLVKGFYPSDIAVAWESNGQPE |
| | | | NNYKTTPPVLDSDGSFFLYSKLTVDKSRWQQGNVFSCSVMHEALHAHYTQKSLSLSPGKGGGGSGGGGSCMEALGMESGEIH |
| | | | SDQITASSQYSTNWSAERSRLNYPENGWTPGADSYREWIQVDLGLLRFVTAVGTQGAISKETKKKYYVKTYKIDVSSNGEDW |
| | | | ITIKEGNKPVLFQGNTNPTDVVVAVFPKPLITRFVRIKPATWETGISMRFEVYGCKITDYPCSGMLGMVSGLISDSQITSSN |
| | | | QGDRNWMPENIRLVTSRSGWALPPAPHSYINEWLQIDLGEEKIVRGIIIQGGKHRENKVFMRKFKIGYSNNGSDWKMIMDDS |
| | | | KRKAKSFEGNNNYDTPELRTFPALSTRFIRIYPERATHGGLGLRMELLGCEVEA |
| 35 | 88 | 866 | CMEALGMESGEIHSDQITASSQYSTNWSAERSRLNYPENGWTPGEDSYREWIQVDLGLLRFVTAVGTQGAISKETKKKYYVK |
| | | | TYKIDVSSNGEDWITIKEGNKPVLFQGNTNPTDVVVAVFPKPLITRFVRIKPATWETGISMRFEVYGCKITDYPCSGMLGMV |
| | | | SGLISDSQITSSNQGDRNWMPENIRLVTSRSGWALPPAPHSYINEWLQIDLGEEKIVRGIIIQGGKHRENKVFMRKFKIGYS |
| | | | NNGSDWKMIMDDSKRKAKSFEGNNNYDTPELRTFPALSTRFIRIYPERATHGGLGLRMELLGCGGGGSGGGGSCMEALGMES |
| | | | GEIHSDQITASSQYSTNWSAERSRLNYPENGWTPGEDSYREWIQVDLGLLRFVTAVGTQGAISKETKKKYYVKTYKIDVSSN |
| | | | GEDWITIKEGNKPVLFQGNTNPTDVVVAVFPKPLITRFVRIKPATWETGISMRFEVYGCKITDYPCSGMLGMVSGLISDSQI |
| | | | TSSNQGDRNWMPENIRLVTSRSGWALPPAPHSYINEWLQIDLGEEKIVRGIIIQGGKHRENKVFMRKFKIGYSNNGSDWKMI |
| | | | MDDSKRKAKSFEGNNNYDTPELRTFPALSTRFIRIYPERATHGGLGLRMELLGCGGGGSGGGGSERKCCVECPPCPAPPVAG |
| | | | PSVFLFPPKPKDQLMISRTPEVTCVVVDVSHEDPEVQFNWYVDGVEVHNAKTKPREEQFNSTFRVVSVLTVVHQDWLNGKEY |
| | | | KCKVSNKGLPAPIEKTISKTKGQPREPQVYTLPPSREEMTKNQVSLTCLVKGFYPSDIAVEWESNGQPENNYKTTPPMLDSD |
| | | | GSFFLYSKLTVDKSRWQQGNVFSCSVLHEALHNHYTQKSLSLSPGK |
| 36 | 89 | 870 | CMEALGMESGEIHSDQITASSQYSTNWSAERSRLNYPENGWTPGEDSYREWIQVDLGLLRFVTAVGTQGAISKETKKKYYVK |
| | | | SGLISDSQITSSNQGDRNWMPENIRLVTSRSGWALPPAPHSYINEWLQIDLGEEKIVRGIIIQGGKHRENKVFMRKFKIGYS |
| | | | NNGSDWKMIMDDSKRKAKSFEGNNNYDTPELRTFPALSTRFIRIYPERATHGGLGLRMELLGCGGGGSGGGGSERKCCVECP |
| | | | PCPAPPVAGPSVFLFPPKPKDQLMISRTPEVTCVVVDVSHEDPEVQFNWYVDGVEVHNAKTKPREEQFNSTFRVVSVLTVVH |
| | | | QDWLNGKEYKCKVSNKGLPAPIEKTISKTKGQPREPQVYTLPPSREEMTKNQVSLTCLVKGFYPSDIAVEWESNGQPENNYK |
| | | | TTPPMLDSDGSFFLYSKLTVDKSRWQQGNVFSCSVLHEALHNHYTQKSLSLSPGKGGGGSGGGGSCMEALGMESGEIHSDQI |
| | | | TASSQYSTNWSAERSRLNYPENGWTPGEDSYREWIQVDLGLLRFVTAVGTQGAISKETKKKYYVKTYKIDVSSNGEDWITIK |
| | | | EGNKPVLFQGNTNPTDVVVAVFPKPLITRFVRIKPATWETGISMRFEVYGCKITDYPCSGMLGMVSGLISDSQITSSNQGDR |
| | | | NWMPENIRLVTSRSGWALPPAPHSYINEWLQIDLGEEKIVRGIIIQGGKHRENKVFMRKFKIGYSNNGSDWKMIMDDSKRKA |
| | | | KSFEGNNNYDTPELRTFPALSTRFIRIYPERATHGGLGLRMELLGCEVEA |

Neuropilin-Immunoglobulin-ACE2 Constructs

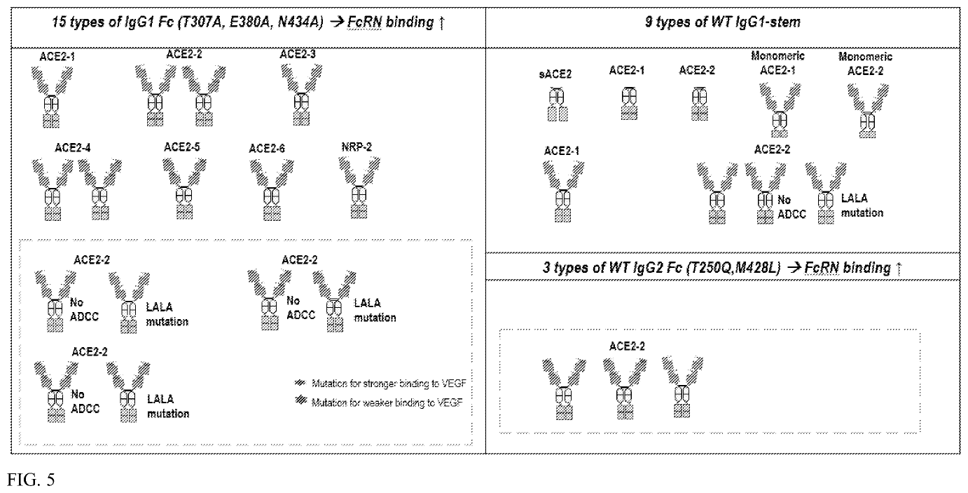

In still another aspect of the current invention, a polypeptide may comprise a b1 domain, or a derivative or fragment thereof, of a neuropilin; an immunoglobulin domain; and an ACE2 domain, or a derivative or a fragment thereof, of an angiotensin converting enzyme 2. Both the b1 and ACE2 domains of these polypeptides are capable of binding to a coat protein of a virus selected from the group consisting of herpesviridae, papillomaviridae, coronaviridae, flaviviridae, togaviridae, bornaviridae, bunyaviridae, filoviridae, orthomyxoviridae, paramyxoviridae, pneumoviridae, and retroviridae. Referring to FIG. 5, a variety of exemplary schematic designs are provided for polypeptides having the b1 domain, or a derivative or fragment thereof, coupled to the ACE2 domain, or a derivative or a fragment thereof, that is additionally coupled to the immunoglobulin domain. Table 8A outlines the general structure and connectivity of several exemplary, but not intending to be limited to, neuropilin-immunoglobulin-ACE2 constructs (Construct Nos. 4-11 and 37-59) or polypeptides including b1, ACE2, linker, and immunoglobulin domains. In some embodiments, the polypeptide has a configuration selected from the group of Construct Nos. 4-11 and 37-59. Table 8B provides a list of these same exemplary constructs from Table 8A including the neuropilin-immunoglobulin-ACE2 polypeptides including b1, ACE2, linker, and immunoglobulin domains and their corresponding peptide sequences.

TABLE 8A
Neuropilin-Immunoglobulin-ACE2 constructs.

| Construct | | | | | | | NRP | IgG |
|---|---|---|---|---|---|---|---|---|
| No. | N-term (NRP1) | Linker | Stem | Linker4 | C-term (ACE2) | Note | type | Subtype |
| 4 | ACE2-1 | (G4S)2 | IgG1 | (G4S)2 | (b1b2)-(G4S)2- | | NRP1 | IgG1 |
| | | | Fc | | (b1b2) | | | |
| 5 | ACE2-2 | (G4S)2 | IgG1 | (G4S)2 | (b1b2)-(G4S)2- | | NRP1 | IgG1 |
| | | | Fc | | (b1b2) | | | |
| 6 | ACE2-3 | (G4S)2 | IgG1 | (G4S)2 | (b1b2)-(G4S)2- | | NRP1 | IgG1 |
| | | | Fc | | (b1b2) | | | |
| 7 | ACE2-4 | (G4S)2 | IgG1 | (G4S)2 | (b1b2)-(G4S)2- | | NRP1 | IgG1 |
| | | | Fc | | (b1b2) | | | |
| 8 | ACE2-2 | (G4S)2 | IgG1 | (G4S)2 | (b1b2)-(G4S)2- | b1(E319A) | NRP1 | IgG1 |
| | | | Fc | | (b1b2) | | | |
| 9 | ACE2-4 | (G4S)2 | IgG1 | (G4S)2 | (b1b2)-(G4S)2- | b1(E319A) | NRP1 | IgG1 |
| | | | Fc | | (b1b2) | | | |
| 10 | ACE2-5 | (G4S)2 | IgG1 | (G4S)2 | (b1b2)-(G4S)2- | b1(E319A) | NRP1 | IgG1 |
| | | | Fc | | (b1b2) | | | |
| 11 | ACE2-6 | (G4S)2 | IgG1 | (G4S)2 | (b1b2)-(G4S)2- | b1(E319A) | NRP1 | IgG1 |
| | | | Fc | | (b1b2) | | | |
| 37 | (b1b2)-(G4S)2- | (G4S)2 | IgG1 | (G4S)2 | ACE2-1 | | NRP1 | IgG1 |
| | (b1b2) | | Fc | | | | | |
| 38 | (b1b2)-(G4S)2- | (G4S)2 | IgG1 | (G4S)2 | ACE2-2 | | NRP1 | IgG1 |
| | (b1b2) | | Fc | | | | | |
| 39 | (b1b2)-(G4S)2- | (G4S)2 | IgG1 | (G4S)2 | ACE2-3 | | NRP1 | IgG1 |
| | (b1b2) | | Fc | | | | | |
| 40 | (b1b2)-(G4S)2- | (G4S)2 | IgG1 | (G4S)2 | ACE2-4 | | NRP1 | IgG1 |
| | (b1b2) | | Fc | | | | | |
| 41 | (b1b2)-(G4S)2- | (G4S)2 | IgG1 | (G4S)2 | ACE2-2 | b1(E319A) | NRP1 | IgG1 |
| | (b1b2) | | Fc | | | | | |
| 42 | (b1b2)-(G4S)2- | (G4S)2 | IgG1 | (G4S)2 | ACE2-4 | b1(E319A) | NRP1 | IgG1 |
| | (b1b2) | | Fc | | | | | |
| 43 | (b1b2)-(G4S)2- | (G4S)2 | IgG1 | (G4S)2 | ACE2-5 | b1(E319A) | NRP1 | IgG1 |
| | (b1b2) | | Fc | | | | | |
| 44 | (b1b2)-(G4S)2- | (G4S)2 | IgG1 | (G4S)2 | ACE2-6 | b1(E319A) | NRP1 | IgG1 |
| | (b1b2) | | Fc | | | | | |
| 45 | (b1b2)-(G4S)2- | (G4S)2 | IgG1 | (G4S)2 | ACE2-2 | b1b2 of NRP-2 | NRP2 | IgG1 |
| | (b1b2) | | Fc | | | | | |
| 46 | (b1b2)-(G4S)2- | (G4S)2 | IgG1 | (G4S)2 | ACE2-1 | | NRP1 | IgG1 |
| | (b1b2) | | Fc | | | | | |
| 47 | (b1b2)-(G4S)2- | (G4S)2 | IgG1 | (G4S)2 | ACE2-2 | | NRP1 | IgG1 |
| | (b1b2) | | Fc | | | | | |
| 48 | (b1b2)-(G4S)2- | (G4S)2 | IgG1 | (G4S)2 | ACE2-2 | No ADCC | NRP1 | IgG1 |
| | (b1b2) | | Fc | | | | | |
| 49 | (b1b2)-(G4S)2- | (G4S)2 | IgG1 | (G4S)2 | ACE2-2 | LALA mutation | NRP1 | IgG1 |
| | (b1b2) | | Fc | | | | | |
| 50 | (b1b2)-(G4S)2- | (G4S)2 | IgG1 | (G4S)2 | ACE2 α1β | Monomeric ACE2-1 | NRP1 | IgG1 |
| | (b1b2) | | Fc | | | | | |
| 51 | (b1b2)-(G4S)2- | (G4S)2 | IgG1 | (G4S)2 | ACE2 α1α2β | Monomeric ACE2-2 | NRP1 | IgGI |
| | (b1b2) | | Fc | | | | | |
| 52 | (b1b2)-(G4S)2- | (G4S)2 | IgG1 | (G4S)2 | ACE2-2 | No ADCC | NRP1 | IgG1 |
| | (b1b2) | | Fc | | | | | |
| 53 | (b1b2)-(G4S)2- | (G4S)2 | IgG1 | (G4S)2 | ACE2-2 | LALA mutation | NRP1 | IgG1 |
| | (b1b2) | | Fc | | | | | |
| 54 | (b1b2)-(G4S)2- | (G4S)2 | IgG1 | (G4S)2 | ACE2-2 | b1(E319A)-No ADCC | NRP1 | IgG1 |
| | (b1b2) | | Fc | | | | | |
| 55 | (b1b2)-(G4S)2- | (G4S)2 | IgG1 | (G4S)2 | ACE2-2 | b1(E319A)-LALA | NRP1 | IgG1 |
| | (b1b2) | | Fc | | | mutation | | |
| 56 | (b1b2)-(G4S)2- | (G4S)2 | IgG2 | (G4S)2 | ACE2-2 | | NRP1 | IgG2 |
| | (b1b2) | | Fc | | | | | |
| 57 | (b1b2)-(G4S)2- | (G4S)2 | IgG2 | (G4S)2 | ACE2-2 | b1(E319A) | NRP1 | IgG2 |
| | (b1b2) | | Fc | | | | | |
| 58 | (b1b2)-(G4S)2- | (G4S)2 | IgG1 | (G4S)2 | ACE2-2 | b1(Y297A, S346A, | NRP1 | IgG1 |
| | (b1b2) | | Fc | | | Y353A)-No ADCC | | |
| 59 | (b1b2)-(G4S)2- | (G4S)2 | IgG1 | (G4S)2 | ACE2-2 | b1(Y297A, S346A, | NRP1 | IgG1 |
| | (b1b2) | | Fc | | | Y353A)-LALA | | |
| | | | | | | mutation | | |

TABLE 8B
Neuropilin-Immunoglobulin-ACE2 constructs (SEQ ID NOS. 57-64, 90-112).

| | SEQ | | |
|---|---|---|---|
| Construct | ID | | |
| No. | NO: | Length | Sequence |
| 4 | 57 | 637 | EEQAKTFLDKFNHEAEDLFYQSSGLGKGDFRGGGGSGGGGSEEQAKTFLDKFNHEAEDLFYQSSGLGKGDFRGGGGSGG |
| | | | GGSEPKSCDKTHTCPPCPAPELLGGPSVFLFPPKPKDTLMISRTPEVTCVVVDVSHEDPEVKFNWYVDGVEVHNAKTKP |
| | | | REEQYNSTYRVVSVLTVLHQDWLNGKEYKCKVSNKALPAPIEKTISKAKGQPREPQVYTLPPSRDELTKNQVSLTCLVK |
| | | | GFYPSDIAVEWESNGQPENNYKTTPPVLDSDGSFFLYSKLTVDKSRWQQGNVFSCSVMHEALHNHYTQKSLSLSPGKGG |
| | | | GGSGGGGSCMEALGMESGEIHSDQITASSQYSTNWSAERSRLNYPENGWTPGEDSYREWIQVDLGLLRFVTAVGTQGAI |
| | | | SKETKKKYYVKTYKIDVSSNGEDWITIKEGNKPVLFQGNTNPTDVVVAVFPKPLITRFVRIKPATWETGISMRFEVYGC |
| | | | KITDYPCSGMLGMVSGLISDSQITSSNQGDRNWMPENIRLVTSRSGWALPPAPHSYINEWLQIDLGEEKIVRGIIIQGG |
| | | | KHRENKVFMRKFKIGYSNNGSDWKMIMDDSKRKAKSFEGNNNYDTPELRTFPALSTRFIRIYPERATHGGLGLRMELLG |
| | | | CEVEA |
| 5 | 58 | 729 | IEEQAKTFLDKFNHEAEDLFYQSSLASWNYNTNITEENVQNMNNAGDKWSAFLKEQSTLAQMYPLQEIWDLGKGDFRGG |
| | | | GGSGGGGSIEEQAKTFLDKFNHEAEDLFYQSSLASWNYNTNITEENVQNMNNAGDKWSAFLKEQSTLAQMYPLQEIWDL |
| | | | GKGDFRGGGGSGGGGSEPKSCDKTHTCPPCPAPELLGGPSVFLFPPKPKDTLMISRTPEVTCVVVDVSHEDPEVKFNWY |
| | | | VDGVEVHNAKTKPREEQYNSTYRVVSVLTVLHQDWLNGKEYKCKVSNKALPAPIEKTISKAKGQPREPQVYTLPPSRDE |
| | | | LTKNQVSLTCLVKGFYPSDIAVEWESNGQPENNYKTTPPVLDSDGSFFLYSKLTVDKSRWQQGNVFSCSVMHEALHNHY |
| | | | TQKSLSLSPGKGGGGSGGGGSCMEALGMESGEIHSDQITASSQYSTNWSAERSRLNYPENGWTPGEDSYREWIQVDLGL |
| | | | LRFVTAVGTQGAISKETKKKYYVKTYKIDVSSNGEDWITIKEGNKPVLFQGNTNPTDVVVAVFPKPLITRFVRIKPATW |
| | | | ETGISMRFEVYGCKITDYPCSGMLGMVSGLISDSQITSSNQGDRNWMPENIRLVTSRSGWALPPAPHSYINEWLQIDLG |
| | | | EEKIVRGIIIQGGKHRENKVFMRKFKIGYSNNGSDWKMIMDDSKRKAKSFEGNNNYDTPELRTFPALSTRFIRIYPERA |
| | | | THGGLGLRMELLGCEVEA |
| 6 | 59 | 637 | EEQAKTWLDKFNHEAEDLFYQSSGLGKGDFRGGGGSGGGGSEEQAKTWLDKFNHEAEDLFYQSSGLGKGDFRGGGGSGG |
| | | | GGSEPKSCDKTHTCPPCPAPELLGGPSVFLFPPKPKDTLMISRTPEVTCVVVDVSHEDPEVKFNWYVDGVEVHNAKTKP |
| | | | REEQYNSTYRVVSVLTVLHQDWLNGKEYKCKVSNKALPAPIEKTISKAKGQPREPQVYTLPPSRDELTKNQVSLTCLVK |
| | | | GFYPSDIAVEWESNGQPENNYKTTPPVLDSDGSFFLYSKLTVDKSRWQQGNVFSCSVMHEALHNHYTQKSLSLSPGKGG |
| | | | GGSGGGGSCMEALGMESGEIHSDQITASSQYSTNWSAERSRLNYPENGWTPGEDSYREWIQVDLGLLRFVTAVGTQGAI |
| | | | SKETKKKYYVKTYKIDVSSNGEDWITIKEGNKPVLFQGNTNPTDVVVAVFPKPLITRFVRIKPATWETGISMRFEVYGC |
| | | | KITDYPCSGMLGMVSGLISDSQITSSNQGDRNWMPENIRLVTSRSGWALPPAPHSYINEWLQIDLGEEKIVRGIIIQGG |
| | | | KHRENKVFMRKFKIGYSNNGSDWKMIMDDSKRKAKSFEGNNNYDTPELRTFPALSTRFIRIYPERATHGGLGLRMELLG |
| | | | CEVEA |
| 7 | 60 | 729 | IEEQAKTWLDKFNHEAEDLFYQSSLASWNYNTNITEENVQNMNNAGDKWSAFLKEQSTLAQMYPLQEIWDLGKGDFRGG |
| | | | GGSGGGGSIEEQAKTWLDKFNHEAEDLFYQSSLASWNYNTNITEENVQNMNNAGDKWSAFLKEQSTLAQMYPLQEIWDL |
| | | | GKGDFRGGGGSGGGGSEPKSCDKTHTCPPCPAPELLGGPSVFLFPPKPKDTLMISRTPEVTCVVVDVSHEDPEVKFNWY |
| | | | VDGVEVHNAKTKPREEQYNSTYRVVSVLTVLHQDWLNGKEYKCKVSNKALPAPIEKTISKAKGQPREPQVYTLPPSRDE |
| | | | LTKNQVSLTCLVKGFYPSDIAVEWESNGQPENNYKTTPPVLDSDGSFFLYSKLTVDKSRWQQGNVFSCSVMHEALHNHY |
| | | | TQKSLSLSPGKGGGGSGGGGSCMEALGMESGEIHSDQITASSQYSTNWSAERSRLNYPENGWTPGEDSYREWIQVDLGL |
| | | | LRFVTAVGTQGAISKETKKKYYVKTYKIDVSSNGEDWITIKEGNKPVLFQGNTNPTDVVVAVFPKPLITRFVRIKPATW |
| | | | ETGISMRFEVYGCKITDYPCSGMLGMVSGLISDSQITSSNQGDRNWMPENIRLVTSRSGWALPPAPHSYINEWLQIDLG |
| | | | EEKIVRGIIIQGGKHRENKVFMRKFKIGYSNNGSDWKMIMDDSKRKAKSFEGNNNYDTPELRTFPALSTRFIRIYPERA |
| | | | THGGLGLRMELLGCEVEA |
| 8 | 61 | 1048 | IEEQAKTFLDKFNHEAEDLFYQSSLASWNYNTNITEENVQNMNNAGDKWSAFLKEQSTLAWMYPLQEIWDLGKGDFRGG |
| | | | GGSGGGGSIEEQAKTFLDKFNHEAEDLFYQSSLASWNYNTNITEENVQNMNNAGDKWSAFLKEQSTLAQMYPLQEIWDL |
| | | | GKGDFRGGGGSGGGGSEPKSCDKTHTCPPCPAPELLGGPSVFLFPPKPKDTLMISRTPEVTCVVVDVSHEDPEVKFNWY |
| | | | VDGVEVHNAKTKPREEQYNSTYRVVSVLTVLHQDWLNGKEYKCKVSNKALPAPIEKTISKAKGQPREPQVYTLPPSRDE |
| | | | LTKNQVSLTCLVKGFYPSDIAVEWESNGQPENNYKTTPPVLDSDGSFFLYSKLTVDKSRWQQGNVFSCSVMHEALHNHY |
| | | | TQKSLSLSPGKGGGGSGGGGSCMEALGMESGEIHSDQITASSQYSTNWSAERSRLNYPENGWTPGADSYREWIQVDLGL |
| | | | LRFVTAVGTQGAISKETKKKYYVKTYKIDVSSNGEDWITIKEGNKPVLFQGNTNPTDVVVAVFPKPLITRFVRIKPATW |
| | | | ETGISMRFEVYGCKITDYPCSGMLGMVSGLISDSQITSSNQGDRNWMPENIRLVTSRSGWALPPAPHSYINEWLQIDLG |
| | | | EEKIVRGIIIQGGKHRENKVFMRKFKIGYSNNGSDWKMIMDDSKRKAKSFEGNNNYDTPELRTFPALSTRFIRIYPERA |
| | | | THGGLGLRMELLGCGGGGSGGGGSCMEALGMESGEIHSDQITASSQYSTNWSAERSRLNYPENGWTPGADSYREWIQVD |
| | | | LGLLRFVTAVGTQGAISKETKKKYYVKTYKIDVSSNGEDWITIKEGNKPVLFQGNTNPTDVVVAVFPKPLITRFVRIKP |
| | | | ATWETGISMRFEVYGCKITDYPCSGMLGMVSGLISDSQITSSNQGDRNWMPENIRLVTSRSGWALPPAPHSYINEWLQI |
| | | | DLGEEKIVRGIIIQGGKHRENKVFMRKFKIGYSNNGSDWKMIMDDSKRKAKSFEGNNNYDTPELRTFPALSTRFIRIYP |
| | | | ERATHGGLGLRMELLGCEVEA |
| 9 | 62 | 1048 | IEEQAKTWLDKFNHEAEDLFYQSSLASWNYNTNITEENVQNMNNAGDKWSAFLKEQSTLAQMYPLQEIWDLGKGDFRGG |
| | | | GGSGGGGSIEEQAKTWLDKFNHEAEDLFYQSSLASWNYNTNITEENVQNMNNAGDKWSAFLKEQSTLAQMYPLQEIWDL |
| | | | GKGDFRGGGGSGGGGSEPKSCDKTHTCPPCPAPELLGGPSVFLFPPKPKDTLMISRTPEVTCVVVDVSHEDPEVKFNWY |
| | | | VDGVEVHNAKTKPREEQYNSTYRVVSVLTVLHQDWLNGKEYKCKVSNKALPAPIEKTISKAKGQPREPQVYTLPPSRDE |
| | | | LTKNQVSLTCLVKGFYPSDIAVEWESNGQPENNYKTTPPVLDSDGSFFLYSKLTVDKSRWQQGNVFSCSVMHEALHNHY |
| | | | TQKSLSLSPGKGGGGSGGGGSCMEALGMESGEIHSDQITASSQYSTNWSAERSRLNYPENGWTPGADSYREWIQVDLGL |
| | | | LRFVTAVGTQGAISKETKKKYYVKTYKIDVSSNGEDWITIKEGNKPVLFQGNTNPTDVVVAVFPKPLITRFVRIKPATW |
| | | | ETGISMRFEVYGCKITDYPCSGMLGMVSGLISDSQITSSNQGDRNWMPENIRLVTSRSGWALPPAPHSYINEWLQIDLG |
| | | | EEKIVRGIIIQGGKHRENKVFMRKFKIGYSNNGSDWKMIMDDSKRKAKSFEGNNNYDTPELRTFPALSTRFIRIYPERA |
| | | | THGGLGLRMELLGCGGGGSGGGGSCMEALGMESGEIHSDQITASSQYSTNWSAERSRLNYPENGWTPGADSYREWIQVD |
| | | | LGLLRFVTAVGTQGAISKETKKKYYVKTYKIDVSSNGEDWITIKEGNKPVLFQGNTNPTDVVVAVFPKPLITRFVRIKP |
| | | | ATWETGISMRFEVYGCKITDYPCSGMLGMVSGLISDSQITSSNQGDRNWMPENIRLVTSRSGWALPPAPHSYINEWLQI |
| | | | DLGEEKIVRGIIIQGGKHRENKVFMRKFKIGYSNNGSDWKMIMDDSKRKAKSFEGNNNYDTPELRTFPALSTRFIRIYP |
| | | | ERATHGGLGLRMELLGCEVEA |
| 10 | 63 | 1048 | IEEQAKTFLAKFNHEAEDLFYQSSLASWNYNTNITEENVQNMNNAGDKWSAFLKEQSTLAQMYPLQEIWDLGKGDFRGG |
| | | | GGSGGGGSIEEQAKTFLAKFNHEAEDLFYQSSLASWNYNTNITEENVQNMNNAGDKWSAFLKEQSTLAQMYPLQEIWDL |
| | | | GKGDFRGGGGSGGGGSEPKSCDKTHTCPPCPAPELLGGPSVFLFPPKPKDTLMISRTPEVTCVVVDVSHEDPEVKFNWY |
| | | | VDGVEVHNAKTKPREEQYNSTYRVVSVLTVLHQDWLNGKEYKCKVSNKALPAPIEKTISKAKGQPREPQVYTLPPSRDE |
| | | | LTKNQVSLTCLVKGFYPSDIAVEWESNGQPENNYKTTPPVLDSDGSFFLYSKLTVDKSRWQQGNVFSCSVMHEALHNHY |
| | | | TQKSLSLSPGKGGGGSGGGGSCMEALGMESGEIHSDQITASSQYSTNWSAERSRLNYPENGWTPGADSYREWIQVDLGL |
| | | | LRFVTAVGTQGAISKETKKKYYVKTYKIDVSSNGEDWITIKEGNKPVLFQGNTNPTDVVVAVFPKPLITRFVRIKPATW |
| | | | ETGISMRFEVYGCKITDYPCSGMLGMVSGLISDSQITSSNQGDRNWMPENIRLVTSRSGWALPPAPHSYINEWLQIDLG |
| | | | EEKIVRGIIIQGGKHRENKVFMRKFKIGYSNNGSDWKMIMDDSKRKAKSFEGNNNYDTPELRTFPALSTRFIRIYPERA |
| | | | THGGLGLRMELLGCGGGGSGGGGSCMEALGMESGEIHSDQITASSQYSTNWSAERSRLNYPENGWTPGADSYREWIQVD |
| | | | ATWETGISMRFEVYGCKITDYPCSGMLGMVSGLISDSQITSSNQGDRNWMPENIRLVTSRSGWALPPAPHSYINEWLQI |
| | | | DLGEEKIVRGIIIQGGKHRENKVFMRKFKIGYSNNGSDWKMIMDDSKRKAKSFEGNNNYDTPELRTFPALSTRFIRIYP |
| | | | ERATHGGLGLRMELLGCEVEA |
| 11 | 64 | 1048 | IEEQAKTFLDKFNHEAEDLFYQSSLASWNYNTNITEENVQNMNNAGDKWSAFLKEQSTTAQMYPLQEIWDLGKGDFRGG |
| | | | GGSGGGGSIEEQAKTFLDKFNHEAEDLFYQSSLASWNYNTNITEENVQNMNNAGDKWSAFLKEQSTTAQMYPLQEIWDL |
| | | | GKGDFRGGGGSGGGGSEPKSCDKTHTCPPCPAPELLGGPSVFLFPPKPKDTLMISRTPEVTCVVVDVSHEDPEVKFNWY |
| | | | VDGVEVHNAKTKPREEQYNSTYRVVSVLTVLHQDWLNGKEYKCKVSNKALPAPIEKTISKAKGQPREPQVYTLPPSRDE |
| | | | LTKNQVSLTCLVKGFYPSDIAVEWESNGQPENNYKTTPPVLDSDGSFFLYSKLTVDKSRWQQGNVFSCSVMHEALHNHY |
| | | | TQKSLSLSPGKGGGGSGGGGSCMEALGMESGEIHSDQITASSQYSTNWSAERSRLNYPENGWTPGADSYREWIQVDLGL |
| | | | LRFVTAVGTQGAISKETKKKYYVKTYKIDVSSNGEDWITIKEGNKPVLFQGNTNPTDVVVAVFPKPLITRFVRIKPATW |
| | | | ETGISMRFEVYGCKITDYPCSGMLGMVSGLISDSQITSSNQGDRNWMPENIRLVTSRSGWALPPAPHSYINEWLQIDLG |
| | | | EEKIVRGIIIQGGKHRENKVFMRKFKIGYSNNGSDWKMIMDDSKRKAKSFEGNNNYDTPELRTFPALSTRFIRIYPERA |
| | | | THGGLGLRMELLGCGGGGSGGGGSCMEALGMESGEIHSDQITASSQYSTNWSAERSRLNYPENGWTPGADSYREWIQVD |
| | | | LGLLRFVTAVGTQGAISKETKKKYYVKTYKIDVSSNGEDWITIKEGNKPVLFQGNTNPTDVVVAVFPKPLITRFVRIKP |
| | | | ATWETGISMRFEVYGCKITDYPCSGMLGMVSGLISDSQITSSNQGDRNWMPENIRLVTSRSGWALPPAPHSYINEWLQI |
| | | | DLGEEKIVRGIIIQGGKHRENKVFMRKFKIGYSNNGSDWKMIMDDSKRKAKSFEGNNNYDTPELRTFPALSTRFIRIYP |
| | | | ERATHGGLGLRMELLGCEVEA |
| 37 | 90 | 952 | CMEALGMESGEIHSDQITASSQYSTNWSAERSRLNYPENGWTPGEDSYREWIQVDLGLLRFVTAVGTQGAISKETKKKY |
| | | | YVKTYKIDVSSNGEDWITIKEGNKPVLFQGNTNPTDVVVAVFPKPLITRFVRIKPATWETGISMRFEVYGCKITDYPCS |
| | | | GMLGMVSGLISDSQITSSNQGDRNWMPENIRLVTSRSGWALPPAPHSYINEWLQIDLGEEKIVRGIIIQGGKHRENKVF |
| | | | MRKFKIGYSNNGSDWKMIMDDSKRKAKSFEGNNNYDTPELRTFPALSTRFIRIYPERATHGGLGLRMELLGCGGGGSGG |
| | | | GGSCMEALGMESGEIHSDQITASSQYSTNWSAERSRLNYPENGWTPGEDSYREWIQVDLGLLRFVTAVGTQGAISKETK |
| | | | KKYYVKTYKIDVSSNGEDWITIKEGNKPVLFQGNTNPTDVVVAVFPKPLITRFVRIKPATWETGISMRFEVYGCKITDY |
| | | | PCSGMLGMVSGLISDSQITSSNQGDRNWMPENIRLVTSRSGWALPPAPHSYINEWLQIDLGEEKIVRGIIIQGGKHREN |
| | | | KVFMRKFKIGYSNNGSDWKMIMDDSKRKAKSFEGNNNYDTPELRTFPALSTRFIRIYPERATHGGLGLRMELLGCGGGG |
| | | | SGGGGSEPKSCDKTHTCPPCPAPELLGGPSVFLFPPKPKDTLMISRTPEVTCVVVDVSHEDPEVKFNWYVDGVEVHNAK |
| | | | TKPREEQYNSTYRVVSVLAVLHQDWLNGKEYKCKVSNKALPAPIEKTISKAKGQPREPQVYTLPPSRDELTKNQVSLTC |
| | | | LVKGFYPSDIAVAWESNGQPENNYKTTPPVLDSDGSFFLYSKLTVDKSRWQQGNVFSCSVMHEALHAHYTQKSLSLSPG |
| | | | KGGGGSGGGGSEEQAKTFLDKFNHEAEDLFYQSSGLGKGDFRGGGGSGGGGSEEQAKTFLDKFNHEAEDLFYQSSGLGK |
| | | | GDFR |
| 38 | 91 | 1044 | CMEALGMESGEIHSDQITASSQYSTNWSAERSRLNYPENGWTPGEDSYREWIQVDLGLLRFVTAVGTQGAISKETKKKY |
| | | | YVKTYKIDVSSNGEDWITIKEGNKPVLFQGNTNPTDVVVAVFPKPLITRFVRIKPATWETGISMRFEVYGCKITDYPCS |
| | | | GMLGMVSGLISDSQITSSNQGDRNWMPENIRLVTSRSGWALPPAPHSYINEWLQIDLGEEKIVRGIIIQGGKHRENKVF |
| | | | MRKFKIGYSNNGSDWKMIMDDSKRKAKSFEGNNNYDTPELRTFPALSTRFIRIYPERATHGGLGLRMELLGCGGGGSGG |
| | | | GGSCMEALGMESGEIHSDQITASSQYSTNWSAERSRLNYPENGWTPGEDSYREWIQVDLGLLRFVTAVGTQGAISKETK |
| | | | KKYYVKTYKIDVSSNGEDWITIKEGNKPVLFQGNTNPTDVVVAVFPKPLITRFVRIKPATWETGISMRFEVYGCKITDY |
| | | | PCSGMLGMVSGLISDSQITSSNQGDRNWMPENIRLVTSRSGWALPPAPHSYINEWLQIDLGEEKIVRGIIIQGGKHREN |
| | | | KVFMRKFKIGYSNNGSDWKMIMDDSKRKAKSFEGNNNYDTPELRTFPALSTRFIRIYPERATHGGLGLRMELLGCGGGG |
| | | | SGGGGSEPKSCDKTHTCPPCPAPELLGGPSVFLFPPKPKDTLMISRTPEVTCVVVDVSHEDPEVKFNWYVDGVEVHNAK |
| | | | TKPREEQYNSTYRVVSVLAVLHQDWLNGKEYKCKVSNKALPAPIEKTISKAKGQPREPQVYTLPPSRDELTKNQVSLTC |
| | | | LVKGFYPSDIAVAWESNGQPENNYKTTPPVLDSDGSFFLYSKLTVDKSRWQQGNVFSCSVMHEALHAHYTQKSLSLSPG |
| | | | KGGGGSGGGGSIEEQAKTFLDKFNHEAEDLFYQSSLASWNYNTNITEENVQNMNNAGDKWSAFLKEQSTLAQMYPLQEI |
| | | | WDLGKGDFRGGGGSGGGGSIEEQAKTFLDKFNHEAEDLFYQSSLASWNYNTNITEENVQNMNNAGDKWSAFLKEQSTLA |
| | | | QMYPLQEIWDLGKGDFR |
| 39 | 92 | 952 | CMEALGMESGEIHSDQITASSQYSTNWSAERSRLNYPENGWTPGEDSYREWIQVDLGLLRFVTAVGTQGAISKETKKKY |
| | | | YVKTYKIDVSSNGEDWITIKEGNKPVLFQGNTNPTDVVVAVFPKPLITRFVRIKPATWETGISMRFEVYGCKITDYPCS |
| | | | GMLGMVSGLISDSQITSSNQGDRNWMPENIRLVTSRSGWALPPAPHSYINEWLQIDLGEEKIVRGIIIQGGKHRENKVF |
| | | | MRKFKIGYSNNGSDWKMIMDDSKRKAKSFEGNNNYDTPELRTFPALSTRFIRIYPERATHGGLGLRMELLGCGGGGSGG |
| | | | GGSCMEALGMESGEIHSDQITASSQYSTNWSAERSRLNYPENGWTPGEDSYREWIQVDLGLLRFVTAVGTQGAISKETK |
| | | | KKYYVKTYKIDVSSNGEDWITIKEGNKPVLFQGNTNPTDVVVAVFPKPLITRFVRIKPATWETGISMRFEVYGCKITDY |
| | | | PCSGMLGMVSGLISDSQITSSNQGDRNWMPENIRLVTSRSGWALPPAPHSYINEWLQIDLGEEKIVRGIIIQGGKHREN |
| | | | KVFMRKFKIGYSNNGSDWKMIMDDSKRKAKSFEGNNNYDTPELRTFPALSTRFIRIYPERATHGGLGLRMELLGCGGGG |
| | | | SGGGGSEPKSCDKTHTCPPCPAPELLGGPSVFLFPPKPKDTLMISRTPEVTCVVVDVSHEDPEVKFNWYVDGVEVHNAK |
| | | | TKPREEQYNSTYRVVSVLAVLHQDWLNGKEYKCKVSNKALPAPIEKTISKAKGQPREPQVYTLPPSRDELTKNQVSLTC |
| | | | LVKGFYPSDIAVAWESNGQPENNYKTTPPVLDSDGSFFLYSKLTVDKSRWQQGNVFSCSVMHEALHAHYTQKSLSLSPG |
| | | | KGGGGSGGGGSEEQAKTWLDKFNHEAEDLFYQSSGLGKGDFRGGGGSGGGGSEEQAKTWLDKFNHEAEDLFYQSSGLGK |
| | | | GDFR |
| 40 | 93 | 1044 | CMEALGMESGEIHSDQITASSQYSTNWSAERSRLNYPENGWTPGEDSYREWIQVDLGLLRFVTAVGTQGAISKETKKKY |
| | | | YVKTYKIDVSSNGEDWITIKEGNKPVLFQGNTNPTDVVVAVFPKPLITRFVRIKPATWETGISMRFEVYGCKITDYPCS |
| | | | GMLGMVSGLISDSQITSSNQGDRNWMPENIRLVTSRSGWALPPAPHSYINEWLQIDLGEEKIVRGIIIQGGKHRENKVF |
| | | | MRKFKIGYSNNGSDWKMIMDDSKRKAKSFEGNNNYDTPELRTFPALSTRFIRIYPERATHGGLGLRMELLGCGGGGSGG |
| | | | GGSCMEALGMESGEIHSDQITASSQYSTNWSAERSRLNYPENGWTPGEDSYREWIQVDLGLLRFVTAVGTQGAISKETK |
| | | | KKYYVKTYKIDVSSNGEDWITIKEGNKPVLFQGNTNPTDVVVAVFPKPLITRFVRIKPATWETGISMRFEVYGCKITDY |
| | | | PCSGMLGMVSGLISDSQITSSNQGDRNWMPENIRLVTSRSGWALPPAPHSYINEWLQIDLGEEKIVRGIIIQGGKHREN |
| | | | KVFMRKFKIGYSNNGSDWKMIMDDSKRKAKSFEGNNNYDTPELRTFPALSTRFIRIYPERATHGGLGLRMELLGCGGGG |
| | | | SGGGGSEPKSCDKTHTCPPCPAPELLGGPSVFLFPPKPKDTLMISRTPEVTCVVVDVSHEDPEVKFNWYVDGVEVHNAK |
| | | | TKPREEQYNSTYRVVSVLAVLHQDWLNGKEYKCKVSNKALPAPIEKTISKAKGQPREPQVYTLPPSRDELTKNQVSLTC |
| | | | LVKGFYPSDIAVAWESNGQPENNYKTTPPVLDSDGSFFLYSKLTVDKSRWQQGNVFSCSVMHEALHAHYTQKSLSLSPG |
| | | | KGGGGSGGGGSIEEQAKTWLDKFNHEAEDLFYQSSLASWNYNTNITEENVQNMNNAGDKWSAFLKEQSTLAQMYPLQEI |
| | | | WDLGKGDFRGGGGSGGGGSIEEQAKTWLDKFNHEAEDLFYQSSLASWNYNTNITEENVQNMNNAGDKWSAFLKEQSTLA |
| | | | QMYPLQEIWDLGKGDFR |
| 41 | 94 | 1044 | CMEALGMESGEIHSDQITASSQYSTNWSAERSRLNYPENGWTPGADSYREWIQVDLGLLRFVTAVGTQGAISKETKKKY |
| | | | YVKTYKIDVSSNGEDWITIKEGNKPVLFQGNTNPTDVVVAVFPKPLITRFVRIKPATWETGISMRFEVYGCKITDYPCS |
| | | | GMLGMVSGLISDSQITSSNQGDRNWMPENIRLVTSRSGWALPPAPHSYINEWLQIDLGEEKIVRGIIIQGGKHRENKVF |
| | | | MRKFKIGYSNNGSDWKMIMDDSKRKAKSFEGNNNYDTPELRTFPALSTRFIRIYPERATHGGLGLRMELLGCGGGGSGG |
| | | | GGSCMEALGMESGEIHSDQITASSQYSTNWSAERSRLNYPENGWTPGADSYREWIQVDLGLLRFVTAVGTQGAISKETK |
| | | | KKYYVKTYKIDVSSNGEDWITIKEGNKPVLFQGNTNPTDVVVAVFPKPLITRFVRIKPATWETGISMRFEVYGCKITDY |
| | | | PCSGMLGMVSGLISDSQITSSNQGDRNWMPENIRLVTSRSGWALPPAPHSYINEWLQIDLGEEKIVRGIIIQGGKHREN |
| | | | KVFMRKFKIGYSNNGSDWKMIMDDSKRKAKSFEGNNNYDTPELRTFPALSTRFIRIYPERATHGGLGLRMELLGCGGGG |
| | | | SGGGGSEPKSCDKTHTCPPCPAPELLGGPSVFLFPPKPKDTLMISRTPEVTCVVVDVSHEDPEVKFNWYVDGVEVHNAK |
| | | | TKPREEQYNSTYRVVSVLAVLHQDWLNGKEYKCKVSNKALPAPIEKTISKAKGQPREPQVYTLPPSRDELTKNQVSLTC |
| | | | LVKGFYPSDIAVAWESNGQPENNYKTTPPVLDSDGSFFLYSKLTVDKSRWQQGNVFSCSVMHEALHAHYTQKSLSLSPG |
| | | | KGGGGSGGGGSIEEQAKTFLDKFNHEAEDLFYQSSLASWNYNTNITEENVQNMNNAGDKWSAFLKEQSTLAQMYPLQEI |
| | | | WDLGKGDFRGGGGSGGGGSIEEQAKTFLDKFNHEAEDLFYQSSLASWNYNTNITEENVQNMNNAGDKWSAFLKEQSTLA |
| | | | QMYPLQEIWDLGKGDFR |
| 42 | 95 | 1044 | CMEALGMESGEIHSDQITASSQYSTNWSAERSRLNYPENGWTPGADSYREWIQVDLGLLRFVTAVGTQGAISKETKKKY |
| | | | YVKTYKIDVSSNGEDWITIKEGNKPVLFQGNTNPTDVVVAVFPKPLITRFVRIKPATWETGISMRFEVYGCKITDYPCS |
| | | | GMLGMVSGLISDSQITSSNQGDRNWMPENIRLVTSRSGWALPPAPHSYINEWLQIDLGEEKIVRGIIIQGGKHRENKVF |
| | | | MRKFKIGYSNNGSDWKMIMDDSKRKAKSFEGNNNYDTPELRTFPALSTRFIRIYPERATHGGLGLRMELLGCGGGGSGG |
| | | | GGSCMEALGMESGEIHSDQITASSQYSTNWSAERSRLNYPENGWTPGADSYREWIQVDLGLLRFVTAVGTQGAISKETK |
| | | | KKYYVKTYKIDVSSNGEDWITIKEGNKPVLFQGNTNPTDVVVAVFPKPLITRFVRIKPATWETGISMRFEVYGCKITDY |
| | | | PCSGMLGMVSGLISDSQITSSNQGDRNWMPENIRLVTSRSGWALPPAPHSYINEWLQIDLGEEKIVRGIIIQGGKHREN |
| | | | KVFMRKFKIGYSNNGSDWKMIMDDSKRKAKSFEGNNNYDTPELRTFPALSTRFIRIYPERATHGGLGLRMELLGCGGGG |
| | | | SGGGGSEPKSCDKTHTCPPCPAPELLGGPSVFLFPPKPKDTLMISRTPEVTCVVVDVSHEDPEVKFNWYVDGVEVHNAK |
| | | | TKPREEQYNSTYRVVSVLAVLHQDWLNGKEYKCKVSNKALPAPIEKTISKAKGQPREPQVYTLPPSRDELTKNQVSLTC |
| | | | LVKGFYPSDIAVAWESNGQPENNYKTTPPVLDSDGSFFLYSKLTVDKSRWQQGNVFSCSVMHEALHAHYTQKSLSLSPG |
| | | | KGGGGSGGGGSIEEQAKTWLDKFNHEAEDLFYQSSLASWNYNTNITEENVQNMNNAGDKWSAFLKEQSTLAQMYPLQEI |
| | | | WDLGKGDFRGGGGSGGGGSIEEQAKTWLDKFNHEAEDLFYQSSLASWNYNTNITEENVQNMNNAGDKWSAFLKEQSTLA |
| | | | QMYPLQEIWDLGKGDFR |
| 43 | 96 | 1044 | CMEALGMESGEIHSDQITASSQYSTNWSAERSRLNYPENGWTPGADSYREWIQVDLGLLRFVTAVGTQGAISKETKKKY |
| | | | YVKTYKIDVSSNGEDWITIKEGNKPVLFQGNTNPTDVVVAVFPKPLITRFVRIKPATWETGISMRFEVYGCKITDYPCS |
| | | | GMLGMVSGLISDSQITSSNQGDRNWMPENIRLVTSRSGWALPPAPHSYINEWLQIDLGEEKIVRGIIIQGGKHRENKVF |
| | | | MRKFKIGYSNNGSDWKMIMDDSKRKAKSFEGNNNYDTPELRTFPALSTRFIRIYPERATHGGLGLRMELLGCGGGGSGG |
| | | | GGSCMEALGMESGEIHSDQITASSQYSTNWSAERSRLNYPENGWTPGADSYREWIQVDLGLLRFVTAVGTQGAISKETK |
| | | | KKYYVKTYKIDVSSNGEDWITIKEGNKPVLFQGNTNPTDVVVAVFPKPLITRFVRIKPATWETGISMRFEVYGCKITDY |
| | | | PCSGMLGMVSGLISDSQITSSNQGDRNWMPENIRLVTSRSGWALPPAPHSYINEWLQIDLGEEKIVRGIIIQGGKHREN |
| | | | KVFMRKFKIGYSNNGSDWKMIMDDSKRKAKSFEGNNNYDTPELRTFPALSTRFIRIYPERATHGGLGLRMELLGCGGGG |
| | | | SGGGGSEPKSCDKTHTCPPCPAPELLGGPSVFLFPPKPKDTLMISRTPEVTCVVVDVSHEDPEVKFNWYVDGVEVHNAK |
| | | | TKPREEQYNSTYRVVSVLAVLHQDWLNGKEYKCKVSNKALPAPIEKTISKAKGQPREPQVYTLPPSRDELTKNQVSLTC |
| | | | LVKGFYPSDIAVAWESNGQPENNYKTTPPVLDSDGSFFLYSKLTVDKSRWQQGNVFSCSVMHEALHAHYTQKSLSLSPG |
| | | | KGGGGSGGGGSIEEQAKTFLAKFNHEAEDLFYQSSLASWNYNTNITEENVQNMNNAGDKWSAFLKEQSTLAQMYPLQEI |
| | | | WDLGKGDFRGGGGSGGGGSIEEQAKTFLAKFNHEAEDLFYQSSLASWNYNTNITEENVQNMNNAGDKWSAFLKEQSTLA |
| | | | QMYPLQEIWDLGKGDFR |
| 44 | 97 | 1044 | CMEALGMESGEIHSDQITASSQYSTNWSAERSRLNYPENGWTPGADSYREWIQVDLGLLRFVTAVGTQGAISKETKKKY |
| | | | YVKTYKIDVSSNGEDWITIKEGNKPVLFQGNTNPTDVVVAVFPKPLITRFVRIKPATWETGISMRFEVYGCKITDYPCS |
| | | | GMLGMVSGLISDSQITSSNQGDRNWMPENIRLVTSRSGWALPPAPHSYINEWLQIDLGEEKIVRGIIIQGGKHRENKVF |
| | | | MRKFKIGYSNNGSDWKMIMDDSKRKAKSFEGNNNYDTPELRTFPALSTRFIRIYPERATHGGLGLRMELLGCGGGGSGG |
| | | | GGSCMEALGMESGEIHSDQITASSQYSTNWSAERSRLNYPENGWTPGADSYREWIQVDLGLLRFVTAVGTQGAISKETK |
| | | | KKYYVKTYKIDVSSNGEDWITIKEGNKPVLFQGNTNPTDVVVAVFPKPLITRFVRIKPATWETGISMRFEVYGCKITDY |
| | | | PCSGMLGMVSGLISDSQITSSNQGDRNWMPENIRLVTSRSGWALPPAPHSYINEWLQIDLGEEKIVRGIIIQGGKHREN |
| | | | KVFMRKFKIGYSNNGSDWKMIMDDSKRKAKSFEGNNNYDTPELRTFPALSTRFIRIYPERATHGGLGLRMELLGCGGGG |
| | | | SGGGGSEPKSCDKTHTCPPCPAPELLGGPSVFLFPPKPKDTLMISRTPEVTCVVVDVSHEDPEVKFNWYVDGVEVHNAK |
| | | | TKPREEQYNSTYRVVSVLAVLHQDWLNGKEYKCKVSNKALPAPIEKTISKAKGQPREPQVYTLPPSRDELTKNQVSLTC |
| | | | LVKGFYPSDIAVAWESNGQPENNYKTTPPVLDSDGSFFLYSKLTVDKSRWQQGNVFSCSVMHEALHAHYTQKSLSLSPG |
| | | | KGGGGSGGGGSIEEQAKTFLDKFNHEAEDLFYQSSLASWNYNTNITEENVQNMNNAGDKWSAFLKEQSTTAQMYPLQEI |
| | | | WDLGKGDFRGGGGSGGGGSIEEQAKTFLDKFNHEAEDLFYQSSLASWNYNTNITEENVQNMNNAGDKWSAFLKEQSTTA |
| | | | QMYPLQEIWDLGKGDFR |
| 45 | 98 | 1058 | CNVPLGMESGRIANEQISASSTYSDGRWTPQQSRLHGDDNGWTPNLDSNKEYLQVDLRFLTMLTAIATQGAISRETQNG |
| | | | YYVKSYKLEVSTNGEDWMVYRHGKNHKVFQANNDATEVVLNKLHAPLLTRFVRIRPQTWHSGIALRLELFGCRVTDAPC |
| | | | SNMLGMLSGLIADSQISASSTQEYLWSPSAARLVSSRSGWFPRIPQAQPGEEWLQVDLGTPKTVKGVIIQGARGGDSIT |
| | | | AVEARAFVRKFKVSYSLNGKDWEYIQDPRTQQPKLFEGNMHYDTPDIRRFDPIPAQYVRVYPERWSPAGIGMRLEVLGC |
| | | | GGGGSGGGGSCNVPLGMESGRIANEQISASSTYSDGRWTPQQSRLHGDDNGWTPNLDSNKEYLQVDLRFLTMLTAIATQ |
| | | | GAISRETQNGYYVKSYKLEVSTNGEDWMVYRHGKNHKVFQANNDATEVVLNKLHAPLLTRFVRIRPQTWHSGIALRLEL |
| | | | FGCRVTDAPCSNMLGMLSGLIADSQISASSTQEYLWSPSAARLVSSRSGWFPRIPQAQPGEEWLQVDLGTPKTVKGVII |
| | | | QGARGGDSITAVEARAFVRKFKVSYSLNGKDWEYIQDPRTQQPKLFEGNMHYDTPDIRRFDPIPAQYVRVYPERWSPAG |
| | | | IGMRLEVLGCGGGGSGGGGSEPKSCDKTHTCPPCPAPELLGGPSVFLFPPKPKDTLMISRTPEVTCVVVDVSHEDPEVK |
| | | | FNWYVDGVEVHNAKTKPREEQYNSTYRVVSVLAVLHQDWLNGKEYKCKVSNKALPAPIEKTISKAKGQPREPQVYTLPP |
| | | | SRDELTKNQVSLTCLVKGFYPSDIAVAWESNGQPENNYKTTPPVLDSDGSFFLYSKLTVDKSRWQQGNVFSCSVMHEAL |
| | | | HAHYTQKSLSLSPGKGGGGSGGGGSIEEQAKTFLDKFNHEAEDLFYQSSLASWNYNTNITEENVQNMNNAGDKWSAFLK |
| | | | EQSTLAQMYPLQEIWDLGKGDFRGGGGSGGGGSIEEQAKTFLDKFNHEAEDLFYQSSLASWNYNTNITEENVQNMNNAG |
| | | | DKWSAFLKEQSTLAQMYPLQEIWDLGKGDFR |
| 46 | 99 | 952 | CMEALGMESGEIHSDQITASSQYSTNWSAERSRLNYPENGWTPGEDSYREWIQVDLGLLRFVTAVGTQGAISKETKKKY |
| | | | YVKTYKIDVSSNGEDWITIKEGNKPVLFQGNTNPTDVVVAVFPKPLITRFVRIKPATWETGISMRFEVYGCKITDYPCS |
| | | | GMLGMVSGLISDSQITSSNQGDRNWMPENIRLVTSRSGWALPPAPHSYINEWLQIDLGEEKIVRGIIIQGGKHRENKVF |
| | | | MRKFKIGYSNNGSDWKMIMDDSKRKAKSFEGNNNYDTPELRTFPALSTRFIRIYPERATHGGLGLRMELLGCGGGGSGG |
| | | | GGSCMEALGMESGEIHSDQITASSQYSTNWSAERSRLNYPENGWTPGEDSYREWIQVDLGLLRFVTAVGTQGAISKETK |
| | | | KKYYVKTYKIDVSSNGEDWITIKEGNKPVLFQGNTNPTDVVVAVFPKPLITRFVRIKPATWETGISMRFEVYGCKITDY |
| | | | PCSGMLGMVSGLISDSQITSSNQGDRNWMPENIRLVTSRSGWALPPAPHSYINEWLQIDLGEEKIVRGIIIQGGKHREN |
| | | | KVFMRKFKIGYSNNGSDWKMIMDDSKRKAKSFEGNNNYDTPELRTFPALSTRFIRIYPERATHGGLGLRMELLGCGGGG |
| | | | SGGGGSEPKSCDKTHTCPPCPAPELLGGPSVFLFPPKPKDTLMISRTPEVTCVVVDVSHEDPEVKFNWYVDGVEVHNAK |
| | | | TKPREEQYNSTYRVVSVLTVLHQDWLNGKEYKCKVSNKALPAPIEKTISKAKGQPREPQVYTLPPSRDELTKNQVSLTC |
| | | | LVKGFYPSDIAVEWESNGQPENNYKTTPPVLDSDGSFFLYSKLTVDKSRWQQGNVFSCSVMHEALHNHYTQKSLSLSPG |
| | | | KGGGGSGGGGSEEQAKTFLDKFNHEAEDLFYQSSGLGKGDFRGGGGSGGGGSEEQAKTFLDKFNHEAEDLFYQSSGLGK |
| | | | GDFR |
| 47 | 100 | 1044 | CMEALGMESGEIHSDQITASSQYSTNWSAERSRLNYPENGWTPGEDSYREWIQVDLGLLRFVTAVGTQGAISKETKKKY |
| | | | YVKTYKIDVSSNGEDWITIKEGNKPVLFQGNTNPTDVVVAVFPKPLITRFVRIKPATWETGISMRFEVYGCKITDYPCS |
| | | | GMLGMVSGLISDSQITSSNQGDRNWMPENIRLVTSRSGWALPPAPHSYINEWLQIDLGEEKIVRGIIIQGGKHRENKVF |
| | | | MRKFKIGYSNNGSDWKMIMDDSKRKAKSFEGNNNYDTPELRTFPALSTRFIRIYPERATHGGLGLRMELLGCGGGGSGG |
| | | | GGSCMEALGMESGEIHSDQITASSQYSTNWSAERSRLNYPENGWTPGEDSYREWIQVDLGLLRFVTAVGTQGAISKETK |
| | | | KKYYVKTYKIDVSSNGEDWITIKEGNKPVLFQGNTNPTDVVVAVFPKPLITRFVRIKPATWETGISMRFEVYGCKITDY |
| | | | PCSGMLGMVSGLISDSQITSSNQGDRNWMPENIRLVTSRSGWALPPAPHSYINEWLQIDLGEEKIVRGIIIQGGKHREN |
| | | | KVFMRKFKIGYSNNGSDWKMIMDDSKRKAKSFEGNNNYDTPELRTFPALSTRFIRIYPERATHGGLGLRMELLGCGGGG |
| | | | SGGGGSEPKSCDKTHTCPPCPAPELLGGPSVFLFPPKPKDTLMISRTPEVTCVVVDVSHEDPEVKFNWYVDGVEVHNAK |
| | | | TKPREEQYNSTYRVVSVLTVLHQDWLNGKEYKCKVSNKALPAPIEKTISKAKGQPREPQVYTLPPSRDELTKNQVSLTC |
| | | | LVKGFYPSDIAVEWESNGQPENNYKTTPPVLDSDGSFFLYSKLTVDKSRWQQGNVFSCSVMHEALHNHYTQKSLSLSPG |
| | | | KGGGGSGGGGSIEEQAKTFLDKFNHEAEDLFYQSSLASWNYNTNITEENVQNMNNAGDKWSAFLKEQSTLAQMYPLQEI |
| | | | WDLGKGDFRGGGGSGGGGSIEEQAKTFLDKFNHEAEDLFYQSSLASWNYNTNITEENVQNMNNAGDKWSAFLKEQSTLA |
| | | | QMYPLQEIWDLGKGDFR |
| 48 | 101 | 1044 | CMEALGMESGEIHSDQITASSQYSTNWSAERSRLNYPENGWTPGEDSYREWIQVDLGLLRFVTAVGTQGAISKETKKKY |
| | | | YVKTYKIDVSSNGEDWITIKEGNKPVLFQGNTNPTDVVVAVFPKPLITRFVRIKPATWETGISMRFEVYGCKITDYPCS |
| | | | GMLGMVSGLISDSQITSSNQGDRNWMPENIRLVTSRSGWALPPAPHSYINEWLQIDLGEEKIVRGIIIQGGKHRENKVF |
| | | | MRKFKIGYSNNGSDWKMIMDDSKRKAKSFEGNNNYDTPELRTFPALSTRFIRIYPERATHGGLGLRMELLGCGGGGSGG |
| | | | GGSCMEALGMESGEIHSDQITASSQYSTNWSAERSRLNYPENGWTPGEDSYREWIQVDLGLLRFVTAVGTQGAISKETK |
| | | | KKYYVKTYKIDVSSNGEDWITIKEGNKPVLFQGNTNPTDVVVAVFPKPLITRFVRIKPATWETGISMRFEVYGCKITDY |
| | | | PCSGMLGMVSGLISDSQITSSNQGDRNWMPENIRLVTSRSGWALPPAPHSYINEWLQIDLGEEKIVRGIIIQGGKHREN |
| | | | KVFMRKFKIGYSNNGSDWKMIMDDSKRKAKSFEGNNNYDTPELRTFPALSTRFIRIYPERATHGGLGLRMELLGCGGGG |
| | | | SGGGGSEPKSCDKTHTCPPCPAPELLGGPSVFLFPPKPKDTLMISRTPEVTCVVVDVSHEDPEVKFNWYVDGVEVHNAK |
| | | | TKPREEQYASTYRVVSVLTVLHQDWLNGKEYKCKVSNKALPAPIEKTISKAKGQPREPQVYTLPPSRDELTKNQVSLTC |
| | | | LVKGFYPSDIAVEWESNGQPENNYKTTPPVLDSDGSFFLYSKLTVDKSRWQQGNVFSCSVMHEALHNHYTQKSLSLSPG |
| | | | KGGGGSGGGGSIEEQAKTFLDKFNHEAEDLFYQSSLASWNYNTNITEENVQNMNNAGDKWSAFLKEQSTLAQMYPLQEI |
| | | | WDLGKGDFRGGGGSGGGGSIEEQAKTFLDKFNHEAEDLFYQSSLASWNYNTNITEENVQNMNNAGDKWSAFLKEQSTLA |
| | | | QMYPLQEIWDLGKGDFR |
| 49 | 102 | 1044 | CMEALGMESGEIHSDQITASSQYSTNWSAERSRLNYPENGWTPGEDSYREWIQVDLGLLRFVTAVGTQGAISKETKKKY |
| | | | YVKTYKIDVSSNGEDWITIKEGNKPVLFQGNTNPTDVVVAVFPKPLITRFVRIKPATWETGISMRFEVYGCKITDYPCS |
| | | | GMLGMVSGLISDSQITSSNQGDRNWMPENIRLVTSRSGWALPPAPHSYINEWLQIDLGEEKIVRGIIIQGGKHRENKVF |
| | | | MRKFKIGYSNNGSDWKMIMDDSKRKAKSFEGNNNYDTPELRTFPALSTRFIRIYPERATHGGLGLRMELLGCGGGGSGG |
| | | | GGSCMEALGMESGEIHSDQITASSQYSTNWSAERSRLNYPENGWTPGEDSYREWIQVDLGLLRFVTAVGTQGAISKETK |
| | | | KKYYVKTYKIDVSSNGEDWITIKEGNKPVLFQGNTNPTDVVVAVFPKPLITRFVRIKPATWETGISMRFEVYGCKITDY |
| | | | PCSGMLGMVSGLISDSQITSSNQGDRNWMPENIRLVTSRSGWALPPAPHSYINEWLQIDLGEEKIVRGIIIQGGKHREN |
| | | | KVFMRKFKIGYSNNGSDWKMIMDDSKRKAKSFEGNNNYDTPELRTFPALSTRFIRIYPERATHGGLGLRMELLGCGGGG |
| | | | SGGGGSEPKSCDKTHTCPPCPAPEAAGGPSVFLFPPKPKDTLMISRTPEVTCVVVDVSHEDPEVKFNWYVDGVEVHNAK |
| | | | TKPREEQYNSTYRVVSVLTVLHQDWLNGKEYKCKVSNKALPAPIEKTISKAKGQPREPQVYTLPPSRDELTKNQVSLTC |
| | | | LVKGFYPSDIAVEWESNGQPENNYKTTPPVLDSDGSFFLYSKLTVDKSRWQQGNVFSCSVMHEALHNHYTQKSLSLSPG |
| | | | KGGGGSGGGGSIEEQAKTFLDKFNHEAEDLFYQSSLASWNYNTNITEENVQNMNNAGDKWSAFLKEQSTLAQMYPLQEI |
| | | | WDLGKGDFRGGGGSGGGGSIEEQAKTFLDKFNHEAEDLFYQSSLASWNYNTNITEENVQNMNNAGDKWSAFLKEQSTLA |
| | | | QMYPLQEIWDLGKGDFR |
| 50 | 103 | 911 | CMEALGMESGEIHSDQITASSQYSTNWSAERSRLNYPENGWTPGEDSYREWIQVDLGLLRFVTAVGTQGAISKETKKKY |
| | | | YVKTYKIDVSSNGEDWITIKEGNKPVLFQGNTNPTDVVVAVFPKPLITRFVRIKPATWETGISMRFEVYGCKITDYPCS |
| | | | GMLGMVSGLISDSQITSSNQGDRNWMPENIRLVTSRSGWALPPAPHSYINEWLQIDLGEEKIVRGIIIQGGKHRENKVF |
| | | | MRKFKIGYSNNGSDWKMIMDDSKRKAKSFEGNNNYDTPELRTFPALSTRFIRIYPERATHGGLGLRMELLGCGGGGSGG |
| | | | GGSCMEALGMESGEIHSDQITASSQYSTNWSAERSRLNYPENGWTPGEDSYREWIQVDLGLLRFVTAVGTQGAISKETK |
| | | | KKYYVKTYKIDVSSNGEDWITIKEGNKPVLFQGNTNPTDVVVAVFPKPLITRFVRIKPATWETGISMRFEVYGCKITDY |
| | | | PCSGMLGMVSGLISDSQITSSNQGDRNWMPENIRLVTSRSGWALPPAPHSYINEWLQIDLGEEKIVRGIIIQGGKHREN |
| | | | KVFMRKFKIGYSNNGSDWKMIMDDSKRKAKSFEGNNNYDTPELRTFPALSTRFIRIYPERATHGGLGLRMELLGCGGGG |
| | | | SGGGGSEPKSCDKTHTCPPCPAPELLGGPSVFLFPPKPKDTLMISRTPEVTCVVVDVSHEDPEVKFNWYVDGVEVHNAK |
| | | | TKPREEQYNSTYRVVSVLTVLHQDWLNGKEYKCKVSNKALPAPIEKTISKAKGQPREPQVYTLPPSRDELTKNQVSLTC |
| | | | LVKGFYPSDIAVEWESNGQPENNYKTTPPVLDSDGSFFLYSKLTVDKSRWQQGNVFSCSVMHEALHNHYTQKSLSLSPG |
| | | | KGGGGSGGGGSEEQAKTFLDKFNHEAEDLFYQSSGLGKGDFR |
| 51 | 104 | 957 | CMEALGMESGEIHSDQITASSQYSTNWSAERSRLNYPENGWTPGEDSYREWIQVDLGLLRFVTAVGTQGAISKETKKKY |
| | | | YVKTYKIDVSSNGEDWITIKEGNKPVLFQGNTNPTDVVVAVFPKPLITRFVRIKPATWETGISMRFEVYGCKITDYPCS |
| | | | GMLGMVSGLISDSQITSSNQGDRNWMPENIRLVTSRSGWALPPAPHSYINEWLQIDLGEEKIVRGIIIQGGKHRENKVF |
| | | | MRKFKIGYSNNGSDWKMIMDDSKRKAKSFEGNNNYDTPELRTFPALSTRFIRIYPERATHGGLGLRMELLGCGGGGSGG |
| | | | GGSCMEALGMESGEIHSDQITASSQYSTNWSAERSRLNYPENGWTPGEDSYREWIQVDLGLLRFVTAVGTQGAISKETK |
| | | | KKYYVKTYKIDVSSNGEDWITIKEGNKPVLFQGNTNPTDVVVAVFPKPLITRFVRIKPATWETGISMRFEVYGCKITDY |
| | | | PCSGMLGMVSGLISDSQITSSNQGDRNWMPENIRLVTSRSGWALPPAPHSYINEWLQIDLGEEKIVRGIIIQGGKHREN |
| | | | KVFMRKFKIGYSNNGSDWKMIMDDSKRKAKSFEGNNNYDTPELRTFPALSTRFIRIYPERATHGGLGLRMELLGCGGGG |
| | | | SGGGGSEPKSCDKTHTCPPCPAPELLGGPSVFLFPPKPKDTLMISRTPEVTCVVVDVSHEDPEVKFNWYVDGVEVHNAK |
| | | | TKPREEQYNSTYRVVSVLTVLHQDWLNGKEYKCKVSNKALPAPIEKTISKAKGQPREPQVYTLPPSRDELTKNQVSLTC |
| | | | LVKGFYPSDIAVEWESNGQPENNYKTTPPVLDSDGSFFLYSKLTVDKSRWQQGNVFSCSVMHEALHNHYTQKSLSLSPG |
| | | | KGGGGSGGGGSIEEQAKTFLDKFNHEAEDLFYQSSLASWNYNTNITEENVQNMNNAGDKWSAFLKEQSTLAQMYPLQEI |
| | | | WDLGKGDFR |
| 52 | 105 | 1044 | CMEALGMESGEIHSDQITASSQYSTNWSAERSRLNYPENGWTPGEDSYREWIQVDLGLLRFVTAVGTQGAISKETKKKY |
| | | | YVKTYKIDVSSNGEDWITIKEGNKPVLFQGNTNPTDVVVAVFPKPLITRFVRIKPATWETGISMRFEVYGCKITDYPCS |
| | | | GMLGMVSGLISDSQITSSNQGDRNWMPENIRLVTSRSGWALPPAPHSYINEWLQIDLGEEKIVRGIIIQGGKHRENKVF |
| | | | MRKFKIGYSNNGSDWKMIMDDSKRKAKSFEGNNNYDTPELRTFPALSTRFIRIYPERATHGGLGLRMELLGCGGGGSGG |
| | | | GGSCMEALGMESGEIHSDQITASSQYSTNWSAERSRLNYPENGWTPGEDSYREWIQVDLGLLRFVTAVGTQGAISKETK |
| | | | KKYYVKTYKIDVSSNGEDWITIKEGNKPVLFQGNTNPTDVVVAVFPKPLITRFVRIKPATWETGISMRFEVYGCKITDY |
| | | | PCSGMLGMVSGLISDSQITSSNQGDRNWMPENIRLVTSRSGWALPPAPHSYINEWLQIDLGEEKIVRGIIIQGGKHREN |
| | | | KVFMRKFKIGYSNNGSDWKMIMDDSKRKAKSFEGNNNYDTPELRTFPALSTRFIRIYPERATHGGLGLRMELLGCGGGG |
| | | | SGGGGSEPKSCDKTHTCPPCPAPELLGGPSVFLFPPKPKDTLMISRTPEVTCVVVDVSHEDPEVKFNWYVDGVEVHNAK |
| | | | TKPREEQYASTYRVVSVLAVLHQDWLNGKEYKCKVSNKALPAPIEKTISKAKGQPREPQVYTLPPSRDELTKNQVSLTC |
| | | | LVKGFYPSDIAVAWESNGQPENNYKTTPPVLDSDGSFFLYSKLTVDKSRWQQGNVFSCSVMHEALHAHYTQKSLSLSPG |
| | | | KGGGGSGGGGSIEEQAKTFLDKFNHEAEDLFYQSSLASWNYNTNITEENVQNMNNAGDKWSAFLKEQSTLAQMYPLQEI |
| | | | WDLGKGDFRGGGGSGGGGSIEEQAKTFLDKFNHEAEDLFYQSSLASWNYNTNITEENVQNMNNAGDKWSAFLKEQSTLA |
| | | | QMYPLQEIWDLGKGDFR |
| 53 | 106 | 1044 | CMEALGMESGEIHSDQITASSQYSTNWSAERSRLNYPENGWTPGEDSYREWIQVDLGLLRFVTAVGTQGAISKETKKKY |
| | | | YVKTYKIDVSSNGEDWITIKEGNKPVLFQGNTNPTDVVVAVFPKPLITRFVRIKPATWETGISMRFEVYGCKITDYPCS |
| | | | GMLGMVSGLISDSQITSSNQGDRNWMPENIRLVTSRSGWALPPAPHSYINEWLQIDLGEEKIVRGIIIQGGKHRENKVF |
| | | | MRKFKIGYSNNGSDWKMIMDDSKRKAKSFEGNNNYDTPELRTFPALSTRFIRIYPERATHGGLGLRMELLGCGGGGSGG |
| | | | GGSCMEALGMESGEIHSDQITASSQYSTNWSAERSRLNYPENGWTPGEDSYREWIQVDLGLLRFVTAVGTQGAISKETK |
| | | | KKYYVKTYKIDVSSNGEDWITIKEGNKPVLFQGNTNPTDVVVAVFPKPLITRFVRIKPATWETGISMRFEVYGCKITDY |
| | | | PCSGMLGMVSGLISDSQITSSNQGDRNWMPENIRLVTSRSGWALPPAPHSYINEWLQIDLGEEKIVRGIIIQGGKHREN |
| | | | KVFMRKFKIGYSNNGSDWKMIMDDSKRKAKSFEGNNNYDTPELRTFPALSTRFIRIYPERATHGGLGLRMELLGCGGGG |
| | | | SGGGGSEPKSCDKTHTCPPCPAPEAAGGPSVFLFPPKPKDTLMISRTPEVTCVVVDVSHEDPEVKFNWYVDGVEVHNAK |
| | | | TKPREEQYNSTYRVVSVLAVLHQDWLNGKEYKCKVSNKALPAPIEKTISKAKGQPREPQVYTLPPSRDELTKNQVSLTC |
| | | | LVKGFYPSDIAVAWESNGQPENNYKTTPPVLDSDGSFFLYSKLTVDKSRWQQGNVFSCSVMHEALHAHYTQKSLSLSPG |
| | | | KGGGGSGGGGSIEEQAKTFLDKFNHEAEDLFYQSSLASWNYNTNITEENVQNMNNAGDKWSAFLKEQSTLAQMYPLQEI |
| | | | WDLGKGDFRGGGGSGGGGSIEEQAKTFLDKFNHEAEDLFYQSSLASWNYNTNITEENVQNMNNAGDKWSAFLKEQSTLA |
| | | | QMYPLQEIWDLGKGDFR |
| 54 | 107 | 1044 | CMEALGMESGEIHSDQITASSQYSTNWSAERSRLNYPENGWTPGADSYREWIQVDLGLLRFVTAVGTQGAISKETKKKY |
| | | | YVKTYKIDVSSNGEDWITIKEGNKPVLFQGNTNPTDVVVAVFPKPLITRFVRIKPATWETGISMRFEVYGCKITDYPCS |
| | | | GMLGMVSGLISDSQITSSNQGDRNWMPENIRLVTSRSGWALPPAPHSYINEWLQIDLGEEKIVRGIIIQGGKHRENKVF |
| | | | MRKFKIGYSNNGSDWKMIMDDSKRKAKSFEGNNNYDTPELRTFPALSTRFIRIYPERATHGGLGLRMELLGCGGGGSGG |
| | | | GGSCMEALGMESGEIHSDQITASSQYSTNWSAERSRLNYPENGWTPGADSYREWIQVDLGLLRFVTAVGTQGAISKETK |
| | | | KKYYVKTYKIDVSSNGEDWITIKEGNKPVLFQGNTNPTDVVVAVFPKPLITRFVRIKPATWETGISMRFEVYGCKITDY |
| | | | PCSGMLGMVSGLISDSQITSSNQGDRNWMPENIRLVTSRSGWALPPAPHSYINEWLQIDLGEEKIVRGIIIQGGKHREN |
| | | | KVFMRKFKIGYSNNGSDWKMIMDDSKRKAKSFEGNNNYDTPELRTFPALSTRFIRIYPERATHGGLGLRMELLGCGGGG |
| | | | SGGGGSEPKSCDKTHTCPPCPAPELLGGPSVFLFPPKPKDTLMISRTPEVTCVVVDVSHEDPEVKFNWYVDGVEVHNAK |
| | | | TKPREEQYASTYRVVSVLAVLHQDWLNGKEYKCKVSNKALPAPIEKTISKAKGQPREPQVYTLPPSRDELTKNQVSLTC |
| | | | LVKGFYPSDIAVAWESNGQPENNYKTTPPVLDSDGSFFLYSKLTVDKSRWQQGNVFSCSVMHEALHAHYTQKSLSLSPG |
| | | | KGGGGSGGGGSIEEQAKTFLDKFNHEAEDLFYQSSLASWNYNTNITEENVQNMNNAGDKWSAFLKEQSTLAQMYPLQEI |
| | | | WDLGKGDFRGGGGSGGGGSIEEQAKTFLDKFNHEAEDLFYQSSLASWNYNTNITEENVQNMNNAGDKWSAFLKEQSTLA |
| | | | QMYPLQEIWDLGKGDFR |
| 55 | 108 | 1044 | CMEALGMESGEIHSDQITASSQYSTNWSAERSRLNYPENGWTPGADSYREWIQVDLGLLRFVTAVGTQGAISKETKKKY |
| | | | YVKTYKIDVSSNGEDWITIKEGNKPVLFQGNTNPTDVVVAVFPKPLITRFVRIKPATWETGISMRFEVYGCKITDYPCS |
| | | | GMLGMVSGLISDSQITSSNQGDRNWMPENIRLVTSRSGWALPPAPHSYINEWLQIDLGEEKIVRGIIIQGGKHRENKVF |
| | | | MRKFKIGYSNNGSDWKMIMDDSKRKAKSFEGNNNYDTPELRTFPALSTRFIRIYPERATHGGLGLRMELLGCGGGGSGG |
| | | | GGSCMEALGMESGEIHSDQITASSQYSTNWSAERSRLNYPENGWTPGADSYREWIQVDLGLLRFVTAVGTQGAISKETK |
| | | | PCSGMLGMVSGLISDSQITSSNQGDRNWMPENIRLVTSRSGWALPPAPHSYINEWLQIDLGEEKIVRGIIIQGGKHREN |
| | | | KVFMRKFKIGYSNNGSDWKMIMDDSKRKAKSFEGNNNYDTPELRTFPALSTRFIRIYPERATHGGLGLRMELLGCGGGG |
| | | | SGGGGSEPKSCDKTHTCPPCPAPEAAGGPSVFLFPPKPKDTLMISRTPEVTCVVVDVSHEDPEVKFNWYVDGVEVHNAK |
| | | | TKPREEQYNSTYRVVSVLAVLHQDWLNGKEYKCKVSNKALPAPIEKTISKAKGQPREPQVYTLPPSRDELTKNQVSLTC |
| | | | LVKGFYPSDIAVAWESNGQPENNYKTTPPVLDSDGSFFLYSKLTVDKSRWQQGNVFSCSVMHEALHAHYTQKSLSLSPG |
| | | | KGGGGSGGGGSIEEQAKTFLDKFNHEAEDLFYQSSLASWNYNTNITEENVQNMNNAGDKWSAFLKEQSTLAQMYPLQEI |
| | | | WDLGKGDFRGGGGSGGGGSIEEQAKTFLDKFNHEAEDLFYQSSLASWNYNTNITEENVQNMNNAGDKWSAFLKEQSTLA |
| | | | QMYPLQEIWDLGKGDFR |
| 56 | 109 | 1040 | CMEALGMESGEIHSDQITASSQYSTNWSAERSRLNYPENGWTPGEDSYREWIQVDLGLLRFVTAVGTQGAISKETKKKY |
| | | | YVKTYKIDVSSNGEDWITIKEGNKPVLFQGNTNPTDVVVAVFPKPLITRFVRIKPATWETGISMRFEVYGCKITDYPCS |
| | | | GMLGMVSGLISDSQITSSNQGDRNWMPENIRLVTSRSGWALPPAPHSYINEWLQIDLGEEKIVRGIIIQGGKHRENKVF |
| | | | MRKFKIGYSNNGSDWKMIMDDSKRKAKSFEGNNNYDTPELRTFPALSTRFIRIYPERATHGGLGLRMELLGCGGGGSGG |
| | | | GGSCMEALGMESGEIHSDQITASSQYSTNWSAERSRLNYPENGWTPGEDSYREWIQVDLGLLRFVTAVGTQGAISKETK |
| | | | KKYYVKTYKIDVSSNGEDWITIKEGNKPVLFQGNTNPTDVVVAVFPKPLITRFVRIKPATWETGISMRFEVYGCKITDY |
| | | | PCSGMLGMVSGLISDSQITSSNQGDRNWMPENIRLVTSRSGWALPPAPHSYINEWLQIDLGEEKIVRGIIIQGGKHREN |
| | | | KVFMRKFKIGYSNNGSDWKMIMDDSKRKAKSFEGNNNYDTPELRTFPALSTRFIRIYPERATHGGLGLRMELLGCGGGG |
| | | | SGGGGSERKCCVECPPCPAPPVAGPSVFLFPPKPKDQLMISRTPEVTCVVVDVSHEDPEVQFNWYVDGVEVHNAKTKPR |
| | | | EEQFNSTFRVVSVLTVVHQDWLNGKEYKCKVSNKGLPAPIEKTISKTKGQPREPQVYTLPPSREEMTKNQVSLTCLVKG |
| | | | FYPSDIAVEWESNGQPENNYKTTPPMLDSDGSFFLYSKLTVDKSRWQQGNVFSCSVLHEALHNHYTQKSLSLSPGKGGG |
| | | | GSGGGGSIEEQAKTFLDKFNHEAEDLFYQSSLASWNYNTNITEENVQNMNNAGDKWSAFLKEQSTLAQMYPLQEIWDLG |
| | | | KGDFRGGGGSGGGGSIEEQAKTFLDKFNHEAEDLFYQSSLASWNYNTNITEENVQNMNNAGDKWSAFLKEQSTLAQMYP |
| | | | LQEIWDLGKGDFR |
| 57 | 110 | 1040 | CMEALGMESGEIHSDQITASSQYSTNWSAERSRLNYPENGWTPGADSYREWIQVDLGLLRFVTAVGTQGAISKETKKKY |
| | | | YVKTYKIDVSSNGEDWITIKEGNKPVLFQGNTNPTDVVVAVFPKPLITRFVRIKPATWETGISMRFEVYGCKITDYPCS |
| | | | GMLGMVSGLISDSQITSSNQGDRNWMPENIRLVTSRSGWALPPAPHSYINEWLQIDLGEEKIVRGIIIQGGKHRENKVF |
| | | | MRKFKIGYSNNGSDWKMIMDDSKRKAKSFEGNNNYDTPELRTFPALSTRFIRIYPERATHGGLGLRMELLGCGGGGSGG |
| | | | GGSCMEALGMESGEIHSDQITASSQYSTNWSAERSRLNYPENGWTPGADSYREWIQVDLGLLRFVTAVGTQGAISKETK |
| | | | KKYYVKTYKIDVSSNGEDWITIKEGNKPVLFQGNTNPTDVVVAVFPKPLITRFVRIKPATWETGISMRFEVYGCKITDY |
| | | | PCSGMLGMVSGLISDSQITSSNQGDRNWMPENIRLVTSRSGWALPPAPHSYINEWLQIDLGEEKIVRGIIIQGGKHREN |
| | | | KVFMRKFKIGYSNNGSDWKMIMDDSKRKAKSFEGNNNYDTPELRTFPALSTRFIRIYPERATHGGLGLRMELLGCGGGG |
| | | | SGGGGSERKCCVECPPCPAPPVAGPSVFLFPPKPKDQLMISRTPEVTCVVVDVSHEDPEVQFNWYVDGVEVHNAKTKPR |
| | | | EEQFNSTFRVVSVLTVVHQDWLNGKEYKCKVSNKGLPAPIEKTISKTKGQPREPQVYTLPPSREEMTKNQVSLTCLVKG |
| | | | FYPSDIAVEWESNGQPENNYKTTPPMLDSDGSFFLYSKLTVDKSRWQQGNVFSCSVLHEALHNHYTQKSLSLSPGKGGG |
| | | | GSGGGGSIEEQAKTFLDKFNHEAEDLFYQSSLASWNYNTNITEENVQNMNNAGDKWSAFLKEQSTLAQMYPLQEIWDLG |
| | | | KGDFRGGGGSGGGGSIEEQAKTFLDKFNHEAEDLFYQSSLASWNYNTNITEENVQNMNNAGDKWSAFLKEQSTLAQMYP |
| | | | LQEIWDLGKGDFR |
| 58 | 111 | 1044 | CMEALGMESGEIHSDQITASSQASTNWSAERSRLNYPENGWTPGEDSYREWIQVDLGLLRFVTAVGTQGAIAKETKKKA |
| | | | YVKTYKIDVSSNGEDWITIKEGNKPVLFQGNTNPTDVVVAVFPKPLITRFVRIKPATWETGISMRFEVYGCKITDYPCS |
| | | | GMLGMVSGLISDSQITSSNQGDRNWMPENIRLVTSRSGWALPPAPHSYINEWLQIDLGEEKIVRGIIIQGGKHRENKVF |
| | | | MRKFKIGYSNNGSDWKMIMDDSKRKAKSFEGNNNYDTPELRTFPALSTRFIRIYPERATHGGLGLRMELLGCGGGGSGG |
| | | | GGSCMEALGMESGEIHSDQITASSQASTNWSAERSRLNYPENGWTPGEDSYREWIQVDLGLLRFVTAVGTQGAIAKETK |
| | | | KKAYVKTYKIDVSSNGEDWITIKEGNKPVLFQGNTNPTDVVVAVFPKPLITRFVRIKPATWETGISMRFEVYGCKITDY |
| | | | PCSGMLGMVSGLISDSQITSSNQGDRNWMPENIRLVTSRSGWALPPAPHSYINEWLQIDLGEEKIVRGIIIQGGKHREN |
| | | | KVFMRKFKIGYSNNGSDWKMIMDDSKRKAKSFEGNNNYDTPELRTFPALSTRFIRIYPERATHGGLGLRMELLGCGGGG |
| | | | SGGGGSEPKSCDKTHTCPPCPAPELLGGPSVFLFPPKPKDTLMISRTPEVTCVVVDVSHEDPEVKFNWYVDGVEVHNAK |
| | | | TKPREEQYASTYRVVSVLAVLHQDWLNGKEYKCKVSNKALPAPIEKTISKAKGQPREPQVYTLPPSRDELTKNQVSLTC |
| | | | LVKGFYPSDIAVAWESNGQPENNYKTTPPVLDSDGSFFLYSKLTVDKSRWQQGNVFSCSVMHEALHAHYTQKSLSLSPG |
| | | | KGGGGSGGGGSIEEQAKTFLDKFNHEAEDLFYQSSLASWNYNTNITEENVQNMNNAGDKWSAFLKEQSTLAQMYPLQEI |
| | | | WDLGKGDFRGGGGSGGGGSIEEQAKTFLDKFNHEAEDLFYQSSLASWNYNTNITEENVQNMNNAGDKWSAFLKEQSTLA |
| | | | QMYPLQEIWDLGKGDFR |
| 59 | 112 | 1044 | CMEALGMESGEIHSDQITASSQASTNWSAERSRLNYPENGWTPGEDSYREWIQVDLGLLRFVTAVGTQGAIAKETKKKA |
| | | | YVKTYKIDVSSNGEDWITIKEGNKPVLFQGNTNPTDVVVAVFPKPLITRFVRIKPATWETGISMRFEVYGCKITDYPCS |
| | | | GMLGMVSGLISDSQITSSNQGDRNWMPENIRLVTSRSGWALPPAPHSYINEWLQIDLGEEKIVRGIIIQGGKHRENKVF |
| | | | MRKFKIGYSNNGSDWKMIMDDSKRKAKSFEGNNNYDTPELRTFPALSTRFIRIYPERATHGGLGLRMELLGCGGGGSGG |
| | | | GGSCMEALGMESGEIHSDQITASSQASTNWSAERSRLNYPENGWTPGEDSYREWIQVDLGLLRFVTAVGTQGAIAKETK |
| | | | KKAYVKTYKIDVSSNGEDWITIKEGNKPVLFQGNTNPTDVVVAVFPKPLITRFVRIKPATWETGISMRFEVYGCKITDY |
| | | | PCSGMLGMVSGLISDSQITSSNQGDRNWMPENIRLVTSRSGWALPPAPHSYINEWLQIDLGEEKIVRGIIIQGGKHREN |
| | | | KVFMRKFKIGYSNNGSDWKMIMDDSKRKAKSFEGNNNYDTPELRTFPALSTRFIRIYPERATHGGLGLRMELLGCGGGG |
| | | | SGGGGSEPKSCDKTHTCPPCPAPEAAGGPSVFLFPPKPKDTLMISRTPEVTCVVVDVSHEDPEVKFNWYVDGVEVHNAK |
| | | | TKPREEQYNSTYRVVSVLAVLHQDWLNGKEYKCKVSNKALPAPIEKTISKAKGQPREPQVYTLPPSRDELTKNQVSLTC |
| | | | LVKGFYPSDIAVAWESNGQPENNYKTTPPVLDSDGSFFLYSKLTVDKSRWQQGNVFSCSVMHEALHAHYTQKSLSLSPG |
| | | | KGGGGSGGGGSIEEQAKTFLDKFNHEAEDLFYQSSLASWNYNTNITEENVQNMNNAGDKWSAFLKEQSTLAQMYPLQEI |
| | | | WDLGKGDFRGGGGSGGGGSIEEQAKTFLDKFNHEAEDLFYQSSLASWNYNTNITEENVQNMNNAGDKWSAFLKEQSTLA |
| | | | QMYPLQEIWDLGKGDFR |

Neuropilin-ACE2 Constructs

In yet another aspect of the current invention, a polypeptide may comprise a b1 domain, or a derivative or fragment thereof, of a neuropilin; and an ACE2 domain, or a derivative or fragment thereof, of angiotensin converting enzyme 2. The b1 domain and ACE2 domain in these embodiments are each capable of binding to a coat protein of a virus selected from the group consisting of herpesviridae, papillomaviridae, coronaviridae, flaviviridae, togaviridae, bornaviridae, bunyaviridae, filoviridae, orthomyxoviridae, paramyxoviridae, pneumoviridae, and retroviridae.

In some embodiments, the b1 domain, or a derivative or fragment thereof, of a neuropilin may be selected from the group including one or more of SEQ ID NOS: 1-22 coupled to the ACE2 domain, or a derivative or fragment thereof, of angiotensin converting enzyme 2 including one or more of SEQ ID NOS: 32-43. In some embodiments, the b1 domain is attached to the C-terminus of the ACE2 domain. In other embodiments, the b1 domain is attached to the N-terminus of the ACE2 domain. In still other embodiments, a linker selected from the group including one or more of SEQ ID NOS: 44-50 may be coupled between any combinations of the one or more b1 domains fused to the one or more ACE2 domains.

In some embodiments, the neuropilin-ACE2 polypeptides comprising a b1 domain, or a derivative or fragment thereof, and an ACE2 domain, or a derivative or fragment thereof, may be used in nasal spray compositions.

Pharmaceutical Compositions

The polypeptides described herein can be formulated into pharmaceutical compositions that further comprise a pharmaceutically acceptable carrier, diluent, adjuvant or vehicle. In one embodiment, the present invention relates to a pharmaceutical composition comprising a polypeptide of the invention described above, and a pharmaceutically acceptable carrier, diluent, adjuvant or vehicle. In one embodiment, the present invention is a pharmaceutical composition comprising an effective amount of a polypeptide of the present invention or a pharmaceutically acceptable salt thereof and a pharmaceutically acceptable carrier, diluent, adjuvant or vehicle. Pharmaceutically acceptable carriers include, for example, pharmaceutical diluents, excipients or carriers suitably selected with respect to the intended form of administration, and consistent with conventional pharmaceutical practices.

An “effective amount” includes a “therapeutically effective amount” and a “prophylactically effective amount”. The term “therapeutically effective amount” refers to an amount effective in treating and/or ameliorating a virus infection in a patient infected with a viral infection, e.g., SARS-CoV-2. The term “prophylactically effective amount” refers to an amount effective in preventing and/or substantially lessening the chances or the size of the virus infection outbreak.

A pharmaceutically acceptable carrier may contain inert ingredients which do not unduly inhibit the biological activity of the polypeptides. The pharmaceutically acceptable carriers should be biocompatible, e.g., non-toxic, non-inflammatory, non-immunogenic or devoid of other undesired reactions or side-effects upon the administration to a subject. Standard pharmaceutical formulation techniques can be employed.

The pharmaceutically acceptable carrier, adjuvant, or vehicle, as used herein, includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutically acceptable compositions and known techniques for the preparation thereof. Except insofar as any conventional carrier medium is incompatible with the polypeptides described herein, such as by producing any undesirable biological effect or otherwise interacting in a deleterious manner with any other component(s) of the pharmaceutically acceptable composition, its use is contemplated to be within the scope of this invention. As used herein, the phrase “side effects” encompasses unwanted and adverse effects of a therapy (e.g., a prophylactic or therapeutic agent). Side effects are always unwanted, but unwanted effects are not necessarily adverse. An adverse effect from a therapy (e.g., prophylactic or therapeutic agent) might be harmful or uncomfortable or risky. Side effects include, but are not limited to fever, chills, lethargy, gastrointestinal toxicities (including gastric and intestinal ulcerations and erosions), nausea, vomiting, neurotoxicities, nephrotoxicities, renal toxicities (including such conditions as papillary necrosis and chronic interstitial nephritis), hepatic toxicities (including elevated serum liver enzyme levels), myelotoxicities (including leukopenia, myelosuppression, thrombocytopenia and anemia), dry mouth, metallic taste, prolongation of gestation, weakness, somnolence, pain (including muscle pain, bone pain and headache), hair loss, asthenia, dizziness, extra-pyramidal symptoms, akathisia, cardiovascular disturbances and sexual dysfunction.

Some examples of materials which can serve as pharmaceutically acceptable carriers include, but are not limited to, ion exchangers, alumina, aluminum stearate, lecithin, serum proteins (such as human serum albumin), buffer substances (such as twin 80, phosphates, glycine, sorbic acid, or potassium sorbate), partial glyceride mixtures of saturated vegetable fatty acids, water, salts or electrolytes (such as protamine sulfate, disodium hydrogen phosphate, potassium hydrogen phosphate, sodium chloride, or zinc salts), colloidal silica, magnesium trisilicate, polyvinyl pyrrolidone, polyacrylates, waxes, polyethylene-polyoxypropylene-block polymers, methylcellulose, hydroxypropyl methylcellulose, wool fat, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol or polyethylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

In some embodiments, a composition of the present invention comprises a pharmaceutically acceptable salt.

The term “pharmaceutically acceptable salts” is meant to include salts of the active compounds that are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present invention contain both basic and acidic functionalities that allow the compounds to be converted into either base or acid addition salts.

Thus, the compounds of the present invention may exist as salts, such as with pharmaceutically acceptable acids. The present invention includes such salts. Non-limiting examples of such salts include hydrochlorides, hydrobromides, phosphates, sulfates, methanesulfonates, nitrates, maleates, acetates, citrates, fumarates, proprionates, tartrates (e.g., (+)-tartrates, (−)-tartrates, or mixtures thereof including racemic mixtures), succinates, benzoates, and salts with amino acids such as glutamic acid, and quaternary ammonium salts (e.g. methyl iodide, ethyl iodide, and the like). These salts may be prepared by methods known to those skilled in the art.

The neutral forms of the compounds are preferably regenerated by contacting the salt with a base or acid and isolating the parent compound in the conventional manner. The parent form of the compound may differ from the various salt forms in certain physical properties, such as solubility in polar solvents.

In addition to salt forms, the present invention provides compounds, which are in a prodrug form. Prodrugs of the compounds described herein are those compounds that readily undergo chemical changes under physiological conditions to provide the compounds of the present invention. Prodrugs of the compounds described herein may be converted in vivo after administration. Additionally, prodrugs can be converted to the compounds of the present invention by chemical or biochemical methods in an ex vivo environment, such as, for example, when contacted with a suitable enzyme or chemical reagent.

Certain compounds of the present invention can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention.

Administration Methods

In some embodiments, the compositions of the present invention may be administered to a subject in need thereof. In some embodiments, the compositions of the present invention may be co-administered with one or more additional therapies.

The terms “administration” or “administering” refer to the act of providing an composition of the present invention, e.g., a polypeptide or pharmaceutically acceptable salt thereof, to a subject in need of treatment thereof.

In some embodiments, the compositions of the present invention can be administered as follows: oral administration, administration as a suppository, topical contact, intravenous, parenteral, intraperitoneal, intramuscular, intralesional, intrathecal, intranasal or subcutaneous administration, or the implantation of a slow-release device, e.g., a mini-osmotic pump, to a subject. Accordingly, administration can be by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and. Other modes of delivery include, but are not limited to, the use of liposomal formulations, intravenous infusion, transdermal patches, etc.

By “co-administer” it is meant that a composition described herein is administered at the same time, just prior to, or just after the administration of additional therapies. The therapeutic drugs can be administered alone or can be co-administered to the patient. Co-administration is meant to include simultaneous or sequential administration of the components individually or in combination. Thus, the preparations can also be combined, when desired, with other active substances. As used herein, “sequential administration” includes that the administration of two agents (e.g., the agents described herein) do not occur on a same day.

As used herein, “concurrent administration” includes overlapping in duration at least in part. For example, when two compositions (e.g., any of the compositions described herein) are administered concurrently, their administration occurs within a certain desired time. The administration of the compositions may begin and end on the same day. The administration of one composition can also precede the administration of a second composition by day(s) as long as both compositions are taken on the same day at least once. Similarly, the administration of one composition can extend beyond the administration of a second composition as long as both agents are taken on the same day at least once. The composition do not have to be taken at the same time each day to include concurrent administration.

As used herein, “intermittent administration includes the administration of an agent for a period of time (which can be considered a “first period of administration”), followed by a time during which the composition is not taken or is taken at a lower maintenance dose (which can be considered “off-period”) followed by a period during which the composition is administered again (which can be considered a “second period of administration”). Generally, during the second phase of administration, the dosage level of the agent will match that administered during the first period of administration but can be increased or decreased as medically necessary.

The polypeptides and pharmaceutically acceptable compositions described above can be administered to humans and other animals orally, rectally, parenterally, intracisternally, intravaginally, intraperitoneally, topically (as by powders, ointments, or drops), buccally, as an oral or nasal spray, or the like, depending on the severity of the infection being treated.

In some embodiments of the invention, when treating a subject, an inhibitory agent is administered by systemic intravenous (IV) or by a local intranasal route, such as an intranasal spray, a metered-dose inhaler, a nebulizer, or a dry powder inhaler. Formulations for delivery by a particular method (e.g., solutions, buffers, and preservatives, as well as droplet or particle size for intranasal administration) can be optimized by routine, conventional methods that are well-known in the art. For inhibitory agents that are in the form of aerosol formulations to be administered via inhalation, the aerosol formulations can be placed into pressurized acceptable propellants, such as dichlorodifluoromethane, propane, nitrogen or the like.

Liquid dosage forms for oral administration include, but are not limited to, pharmaceutically acceptable emulsions, microemulsions, solutions, suspensions, syrups and elixirs. In addition to the active polypeptides, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a polypeptide described herein, it is often desirable to slow the absorption of the polypeptide from subcutaneous or intramuscular injection. This may be accomplished by the use of a liquid suspension of crystalline or amorphous material with poor water solubility. The rate of absorption of the polypeptide then depends upon its rate of dissolution that, in turn, may depend upon crystal size and crystalline form. Alternatively, delayed absorption of a parenterally administered polypeptide form is accomplished by dissolving or suspending the polypeptide in an oil vehicle. Injectable depot forms are made by forming microencapsule matrices of the polypeptide in biodegradable polymers such as polylactide-polyglycolide. Depending upon the ratio of polypeptide to polymer and the nature of the particular polymer employed, the rate of polypeptide release can be controlled. Examples of other biodegradable polymers include poly(orthoesters) and poly(anhydrides). Depot injectable formulations are also prepared by entrapping the polypeptide in liposomes or microemulsions that are compatible with body tissues.

Compositions for rectal or vaginal administration are specifically suppositories which can be prepared by mixing the polypeptides described herein with suitable non-irritating excipients or carriers such as cocoa butter, polyethylene glycol or a suppository wax which are solid at ambient temperature but liquid at body temperature and therefore melt in the rectum or vaginal cavity and release the active polypeptide.

Solid dosage forms for oral administration include capsules, tablets, pills, powders, and granules. In such solid dosage forms, the polypeptide (i.e., active polypeptide) is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polyethylene glycols and the like.

The active polypeptides can also be in microencapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active polypeptide may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a polypeptide described herein include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, eardrops, and eye drops are also contemplated as being within the scope of this invention. Additionally, the present invention contemplates the use of transdermal patches, which have the added advantage of providing controlled delivery of a polypeptide to the body. Such dosage forms can be made by dissolving or dispensing the polypeptide in the proper medium. Absorption enhancers can also be used to increase the flux of the polypeptide across the skin. The rate can be controlled by either providing a rate controlling membrane or by dispersing the polypeptide in a polymer matrix or gel.

The compositions described herein may be administered orally, parenterally, by inhalation spray, topically, rectally, nasally, buccally, vaginally or via an implanted reservoir. The term “parenteral” as used herein includes, but is not limited to, subcutaneous, intravenous, intramuscular, intra-articular, intra-synovial, intrasternal, intrathecal, intrahepatic, intralesional and intracranial injection or infusion techniques. Specifically, the compositions are administered orally, intraperitoneally or intravenously.

Sterile injectable forms of the compositions described herein may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents which are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

The pharmaceutical compositions described herein may be orally administered in any orally acceptable dosage form including, but not limited to, capsules, tablets, aqueous suspensions or solutions. In the case of tablets for oral use, carriers commonly used include, but are not limited to, lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, the pharmaceutical compositions described herein may be administered in the form of suppositories for rectal administration. These can be prepared by mixing the agent with a suitable non-irritating excipient which is solid at room temperature but liquid at rectal temperature and therefore will melt in the rectum to release the drug. Such materials include, but are not limited to, cocoa butter, beeswax and polyethylene glycols.

The pharmaceutical compositions described herein may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, the pharmaceutical compositions may be formulated in a suitable ointment containing the active component suspended or dissolved in one or more carriers. Carriers for topical administration of the polypeptides of this invention include, but are not limited to, mineral oil, liquid petrolatum, white petrolatum, propylene glycol, polyoxyethylene, polyoxypropylene compound, emulsifying wax and water. Alternatively, the pharmaceutical compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2 octyldodecanol, benzyl alcohol and water.

For ophthalmic use, the pharmaceutical compositions may be formulated as micronized suspensions in isotonic, pH adjusted sterile saline, or, specifically, as solutions in isotonic, pH adjusted sterile saline, either with or without a preservative such as benzylalkonium chloride. Alternatively, for ophthalmic uses, the pharmaceutical compositions may be formulated in an ointment such as petrolatum.

The pharmaceutical compositions may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

The polypeptides for use in the methods of the invention can be formulated in unit dosage form. The term “unit dosage form” refers to physically discrete units suitable as unitary dosage for subjects undergoing treatment, with each unit containing a predetermined quantity of active material calculated to produce the desired therapeutic effect, optionally in association with a suitable pharmaceutical carrier. The unit dosage form can be for a single daily dose or one of multiple daily doses (e.g., about 1 to 4 or more times per day). When multiple daily doses are used, the unit dosage form can be the same or different for each dose.

Pulmonary/Nasal Administration

For pulmonary administration, preferably, at least one polypeptide composition is delivered in a particle size effective for reaching the lower airways of the lung or sinuses. According to the invention, at least one polypeptide as disclosed herein can be delivered by any of a variety of inhalation or nasal devices known in the art for administration of a therapeutic agent by inhalation. These devices are capable of depositing aerosolized formulations in the sinus cavity or alveoli of a patient include metered dose inhalers, nebulizers, dry powder generators, sprayers, and the like. Other devices suitable for directing the pulmonary or nasal administration of polypeptides are also known in the art. Many of such devices can use formulations suitable for the administration for the dispensing of polypeptides in an aerosol. Such aerosols can be comprised of either solutions (both aqueous and non-aqueous) or solid particles.

Metered dose inhalers like the Ventolin® metered dose inhaler, typically use a propellant gas and require actuation during inspiration (See, e.g., WO 94/16970, WO 98/35888). Dry powder inhalers like TURBUHALER™ (Astra), ROTAHALER® (Glaxo), DISKUS® (Glaxo), SPIROS™ inhaler (Dura), devices marketed by Inhale Therapeutics, and the SPINHALER® powder inhaler (Fisons), use breath-actuation of a mixed powder (U.S. Pat. No. 4,668,218 Astra, EP 237507 Astra, WO 97/25086 Glaxo, WO 94/08552 Dura, U.S. Pat. No. 5,458,135 Inhale, WO 94/06498 Fisons, entirely incorporated herein by reference). Nebulizers like AERX™ Aradigm, the ULTRAVENT® nebulizer (Mallinckrodt), and the ACORN II® nebulizer (Marquest Medical Products) (U.S. Pat. No. 5,404,871 Aradigm, WO 97/22376), the above references are entirely incorporated herein by reference, produce aerosols from solutions, while metered dose inhalers, dry powder inhalers, etc. generate small particle aerosols. These specific examples of commercially available inhalation devices are intended to be a representative of specific devices suitable for the practice of this invention, and are not intended as limiting the scope of the invention.

In some embodiments, a composition comprising at least one polypeptide as disclosed herein is delivered by a dry powder inhaler or a sprayer. There are several desirable features of an inhalation device for administering at least one polypeptide of the present invention. For example, delivery by the inhalation device is advantageously reliable, reproducible, and accurate. The inhalation device can optionally deliver small dry particles, e.g., less than about 10 μm, preferably about 1-5 μm, for good respirability.

A spray including the polypeptide composition can be produced by forcing a suspension or solution of at least one polypeptide through a nozzle under pressure. The nozzle size and configuration, the applied pressure, and the liquid feed rate can be chosen to achieve the desired output and particle size. An electrospray can be produced, for example, by an electric field in connection with a capillary or nozzle feed. Advantageously, particles of at least one polypeptide delivered by a sprayer have a particle size less than about 10 μm, in some embodiments, in the range of about 1 μm to about 5 μm, of from about 2 μm to about 3 μm.

Formulations having at least one polypeptide suitable for use with a sprayer typically include a polypeptide composition in an aqueous solution at a concentration of about 0.1 mg to about 100 mg of at least one polypeptide per ml of solution or mg/gm, or any range, value, or fraction therein. The formulation can include agents, such as an excipient, a buffer, an isotonicity agent, a preservative, a surfactant, and, preferably, zinc. The formulation can also include an excipient or agent for stabilization of the polypeptide composition, such as a buffer, a reducing agent, a bulk protein, or a carbohydrate. Bulk proteins useful in formulating polypeptide compositions include albumin, protamine, or the like. Typical carbohydrates useful in formulating polypeptide compositions include sucrose, mannitol, lactose, trehalose, glucose, or the like. The polypeptide composition formulation can also include a surfactant, which can reduce or prevent surface-induced aggregation of the polypeptide composition caused by atomization of the solution in forming an aerosol. Various conventional surfactants can be employed, such as polyoxyethylene fatty acid esters and alcohols, and polyoxyethylene sorbitol fatty acid esters. Amounts will generally range between 0.001 and 14% by weight of the formulation. Especially preferred surfactants for purposes of this invention are polyoxyethylene sorbitan monooleate, polysorbate 80, polysorbate 20, or the like. Additional agents known in the art for formulation of polypeptides, such as IL-23p19 antibodies, or specified portions or variants, can also be included in the formulation.

Administration of the Polypeptide Compositions by a Nebulizer

Polypeptide compositions of the invention can be administered by a nebulizer, such as jet nebulizer or an ultrasonic nebulizer. Typically, in a jet nebulizer, a compressed air source is used to create a high-velocity air jet through an orifice. As the gas expands beyond the nozzle, a low-pressure region is created, which draws a solution of polypeptide composition through a capillary tube connected to a liquid reservoir. The liquid stream from the capillary tube is sheared into unstable filaments and droplets as it exits the tube, creating the aerosol. A range of configurations, flow rates, and baffle types can be employed to achieve the desired performance characteristics from a given jet nebulizer. In an ultrasonic nebulizer, high-frequency electrical energy is used to create vibrational, mechanical energy, typically employing a piezoelectric transducer. This energy is transmitted to the formulation of the polypeptide composition either directly or through a coupling fluid, creating an aerosol including the polypeptide composition. Advantageously, particles of the polypeptide composition delivered by a nebulizer have a particle size less than about 10 μm, in some embodiments, in the range of about 1 μm to about 5 μm, or from about 2 μm to about 3 μm.

Formulations of at least one polypeptide suitable for use with a nebulizer, either jet or ultrasonic, typically include a concentration of about 0.1 mg to about 100 mg of at least one polypeptide per ml of solution. The formulation can include agents, such as an excipient, a buffer, an isotonicity agent, a preservative, a surfactant, and, preferably, zinc. The formulation can also include an excipient or agent for stabilization of the at least one polypeptide composition, such as a buffer, a reducing agent, a bulk protein, or a carbohydrate. Bulk proteins useful in formulating at least one polypeptide compositions include albumin, protamine, or the like. Typical carbohydrates useful in formulating at least one polypeptide include sucrose, mannitol, lactose, trehalose, glucose, or the like. The at least one polypeptide formulation can also include a surfactant, which can reduce or prevent surface-induced aggregation of the at least one polypeptide caused by atomization of the solution in forming an aerosol. Various conventional surfactants can be employed, such as polyoxyethylene fatty acid esters and alcohols, and polyoxyethylene sorbital fatty acid esters. Amounts will generally range between about 0.001 and 4% by weight of the formulation. Especially preferred surfactants for purposes of this invention are polyoxyethylene sorbitan mono-oleate, polysorbate 80, polysorbate 20, or the like. Additional agents known in the art for formulation of a polypeptide, such as antibody protein, can also be included in the formulation.

Administration of the Polypeptide Compositions by a Metered Dose Inhaler

In a metered dose inhaler (MDI), a propellant, at least one polypeptide as disclosed herein, and any excipients or other additives are contained in a canister as a mixture including a liquefied compressed gas. Actuation of the metering valve releases the mixture as an aerosol, preferably containing particles in the size range of less than about 10 μm, in some embodiments, about 1 μm to about 5 μm, or from about 2 μm to about 3 μm. The desired aerosol particle size can be obtained by employing a formulation of polypeptide composition produced by various methods known to those of skill in the art, including jet-milling, spray drying, critical point condensation, or the like. Preferred metered dose inhalers include those manufactured by 3M or Glaxo and employing a hydrofluorocarbon propellant. Formulations of at least one polypeptide for use with a metered-dose inhaler device will generally include a finely divided powder containing at least one polypeptide as a suspension in a non-aqueous medium, for example, suspended in a propellant with the aid of a surfactant. The propellant can be any conventional material employed for this purpose, such as chlorofluorocarbon, a hydrochlorofluorocarbon, a hydrofluorocarbon, or a hydrocarbon, including trichlorofluoromethane, dichlorodifluoromethane, dichlorotetrafluoroethanol and 1,1,1,2-tetrafluoroethane, HFA-134a (hydrofluroalkane-134a), HFA-227 (hydrofluroalkane-227), or the like. Preferably, the propellant is a hydrofluorocarbon. The surfactant can be chosen to stabilize the at least one polypeptide as a suspension in the propellant, to protect the active agent against chemical degradation, and the like. Suitable surfactants include sorbitan trioleate, soya lecithin, oleic acid, or the like. In some cases, solution aerosols are preferred using solvents, such as ethanol. Additional agents known in the art for formulation of a polypeptide can also be included in the formulation. One of ordinary skill in the art will recognize that the methods of the current invention can be achieved by pulmonary administration of at least one polypeptide composition via devices not described herein.

Combination Therapy

An effective amount can be achieved in the method or pharmaceutical composition of the invention employing the polypeptide or a pharmaceutically acceptable salt or solvate (e.g., hydrate) thereof alone or in combination with an additional suitable therapeutic agent, for example, an antiviral agent or a vaccine. When “combination therapy” is employed, an effective amount can be achieved using a first amount of the polypeptide, or a pharmaceutically acceptable salt or solvate (e.g., hydrate) thereof, and a second amount of an additional suitable therapeutic agent (e.g. an antiviral agent or vaccine).

In other embodiments of this invention, the polypeptide and the additional therapeutic agent, are each administered in an effective amount (i.e., each in an amount which would be therapeutically effective if administered alone). In another embodiment, the polypeptide and the additional therapeutic agent are each administered in an amount which alone does not provide a therapeutic effect (a sub-therapeutic dose). In yet another embodiment, the polypeptide can be administered in an effective amount, while the additional therapeutic agent is administered in a sub-therapeutic dose. In still another embodiment, the polypeptide can be administered in a sub-therapeutic dose, while the additional therapeutic agent, for example, a suitable anti-viral therapeutic agent is administered in an effective amount.

As used herein, the terms “in combination” or “co-administration” can be used interchangeably to refer to the use of more than one therapy (e.g., one or more prophylactic and/or therapeutic agents). The use of the terms does not restrict the order in which therapies (e.g., prophylactic and/or therapeutic agents) are administered to a subject.

Co-administration encompasses administration of the first and second amounts of the polypeptides of the co-administration in an essentially simultaneous manner, such as in a single pharmaceutical composition, for example, capsule or tablet having a fixed ratio of first and second amounts, or in multiple, separate capsules or tablets for each. In addition, such co-administration also encompasses use of each polypeptide in a sequential manner in either order.

In some embodiments, the present invention is directed to methods of combination therapy for treating a viral infection (e.g., COVID-19) by inhibiting the virus's replication in biological samples or patients, or for treating or preventing virus infections in patients using the polypeptides or pharmaceutical compositions of the invention. Accordingly, pharmaceutical compositions of the invention also include those comprising an inhibitor of virus replication of this invention in combination with an anti-viral polypeptide exhibiting anti-viral activity.

When co-administration involves the separate administration of the first amount of the polypeptide and a second amount of an additional therapeutic agent, the polypeptides are administered sufficiently close in time to have the desired therapeutic effect. For example, the period of time between each administration which can result in the desired therapeutic effect, can range from minutes to hours and can be determined taking into account the properties of each polypeptide such as potency, solubility, bioavailability, plasma half-life and kinetic profile. For example, the polypeptide and the second therapeutic agent can be administered in any order within about 24 hours of each other, within about 16 hours of each other, within about 8 hours of each other, within about 4 hours of each other, within about 1 hour of each other or within about 30 minutes of each other.

More, specifically, a first therapy (e.g., a prophylactic or therapeutic agent such as any one of the polypeptides of the invention) can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy (e.g., a prophylactic or therapeutic agent such as an anti-viral agent) to a subject.

It is understood that the method of co-administration of a first amount of the polypeptide and a second amount of an additional therapeutic agent can result in an enhanced or synergistic therapeutic effect, wherein the combined effect is greater than the additive effect that would result from separate administration of the first amount of the polypeptide and the second amount of the additional therapeutic agent.

As used herein, the term “synergistic” refers to a combination of a polypeptide of the invention and another therapy (e.g., a prophylactic or therapeutic agent), which is more effective than the additive effects of the therapies. A synergistic effect of a combination of therapies (e.g., a combination of prophylactic or therapeutic agents) can permit the use of lower dosages of one or more of the therapies and/or less frequent administration of said therapies to a subject. The ability to utilize lower dosages of a therapy (e.g., a prophylactic or therapeutic agent) and/or to administer said therapy less frequently can reduce the toxicity associated with the administration of said therapy to a subject without reducing the efficacy of said therapy in the prevention, management or treatment of a disorder. In addition, a synergistic effect can result in improved efficacy of agents in the prevention, management or treatment of a disorder. Finally, a synergistic effect of a combination of therapies (e.g., a combination of prophylactic or therapeutic agents) may avoid or reduce adverse or unwanted side effects associated with the use of either therapy alone.

When the combination therapy using polypeptides of the present invention is in combination with a vaccine, both therapeutic agents can be administered so that the period of time between each administration can be longer (e.g. days, weeks or months).

The presence of a synergistic effect can be determined using suitable methods for assessing drug interaction. Suitable methods include, for example, the Sigmoid-Emax equation (Holford, N. H. G. and Schemer, L. B., Clin. Pharmacokinet. 6: 429-453 (1981)), the equation of Loewe additivity (Loewe, S. and Muischnek, H., Arch. Exp. Pathol Pharmacol. 114: 313-326 (1926)) and the median-effect equation (Chou, T. C. and Talalay, P., Adv. Enzyme Regul. 22: 27-55 (1984)). Each equation referred to above can be applied with experimental data to generate a corresponding graph to aid in assessing the effects of the drug combination. The corresponding graphs associated with the equations referred to above are the concentration-effect curve, isobologram curve and combination index curve, respectively.

Specific examples that can be co-administered with a polypeptide described herein include Remdesivir, neuraminidase inhibitors, such as oseltamivir (Tamiflu®) and Zanamivir (RLENZA®), viral ion channel (M2 protein) blockers, such as amantadine (SYMMETREL®) and rimantadine (FLUMADINE®), and antiviral drugs described in WO 2003/015798, including T-705 under development by Toyama Chemical of Japan. (See also Ruruta et al., Antiviral Research, 82: 95-102 (2009), “T-705 (flavipiravir) and related compounds: Novel broad-spectrum inhibitors of RNA viral infections.”) In some embodiments, the polypeptides described herein can be co-administered with a traditional influenza vaccine.

### Exemplary Embodiments

In some embodiments, the present invention comprises, consists essentially of, or consists of, a recombinant polypeptide comprising: (a) one or more mutant neuropilin (NRP) domains, or fragments thereof, and (b) an immunoglobulin domain; wherein the one or more mutant NRP domains result in reduced binding of the recombinant polypeptide to heparin or heparan sulfate relative to a wild-type NRP domain.

In some embodiments, the recombinant polypeptide comprises one or more mutant NRP domains are derived from an NRP1 or an NRP2 protein.

In some embodiments, the one or more mutant NRP domains are one or more mutant b1 domains, or one or more mutant b2 domains.

In some embodiments, the one or more mutant NRP domains has one or more amino substitutions selected from groups consisting of: K373E, K351A, E319A, K358E, R513E, K514E, K516E, R513A, K514A, K516A, Y297A, S345A, and Y353A, relative to the wild-type amino acid sequence set forth in SEQ ID NO: 1.

In some embodiments, the one or more mutant NRP domains result in reduced binding of the recombinant polypeptide to heparin.

In some embodiments, the one or more mutant NRP domains result in reduced binding of the recombinant polypeptide to heparan sulfate.

In some embodiments, the immunoglobin domain is an Fc domain.

In some embodiments, the present invention comprises, consists essentially of, or consists of, a recombinant polypeptide comprising: (a) one or more mutant neuropilin (NRP) b1 domains, NRP b2 domains, or fragments thereof, and (b) an Fc domain; wherein the one or more mutant NRP b1 domains, NRP b2 domains, or fragments thereof are derived from an NRP1 or an NRP2 protein; wherein the one or more mutant NRP b1 domains, NRP b2 domains, or fragments thereof have one or more amino substitutions selected from groups consisting of: K373E, K351A, E319A, K358E, R513E, K514E, K516E, R513A, K514A, K516A, Y297A, S345A, and Y353A, relative to the wild-type amino acid sequence set forth in SEQ ID NO: 1; and wherein the one or more one or more amino substitutions result in reduced binding of the recombinant polypeptide to heparin or heparan sulfate.

In some embodiments, the present invention comprises, consists essentially of, or consists of, a recombinant polypeptide comprising: (a) one or more mutant neuropilin (NRP) b1 domains (b1), NRP b2 domains (b2), or fragments thereof; and (b) an Fc domain; wherein (a) and (b) comprise a construct having an orientation of: b1-Fc; b1b1-Fc; b1b1b1-Fc; b1-Fc; b1b1b1-Fc; b1b2-Fc; b1b2-Fc; b1b2-Fc; b1b2-Fc; Fc-b1b2; Fc-b1b2; b1-Fc-b1; b1b1-Fc-b1; b1-Fc; b1b1-Fc; b1b1b1-Fc; b1-Fc; b1b1b1-Fc; b1-Fc; b1b1b1-Fc; b1b2-Fc; b1b2-Fc; Fc-b1b2; Fc-b1b2; b1-Fc-b1; b1b1-Fc-b1; wherein the one or more b1, b2, or fragments thereof, are derived from an NRP1 or an NRP2 protein; wherein the one or more b1, b2, or fragments thereof have one or more amino substitutions selected from groups consisting of: K373E, K351A, E319A, K358E, R513E, K514E, K516E, R513A, K514A, K516A, Y297A, S345A, and Y353A, relative to the wild-type amino acid sequence set forth in SEQ ID NO: 1; and wherein the one or more one or more amino substitutions result in reduced binding of the recombinant polypeptide to heparin or heparan sulfate.

In some embodiments, the present invention comprises, consists essentially of, or consists of, a recombinant polypeptide comprising: (a) one or more mutant neuropilin (NRP) domains, or fragments thereof, and (b) an immunoglobulin domain; wherein the recombinant polypeptide is operable to bind to virus having an —Z₁-X₁-X₂—Z₂- (CendR) motif, wherein Z₁ and Z₂ are arginine or lysine, and X₁ and X₂ are any amino acid.

In some embodiments, the recombinant polypeptide has one or more mutant NRP domains, or fragments thereof, that are derived from an NRP1 or an NRP2 protein.

In some embodiments, the one or more mutant NRP domains, or fragments thereof, are one or more mutant b1 domains, or one or more mutant b2 domains.

In some embodiments, the present invention comprises, consists essentially of, or consists of, a recombinant polypeptide comprising: (a) one or more mutant neuropilin (NRP) domains, or fragments thereof, and (b) an immunoglobulin domain; wherein the recombinant polypeptide is operable to bind to virus having an —Z₁-X₁-X₂—Z₂- (CendR) motif, wherein Z₁ and Z₂ are arginine or lysine, and X₁ and X₂ are any amino acid, wherein the virus is a virus belonging to the Realm: Duplodnaviria; Monodnaviria; Riboviria; or Varidnaviria.

In some embodiments, the virus is a virus belonging to the Kingdom: Bamfordvirae; Heunggongvirae; Orthornavirae; Pararnavirae; or Shotokuvirae.

In some embodiments, the virus is a virus belonging to the Phylum: Artverviricota; Cossaviricota; Kitrinoviricota; Negarnaviricota; Nucleocytoviricota; Peploviricota; or Pisuviricota.

In some embodiments, the virus is a virus belonging to the Class: Alsuviricetes; Ellioviricetes; Flasuviricetes; Herviviricetes; Insthoviricetes; Monjiviricetes; Papovaviricetes; Pisoniviricetes; Pokkesviricetes; Revtraviricetes; or Stelpaviricetes.

In some embodiments, the virus is a virus belonging to the Order: Amarillovirales; Articulavirales; Bunyavirales; Chitovirales; Hepelivirales; Herpesvirales; Jingchuvirales; Martellivirales; Mononegavirales; Nidovirales; Ortervirales; Stellavirales; or Zurhausenvirales.

In some embodiments, the virus is a virus belonging to the Family: Astroviridae; Bunyaviridae; Bornaviridae; Chuviridae; Coronaviridae; Flaviviridae; Filoviridae; Hantaviridae; Hepeviridae; Herpesviridae; Nairoviridae; Orthomyxoviridae; Papillomaviridae; Paramyxoviridae; Peribunyaviridae; Phenuiviridae; Pneumoviridae; Poxviridae; Retroviridae; Rhabdoviridae; or Togaviridae.

In some embodiments, the virus is selected from the group consisting of: Dengue; respiratory syncytial virus (RSV); Hantavirus; Epstein-Barr virus (EBV); EBV (uncleaved); SARS-CoV-2 Wuhan; SARS-CoV-2 Wuhan (uncleaved); SARS-CoV-2 UK; SARS-CoV-2 India; SARS-CoV-2 India (uncleaved); HCoV-OC43; MERS-CoV; MERS-CoV (uncleaved); Herpes simplex virus (HSV) 1; HSV 1 (uncleaved); influenza A H5N1 virus (IAV H5N1); human papillomavirus (HPV); Human Metapneumovirus; and human immunodeficiency virus (HIV).

In some embodiments, the virus has a CendR motif selected from the group consisting of:

GTCTQSGERRREKR;
KNTNVTLSKKRKRR;
LTHKMIEESHRLRR;
VSFKPPPPPSRRRR;
VSFKPPPPPSRRRRGACVVY;
CASYQTQTNSPRRAR;
CASYQTQTNSPRRARSVASQSIIAYTMSLG;
ASYQTQTNSHRRAR;
ASYQTQTNSRRRAR;
ASYQTQTNSRRRARSVASQSIIAY;
GSGYCVDYSKNRRSR;
LLEPVSISTGSRSAR;
LLEPVSISTGSRSARSAIEDLLFDK;
ERPRAPARSASRPRR;
ERPRAPARSASRPRRPV;
VLATGLRNVPQRKKR;
PTTSSTSTTAKRKKR;
IDMLKARVKNRVAR;
AKRRVVQREKR;
and
AKRRVVQREKRAVGIGALFLG.

In some embodiments, the present invention comprises, consists essentially of, or consists of, a recombinant polypeptide comprising: (a) one or more mutant neuropilin (NRP) b1 domains (b1), NRP b2 domains (b2), or fragments thereof; and (b) an Fc domain; wherein the one or more b1, b2, or fragments thereof, are derived from an NRP1 or an NRP2 protein; wherein the recombinant polypeptide is operable to bind to virus having an —Z₁-X₁-X₂—Z₂- (CendR) motif, wherein Z₁ and Z₂ are arginine or lysine, and X₁ and X₂ are any amino acid; and wherein the virus is selected from the group consisting of: Dengue; respiratory syncytial virus (RSV); Hantavirus; Epstein-Barr virus (EBV); EBV (uncleaved); SARS-CoV-2 Wuhan; SARS-CoV-2 Wuhan (uncleaved); SARS-CoV-2 UK; SARS-CoV-2 India; SARS-CoV-2 India (uncleaved); HCoV-OC43; MERS-CoV; MERS-CoV (uncleaved); Herpes simplex virus (HSV) 1; HSV 1 (uncleaved); influenza A H5N1 virus (IAV H5N1); human papillomavirus (HPV); Human Metapneumovirus; and human immunodeficiency virus (HIV).

In some embodiments, the present invention comprises, consists essentially of, or consists of, a recombinant polypeptide comprising: (a) one or more mutant neuropilin (NRP) b1 domains (b1), NRP b2 domains (b2), or fragments thereof; and (b) an Fc domain; wherein the one or more b1, b2, or fragments thereof, are derived from an NRP1 or an NRP2 protein; wherein the recombinant polypeptide is operable to bind to virus having an —Z₁-X₁-X₂—Z₂- (CendR) motif, wherein Z₁ and Z₂ are arginine or lysine, and X₁ and X₂ are any amino acid; and wherein the virus has a CendR motif selected from the group consisting of:

GTCTQSGERRREKR;
KNTNVTLSKKRKRR;
LTHKMIEESHRLRR;
VSFKPPPPPSRRRR;
VSFKPPPPPSRRRRGACVVY;
CASYQTQTNSPRRAR;
CASYQTQTNSPRRARSVASQSIIAYTMSLG;
ASYQTQTNSHRRAR;
ASYQTQTNSRRRAR;
ASYQTQTNSRRRARSVASQSIIAY;
GSGYCVDYSKNRRSR;
LLEPVSISTGSRSAR;
LLEPVSISTGSRSARSAIEDLLFDK;
ERPRAPARSASRPRR;
ERPRAPARSASRPRRPV;
VLATGLRNVPQRKKR;
PTTSSTSTTAKRKKR;
IDMLKARVKNRVAR;
AKRRVVQREKR;
and
AKRRVVQREKRAVGIGALFLG.

In some embodiments, the present invention comprises, consists essentially of, or consists of, a recombinant polypeptide comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence set forth in any one of the SEQ ID NOs. listed in Table 21, or a pharmaceutically acceptable salt thereof.

In some embodiments, the present invention comprises, consists essentially of, or consists of, a recombinant polypeptide comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence set forth in any one of the SEQ ID NOs. listed in Table 21, or a pharmaceutically acceptable salt thereof, and further comprising an excipient.

In some embodiments, the present invention comprises, consists essentially of, or consists of, a recombinant polypeptide comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence set forth in any one of SEQ ID NOs: 113-116, 121-122, 133-137, 148-149, 154, 162, and 193-201, or a pharmaceutically acceptable salt thereof.

In some embodiments, the present invention comprises, consists essentially of, or consists of, a recombinant polypeptide comprising an amino acid sequence that is at least 50% identical, at least 55% identical, at least 60% identical, at least 65% identical, at least 70% identical, at least 75% identical, at least 80% identical, at least 81% identical, at least 82% identical, at least 83% identical, at least 84% identical, at least 85% identical, at least 86% identical, at least 87% identical, at least 88% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical, at least 99.6% identical, at least 99.7% identical, at least 99.8% identical, at least 99.9% identical, or 100% identical to an amino acid sequence set forth in any one of SEQ ID NOs: 113-116, 121-122, 133-137, 148-149, 154, 162, and 193-201, or a pharmaceutically acceptable salt thereof, and further comprising an excipient.

In some embodiments, the present invention comprises, consists essentially of, or consists of, a recombinant polypeptide comprising an amino acid sequence that is at least 90% identical to an amino acid sequence set forth in any one of SEQ ID NOs: 113-116, 121-122, 133-137, 148-149, 154, 162, and 193-201, or a pharmaceutically acceptable salt thereof.

In some embodiments, the present invention comprises, consists essentially of, or consists of, a recombinant polypeptide consisting of an amino acid sequence that is at least 90% identical to an amino acid sequence according to set forth in any one of SEQ ID NOs: 113-116, 121-122, 133-137, 148-149, 154, 162, and 193-201, or a pharmaceutically acceptable salt thereof.

In some embodiments, the present invention comprises, consists essentially of, or consists of, a recombinant polypeptide consisting of an amino acid sequence set forth in any one of SEQ ID NOs: 113-116, 121-122, 133-137, 148-149, 154, 162, and 193-201, or a pharmaceutically acceptable salt thereof.

In some embodiments, the present invention comprises, consists essentially of, or consists of, a method of limiting the occurrence of, reducing the risk of, reducing the severity of, or treating a viral infection, in a subject in need thereof, said method comprising administering to the subject a composition comprising a therapeutically effective amount of a recombinant polypeptide comprising: (a) one or more mutant neuropilin (NRP) domains, or fragments thereof, and (b) an immunoglobulin domain; wherein the recombinant polypeptide is operable to bind to virus having an —Z₁-X₁-X₂—Z₂- (CendR) motif, wherein Z₁ and Z₂ are arginine or lysine, and X₁ and X₂ are any amino acid.

In some embodiments of the method, the one or more mutant NRP domains, or fragments thereof, are derived from an NRP1 or an NRP2 protein.

In some embodiments of the method, the one or more mutant NRP domains, or fragments thereof, are one or more mutant b1 domains, or one or more mutant b2 domains.

In some embodiments of the method, the virus is a virus belonging to the Realm: Duplodnaviria; Monodnaviria; Riboviria; or Varidnaviria.

In some embodiments of the method, the virus is a virus belonging to the Kingdom: Bamfordvirae; Heunggongvirae; Orthornavirae; Pararnavirae; or Shotokuvirae.

In some embodiments of the method, the virus is a virus belonging to the Phylum: Artverviricota; Cossaviricota; Kitrinoviricota; Negarnaviricota; Nucleocytoviricota; Peploviricota; or Pisuviricota.

In some embodiments of the method, the virus is a virus belonging to the Class: Alsuviricetes; Ellioviricetes; Flasuviricetes; Herviviricetes; Insthoviricetes; Monjiviricetes; Papovaviricetes; Pisoniviricetes; Pokkesviricetes; Revtraviricetes; or Stelpaviricetes.

In some embodiments of the method, the virus is a virus belonging to the Order: Amarillovirales; Articulavirales; Bunyavirales; Chitovirales; Hepelivirales; Herpesvirales; Jingchuvirales; Martellivirales; Mononegavirales; Nidovirales; Ortervirales; Stellavirales; or Zurhausenvirales.

In some embodiments of the method, the virus is a virus belonging to the Family: Astroviridae; Bunyaviridae; Bornaviridae; Chuviridae; Coronaviridae; Flaviviridae; Filoviridae; Hantaviridae; Hepeviridae; Herpesviridae; Nairoviridae; Orthomyxoviridae; Papillomaviridae; Paramyxoviridae; Peribunyaviridae; Phenuiviridae; Pneumoviridae; Poxviridae; Retroviridae; Rhabdoviridae; or Togaviridae.

In some embodiments of the method, the virus is selected from the group consisting of: Dengue; respiratory syncytial virus (RSV); Hantavirus; Epstein-Barr virus (EBV); EBV (uncleaved); SARS-CoV-2 Wuhan; SARS-CoV-2 Wuhan (uncleaved); SARS-CoV-2 UK; SARS-CoV-2 India; SARS-CoV-2 India (uncleaved); HCoV-OC43; MERS-CoV; MERS-CoV (uncleaved); Herpes simplex virus (HSV) 1; HSV 1 (uncleaved); influenza A H5N1 virus (IAV H5N1); human papillomavirus (HPV); Human Metapneumovirus; and human immunodeficiency virus (HIV).

In some embodiments of the method, the virus has a CendR motif selected from the group consisting of:

GTCTQSGERRREKR;
KNTNVTLSKKRKRR;
LTHKMIEESHRLRR;
VSFKPPPPPSRRRR;
VSFKPPPPPSRRRRGACVVY;
CASYQTQTNSPRRAR;
CASYQTQTNSPRRARSVASQSIIAYTMSLG;
ASYQTQTNSHRRAR;
ASYQTQTNSRRRAR;
ASYQTQTNSRRRARSVASQSIIAY;
GSGYCVDYSKNRRSR;
LLEPVSISTGSRSAR;
LLEPVSISTGSRSARSAIEDLLFDK;
ERPRAPARSASRPRR;
ERPRAPARSASRPRRPV;
VLATGLRNVPQRKKR;
PTTSSTSTTAKRKKR;
IDMLKARVKNRVAR;
AKRRVVQREKR;
and
AKRRVVQREKRAVGIGALFLG.

In some embodiments, the present invention comprises, consists essentially of, or consists of, a method of limiting the occurrence of, reducing the risk of, reducing the severity of, or treating a viral infection, in a subject in need thereof, said method comprising administering to the subject a composition comprising a therapeutically effective amount of a recombinant polypeptide comprising: (a) one or more mutant neuropilin (NRP) b1 domains (b1), NRP b2 domains (b2), or fragments thereof, and (b) an Fc domain; wherein the one or more b1, b2, or fragments thereof, are derived from an NRP1 or an NRP2 protein; wherein the recombinant polypeptide is operable to bind to virus having an —Z₁-X₁-X₂—Z₂- (CendR) motif, wherein Z₁ and Z₂ are arginine or lysine, and X₁ and X₂ are any amino acid; and wherein the virus is selected from the group consisting of: Dengue; respiratory syncytial virus (RSV); Hantavirus; Epstein-Barr virus (EBV); EBV (uncleaved); SARS-CoV-2 Wuhan; SARS-CoV-2 Wuhan (uncleaved); SARS-CoV-2 UK; SARS-CoV-2 India; SARS-CoV-2 India (uncleaved); HCoV-OC43; MERS-CoV; MERS-CoV (uncleaved); Herpes simplex virus (HSV) 1; HSV 1 (uncleaved); influenza A H5N1 virus (IAV H5N1); human papillomavirus (HPV); Human Metapneumovirus; and human immunodeficiency virus (HIV).

In some embodiments, the present invention comprises, consists essentially of, or consists of, a method of limiting the occurrence of, reducing the risk of, reducing the severity of, or treating a viral infection, in a subject in need thereof, said method comprising administering to the subject a composition comprising a therapeutically effective amount of a recombinant polypeptide comprising: (a) one or more mutant neuropilin (NRP) b1 domains (b1), NRP b2 domains (b2), or fragments thereof, and (b) an Fc domain; wherein the one or more b1, b2, or fragments thereof, are derived from an NRP1 or an NRP2 protein; wherein the recombinant polypeptide is operable to bind to virus having an —Z₁-X₁-X₂—Z₂- (CendR) motif, wherein Z₁ and Z₂ are arginine or lysine, and X₁ and X₂ are any amino acid; and wherein the virus has a CendR motif selected from the group comprising any motif disclosed in Tables 36 or 37.

In some embodiments, the present invention comprises, consists essentially of, or consists of, a method of limiting the occurrence of, reducing the risk of, reducing the severity of, or treating a viral infection, in a subject in need thereof, said method comprising administering to the subject a composition comprising a therapeutically effective amount of a recombinant polypeptide comprising: (a) one or more mutant neuropilin (NRP) b1 domains (b1), NRP b2 domains (b2), or fragments thereof, and (b) an Fc domain; wherein the one or more b1, b2, or fragments thereof, are derived from an NRP1 or an NRP2 protein; wherein the recombinant polypeptide is operable to bind to virus having an —Z₁-X₁-X₂—Z₂- (CendR) motif, wherein Z₁ and Z₂ are arginine or lysine, and X₁ and X₂ are any amino acid; and wherein the virus has a CendR motif selected from the group consisting of:

GTCTQSGERRREKR;
KNTNVTLSKKRKRR;
LTHKMIEESHRLRR;
VSFKPPPPPSRRRR;
VSFKPPPPPSRRRRGACVVY;
CASYQTQTNSPRRAR;
CASYQTQTNSPRRARSVASQSIIAYTMSLG;
ASYQTQTNSHRRAR;
ASYQTQTNSRRRAR;
ASYQTQTNSRRRARSVASQSIIAY;
GSGYCVDYSKNRRSR;
LLEPVSISTGSRSAR;
LLEPVSISTGSRSARSAIEDLLFDK;
ERPRAPARSASRPRR;
ERPRAPARSASRPRRPV;
VLATGLRNVPQRKKR;
PTTSSTSTTAKRKKR;
IDMLKARVKNRVAR;
AKRRVVQREKR;
and
AKRRVVQREKRAVGIGALFLG.

In some embodiments, the present invention comprises, consists essentially of, or consists of, a method of limiting the occurrence of, reducing the risk of, reducing the severity of, or treating a viral infection, in a subject in need thereof, said method comprising administering to the subject a composition comprising a therapeutically effective amount of a recombinant polypeptide comprising an amino acid sequence that is at least 90% identical to an amino acid sequence set forth in any one of SEQ ID NOs: 113-116, 121-122, 133-137, 148-149, 154, 162, and 193-201, or a pharmaceutically acceptable salt thereof.

In some embodiments, the present invention comprises, consists essentially of, or consists of, a method of limiting the occurrence of, reducing the risk of, reducing the severity of, or treating a viral infection, in a subject in need thereof, said method comprising administering to the subject a composition comprising a therapeutically effective amount of a recombinant polypeptide consisting of an amino acid sequence that is at least 90% identical to an amino acid sequence according to set forth in any one of SEQ ID NOs: 113-116, 121-122, 133-137, 148-149, 154, 162, and 193-201, or a pharmaceutically acceptable salt thereof.

In some embodiments, the present invention comprises, consists essentially of, or consists of, a method of limiting the occurrence of, reducing the risk of, reducing the severity of, or treating a viral infection, in a subject in need thereof, said method comprising administering to the subject a composition comprising a therapeutically effective amount of a recombinant polypeptide consisting of an amino acid sequence set forth in any one of SEQ ID NOs: 113-116, 121-122, 133-137, 148-149, 154, 162, and 193-201, or a pharmaceutically acceptable salt thereof.

### Examples

The Examples in this specification are not intended to, and should not be used to, limit the invention; they are provided only to illustrate the invention.

### Example 1. Design and Production of Constructs

Several types of expression vectors have been designed herein. The first type of expression vector comprises a b1 domain of a neuropilin and an immunoglobulin domain (e.g., Fc domain) as described in Tables 7A and 7B. A second type of expression vector comprises an ACE2 domain of an angiotensin-converting enzyme 2 and an immunoglobulin domain (e.g., Fc domain) as described in Table 6. A third type of expression vector comprises a b1 domain of a neuropilin, an ACE2 domain of an angiotensin-converting enzyme 2, and an immunoglobulin domain (e.g., Fc domain) as described in Tables 8A and 8B.

To increase the binding affinity to spike proteins of a particular virus, such as SARS-CoV-2, polypeptides, also referred to herein as fusion proteins, may be designed to include one or more b1 or neuropilin domains and/or one or more ACE2 or angiotensin-converting enzyme 2 domains.

To increase the binding affinity to spike proteins of a particular virus, such as SARS-CoV-2, polypeptides, also referred to herein as fusion proteins, may be designed with mutations in the b1 or neuropilin domain and/or mutations in the ACE2 or angiotensin-converting enzyme 2 domain. In some embodiments, a mutation in the b1 or neuropilin domain may include an E319A mutation. In some embodiments, a mutation of the ACE2 or angiotensin-converting enzyme 2 domain may include a F28W, D30A, L79T mutation.

To decrease the binding affinity to spike proteins of virus, fusion proteins may be designed with mutations in the b1 or neuropilin domain and/or mutations in the angiotensin-converting enzyme 2 domain. In some embodiments, a mutation of the neuropilin fragment may include Y297A/S346A/Y353A, T349A, and K351A mutations of the b1 domain.

To increase the binding affinity to FcRn, fusion proteins may be designed with mutations in the immunoglobulin Fc domain. In some embodiments, a mutation of the immunoglobulin domain may include N434A and T307A/E380A/N434A (AAA) mutations of the Fc domain.

To decrease the Fc-mediated effector function, the polypeptides or fusion proteins may be designed with mutations in the immunoglobulin Fc domain. In some embodiments, this mutation in the immunoglobulin domain may include L235E, F234A/L235A, N297A, N297Q, or N297G mutations of the Fc domain. In other embodiments, the immunoglobulin Fc domain of IgG2 and IgG4 may be utilized to decrease the Fc-mediated effector function of fusion proteins.

To decrease the antibody-dependent enhancement (ADE), some decoys were designed with mutations in the immunoglobulin Fc domain. In some embodiments, the mutation in the immunoglobulin domain may include L235E, F234A/L235A, N297A, N297Q, or N297G mutations in the Fc domain. In other embodiments, the immunoglobulin Fc domain of IgG2 and IgG4 may be utilized to decrease the Fc-mediated effector function of fusion proteins.

Human neuropilin-1 (UniProt ID: 014786, SEQ ID NO: 1) and human neuropilin-2 (UniProt ID: 060462, SEQ ID NO:2) were used as the source of neuropilin fragment. Human angiotensin-converting enzyme 2 (UniProt ID: Q9BYF1, SEQ ID NO: 32) was used as the source of angiotensin-converting enzyme 2 fragment. Human IgG variants were used as the source of immunoglobulin Fc region. The derivatives of these sequences, including amino acid substitutions, are provided in Tables 1-3.

Generally, protein constructs are synthesized as follows: expression constructs are generated by codon-optimized gene synthesis and inserted into pcDNA3.4 as expression vector using the Not I and Hind III restriction enzyme. The constructed expression vectors include signal peptides and for optimized transcription a Kozak sequence are sometimes included in the 5′ untranslated regions. The resulting plasmids containing the gene encoding the protein constructs are transformed into One Shot™ Top10 E. coli competent cells, and transformed cells are cultured overnight. The constructed plasmids are obtained by the PureLink™ HiPure Expi plasmid Megaprep kit (ThermoFisher Scientific, Waltham, Mass.).

Fusion proteins are transiently expressed in the CHO-S system (ThermoFisher Scientific). The proteins are expressed individually according to the manufacturer's recommended conditions. Briefly, a total of 0.8 pg of plasmid DNA at a ratio of 1:1 light to heavy chain per mL of CHO-S culture is prepared with OPTIPRO™ SFM and EXPIFECTAMINE™. The mixture was added to CHO-S cells at a viable cell density of 6×106 cells/mL and greater than 98% viability. The cell culture is incubated overnight at 37° C., 80% humidity, 8% CO₂ in a Nalgene™ Single-Use PETG Erlenmeyer Flasks shaking at 125 RPM with a 19-mm orbit. The next day the culture is enhanced (EXPICHO™ enhancer; ThermoFisher Scientific.) and fed (EXPICHO™ feed; ThermoFisher Scientific) and transferred to 32° C., 80% humidity, 5% CO₂ shaking at 125 RPM with a 19-mm orbit. The second feed is performed on day 5 and the culture returned to 32° C. until harvest on day 12. Harvesting is accomplished via centrifugation at 4000×g for 20 minutes. The clarified supernatant is sterilized using an asymmetrical polyethersulfone (PES) 0.22-μM filter assembly (Nalgene). The filtrate is stored at 4° C. until purification.

All of the antibody sterilized supernatants is purified using MabSelect PRISMA™ resin (GE Healthcare Life Sciences) on an ÅKTApure (GE Healthcare Life Sciences). A 50 mM sodium phosphate, 150 mM NaCl, pH 7.0 buffer is used to equilibrate the resin. The antibody supernatant is then loaded into the column. The resin is washed with 50 mM sodium phosphate, 150 mM NaCl, pH 7.0 buffer until the chromatographic baseline returned to column equilibration levels. Elution is then performed using 100 mM sodium acetate, 20% glycerol, pH 3.0, and fractions are collected. The fractions are immediately neutralized with 1 M Tris, pH 9. The fractions containing predominant absorbance at wavelength 280 nm are pooled into an Amicon 10-kDa ultrafiltration device for buffer exchange. The storage buffer (Phosphate Buffered Saline) is used to remove the elution buffer by centrifugation with half dilution, seven times in the Amicon concentrator. The material is submitted for SEC and then stored at 4° C.

Size exclusion chromatography (SEC) analysis is performed on an Agilent Infinity 1260 II Quatenary Pump high performance liquid chromatographic (HPLC) system with diode array UV detector WR. Twenty (10) pg of antibody material was injected on a TSKgel G3000SWXL, 5 μm, 7.8 mm ID×30 cm column. The mobile phase is Phosphate Buffered Saline, and the flowrate was 1 mL/min. The antibody material is detected at wavelength 220, 280 and 330 nm at 1 Hz sampling rate during a 15 minute acquisition.

### Example 2. In Vitro Testing

The in vitro testing used Fluorescence-based Enzyme-Linked Immuno-Sorbent Assays (ELISAs) to identify the binding properties of Human Neuropilin- and Angiotensin-Converting Enzyme 2 (ACE2)-Based Human IgG Fc constructs to the SARS-CoV-2 Spike Protein. The ELISA methods used herein were used to rank the order the binding affinities of disclosed Neuropilin-1/2 (NP1 or NP2) and ACE2 human IgG Fc fusion constructs. Also described are the binding of two peptides identified from the SAR-CoV-2 S1/S2 interface of the spike protein that provides entry of the virus into cells via the N1 and N2 receptors along with a peptide derived from the ACE2 amino-terminal alpha 1 domain that was demonstrated to bind the SAR-CoV-2 Spike protein.

The reagents and materials used in the fluorescence binding assays are provided below in Table 9. The instrumentation used is provided in Table 10.

TABLE 9
Reagents Used for Fluorescence Binding Assays.

| | | Catalog | Lot |
|---|---|---|---|
| Reagent | Manufacturer | Number | Number |
| N1ab-huIgG Fc (D12A N1E) | Pinetree | | |
| FITC-SEQ9 (CendR Peptide) | WatsonBio | 112551-8 | WB212207- |
| | | | P200623 |
| FITC-SEQ7 (ACE2 α1 | WatsonBio | 112551-4 | WB212203- |
| Peptide) | | | P200623 |
| SARS-CoV-2 (2019-nCoV) | Sino | 40589- | |
| Spike S1 + S2 ECD-His | Biological | V08B1 | |
| hub1b2-His | Samsung | | |
| FITC-Anti-Human IgG | Abcam | | |
| Dulbecco's Phosphate | Corning | 21-031- | 006620012 |
| Buffered Saline (PBS) | | CM | |
| Bovine Serum Albumin | | | |
| (BSA) | | | |
| Nickel Coated Plates, Black, | Pierce | 15342 | |
| 96-Well | | | |

TABLE 10
Instrumentation Used in the Fluorescence Binding Assays.

| | | Catalog | Lot |
|---|---|---|---|
| Equipment | Manufacturer | Number | Number |
| Varioskan Lux Multimode | ThermoFisher | VLB000GD0 | NA |
| Fluorescence Microplate | | | |
| reader | | | |
| 37° Incubator | VWR | | |

### Example 3. Sample Preparation

All protein constructs and peptides were diluted with PBS to the concentrations listed in Table 11. BSA was diluted to 5% in 1×PBS.

TABLE 11
Construct and Peptide Concentration.

| | | Molecular | | Final |
|---|---|---|---|---|
| | | Weight | | Concentration |
| Construct/Peptide | Diluent | (g/mol) | Final Volume | (μg/mL) |
| N1ab-huIgG Fc (D12A N1E) | 1 × PBS | 190,000 | | 2.00 |
| FITC-SEQ9 (CendR Peptide) | | 2,241 | | 0.03 |
| FITC-SEQ7 (ACE2 α1 | | 4,065 | | 0.03 |
| Peptide) | | | | |
| I. SARS-CoV-2 Spike | | 401,241 | 4 mL for plate 1 | 3.30 |
| S1 + S2 ECD-His | | | 6 mL for plate 2 | |
| b1b2-His | | 35,754 | | 0.30 |
| FITC-Anti-Human IgG | | 150,000 | | 3.10 |

### Example 4. Plate Preparation and Sample Addition

In Experiment 1, plate 1 (see Table 12) was coated with 200 μL of SARS-CoV-2 Spike S1+S2 ECD-His protein in wells A1 through B10 (dark shading) for a final coating amount of 0.66 μg per well and wells C1 through D10 were coated with hub1b2-His for a final coating of 0.06 μg per well. The plate was incubated overnight in a 5° C. refrigerator. The next day, all assays preparations were conducted at room temperature, except for incubations that were at 37° C. Plate 1 was removed from the refrigerator and the liquid removed by flicking the plate over a sink. All wells were rinsed 3-times with 250 μL 1×PBS. After the final rinse, the plate was vigorously tapped top side down onto a paper towel to remove any remaining excess liquid. All the wells were then blocked with 200 μL of 5% BSA and the plate was incubated at 37° C. for 1 hour. Following the incubation, the liquid was removed, and the wells washed as described above.

The FITC-labelled ACE2 al peptide was further diluted to 1.15 μg/mL in 800 μL PBS while the FITC-labelled CendR peptide was diluted to 1.54 μg/mL also in 800 μL PBS. 190 μL of 1×PBS was added to wells A2-A10, B2-B10, C2-C10, and D2-D10. 380 μl of the ACE2 al peptide dilution was added to wells A1 and B1, while the same amount of the CendR peptide was added to wells C1 and D1. Afterwards, a multichannel pipettor was used to remove 190 μL from each of the first wells (A1, B1, C1, and D1) and transferred to the next column of wells. The samples were carefully mixed as not to contaminate the neighboring wells. This procedure was completed for all the proceeding wells until wells A10, B10, C10, and D10 were completed. This process resulted in a 2-fold dilution for each well from column 1 to column 10 in rows A, B, C, and D (see Table 12) The excess 190 μL remaining from the final dilution in column 10 was discarded.

TABLE 12
Plate 1 Plate Map.

| | | Amount of Peptide Per Well (ng) | | | | | | | | | | | |
|---|---|---|---|---|---|---|---|---|---|---|---|---|---|
| | | 1 | 2 | 3 | 4 | 5 | 6 | 7 | 8 | 9 | 10 | 11 | 12 |
| ACE2 | A | 218.88 | 109.44 | 54.72 | 27.36 | 13.68 | 6.84 | 3.42 | 1.71 | 0.855 | 0.428 | Blank | Blank |
| Dilution | B | 218.88 | 109.44 | 54.72 | 27.36 | 13.68 | 6.84 | 3.42 | 1.71 | 0.855 | 0.428 | Blank | Blank |
| S1/S2 | C | 291.84 | 145.92 | 72.96 | 36.48 | 18.24 | 9.12 | 4.56 | 2.28 | 1.14 | 0.57 | Blank | Blank |
| Dilution | D | 291.84 | 145.92 | 72.96 | 36.48 | 18.24 | 9.12 | 4.56 | 2.28 | 1.14 | 0.57 | Blank | Blank |

Note:
Only Rows A-D are presented. The others were not used in this study.

The plate was placed in a 37° C. incubator for 1 hour. Following the incubation, the liquid was removed, and the plate washed and dried as described above.

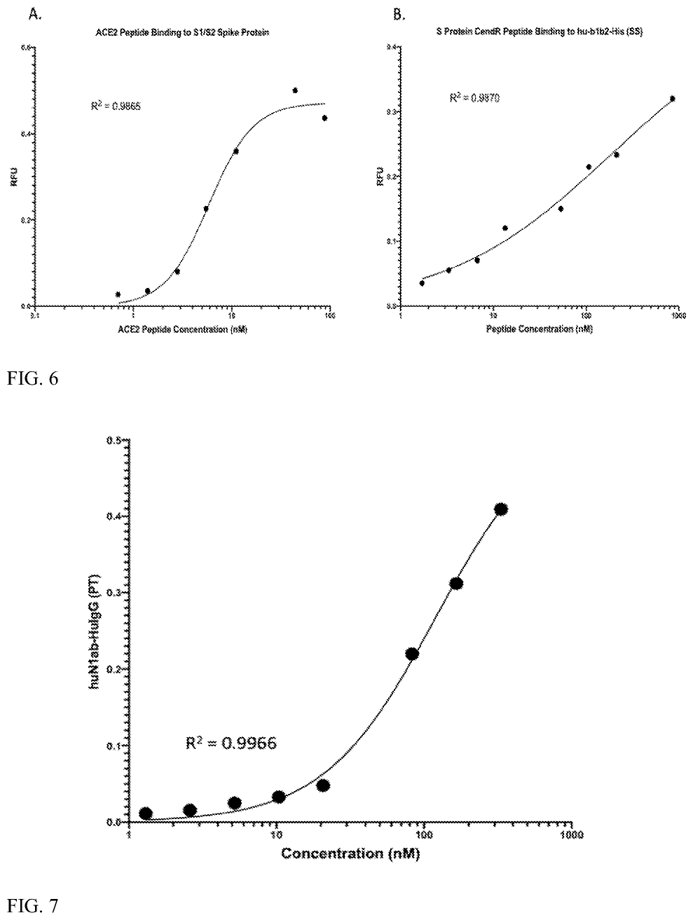

The Relative Fluorescence Units (RFU) from each well was immediately measured using a Varioskan Lux Multimode Fluorescence Microplate reader using the manufacturer suggested conditions with a 495 nm excitation and 519 nm emission, and a graph of the results is presented in FIG. 6.

As provided in FIG. 6, Graph A shows a 1:1 binding relationship for the ACE2 peptide binding to the SAR-CoV-2 virus spike protein over the doses used in this study with an R² value of 0.9865 for the curve fit. Still referring to FIG. 6, Graph B shows a 1:1 binding relationship for the CendR peptide binding to the SARS-CoV-2 virus spike protein over the doses used in the study with an R² value of 0.9870 for the curve fit.

In Experiment 2, plate 2 (see Table 13) was coated with 200 μL of SARS-CoV-2 Spike S1+S2 ECD-His protein in wells A1 through F10 for a final coating amount of 0.66 μg per well The plate was incubated overnight in a 5° C. refrigerator. The next day, all assays preparations were conducted at room temperature, except for incubations that were at 37° C. Plate 2 was removed from the refrigerator and the liquid removed by flicking the plate over a sink. All wells were rinsed 3-times with 250 μL 1×PBS. After the final rinse, the plate was vigorously tapped top side down onto a paper towel to remove any remaining excess liquid. All the wells were then blocked with 200 μL of 5% BSA and the plate was incubated at 37° C. for 1 hour. Following the incubation, the liquid was removed, and the wells washed as described above.

The NRP1ab-huIgG Fc (D12A NIE) protein was further diluted to 64 μg/mL in 1200 μL PBS. 190 μL of 1×PBS was added to wells A2-A10, B2-B10, and C2-C10. 380 μL of the NRP1ab-huIgG Fc (D12A N1E) protein dilution was added to wells A1, B1, and C1. Afterwards, a multichannel pipettor was used to remove 190 μL from each of the first wells (A1, B1, and C1) and transferred to the next column of wells. The samples were carefully mixed as not to contaminate the neighboring wells. This procedure was completed for all the proceeding wells until wells A10, B10, and C10 were completed. This process resulted in a 2-fold dilution for each well from column 1 to column 10 in rows A, B, and C. The excess 190 μL remaining from the final dilution in column 10 was discarded.

TABLE 13
Plate 2 Plate Map

| | | Amount of Peptide Per Well (ng) | | | | | | | | | | | |
|---|---|---|---|---|---|---|---|---|---|---|---|---|---|
| | | 1 | 2 | 3 | 4 | 5 | 6 | 7 | 8 | 9 | 10 | 11 | 12 |
| ACE2 | A | 331.6 | 165.8 | 82.9 | 41.5 | 20.7 | 10.4 | 5.2 | 2.6 | 1.3 | 0.7 | Blank | Blank |
| Dilution | B | 331.6 | 165.8 | 82.9 | 41.5 | 20.7 | 10.4 | 5.2 | 2.6 | 1.3 | 0.7 | Blank | Blank |
| S1/S2 | C | 331.6 | 165.8 | 82.9 | 41.5 | 20.7 | 10.4 | 5.2 | 2.6 | 1.3 | 0.7 | Blank | Blank |
| Dilution | | | | | | | | | | | | | |

Note:
Only rows A-C3 are presented. The others were not used in this study.

The plate was placed in a 37° C. incubator for 1 hour. Following the incubation, the liquid was removed, and the plate washed and dried as described above.

FITC-labelled, anti-human IgG was diluted to 3.1 μg/mL in PBS and 200 μL of this dilution was added to all the wells on the plate except for rows D-H and columns 11 and 12. The plate was placed in a 37° C. incubator for 1 hour. Following the incubation, the liquid was removed, and the plate washed and dried as described above.

The relative fluorescence units (RFU) from each well was measured using a Varioskan Lux Multimode Fluorescence Microplate reader using the manufacturer suggested conditions with a 495 nm excitation and 519 nm emission, and a graph of the results is presented in FIG. 7.

Referring to FIG. 7, the graph shows a 1:1 binding of the huN1ab-huIgG Fc construct to the SARS-CoV-2 virus spike protein over the doses use in this study with an R² value of 0.9966 for the curve fit. It is important to note that the 41.5 and 0.7 ng points were omitted from the graph because they were considered outliers that significantly deviated from the other data points on the curve.

The methods described above are also used to determine the affinity of the various polypeptide constructs described herein for SARS-CoV-2 Spike S1+S2 ECD-His.

### Example 5. Cell-Based Assays

Cell Line Development—Vector Cloning and Engineering of Transgene Expressing Cell Lines

In order to test various therapeutics that may interfere with SARS-CoV-2 infection in a cell-based assay system, HEK-293T and Vero E6 cells are engineered with lentivirus (LV) to stably overexpress ACE2, NRP1, TMPRSS2 or any combination of the three. Table 14 lists the plasmids that were developed in house for lentiviral transduction of cells.

TABLE 14
Plasmid list for cell line development.

| Reagent (plasmid) | Manufacturer |
|---|---|
| LV-ACE2-Zeo | Pinetree |
| LV-NRP1-Puro | Pinetree |
| LV-TMPRSS2-Blast | Pinetree |

Plasmid Development

The full-length ACE2 gene was amplified by PCR using hACE2 plasmid (Addgene #1786) as a DNA template and cloned into a Pinetree lentiviral plasmid, LV-IRES-Zeo using NheI and XhoI. The full length NRP1 gene was amplified by PCR using pcDNA3.4-NRP1, which was synthesized by Genewiz Inc. (Cambridge, MA) and cloned into a Pinetree lentiviral plasmid, LV-2A-Puro using NheI and XhoI. The full length TMPRSS2 gene was amplified by PCR using TMPRSS2 plasmid (Addgene #53887) as a DNA template and cloned into a Pinetree lentiviral plasmid, LV-2A-Blast. DNA sequences of ACE2, NRP1, and TMPRSS2 in lentiviral plasmids was confirmed by Sanger sequencing at Genewiz Inc. (Cambridge, MA).

Lentiviral Packaging of LV-ACE2, LV-NRP1 & LV-TMPRSS2 in HEK-293T Cells

For lentiviral packaging, HEK-293T cells were transfected at 90% confluence in a single well of a 6-well plate. Transfection was performed using LIPOFECTAMINE™ 3000 (ThermoFisher Scientific) according to manufacturer's instructions, using 3.6 μg of pCMV delta plasmid DNA (Pinetree), 2.4 μg of pVSV-G plasmid DNA (Pinetree) together with 5 μg of either LV-ACE2 or LV-NRP1 or LV-TMPRSS2 plasmid DNA per well. 24h after transfection media was changed to 1.5 ml of OPTIMEM™ media (GIBCO™) and supernatant containing lentivirus was harvested 48h and 72h after transfection.

Lentiviral Packaging of LV-ACE2, LV-NRP1 & LV-TMPRSS2 in HEK-293T Cells

For lentiviral packaging, HEK-293T cells were transfected at 90% confluence in a single well of a 6-well plate. Transfection was performed using LIPOFECTAMINE™ 3000 (ThermoFisher Scientific) according to manufacturer's instructions, using 3.6 μg of pCMV delta plasmid DNA (Pinetree), 2.4 μg of pVSV-G plasmid DNA (Pinetree) together with 5 μg of either LV-ACE2 or LV-NRP1 or LV-TMPRSS2 plasmid DNA per well. 24h after transfection media was changed to 1.5 ml of OPTIMEM™ media (GIBCO™) and supernatant containing lentivirus was harvested 48h and 72h after transfection, filter using a 45 μm filter and stored at −80C until further use.

Stable Cell Line Generation

To generate HEK-293T, Vero E6 or other cell lines stably expressing ACE2, NRP1, TMPRSS2 or a combination of the three receptors, cells are plated in their respective complete growth media into a 6-well plate 24 hours prior to viral infection. At the day of infection cells should reach 70-80% confluence. The LV's to be used for transduction are thawed on ice and gradual dilutions (1:10, 1:50, 1:100, 1:500) containing either a single LV or multiple LV's are made in complete growth medium supplemented with polybrene or another transduction enhancing additive. The dilutions are then added to the plated cells (total volume per well 1.5 ml). Following incubation of 48-72h, media is removed and replaced with selection medium containing antibiotics Blasticidin, Zeocin, Puromycin or Hygromycin. The optimal concentration of the respective selection marker varies depending on the cell line and culture conditions and is determined prior to start of selection via treatment of non-transduced parental cells (kill-curve). Cells are selected for 6 to 14 days, or at least as long as it takes the control (untransduced) parental cells to completely die. During selection media containing the respective selection agent is changed every 72h. Once selection is completed, the antibiotic concentration may be reduced or removed entirely.

Following stable pool generation, further serial dilutions followed by single cell clone expansion may be performed. To confirm adequate transgene expression, stable pools or single cell clones are analyzed using flow cytometry or western blotting.

Packaging of Lentivirus Pseudotyped with Either SARS-CoV-2 Spike Protein (S) or with VSV Spike G (Control)

In order to investigate the efficacy of various therapeutics to interfere with SARS-CoV-2 infection, we first generated replication-deficient lentiviruses pseudotyped with either SARS-CoV-2 spike protein (S) or its mutated version, lacking C-terminal 19 amino acids, which is predicted to function as endoplasmic reticulum retention signal. Lentivirus pseudotyped with VSV spike G were packaged in parallel to serve as control. Both the SARS-CoV-2 and the VSV-G pseudotyped lentivirus contain a firefly luciferase and eGFP cassette, which are co-expressed under a CMV promotor in the transduced cells. Therefore, even though both control and SARS-CoV-2 spiked viruses are replication-deficient, viral entry into target cells can be monitored microscopically via detection of eGFP fluorescence as well as via luminescence measurement following incubation of infected cells with media containing luciferin. The plasmids that were used for pseudoviral packaging are listed in Table 15.

TABLE 15
Plasmid list for pseudovirus packaging.

| | | Catalog | |
|---|---|---|---|
| Reagent | Manufacturer | Number | Note |
| pcDNA3.1-SARS-Spike | Addgene | 145032 | Full length S |
| pCMV14-3X-Flag- | Addgene | 145780 | S protein with C- |
| SARS-COV-2 S | | | term 19 aa deletion |
| pCMV delta | Pinetree | | |
| pVSV-G | Pinetree | | |
| II. LV-Fluc-GFP-Puro | Pinetree | | |

To produce adequate amounts of SARS-CoV-2 lentiviral pseudovirus, a large-scale packaging protocol using PEI as transfection agent was employed as follows:

Day 1:

HEK-293T cells are plated into 15 cm plates with approximately 1.4×10⁶ cells.

Day 2:

HEK-293T cells in 15 cm plate should reach 70-80% confluency and the medium should be changed to 15 ml pre-warmed complete growth medium 2h prior to transfection. Plasmid DNA, as shown in Table 16 or Table 17, are added to 5 ml OptiMEM. In parallel, 1 μl of 10 mM PEI (Sigma Aldrich #408727) is mixed with 5 ml OptiMEM and filter sterilize through a 0.22 μm filter. The PEI/OptiMEM solution is then added dropwise to the 5 ml of Opti-MEM/DNA mix followed by incubation at room temperature for 20 minutes. After the incubation the 10 ml of the PEI/OptiMEM/DNA mixture are added to each 15 cm plate, taking care not to disrupt the adherent HEK-293T cells, and incubate at 37C, 5% CO₂ overnight.

TABLE 16
Packaging of SARS-CoV-2 Pseudovirus.

| Reagent | Amount per 15 cm plate | |
|---|---|---|
| OptiMEM | 5 | mL |
| LV-Fluc-GFP-Puro | 25 | μg |
| pCMV delta | 18 | μg |
| pcDNA3.1-SARS-Spike | 25 | μg |
| PEI (10 mM) | 1 | μL |
| OptiMEM | 5 | mL |

TABLE 17
Packaging of VSV Spike G Control Virus.

| Reagent | Amount per 15 cm plate | |
|---|---|---|
| OptiMEM | 5 | mL |
| LV-Fluc-GFP-Puro | 25 | μg |
| pCMV delta | 18 | μg |
| pVSV-G | 12 | μg |
| PEI (10 mM) | 1 | μL |
| OptiMEM | 5 | mL |

Day 3:

Morning: Change medium to 20 mL pre-warmed complete medium Evening (approximately 24h post transfection): change medium to 11 ml OptiMEM (collection medium).

Day 4:

Evening: Collect the medium containing pseudovirus and add 11 mL of fresh pre-warmed collection medium. The collected virus containing medium should be stored at 4° C.

Day 5:

Evening: Collect the medium and pool with first collection. Bleach and discard plates. Filter the virus containing collection media using syringe filter with 0.22 μm filter and 60 mL Syringe.

Concentration and Titration of Lentiviral Vectors

Following collection and filtration the lentivirus pseudotyped with SARS-CoV-2 spike protein (S) or with VSV spike G (control) is concentrated using ultracentrifugation or using a virus precipitation solution as per manufacturer's instructions (e.g. PEG-it, System Biosciences #LV825A-1). Titration of concentrated lentivirus may be performed using quantitative PCR, flow cytometry (lentiviral vectors used in this protocol express GFP) or via determination of relative vector particle number based on virion RNA as described previously.

### Example 6. Cellular Assay for the Quantitative Measurement of Pseudovirus Transduction in the Presence or Absence of Therapeutic Agents

Day 1: Cell Plating

Target cells, such as 293T, 293T-ACE2, 293T-TMPRSS2, 293T-NRP1, 293T-ACE2/TMPRSS2, 293T-ACE2/NRP1, 293T-NRP1/TMPRSS2, which are to be investigated for SARS-CoV-2 lentiviral pseudovirus infectivity, are plated with a density of 5,000 to 10,000 cells per well into black opaque, clear bottom 96-well microplates (ThermoFisher, Nunc #165305) with 50 μl of complete medium and incubated overnight at 37C with 5% CO₂.

Day 2: Viral Transduction

Virus transduction in the absence or presence of anti-viral therapeutics:

Serial dilution of anti-viral agents to be tested are prepared in complete medium.
To test agents binding to SARS-CoV-2 spike protein S:
   5 to 25 μL of concentrated SARS-CoV-2 pseudotyped lentivirus are pre-incubated for 30 minutes with an equal volume of the serial-diluted anti-Spike therapeutic (e.g. anti-Spike monoclonal antibody, ACE2 or NRP1 decoy receptor constructs). After incubation 10 to 40 μL of virus/antibody mixtures containing the different dilutions of anti-Spike therapeutic are added to each well.
To test agents binding to ACE2, NRP1, TMPRSS2 or other receptor expressed on target cells and expected to be involved in viral transduction:
   5 to 25 μL of the serial-diluted anti-receptor therapeutic (e.g. anti-ACE2 mAb) are added to each well containing target cells and incubated for 30 minutes. Following incubation, an equal volume of concentrated SARS-CoV-2 pseudotyped lentivirus is added to each well
Control wells, containing the same amount of target cells are not treated with therapeutics or pseudoviruses.
VSV-G pseudotyped lentivirus contain a firefly luciferase and eGFP cassette may be added in similar fashion and serve as another control.

Following treatment as outlined above, the plates are incubated at 37° C. with 5% CO₂. 48 to 72 hours after transduction, eGFP expression may be observed and quantified using fluorescence microscopy. For luminescence readout, a solution of luciferin in complete medium is prepared of which 50 μL are added per well (final concentration 0.4 mg/mL luciferin) followed by incubation for 20 to 30 minutes. The transduction efficacy is determined via measurement of the transduced cells luciferase activity on a luminescence microplate reader (e.g. ThermoFisher Scientific Varioskan Lux Multimode Microplate Reader). Alternatively, the transduction efficacy may further be measured via flow cytometry and gating for GFP-positive cells.

### Example 7. SARS-CoV-2 In Vitro Vero E6 Based Infectivity and Cytopathic Effects Assay

To test and analyze the anti-viral activity of test articles against replication active SARS-CoV2 virus in Vero E6 cells, an in vitro inhibition assay may be performed to test and analyze the anti-viral activity of compounds of the present invention against replication active SARS-CoV2 virus in Vero E6 cells.

Methods

For example, a modified bioassay protocol of Biological Research Information Center (BRIC) may be used for the testing anti-viral compounds of the present invention. The compounds, e.g., may be those described in Samples 1-4 (described below); all samples may be liquid and soluble in water. The solvent for dilution of Samples 1-3 may be PBS, and for Sample 4 may be DMSO. The cell strain may be Vero E6 cells (ATCC). The virus strain may be SARS-CoV-2 (e.g., NCCP43326; sourced from the Korean CDC).

Cell culture conditions may be as follows: Cells are cultured in an incubator at 37° C., 95% humidity, 5% CO₂, with conditions monitored every 8 hours. Media and reagent: DMEM, 10% FBS, 1% Pen/Strep, 1% L-Glutamine 200 mM, 1% Sodium Pyruvate 100 mM, Nonessential amino acid. Flask and cell density: Cells may be cultured in 96 well plate with 1×10⁴/well, but cell density can be changed if stable virus is not detected.

The antivirus assay using the compounds of the present invention may be performed with the following in mind: because the mechanism of action is different from each anti-viral compound, finding standard assay conditions may not practicable. Accordingly, assay conditions may be coordinated on a case-by-case basis (e.g., entry blocker, replication blocker, etc.).

Virus propagation assays may use active virus with over 10² TCiD50/mL.

The compounds tested may be serially diluted, 2⁻¹ to 2⁻⁶ in DMEM. The virus can be diluted as follows: Titer of SARS-CoV-2 may be measured before use and 1.0 mL of the virus may be used after 10×-diluted in 4° C. PBS.

Treatment condition may be as follows: Culture medium of Vero E6 cells in 96 well plates may be removed and washed by 100 μL of PBS two times. Next, 50 μL of serially diluted test articles may be added and 50 μl of the virus (10² TCID50/ml) added for infection.

Analysis of anti-viral activity of the compounds of the present invention may be evaluated as follows: Cytopathic effect was monitored from 48- to 72-hours post-infection. RNA may be isolated from infected cells and qPCR analysis was used to estimate residual amount of virus

### Example 8. Infectivity Assays

Infectivity assays may be performed as follows: first, the COVID virus used for efficacy evaluation may be a virus adapted by passage of SARS-CoV-2 (e.g., as sourced from the Korean CDC), three times in Vero E6 cells. Virus (minimum 10³TCID₅₀/mL) may be diluted 10-fold, inoculated into Vero E6 cells, and treated with various concentrations of the compounds provided below at the same time:
Sample 1: hNRP1ab-hFc (human NRP1 fragment comprising the a and b domains, fused with human IgG1 Fc);
Sample 2: hNRP1b1b2-his (human NRP1 fragment comprising the b1b2 domain, fused at its C-terminus to a 6× histidine tag);
Sample 3: hNRP1b1b2-aaa-his (human NRP1 fragment comprising the b1b2 domain containing the Y297A/S346A/Y353A mutations, which reduce VEGF binding, fused at its C-terminus to a 6× histidine tag); and
Sample 4: Remdesvir (Control).

Remdesvir is well known to those having ordinary skill in the art, and is available under the tradename “VEKLURY®” (Gilead sciences; CAS No. 1809249-37-3)

Cell viability and morphology may be observed 48-hours to 96-hours after sample treatment.

Cytotoxicity results: No cytotoxicity may be observed in the highest concentrations of any of the test substances (data not shown).

Infectivity results: In the experiment in which the virus culture solution and the test preparation diluent are treated simultaneously, cytopathic effect (CPE) may be observed from 48-hours in the case of the virus-treated group. CPE may not be observed in all dilutions of Samples 1 to 3, and CPE may be observed in Sample 4 at 72-hours after inoculation at a concentration of 1.11 μM.

Freezing and thawing may be performed twice for each sample at 96-hours to quantitatively evaluate the degree of virus proliferation. For Samples 1, 2 and 3, the lowest dilution factor may be 0.00003 μM, and for Sample 4, RNA may be extracted from the 1.11 μM well where CPE was observed, followed by real-time PCR. These results can be interpreted to have significantly inhibited the infection of viruses and the replication of intracellularly infected viruses without toxicity, and it can be considered that it is necessary to clarify the therapeutic effect in infected animals through animal experiments.

### Example 9. Animal Testing

Infectivity assays may be performed as follows: SARS-CoV-2 isolates may be propagated in VeroE6 cells in OptiMEM containing 0.3% bovine serum albumin (BSA) and 1 μg of L-1-tosylamide-2-phenylethyl chloromethyl ketone treated-trypsin per mL or in Vero 76 cells in a minimal essential medium (MEM) supplemented with 2% fetal calf serum at 37° C.

All experiments with SARS-CoV-2 may be performed in enhanced biosafety level 3 (BSL3) containment laboratories or in enhanced BSL3 containment laboratories.

Experimental Infection

One-month-old female Syrian hamsters and 7- to 8-month-old female Syrian hamsters may be used in this study. Baseline body weights may be measured before infection. Under ketamine-xylazine anesthesia, four hamsters per group may be inoculated with 10⁵-6 PFU (in 110 μL) or with 10³ PFU (in 110 L) of a SARS-CoV-2 isolate via a combination of intranasal (100 μL) and ocular (10 μL) routes. Body weight may be monitored daily for 14 days.

For virological and pathological examinations, two, four, or five hamsters per group may be infected with 105.6 PFU (in 110 μL) or with 103 PFU (in 110 μL) of the virus via a combination of the intranasal and ocular routes; 3, 6, and 10 d postinfection, the animals may be killed, and their organs (e.g., nasal turbinates, trachea, lungs, eyelids, brain, heart, liver, spleen, kidneys, jejunum, colon, and blood) may be collected.

For the reinfection experiments, three hamsters per group may be infected with 105.6 PFU (in 110 μL) or with 103 PFU (in 110 μL) of SARS-CoV-2 or PBS (mock) via a combination of the intranasal and ocular routes. On day 20 postinfection, these animals may be reinfected with 105.6 PFU of the virus via a combination of the intranasal and ocular routes. On day 4 after reinfection, the animals can be killed, and the virus titers in the nasal turbinates, trachea, and lungs can be determined by means of plaque assays in VeroE6/TMPRSS2 cells.

For the passive transfer experiments, eight hamsters may be infected with 105.6 PFU (in 110 μL) or with 103 PFU (in 110 μL) of SARS-CoV-2 via a combination of the intranasal and ocular routes. Serum samples may be collected from these infected hamsters on day 38 or 39 postinfection, and may be pooled. Control serum can be obtained from uninfected age-matched hamsters. Three hamsters per group may be inoculated intranasally with 103 PFU of SARS-CoV-2. On day 1 or 2 postinfection, hamsters may be injected intraperitoneally with the postinfection serum or control serum (2 mL per hamster). The animals may be killed on day 4 postinfection, and the virus titers in the nasal turbinates and lungs may be determined by means of plaque assays in VeroE6/TMPRSS2 cells. All experiments with hamsters may be performed in accordance with the Proper Conduct of Animal Experiments and corresponding guidelines in addition to an approved protocol.

### Example 10. In Vivo Efficacy of SEQ ID NO: 122 on SARS-CoV-2 in Hamsters

Methods

The in vivo efficacy of SEQ ID NO: 122 on SARS-CoV-2 in hamsters was evaluated.

The present example included six arms with four hamsters per group. The animals were all male Syrian Golden hamsters obtained from Charles River Laboratories, weighing approximately 60-70 grams. The hamsters were weighed daily from inoculation through day 4, and again on day 7 when euthanized. On days 0, 2, 4 and 7 plethysmography (PFT) recordings were obtained after acclimation, for a period of 20 minutes after which (under isoflurane anesthesia) 100 μL of blood was collected from either the jugular vein or anterior vena cava.

A Blood was collected and plethysmography (PFT) performed on day 0 prior to intra-tracheal viral inoculation with a given challenge. Animals were anesthetized with ketamine/xylazine at a dose of 133 mg/kg and 13.3 mg/kg, respectively, followed by viral challenge. Viral challenge was administered with an intra-tracheal viral inoculum of Severe acute respiratory syndrome-related coronavirus 2 (SARS-CoV-2), isolate USA-WA1/2020 consisting of either 51 plaque forming units (PFU), or 510 PFU in 50 μL of EMEM. Controls included a non-viral control, consisting of 50 μL of EMEM; and a positive control consisting of SAD-S35 (anti-SARS-CoV-2 RBD Neutralizing Antibody, Human IgG1) (see information regarding SAD-S35 below).

The six arms evaluated were as follows:
Arm (1) Non-viral EMEM control followed by 0.5 mL PBS with 250 mM NaCl given at 12- and 24-hours post-inoculation (PI);
Arm (2) 51 PFU followed by 0.5 mL PBS with 250 mM NaCl given at 12- and 24-hours PI;
Arm (3) 510 PFU followed by 0.5 mL PBS with 250 mM NaCl given at 12- and 24-hours PI;
Arm (4) 51 PFU followed by SEQ ID NO: 122 intraperitoneal injection (15 mg/kg) given at 12- and 24-hours PI;
Arm (5) 510 PFU followed by SEQ ID NO: 122 intraperitoneal injection (15 mg/kg) given at 12- and 24-hours PI; and
Arm (6) 510 PFU followed by SAD-S35 intraperitoneal injection (15 mg/kg) given at 12- and 24-hours PI.

SAD-S35 (anti-SARS-CoV-2 RBD Neutralizing Antibody, Human IgG1) was used as positive control. SAD-S35 is a specific antibody against SARS-CoV-2 Spike protein RBD domain. SAD-S35 is isolated from a SARS-CoV-2 infected patient and is recombinantly produced from human 293 cells (HEK293). SAD-S35 is available from ACROBiosystems (1 Innovation Way, Newark, DE 19711; Catalog No. SAD-S35).

On day 7 after plethysmography recording, hamsters were euthanized using 0.1 mL IP Euthasol® (Euthanasia Solution comprising pentobarbital sodium and phenytoin sodium), blood was collected, bronchoalveolar lavage was performed with 1.5 mL PBS, and lungs were inflated with 1.5 mL of 10% neutral buffered formalin and subsequently fixed for Hematoxylin and eosin (H&E) staining.

Animal weight was charted in Excel. Plethysmography data is presented as log Penh, ln Rpef and square root EF50 in the standard format published in rodent viral spirometry literature known to those having ordinary skill in the art.

RT-qPCR was performed on RNA isolated from blood samples and bronchoalveolar lavage fluid (BAL) obtained using Qiagen Viral RNA isolation kits. The CDC TaqMan® assay primer/probe sequences and conditions targeting the SARS-CoV-2 viral nucleocapsid protein were used in an ABI QuantStudio 3 thermal cycler. Promega GoTaq® RT-PCR, and cloned standards of the virus nucleocapsid were used to quantify genome copies.

Results

During viral inoculation hamster No. 8 died in the 51 PFU saline treatment group, and at the end of the experiment data from hamster 4 (media inoculation only, PBS Injections) was digitally corrupt in the nonviral group and could not be used, leaving 3 animals in each of these control groups. All other groups contained four hamsters.

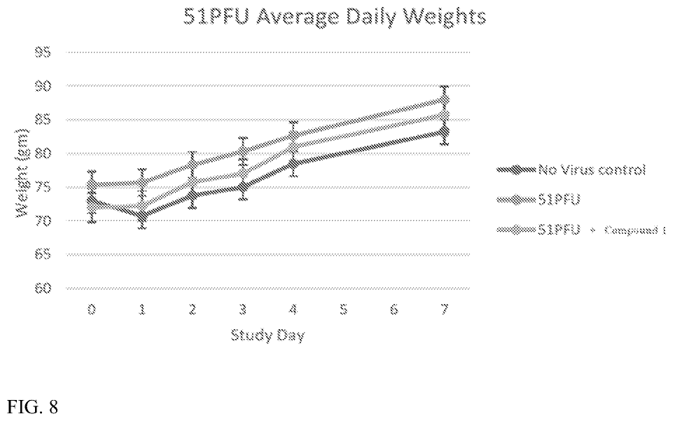

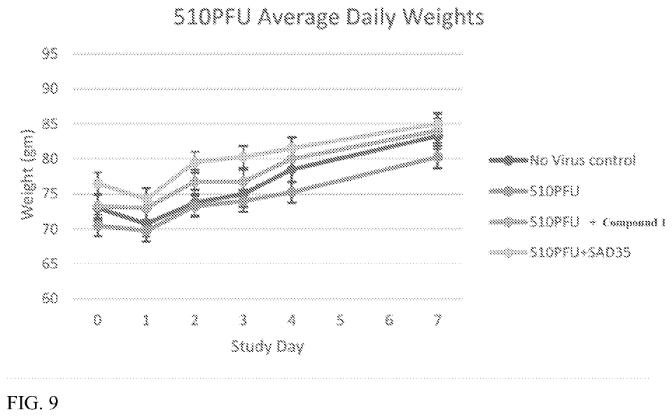

No significant weight changes were observed between groups. FIGS. 8 and 9. Poor weight gain in the first 1-2 days PI was seen in all groups, and weight loss occurred day 1 PI in the non-viral control. However, all animals continued to gain weight throughout the remainder of the study. Because the non-viral control group experienced the only significant change in body weight gain from day 0 to day 1, no adverse effect due to treatments or viral inoculation were detected between groups.

Plethysmography

Analysis of three calculated parameters from whole body plethysmography were used to characterize the effects of viral infection and treatment on respiratory physiology. The three parameters were Enhanced pause (Penh); Rpef, and EF50.

Enhanced pause (Penh) is a unit-less index of calculated airway function. Penh calculations were performed according to the following formula:

$$Penh = \frac{PEF}{PIF} \times \frac{Te - Tr}{Tr} \qquad Formula ( I )$$

Wherein PEF is peak expiratory flow of breath; PIF is peak inspiratory flow of breath; Te is time of expiratory portion of breath; and Tr is time required to exhale 65% of breath volume. Penh serves as an indirect measure of airway resistance and provides a non-specific assessment of breathing patterns.

The equation for Penh takes into account four breathing parameters including: peak expiratory flow of breath (PEF); peak inspiratory flow of breath (PIF); time of expiratory portion of breath (Te); and time required to exhale 65% of breath volume (Tr). Penh serves as an indirect measure of airway resistance and provides a non-specific assessment of breathing patterns.

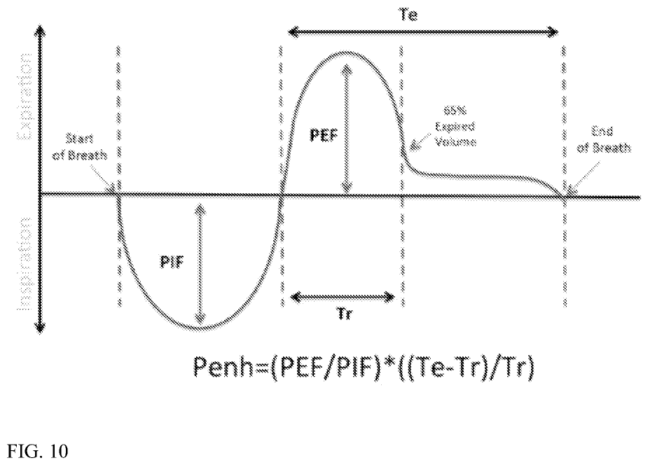

FIG. 10 is a graphical representation of the respiratory cycle, showing various measurements that are used for calculation of the respiratory parameters used for comparison between the groups in this study.

“Rpef” measures the ratio of the time to peak expiratory follow (PEF) relative to the total expiratory time. Rpef is calculated according to the following equation:

$$Rpef = \frac{Time to PEF}{Te} \qquad Formula ( II )$$

Wherein PEF is peak expiratory follow; and Te is total time of expiration.

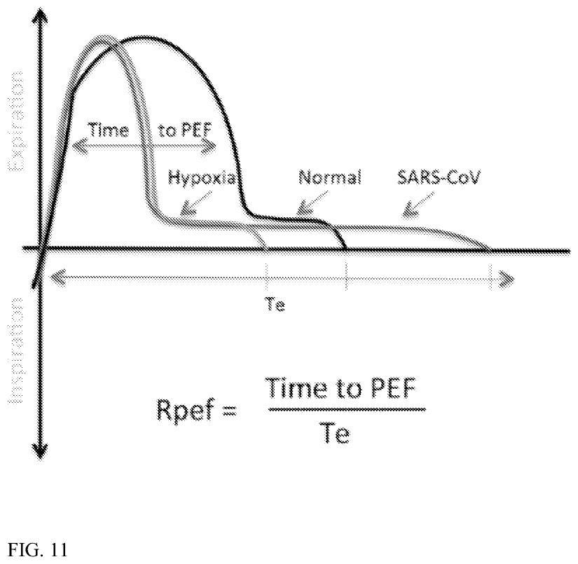

This calculated parameter is lower in chronic obstructive pulmonary disease and SARS-CoV-1 infection when the terminal portion of a breath is obstructed from volume depletion due to airway constriction. FIG. 11.

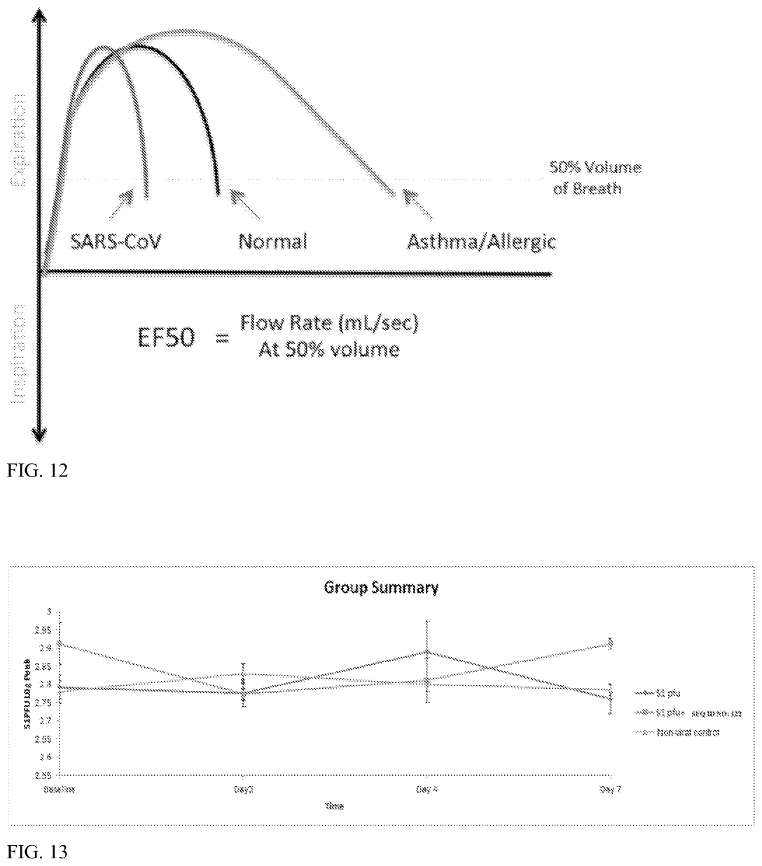

EF50 is flow rate (mL/seconds) at 50% volume. EF50 is similar to Rpef, and is sensitive to airway constriction during expiration; however, changes in expiration are detected in the later portion of the expiratory cycle. This calculated parameter is shortened in SARS-CoV-1 and lengthened with asthma. FIG. 12.

The three calculated parameters were mathematically transformed: Penh to log Penh, Rpef to ln Rpef, and EF50 to the square root of EF50, pursuant to conventional means known by those in the art when presenting data of this type.

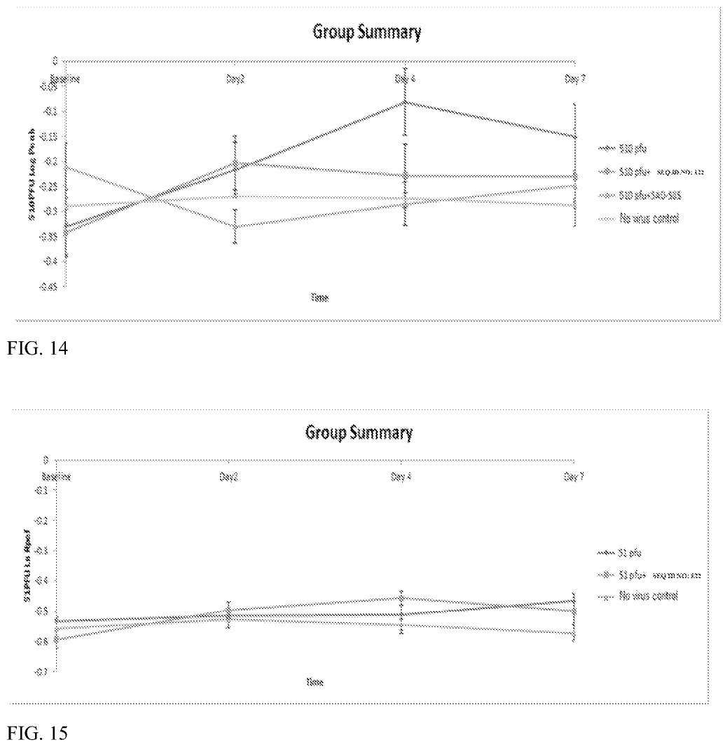

The results of the plethysmography data from the 51 PFU-treated and 510 PFU-treated group of hamsters are shown in FIGS. 13 and 14.

Log Penh data in the 51 PFU groups demonstrate that both SEQ ID NO: 122 and untreated arms returned to baseline values after 7 days with no residual significant change in respiratory parameters. FIG. 13. A greater deviation from its own baseline values was seen on day 2 in the SEQ ID NO: 122 group than in the untreated group that deviated most on day 4, however this was not statistically significant. There is a significant difference between the 51 PFU treated and untreated groups on day 7 (T-test, T=3.998, p<0.01), meaning that the SEQ ID NO: 122 treated group continued to return toward baseline whereas the untreated group changed progressively at the end of the study from their baseline values.

A significant difference (T-test, T=6.058, P<0.001) exists on day 7 between the SEQ ID NO: 122 treated group and the non-viral challenge control. The log Penh parameter remained relatively unchanged in the non-viral control group over the duration of the experiment. In contrast the 510 PFU challenge resulted in a persistent deviation of log Penh from baseline greatest in the untreated arm, to a lesser extent in the SEQ ID NO: 122 treated arm, and even less in the SAD-S35 antibody treated arm. A significant difference exists between log Penh in the SAD-S35 treated group and the untreated viral group on day 4 (T-test, T=2.544, P<0.05), no other statistical relevance was found between arms in the 510 PFU challenge.

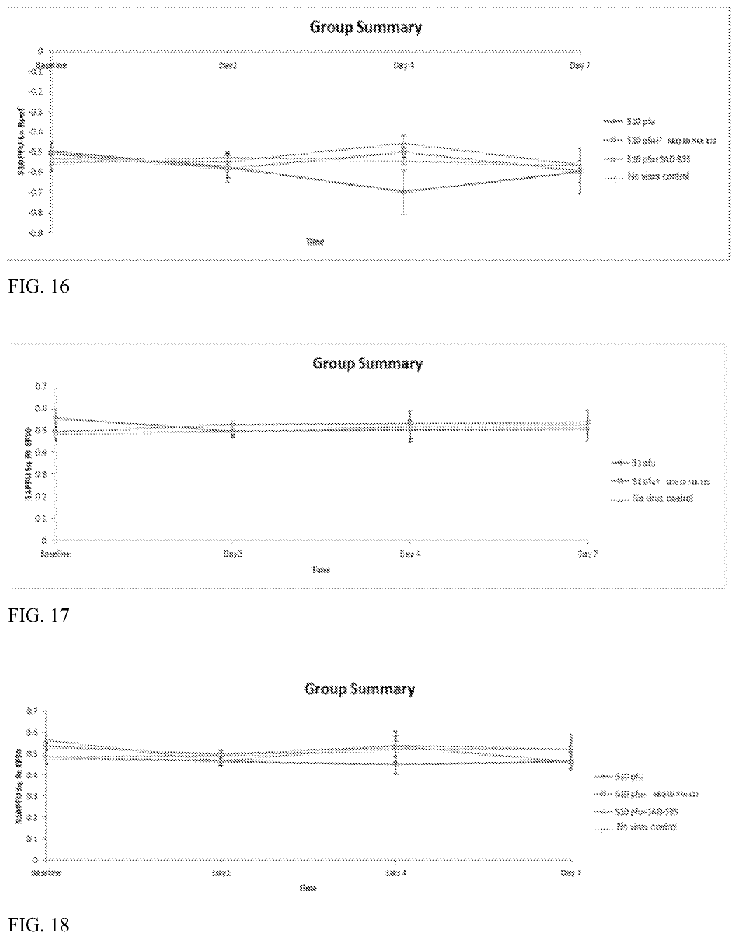

In the ln Rpef data, the 510 PFU challenge reveals an effect of treatments more accentuated than measured in the 51PFU challenge group. FIGS. 15 and 16. 51 PFU challenge without treatment shows a persistent change in the ln Rpef parameter when compared to the SEQ ID NO: 122 treated arm, which is returning toward baseline values. On day 4 there is a statistically significant difference between the non-viral control arm and the 51 PFU arm treated with SEQ ID NO: 122 (T-test, T=2.75, P<0.05). On day 7 there is a statistically significant difference between ln Rpef of the non-viral control and the 51 PFU challenge arms without treatment (T-test, T=2.775, P<0.05). The 510 PFU challenge shows lesser deviation from baseline values for treated groups (SEQ ID NO: 122 and SAD-S35) when compared to the untreated arm. Rpef is largely derived from changes in the final portion of expiration when airway constriction slows the time to final expiration.

The final parameter examined, square root of EF50, demonstrated little to no changes in both challenge groups, with and without treatments. There are no statistically significant differences on any day between arms in the 51 PFU and 510 PFU challenge arms. FIGS. 17 and 18. This parameter is sensitive to the initial portion of expiration that changed little if at all during SARS-CoV-2 infection.

RT-qPCR

Heparinized blood was diluted 1:10 in PBS prior to storage at −80° C. 140 μL was used to isolate total RNA from non-viral control group, 51 PFU PBS-treated and 510 PFU PBS-treated controls. One microliter of the isolated RNA was subjected to CDC RT-qPCR for viral nucleocapsid and the results compared to cloned standards. No SARS-CoV-2 genomic copies were detected in any of the samples, whereas the controls functioned as expected, and so the remaining samples were not tested.

Histology

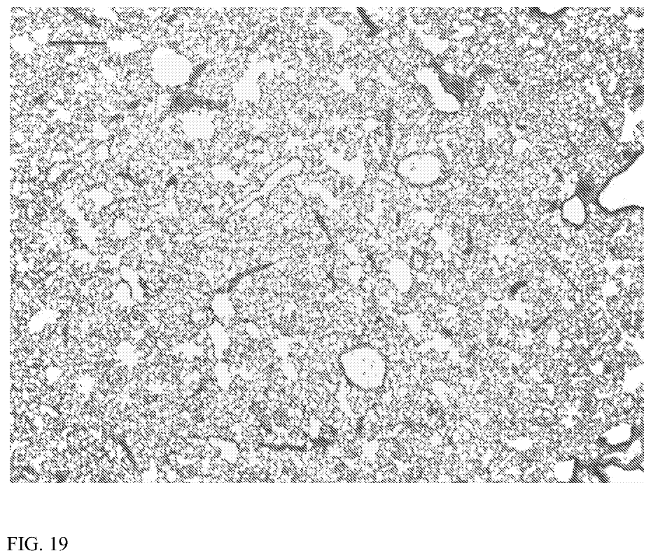

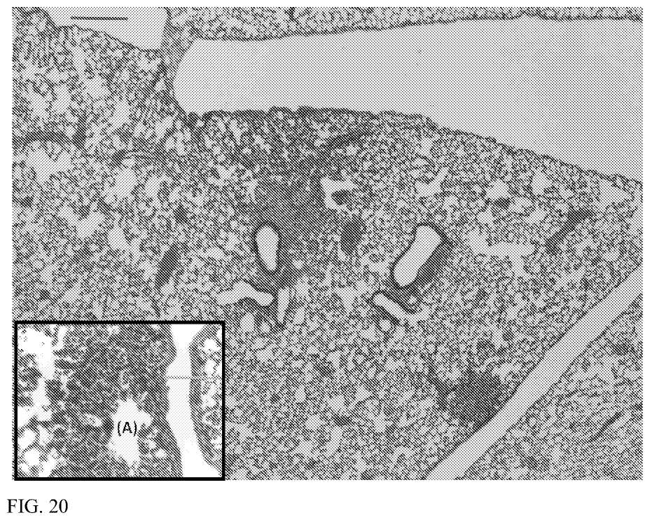

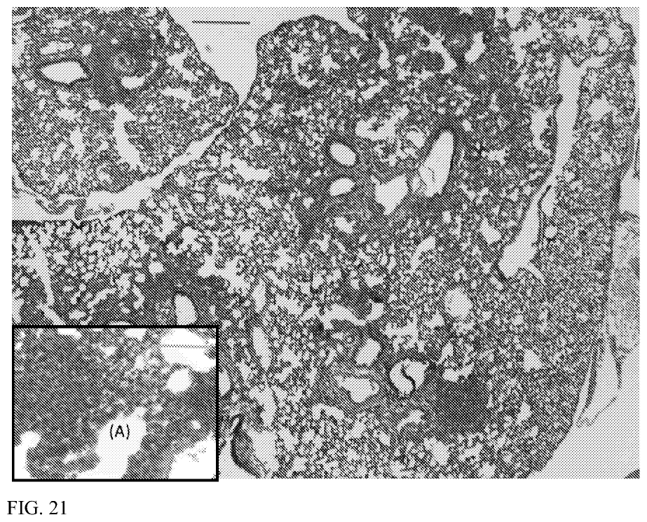

Histology slices obtained from the lungs of a representative hamster selected from of each of the study arms are shown in FIGS. 19-24. The histology sections were fixed and stained seven days from viral or media challenge after bronchoalveolar lavage was performed. No inflammation was present in the media inoculated control arm (FIG. 19) compared with a few areas of mild-moderate chronic-active inflammation in the 51 PFU arm (FIG. 20). Several areas of more severed chronic-active inflammation were observed in the 510 PFU arm. FIG. 21.

In the 51 PFU- and 510 PFU-treated tissue, areas of inflammation contained lymphocytes and plasma cells predominantly, and in lesser numbers macrophages containing cellular debris and neutrophils. FIGS. 20-21. Rarely syncytia of terminal bronchiolar epithelial cells could be seen.

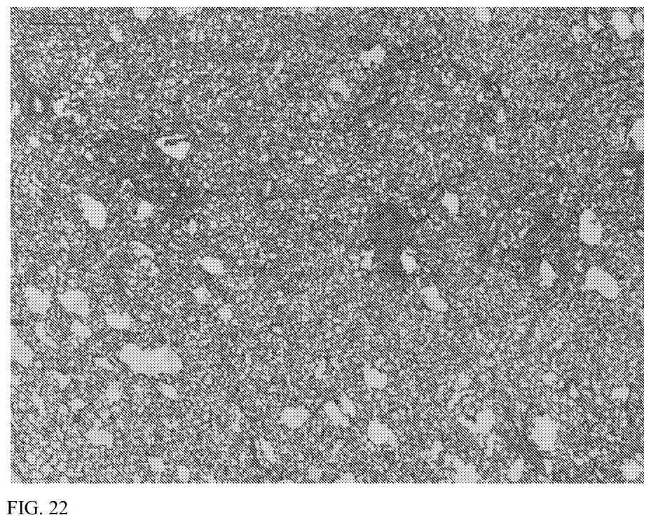

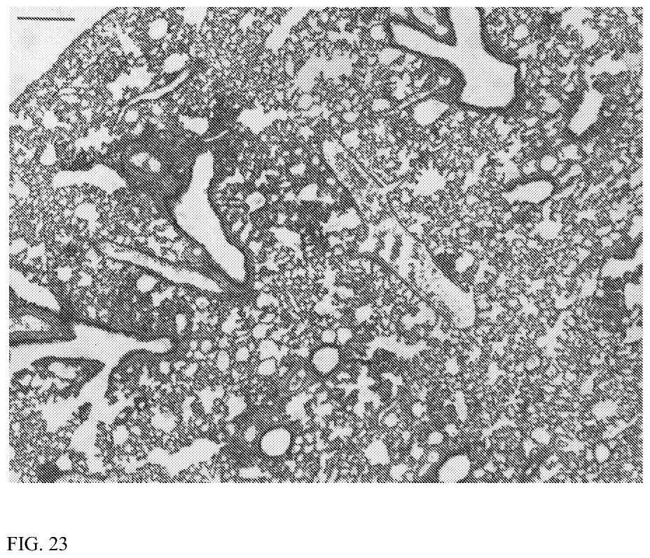

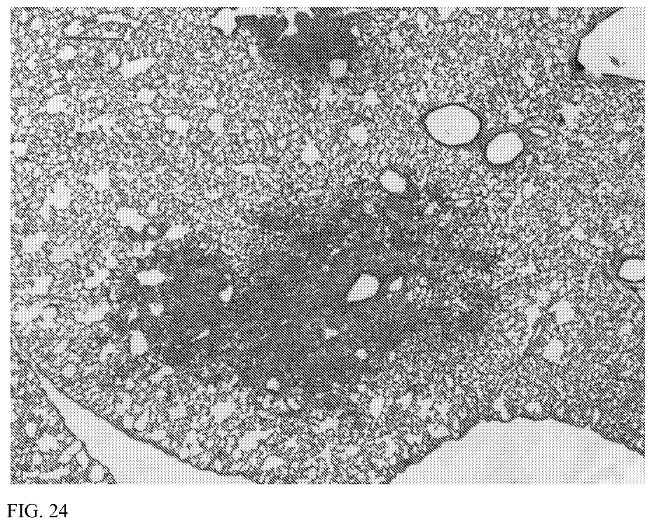

Treatment with SEQ ID NO: 122 did not reduce the areas of inflammation in the 51 PFU or the 510 PFU arms. FIGS. 22-23. Similarly, treatment with the antibody, SAD-S35, did not reduce the size or severity of inflammation in the 510 PFU arm. FIG. 24.

Quantitative RT-PCR on Bronchoalveolar Lavage (BAL) Fluid

One mL of PBS was used to lavage the lungs after euthanasia on day 7. Total RNA was isolated from 140 μL of BAL, and eluted in 50 μL; from this, 1 μL was subjected to RT-qPCR for SARS CoV-2 nucleocapsid gene using the U.S. CDC Real-Time Reverse Transcription PCR Panel for Detection of Severe Acute Respiratory Syndrome Coronavirus 2 (https://www.cdc.gov/coronavirus/2019-ncov/lab/rt-pcr-panel-primer-probes.html).

The results of the RT-qPCR assay are shown in the table below.

TABLE 18
Quantitative RT-PCR results. Group No. corresponds
to six arms described in the methods above, and
briefly summarized in the treatment column.

| Group | | Animal | Copies/ | Average | STD. DEV. |
|---|---|---|---|---|---|
| No. | Treatment | ID | μL | Copy/μL | Copies/μL |
| 1 | EMEM control | 1 | 0 | 0 | 0 |
| | PBS treatment | 2 | 0 | | |
| | | 4 | 0 | | |
| 2 | 51 PFU | 5 | 1144 | 389 | 654 |
| | challenge | 6 | 21 | | |
| | PBS treatment | 7 | 1.5 | | |
| 3 | 510 PFU | 9 | 36 | 720 | 1211 |
| | challenge | 10 | 214 | | |
| | PBS treatment | 11 | 2534 | | |
| | | 12 | 97 | | |
| 4 | 51 PFU | 13 | 70 | 47825 | 93777 |
| | challenge | 14 | 71 | | |
| | SEQ ID NO: | 15 | 2680 | | |
| | 122 treatment | 16 | 188478 | | |
| 5 | 510 PFU | 17 | 343 | 5883 | 11078 |
| | challenge | 18 | 122 | | |
| | SEQ ID NO: | 19 | 22498 | | |
| | 122 treatment | 20 | 569 | | |
| 6 | 510 PFU | 21 | 43 | 809 | 1335 |
| | challenge | 22 | 391 | | |
| | SAD-S35 | 23 | 7 | | |
| | treatment | 24 | 2794 | | |

No virus was detected in the EMEM inoculated control group (Group 1). No significant difference was detected by single factor ANOVA (F=0.788, F critical=3.326, df within groups=5, df between groups=10). The lack of statistical difference is the result of substantial standard deviation in copy numbers of the nucleocapsid gene. In general, the standard error of each group was greater than or equal to the group average, due to a single outlier in copy number in BAL. Individual one-tailed T-test comparison of challenge vs challenge+treatment failed to demonstrate a significant difference in the quantity of nucleocapsid copies per microliter of RNA isolated from BAL.

Discussion

The beneficial effects of treatment with SEQ ID NO: 122 and SAD35 that were noted in the plethysmography data do not seem to correlate with the degree of inflammation seen histologically. There could be several reasons for this, including: the drugs diminished the inflammatory response earlier in the infection when histologic evaluation was not done; the airway resistance during expiration was diminished by the drug treatment, alleviating some degree of airway constriction, while having no effect on inflammation; and/or the treatments changed the development of early fibrosis that began during the bronchopneumonia allowing better pulmonary compliance.

The small but significant beneficial effects of the treatments may have been muted since the pharmacokinetics of SEQ ID NO: 122 and SAD35 in the hamster are not known. If the half-life of these treatments in the hamster is short, or if the minimum inhibitory concentration for viral replication of the compounds is higher than was achieved, then SARS-CoV-2 viral replication may have been temporarily slowed or impaired in a manner insufficient to stop the ensuing inflammatory response. The SAD35 IgG antibody that blocked SARS-CoV-2 viral replication in vitro likely did not achieve sufficient levels in the alveolar spaces to completely inhibit viral replication since this is not a secreted antibody.

Analysis of BAL fluid for SARS CoV-2 nucleocapsid gene targets per microliter of RNA isolated from by RT-qPCR showed no significant difference between the titer of viral challenge with PBS vs. SEQ ID NO: 122 or SADS35 treatment. In general, one hamster in each group showed a high copy number this being most significant in the SEQ ID NO: 122 treated animals.

### Example 11. In Vivo Efficacy of VT116, VT114, and VT130 on SARS-CoV-2 in Hamsters

In this example, young male hamsters weighing 95-100 grams were purchased with jugular catheters, and treated by intravenous (IV) administration of SEQ ID NO: 122, SEQ ID NO: 154, and SEQ ID NO: 192, after being confronted with 1500 PFU SARS-CoV-2 intra-tracheal challenge.

Challenge virus was diluted to a concentration of 100 PFU/μL, and 15 μL were given intra-tracheal (IT) per os. The present example had five arms (three drug administration groups n=5 each, and two control groups n=4). Hamsters were monitored daily measuring body weight, determining a clinical illness score. Whole body plethysmography (PFT) was recorded for each hamster on days 0, 2, 4 and 7. Serum levels of Interferon gamma (IFNγ) were determined from blood collected on days 0, 2, 4 and 7. RT-qPCR targeting the nucleocapsid gene was performed on the following samples: oropharyngeal swabs taken on days 2 and 4, bronchoalveolar lavage fluid (BAL) collected from one hamster in each group on day 4 and the remaining hamsters on day 7. RT-qPCR was also performed on RNA extracted from samples of olfactory bulbs.

All hamsters were euthanized on day 7; bronchoalveolar lavage was performed using 1.5 mL PBS, lungs were then fixed for histology, and olfactory bulbs were dissected. The virus control group was administered IT virus and received IV saline administration after SARS-CoV-2 challenge. The sham infection control group was administered 15 μL DMEM IT followed by IV saline treatment.

Each of the compounds were given at a dosage of 15 mg/kg, which was in a volume of less than 0.7 mL. Control animals received 0.5 mL of IV 250 mM saline. The compounds, or saline, were administered IV at 12 hours, 24 hours, and 48 hours after IT virus challenge.

Methods

All hamsters were weighed and observed at least once daily throughout the study. PFT was performed on days 0, 2, 4, and 7, after weighing the animals. The morning of day 0, after weighing and obtaining PFT, the hamsters were anesthetized with intraperitoneal ketamine/xylazine anesthesia (133 mg/kg and 13.3 mg/kg, respectively). Once anesthetized, the hamsters were suspended by their incisor teeth on an inclined board and the glottis was visualized using trans-tracheal illumination. Next, 5 μL of a 2% lidocaine solution was placed on the glottis for 30 seconds, followed by the placement of an IT 18 gauge catheter. The position of the catheter in the trachea was confirmed by observing breath condensate on a chilled dental mirror held at the catheter opening. After confirmation of catheter position, 15 μL of viral inoculum containing 1500 PFU, or 15 μL of DMEM for the non-viral control, were administered through the IT catheter using a 100 μL gel-loading pipet tip. After the gel loading tip was removed, one milliliter of air was forced through the IT catheter into the lung to help disseminate the inoculum, the catheter was removed and the hamster allowed to recover from anesthesia

Viral inoculum was severe acute respiratory syndrome-related coronavirus 2 (SARS-CoV-2), isolate USA-WA1/2020 obtained from BEI Resources (www.beiresources.org).

The hamsters had jugular catheters that were maintained by removal of anti-coagulant solution (25% dextrose with heparin at 500 U/mL) at each manipulation (catheter volume 41 μL); then, after drawing blood or administering treatments, the catheter was flushed with 250 μL saline and the heparin-dextrose solution replaced.

Each time the hamsters were manipulated for blood collection or substance injection, they were anesthetized with isoflurane. The hamsters were weighed and administered 15 mg/kg of a given treatment, or saline, via the jugular catheter at 12-hours, 24-hours and 48-hours after viral inoculation. On days 0, 2, 4 and 7, after body weight was collected, PFT was recorded for a period of 20 minutes; this was then followed by isoflurane anesthesia and collection of 500 μL of blood for analysis of select cytokines. Finally, a throat swab was taken (on day 2 and day 4), or BAL (day 7) after euthanasia. The five study arms were as follows
Arm (1) Non-viral DMEM control given IT followed by IV 0.5 mL saline;
Arm (2) Non-treatment control, 1500 PFU SARS CoV-2 (WA-1) IT followed by IV sterile 250 mM NaCl;
Arm (3) 1500 PFU followed by SEQ ID NO: 122 IV at 15 mg/kg;
Arm (4) 1500 PFU followed by SEQ ID NO: 154 IV at 15 mg/kg; and
Arm (5) 1500 PFU followed by SEQ ID NO: 192 IV at 15 mg/kg;

Hamsters were euthanized and samples collected on days 4 and 7. On day 4, after recording plethysmography and weight, one hamster in each group was euthanized using 0.1 mL IP Euthasol®; the remaining hamsters were euthanized on post-inoculation day 7. After euthanasia, blood was collected for IFNγ determination, and the trachea was dissected and cannulated followed by BAL collection and fixation of the lung. BAL collection consisted of 1.5 mL saline infused into the lungs and then withdrawn. RNA extracted from BAL was used for RT-qPCR to determine a relative quantity of SARS-CoV-2 in the lungs. After BAL collection, the lungs were distended with 3 mL of 10% neutral buffered formalin via the tracheal cannula and submitted for paraffin embedding and H&E staining. Additionally, the olfactory bulb was dissected from the brain for RNA extraction and SARS CoV-2 detection.

PFT is presented as Penh, Rpef and EF50 in a standard format published for rodent viral spirometry, and as calculated in the example above. Throat swabs (cotton tip) were placed in 175 μL of PBS and vortexed for 15 seconds; next, 140 μL was used for RNA extraction. RNA was extracted from throat swabs and BAL using Qiagen Viral RNA isolation kits. RNA was extracted from olfactory bulbs using Qiagen RNeasy and Qia-shredder columns.

The TaqMan® assay primer/probe sequences and reaction conditions targeting the SARS-CoV-2 viral nucleocapsid used in this study was published by the CDC (see above). The TaqMan® assay was performed on an ABI QuantStudio 3 thermal cycler using Promega GoTaq® RT-PCR. The amplicon produced by the primers was cloned into the plasmid pCR4 and used as quantitative standards to determine the number of genome copies in one microliter of RNA extracted from swabs, BAL or tissue. Cytokine analysis for IFNγ, Tumor Necrosis Factor Alpha (TNFα), angiotensin II, and angiotensin 1-7 was performed by antigen capture ELISA using kits obtained from MyBioSource and Genorise Scientific.

Results

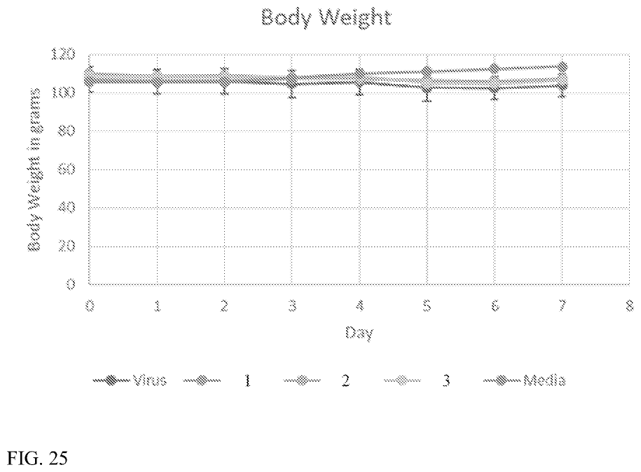

No significant weight loss occurred in any of the experimental groups (or individuals). FIG. 25.

Viral Titer

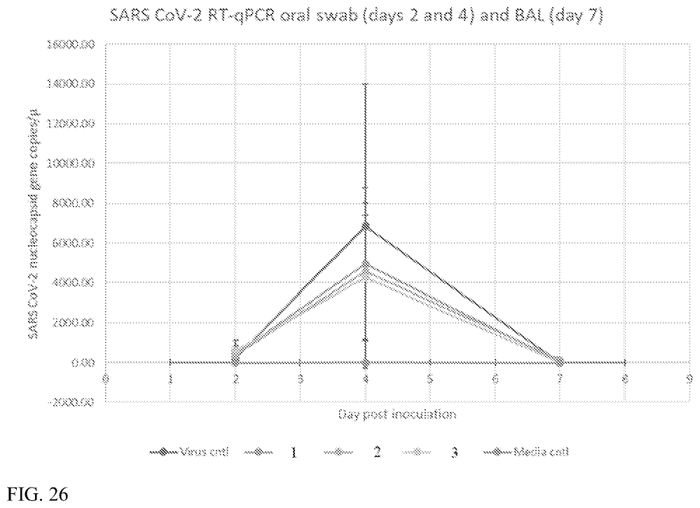

Viral titer detected as nucleocapsid gene copies/μL RNA extracted from throat swabs or BAL was highest on day four after IT inoculation. FIG. 26. The viral control group had a large standard deviation of titer ranging from 807-17,158 PFU eluted from throat swabs taken on day four. On day 7 RT-qPCR was performed on 140 μL of BAL, however by that time the copy number was minimal in all groups (<17PFU/μL). Each compound treatment groups had maximal viral copy numbers in samples taken on post-inoculation day 4 post-inoculation and those copy numbers were lower than detected in the viral challenged non-treatment control. FIG. 26.

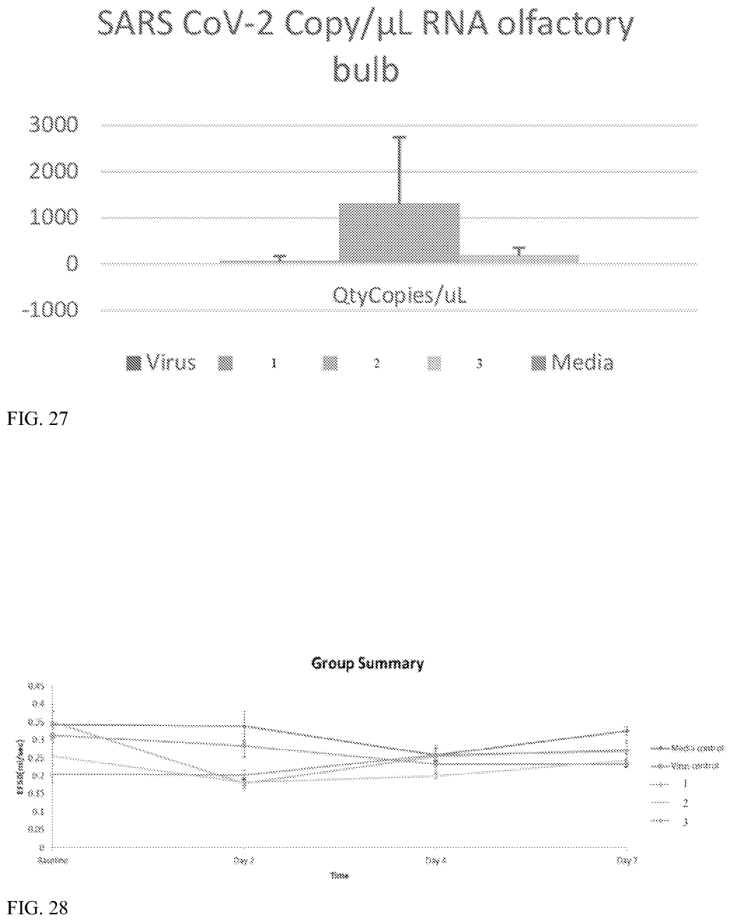

Additionally, RT-qPCR detected the presence of SARS CoV-2 genome in the olfactory bulb portion of the brain in all groups except the medium control group. Treatment with the compounds did not result in a decrease of viral nucleocapsid gene copy number detected by RT-qPCR in the RNA extracted from olfactory bulb when compared with the untreated virus controls. FIG. 27. The copy number was greatest in RNA extracted from VT116 treated hamsters, followed by VT130 treated, SEQ ID NO: 122 and virus challenge untreated control. Whether this represents infectious, replication competent, SARS CoV-2 in the brain was not determined, but the greater copy number in treatment groups is noteworthy.

On Day 7 when SARS CoV-2 could no longer be detected in the BAL collected from the mice, the genome was present in the olfactory bulb of the brain (see below).

Whole Body Plethysmography

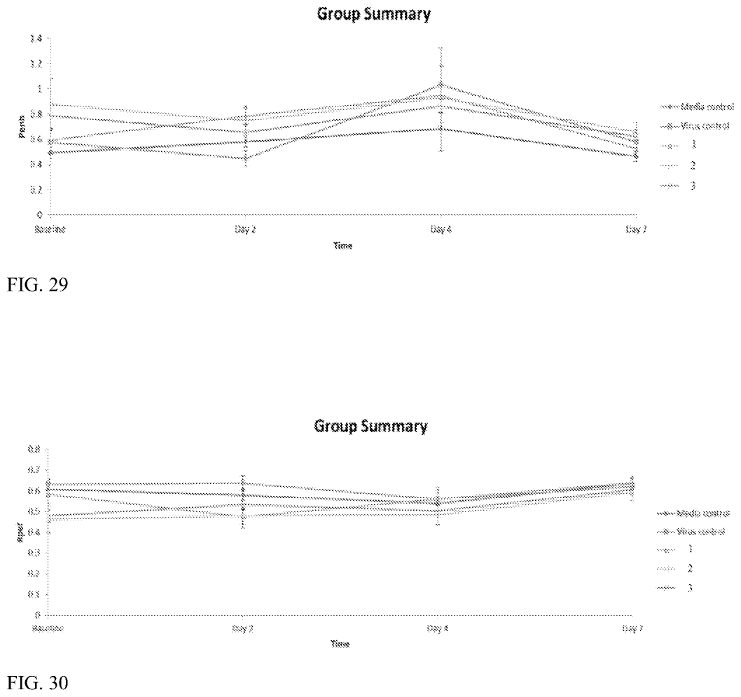

There was a significant difference between PFT testing results between the compound treatment groups and the viral control group on day 2 post-inoculation (T-test, P<0.01 SEQ ID NO: 122 and SEQ ID NO: 154, p<0.05 VT130). FIGS. 28-30. This difference was not retained on days 4 and 7 when comparing groups. The significant difference detected on day 2 may be related to the administration of the test compounds since a single adverse reaction was seen to SEQ ID NO: 192 in a single animal. The decrease in EF50 value of treatment groups compared with controls, is related to a shorter duration of time to expire 50% of a breath in the treatment groups. FIG. 28. This shorter duration is physiologically related to decreased resistance to moving the breath, an absence of bronchoconstriction, this could be a direct effect on the airway smooth muscle, or an indirect effect in diminishing pulmonary inflammation.

Plasma Cytokine Values

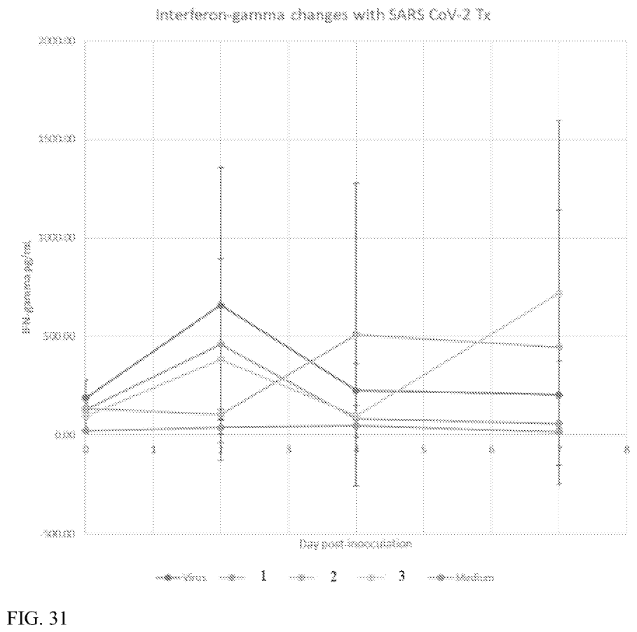

Interferon-gamma (IFNγ) levels increased in all virus inoculated groups reaching peak values on day 2 in the virus control group, and in the SEQ ID NO: 122 treated and SEQ ID NO: 192 groups. FIG. 31.

The IFNγ plasma levels peaked on day four in the SEQ ID NO: 154treatment group (500 μg/mL plasma). These levels decreased to near baseline in the SEQ ID NO: 122 group by day 7, and in the SEQ ID NO: 192 group by day 4, however in the SEQ ID NO: 192 and SEQ ID NO: 154 groups there was a paradoxical rise in IFNγ levels on day 7. This may be related to the copies of the viral genome detected in the brain of hamsters on day 7 in the SEQ ID NO: 154 and SEQ ID NO: 192 treatment groups. The virus control group did not return to baseline IFNγ levels values by the end of the study. Tumor Necrosis Factor alpha (TNFα) levels were also measured but were inconclusive (data not shown).

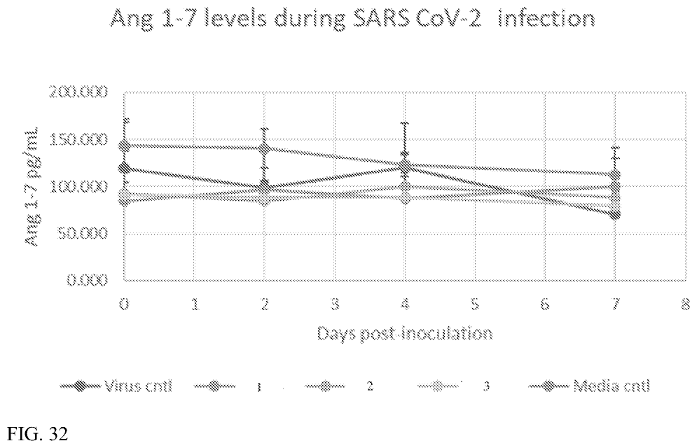

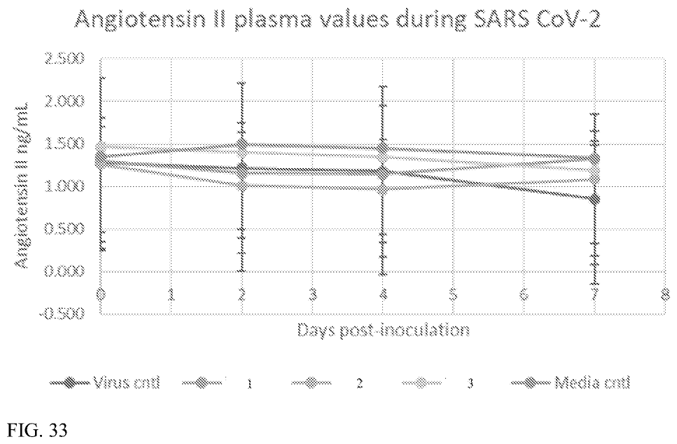

Angiotensin 1-7 levels decreased in all groups through the study, the decrease was significant between the virus control group and the media control groups (T-test, two-tailed, p<0.05). FIG. 32. The decrease was statistically significant between the media control group and all other groups on post-inoculation day 2, but only between levels determined in the two control groups on day 7. By comparison angiotensin II levels decreased in a similar manner in all groups over the course of the study, but these decreases were not statistically significant. FIG. 33.

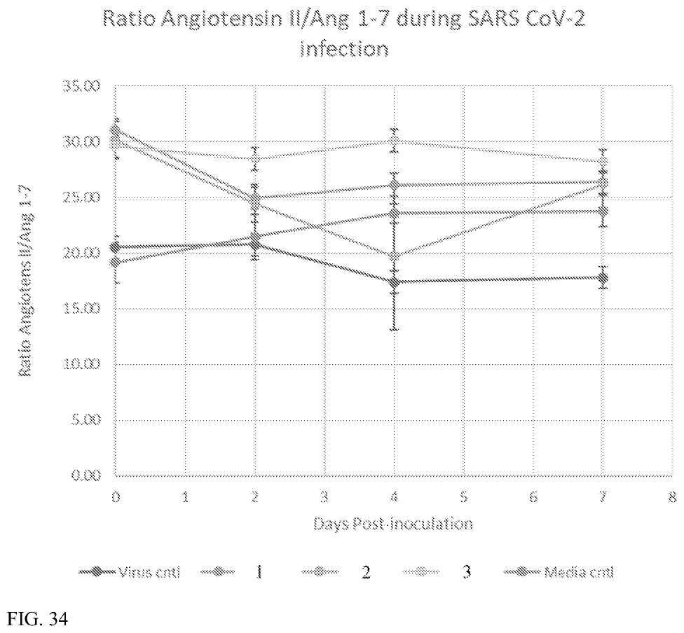

Angiotensin II (Ang II) is involved in regulation of blood pressure and is converted to angiotensin 1-7 by the angiotensin converting enzyme type 2 (ACE2), the receptor used by SARS CoV-2 to attach to cells. People with hypertension are more susceptible to adverse outcomes from the coronavirus, so we felt it prudent to examine these metabolites. The ratio of these metabolites (Ang II/Ang 1-7) is shown in FIG. 34, and is significantly different between the control groups, with the ratio resolving in the treatment groups closer to that of the media control throughout the experiment.

Histopathology

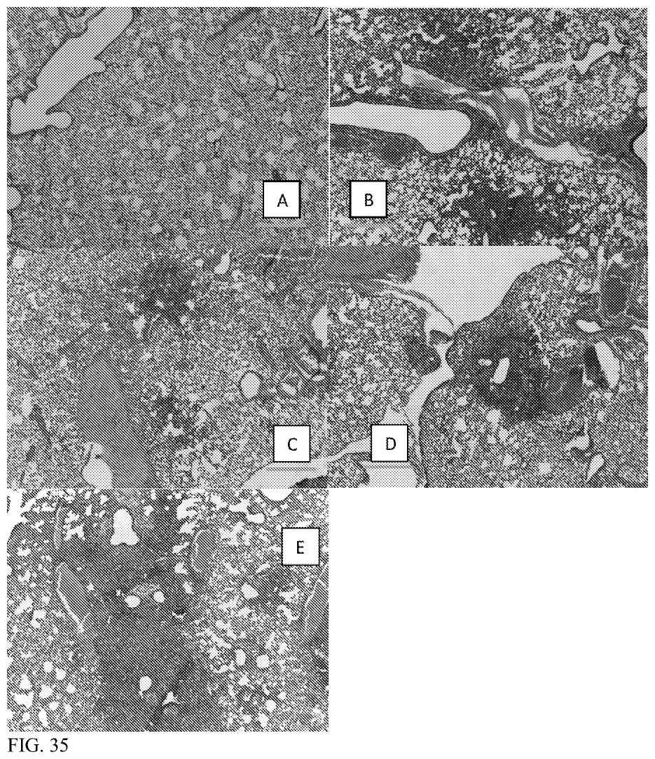

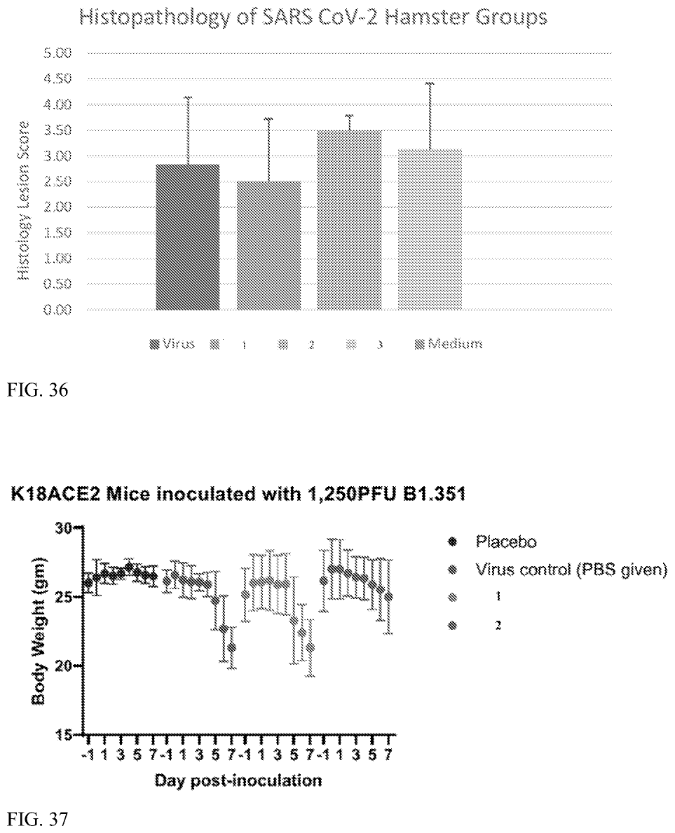

Hamsters in all groups, with the exception of the medium sham inoculated controls, had pulmonary lesions best described as chronic-active multifocal bronchopneumonia with perivascular edema. There was substantial type 2 pneumocyte proliferation in the affected areas of lung. Very infrequently found was thrombosis in small arteries in areas where the vessels were surrounded by focal intense inflammation. The histopathologic lesions were scored using the following scheme: (a) Lesion distribution: none=0, focal=1, multifocal=2, diffuse=3; (b) Inflammation intensity: none=0, mild (2-3 inflammatory cells thick)=1, moderate (inflammatory cells=3-20 cells thick), severe (inflammatory cells>20 cells thick); (c) Small vessel thrombosis: absent=0, present=1. FIGS. 35-36.

Conclusion

Several things are notable in this study. First is the initial spike in IFNγ two days after viral challenge, which decreased prior to the peak viral load detected on day 4 post-inoculation. Typically IFNγ levels will continue to be elevated during viral replication. Second is that viral copy number per microliter on day 7 diminishes to near zero in BAL but inflammation of the lung continues to be a florid level in the absence of the virus. Third, although viral copy number diminished on day 7 to near zero in BAL, viral copies were detected in the olfactory bulbs of the animals, although we did not test if these copies are associated with infectious virus or only detecting viral nucleic acid.

Treatment of hamsters with any of the three compounds decreased the IFNγ level when compared with viral controls (however, statistical significance could not be determined due to small group size and variability of measurements between individuals). Plethysmography data shows that SEQ ID NO: 122 and SEQ ID NO: 192 help the hamsters achieve respiration closer to the normal when compared to media control and virus control groups. Finally, viral titers in olfactory bulb lingered at higher values in SEQ ID NO: 192 treated hamsters on day 7 when compared with viral controls and SEQ ID NO: 122 or SEQ ID NO: 154. Viral titers on day 4 were diminished in treatment groups when compared viral controls (when corrected by removal of data from a single outlier in the SEQ ID NO: 192 treatment group). Histologically, no difference was apparent between treatments and viral control groups in the distribution or severity of pulmonary inflammation-perhaps due to the on-going inflammatory response even in the absence of virus in BAL. Because the treatments being administered focus on interference with viral entry into cells, the inflammatory response once initiated seems to be disconnected from the on-going presence of the virus and therefore inflammation once started is unaffected by the treatment.

Finally, the significance of the imbalance of angiotensin II to angiotensin 1-7 conversion as a potential cause of the residual inflammation of the lung when the virus is minimal or no longer present was assessed via the Ang II/Ang 1-7 ratio. Treatment with SEQ ID NO: 122 and to a greater extent SEQ ID NO: 192 kept that ratio normal or improved, when compared to virus control; albeit without apparent decrease in pulmonary inflammation.

### Example 12. K18-ACE2 Mouse Study

Introduction

Young female K18-ACE2 (JAX) mice weighing between 20-25 grams were treated with intraperitoneal (IP) administration of SEQ ID NO: 113; an antibody that binds a SARS-CoV-2 spike protein, said antibody having a heavy chain comprising the amino acid sequence set forth in SEQ ID NO: 189 and a light chain comprising the amino acid sequence set forth in SEQ ID NO: 190 (anti-SARS-CoV-2 spike protein antibody); or saline after 1,250 PFU SARS-CoV-2 (Strain B1.351) given by intranasal challenge. The intraperitoneal (IP) drug administration began 12-hours prior to viral challenge, and was continued with a subsequent dose 12-hours after challenge, and 24-hours after challenge.

At viral challenge the compound was mixed with the virus in a volume of 50 μL and incubated for 15 minutes at 37° C. prior to intranasal administration to mice under isoflurane anesthesia. For SEQ ID NO: 113, due to the concentration of the compound, the volume of intranasal administration was 83 μL to achieve 15 mg/kg.

The following four arms were evaluated:
Arm (1): Virus inoculation with SEQ ID NO: 113 (15 mg/kg) administration group n=13 each;
Arm (2): Virus inoculation with anti-SARS-CoV-2 spike protein antibody (1.2 mg/kg) administration group n=13 each;
Arm (3): Cell culture medium intranasal control with IP saline administration n=6;
Arm (4): Virus inoculated with IP saline administration n=6.

Mice were monitored daily measuring body weight and assessing an objective clinical illness score.

Mice were euthanized on the following schedule: Day 0 two hours after drug administration; day 1, day 2, and day 4: arm 1 n=2; arm 2 n=2, arm 3 n=1 and arm 4 n=1. The remaining mice were euthanized on day 7.

Prior to euthanasia mice were weighed and clinically scored. Under anesthesia blood was collected. After euthanasia bronchoalveolar lavage (BAL) was performed using 1 mL of sterile phosphate buffered saline, and lungs were insufflated with 1 mL formalin and fixed.

RNA was isolated from all BAL samples using Qiagen RNeasy for Viral RNA and quantified and heated to 60° C. for 20 minutes. Serum was tested for mouse fibrin degradation products and D-dimer using kits from MyBiosource.

Results

FIG. 37 shows the body weights of the mice in the four arms. Viral inoculated mice in arms 1 and 4 began to show signs clinical signs of illness by day 4, a few succumbed to the viral infection, and the remainder were all ill by day 7 at study termination.

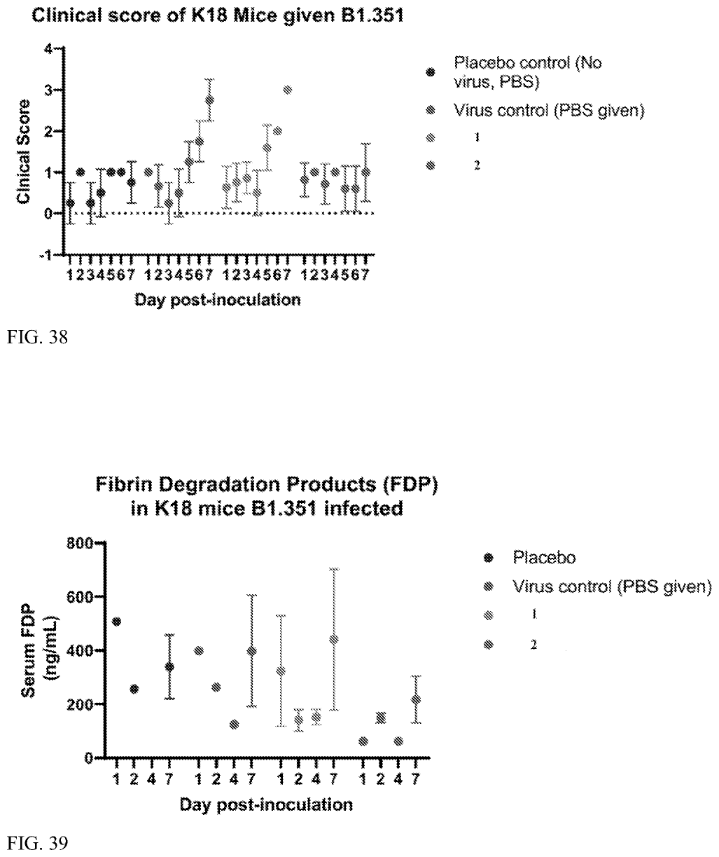

The clinical scores paralleled the changes in body weight as the mice began to show signs of illness on day 5. FIG. 38. This slower progression to clinical illness and weight loss was due to the slower nature of the South African variant of SARS CoV-2 both in vitro and in vivo compared to the WA-1 strain of the virus.

The clinical scores paralleled the changes in body weight as the mice began to show signs of illness on day 5. This slower progression to clinical illness and weight loss was due to the slower nature of the South African variant of SARS CoV-2 both in vitro and in vivo compared to the WA-1 strain of the virus.

Using a mixed model statistical analysis on the body weight data there is a significant difference between the placebo control and the virus control (p<0.001), and between the placebo control and the SEQ ID NO: 113 treated mice (p<0.05). There no statistical difference between the placebo control and the anti-SARS-CoV-2 spike protein antibody treated mice, though the data was trending in the direction of a difference were the study to have gone on longer.

Using a mixed model statistical analysis on the clinical score data there is a significant difference between the placebo control and the virus control (p<0.01) and between the placebo control and the SEQ ID NO: 113 treated mice (p<0.05) on day 6. On day 7 significant differences existed between the following groups: placebo vs. virus control (p<0.01), placebo vs. SEQ ID NO: 113 (P<0.01), SEQ ID NO: 113 vs. virus control (p<0.05) and SEQ ID NO: 113 vs. anti-SARS-CoV-2 spike protein antibody (p<0.05). There was no statistical difference between the SEQ ID NO: 113 vs. virus control, or between placebo vs. anti-SARS-CoV-2 spike protein antibody treated mice on any day.

TABLE 19
BAL RNA concentrations (ng/μL). Here, RNA
isolated from BAL fluid from each mouse is shown.

| Mouse No. | Treatment | Day 0 | Day 1 | Day 2 | Day 4 | Day 7 |
|---|---|---|---|---|---|---|
| 6 | Placebo | 93.6 | | | | |
| 7 | Virus | 105.8 | | | | |
| 24 | VT01 | 88.7 | | | | |
| 27 | VT01 | 102.5 | | | | |
| 39 | VT01R | 100.9 | | | | |
| 40 | VT01R | 103.7 | | | | |
| 38 | VT01R | | 102.1 | | | |
| 37 | VT01R | | 95.1 | | | |
| 25 | VT01 | | 102.3 | | | |
| 26 | VT01 | | 105.7 | | | |
| 14 | Virus | | 84.3 | | | |
| 5 | Placebo | | | 93.4 | | |
| 8 | Virus | | | 91.5 | | |
| 23 | VT01 | | | 97.1 | | |
| 22 | VT01 | | | 95.9 | | |
| 35 | VT01R | | | 92.4 | | |
| 36 | VT01R | | | 98.1 | | |
| 13 | Virus | | | | 100.2 | |
| 20 | VT01 | | | | 92.6 | |
| 21 | VT01 | | | | 106.3 | |
| 33 | VT01R | | | | 101.4 | |
| 34 | VT01R | | | | 90.3 | |
| 15 | VT01 | | | | | 100.3 |
| 1 | Placebo | | | | | 93.4 |
| 2 | Placebo | | | | | 92.2 |
| 3 | Placebo | | | | | 98.3 |
| 4 | Placebo | | | | | 93.1 |
| 9 | Virus | | | | | 76 |
| 10 | Virus | | | | | 95.5 |
| 11 | Virus | | | | | 102.6 |
| 12 | Virus | | | | | 95.9 |
| 16 | VT01 | | | | | 47.2 |
| 17 | VT01 | | | | | 114.4 |
| 18 | VT01 | | | | | 123.5 |
| 19 | VT01 | | | | | 92.6 |
| 28 | VT01R | | | | | 44.8 |
| 29 | VT01R | | | | | 93.3 |
| 30 | VT01R | | | | | 96.4 |
| 31 | VT01R | | | | | 96.7 |
| 32 | VT01R | | | | | 93.9 |

Microthrombosis

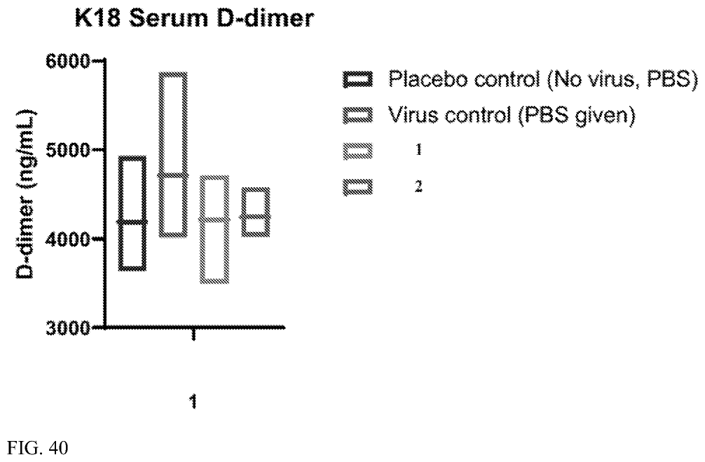

Fibrin degradation products (FDP) and D-dimer were used as an indicator of microthrombosis. Fibrin degradation products and D-dimer were measured in serum of all mice (except those found dead). FIGS. 39-40.

TABLE 20
D-dimer of K18ACE2 mice taken at the end of the
study after B1.351. strain SARS CoV-2 infection.

| Treatment | Mouse No. | D-dimer (ng/mL) |
|---|---|---|
| Placebo | 1 | 4923.78 |
| | 2 | 3853.34 |
| | 3 | 3640 |
| | 4 | 4333.34 |
| | 5 | 3853.34 |
| | 6 | 3853.34 |
| Virus | 7 | 3920 |
| | 8 | 4373.34 |
| | 9 | 4120 |
| | 10 | 4013.34 |
| | 11 | 5872.78 |
| | 12 | 4841.26 |
| | 13 | 4066.66 |
| | 14 | 3453.34 |
| SEQ ID NO: 113 | 16 | 3493.34 |
| | 17 | 3946.66 |
| | 18 | 4706.66 |
| | 19 | 4253.34 |
| | 20 | 4666.66 |
| | 21 | 4653.34 |
| | 22 | 4613.34 |
| | 23 | 5212.6 |
| | 24 | 6615.48 |
| | 25 | 5707.74 |
| | 26 | 4386.66 |
| | 27 | 4440 |
| anti-SARS-CoV-2 | 28 | 4026.66 |
| spike protein antibody | 29 | 4160 |
| | 30 | 4573.34 |
| | 31 | 4053.34 |
| | 32 | 4400 |
| | 33 | 4706.66 |
| | 34 | 5198.86 |
| | 35 | 4480 |
| | 36 | 4413.34 |
| | 37 | 4440 |
| | 38 | 4520 |
| | 39 | 6175.36 |
| | 40 | 7014.32 |

Using a mixed model statistical analysis on the FDP data, and separately on the D-dimer data, there were no significant differences between any groups in both these measurements of serum constituents.

Conclusion

The anti-SARS-CoV-2 spike protein antibody provided some protection from weight loss and clinical signs of illness. The body weights and clinical scores of SEQ ID NO: 113 did not differ significantly from the virus infected control group.

Serum levels of D-dimer were not statistically different between placebo and viral controls or the SEQ ID NO: 113 and anti-SARS-CoV-2 spike protein antibody treatment groups when all comparisons of these groups were analyzed. However, the D-dimer graph does give the impression that SEQ ID NO: 113 and anti-SARS-CoV-2 spike protein antibody average values for animals euthanized on day 8 were closer to that of the placebo control, suggesting there may have been insufficient numbers of mice in each group to demonstrate statistical significance.

### Example 13. NRP Molecules with Modified Heparin Binding

Neuropilin has acidic polysaccharide binding sites that interact with heparin or heparin sulfate to enhance the interaction of Neuropilin and VEGF. The acidic polysaccharide binding sites are on both b1 and b2, which define a continuous electropositive region.

To decrease the binding affinity to acidic polysaccharide such as heparin and heparan sulfate, fusion proteins were designed with mutations in neuropilin fragment (e.g., K358E, K373E of b1 domain, and R513E, K514E, K516E of b2 domain).

All designed proteins were generated by codon-optimized gene synthesis and inserted into pcDNA3.4 as expression vector using Not I and Hind III restriction enzyme. The constructed expression vectors include signal peptides and for optimized transcription a Kozak sequence may be included in the 5′ untranslated region.

To obtain the amount of the constructed plasmids for transfection, the constructed plasmids were transformed into One Shot™Top10 E. coli competent cells followed by culturing overnight. The constructed plasmids were obtained by PureLink™ HiPure Expi plasmid Megaprep kit.

Fusion proteins were transiently expressed in the CHO-S system (ThermoFisher Scientific Inc.). The proteins were expressed individually as per the manufacturer's instructions. Briefly, a total of 0.8 μg of plasmid DNA at a ratio of 1:1 light to heavy chain per mL of CHO-S culture was prepared with OPTIPRO™ SFM and ExpiFectamine™. The mixture was added to CHO-S cells at a viable cell density of 6×10⁶ cells/mL and greater than 98% viability. The cell culture was incubated overnight at 37° C., 80% humidity, 8% CO₂ in a Nalgene™ Single-Use PETG Erlenmeyer Flasks shaking at 125 RPM with a 19-mm orbit. The next day the culture was enhanced (ExpiCHO™ enhancer; ThermoFisher Scientific Inc.) and fed (ExpiCHO™ feed; ThermoFisher Scientific Inc.) and transferred to 32° C., 80% humidity, 5% CO₂ shaking at 125 RPM with a 19-mm orbit. The second feed was performed on day 5 and the culture returned to 32° C. until harvest on day 12.

Harvesting was accomplished via centrifugation at 4000×g for 20 minutes. The clarified supernatant was sterilized using an asymmetrical polyethersulfone (PES) 0.22-μM filter assembly (Nalgene). The filtrate was stored at 4° C. until purification the next day.

All of the antibody sterilized supernatants were purified using MabSelect prismA™ resin (GE Healthcare Life Sciences) on an ÅKTApure (GE Healthcare Life Sciences). A 50 mM sodium phosphate, 150 mM NaCl, pH 7.0 buffer was used to equilibrate the resin. The antibody supernatant was then loaded into the column.

Next, the resin was washed with 50 mM sodium phosphate, 150 mM NaCl, pF 7.0 buffer until the chromatographic baseline returned to column equilibration levels. Elution is then performed using 100 mM sodium acetate, 20% glycerol, pH 3.0, and fractions were collected. The fractions were then immediately neutralized with 1 M Tris, pH 9.

After the fractions were obtained, ion exchange chromatography was performed. A cationic exchange chromatography (CEX) column (Capto S ImpAct), or an anion exchange chromatography (AEX) column (Capto Q Impres) were sanitized with 1 M NaOH and rinsed with MQ. Equilibration was done with 50 mM NaAc pH 5.5 (starting buffer), and 50 mM NaAc pH5.5, 1M NaCl (elution buffer) for CEX or 50 mM Tris pH 8.0 (starting buffer) and 50 mM Tris-HCl pH 8.0, 1M NaCl (elution buffer) for AEX.

The pH of starting buffer and elution buffer were sometimes Na-pi pH 7.0, Bicine pH 8.0 for CEX; and Tris 8.0, Tr 8.5 for AEX, according to pI value of proteins. The protein A purified antibody was loaded with a concentration of 1-2 g antibody/mL resin. The column was then washed with the starting buffer. The antibody product was then eluted using a gradient of 0-60% elution buffer in 25 column volumes. Each peak was collected separately and concentrated via centrifugation at 4000×g using Amicon® Ultra-15 Centrifugal Filter Units followed by buffer change into PBS.

Heparin Binding

To evaluate heparin binding, a heparin affinity column (HiTrap Heparin HP) was sanitized with 1 M NaOH and rinsed with MQ. Equilibration was done with 50 mM Na phosphate pH 7.0 (starting buffer) and 50 mM Na phosphate pH 7.0, 1M NaCl (elution buffer). The construct molecules of the present invention were loaded with a concentration of 1 mg antibody/mL resin. The column was then washed with the starting buffer. The construct molecules of the present invention were then eluted using a gradient of 100% elution buffer in 15 column volumes. A summary of the constructs is presented in tables below.

TABLE 21
Binding assay constructs. Constructs evaluated herein are presented below.

| SEQ ID | |
|---|---|
| NO. | Sequence |
| 113 | EDFKCMEALGMESGEIHSDQITASSQYSTNWSAERSRLNYPENGWTPGEDSYREWIQVDLGLLRFVTAVGTQGAISKE |
| | TKKKYYVKTYKIDVSSNGEDWITIKEGNKPVLFQGNTNPTDVVVAVFPKPLITRFVRIKPATWETGISMRFEVYGCGG |
| | GGSGGGGSEPKSCDKTHTCPPCPAPELLGGPSVFLFPPKPKDTLMISRTPEVTCVVVDVSHEDPEVKFNWYVDGVEVH |
| | NAKTKPREEQYNSTYRVVSVLAVLHQDWLNGKEYKCKVSNKALPAPIEKTISKAKGQPREPQVYTLPPSRDELTKNQV |
| | SLTCLVKGFYPSDIAVAWESNGQPENNYKTTPPVLDSDGSFFLYSKLTVDKSRWQQGNVFSCSVMHEALHAHYTQKSL |
| | SLSPGK |
| 114 | EDFKCMEALGMESGEIHSDQITASSQYSTNWSAERSRLNYPENGWTPGEDSYREWIQVDLGLLRFVTAVGTQGAISKE |
| | TKKKYYVKTYKIDVSSNGEDWITIEEGNKPVLFQGNTNPTDVVVAVFPKPLITRFVRIKPATWETGISMRFEVYGCGG |
| | GGSGGGGSGGGGSGGGGSEPKSCDKTHTCPPCPAPELLGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALPAPIEKTISKAKGQPREPQVYTLPP |
| | SRDELTKNQVSLTCLVKGFYPSDIAVEWESNGQPENNYKTTPPVLDSDGSFFLYSKLTVDKSRWQQGNVFSCSVMHEA |
| | LHNHYTQKSLSLSPGK |
| 115 | EDFKCMEALGMESGEIHSDQITASSQYSTNWSAERSRLNYPENGWTPGEDSYREWIQVDLGLLRFVTAVGTQGAISKE |
| | TKKKYYVKTYKIDVSSNGEDWITIEEGNKPVLFQGNTNPTDVVVAVFPKPLITRFVRIKPATWETGISMRFEVYGCGG |
| | GGSGGGGSGGGGSGGGGSCMEALGMESGEIHSDQITASSQYSTNWSAERSRLNYPENGWTPGEDSYREWIQVDLGLLR |
| | FVTAVGTQGAISKETKKKYYVKTYKIDVSSNGEDWITIEEGNKPVLFQGNTNPTDVVVAVFPKPLITRFVRIKPATWE |
| | TGISMRFEVYGCGGGGSGGGGSGGGGSGGGGSEPKSCDKTHTCPPCPAPELLGGPSVFLFPPKPKDTLMISRTPEVTC |
| | VVVDVSHEDPEVKFNWYVDGVEVHNAKTKPREEQYNSTYRVVSVLTVLHQDWLNGKEYKCKVSNKALPAPIEKTISKA |
| | KGQPREPQVYTLPPSRDELTKNQVSLTCLVKGFYPSDIAVEWESNGQPENNYKTTPPVLDSDGSFFLYSKLTVDKSRW |
| | QQGNVFSCSVMHEALHNHYTQKSLSLSPGK |
| 116 | EDFKCMEALGMESGEIHSDQITASSQYSTNWSAERSRLNYPENGWTPGEDSYREWIQVDLGLLRFVTAVGTQGAISKE |
| | TKKKYYVKTYKIDVSSNGEDWITIEEGNKPVLFQGNTNPTDVVVAVFPKPLITRFVRIKPATWETGISMRFEVYGCGG |
| | GGSGGGGSGGGGSGGGGSCMEALGMESGEIHSDQITASSQYSTNWSAERSRLNYPENGWTPGEDSYREWIQVDLGLLR |
| | FVTAVGTQGAISKETKKKYYVKTYKIDVSSNGEDWITIEEGNKPVLFQGNTNPTDVVVAVFPKPLITRFVRIKPATWE |
| | TGISMRFEVYGCGGGGSGGGGSGGGGSGGGGSCMEALGMESGEIHSDQITASSQYSTNWSAERSRLNYPENGWTPGED |
| | SYREWIQVDLGLLRFVTAVGTQGAISKETKKKYYVKTYKIDVSSNGEDWITIEEGNKPVLFQGNTNPTDVVVAVFPKP |
| | LITREVRIKPATWETGISMRFEVYGCGGGGSGGGGSGGGGSGGGGSEPKSCDKTHTCPPCPAPELLGGPSVFLFPPKP |
| | KDTLMISRTPEVTCVVVDVSHEDPEVKFNWYVDGVEVHNAKTKPREEQYNSTYRVVSVLTVLHQDWLNGKEYKCKVSN |
| | KALPAPIEKTISKAKGQPREPQVYTLPPSRDELTKNQVSLTCLVKGFYPSDIAVEWESNGQPENNYKTTPPVLDSDGS |
| | FFLYSKLTVDKSRWQQGNVFSCSVMHEALHNHYTQKSLSLSPGK |
| 117 | EDFKCMEALGMESGEIHSDQITASSQASTNWSAERSRLNYPENGWTPGEDSYREWIQVDLGLLRFVTAVGTQGAIAKE |
| | TKKKAYVKTYKIDVSSNGEDWITIEEGNKPVLFQGNTNPTDVVVAVFPKPLITRFVRIKPATWETGISMRFEVYGCGG |
| | GGSGGGGSGGGGSGGGGSEPKSCDKTHTCPPCPAPELLGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALPAPIEKTISKAKGQPREPQVYTLPP |
| | SRDELTKNQVSLTCLVKGFYPSDIAVEWESNGQPENNYKTTPPVLDSDGSFFLYSKLTVDKSRWQQGNVFSCSVMHEA |
| | LHNHYTQKSLSLSPGK |
| 118 | EDFKCMEALGMESGEIHSDQITASSQASTNWSAERSRLNYPENGWTPGEDSYREWIQVDLGLLRFVTAVGTQGAIAKE |
| | TKKKAYVKTYKIDVSSNGEDWITIEEGNKPVLFQGNTNPTDVVVAVFPKPLITRFVRIKPATWETGISMRFEVYGCGG |
| | GGSGGGGSGGGGSGGGGSCMEALGMESGEIHSDQITASSQASTNWSAERSRLNYPENGWTPGEDSYREWIQVDLGLLR |
| | FVTAVGTQGAIAKETKKKAYVKTYKIDVSSNGEDWITIEEGNKPVLFQGNTNPTDVVVAVFPKPLITRFVRIKPATWE |
| | TGISMRFEVYGCGGGGSGGGGSGGGGSGGGGSCMEALGMESGEIHSDQITASSQASTNWSAERSRLNYPENGWTPGED |
| | SYREWIQVDLGLLRFVTAVGTQGAIAKETKKKAYVKTYKIDVSSNGEDWITIEEGNKPVLFQGNTNPTDVVVAVFPKP |
| | LITREVRIKPATWETGISMRFEVYGCGGGGSGGGGSGGGGSGGGGSEPKSCDKTHTCPPCPAPELLGGPSVFLFPPKP |
| | KDTLMISRTPEVTCVVVDVSHEDPEVKFNWYVDGVEVHNAKTKPREEQYNSTYRVVSVLTVLHQDWLNGKEYKCKVSN |
| | KALPAPIEKTISKAKGQPREPQVYTLPPSRDELTKNQVSLTCLVKGFYPSDIAVEWESNGQPENNYKTTPPVLDSDGS |
| | FFLYSKLTVDKSRWQQGNVFSCSVMHEALHNHYTQKSLSLSPGK |
| 119 | EDFKCMEALGMESGEIHSDQITASSQYSTNWSAERSRLNYPENGWTPGEDSYREWIQVDLGLLRFVTAVGTQGAISKE |
| | TKKKYYVKTYEIDVSSNGEDWITIEEGNKPVLFQGNTNPTDVVVAVFPKPLITRFVRIKPATWETGISMRFEVYGCGG |
| | GGSGGGGSGGGGSGGGGSEPKSCDKTHTCPPCPAPELLGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALPAPIEKTISKAKGQPREPQVYTLPP |
| | SRDELTKNQVSLTCLVKGFYPSDIAVEWESNGQPENNYKTTPPVLDSDGSFFLYSKLTVDKSRWQQGNVFSCSVMHEA |
| | LHNHYTQKSLSLSPGK |
| 120 | EDFKCMEALGMESGEIHSDQITASSQYSTNWSAERSRLNYPENGWTPGEDSYREWIQVDLGLLRFVTAVGTQGAISKE |
| | TKKKYYVKTYEIDVSSNGEDWITIEEGNKPVLFQGNTNPTDVVVAVFPKPLITRFVRIKPATWETGISMRFEVYGCGG |
| | GGSGGGGSGGGGSGGGGSCMEALGMESGEIHSDQITASSQYSTNWSAERSRLNYPENGWTPGEDSYREWIQVDLGLLR |
| | FVTAVGTQGAISKETKKKYYVKTYEIDVSSNGEDWITIEEGNKPVLFQGNTNPTDVVVAVFPKPLITRFVRIKPATWE |
| | TGISMRFEVYGCGGGGSGGGGSGGGGSGGGGSCMEALGMESGEIHSDQITASSQYSTNWSAERSRLNYPENGWTPGED |
| | SYREWIQVDLGLLRFVTAVGTQGAISKETKKKYYVKTYEIDVSSNGEDWITIEEGNKPVLFQGNTNPTDVVVAVFPKP |
| | LITRFVRIKPATWETGISMRFEVYGCGGGGSGGGGSGGGGSGGGGSEPKSCDKTHTCPPCPAPELLGGPSVFLFPPKP |
| | KDTLMISRTPEVTCVVVDVSHEDPEVKFNWYVDGVEVHNAKTKPREEQYNSTYRVVSVLTVLHQDWLNGKEYKCKVSN |
| | KALPAPIEKTISKAKGQPREPQVYTLPPSRDELTKNQVSLTCLVKGFYPSDIAVEWESNGQPENNYKTTPPVLDSDGS |
| | FFLYSKLTVDKSRWQQGNVFSCSVMHEALHNHYTQKSLSLSPGK |
| 121 | EDFKCMEALGMESGEIHSDQITASSQYSTNWSAERSRLNYPENGWTPGEDSYREWIQVDLGLLRFVTAVGTQGAISKE |
| | TKKKYYVKTYKIDVSSNGEDWITIKEGNKPVLFQGNTNPTDVVVAVFPKPLITRFVRIKPATWETGISMRFEVYGCKI |
| | TDYPCMEALGMESGEIHSDQITASSQYSTNWSAERSRLNYPENGWTPGEDSYREWIQVDLGLLRFVTAVGTQGAISKE |
| | TKKKYYVKTYKIDVSSNGEDWITIKEGNKPVLFQGNTNPTDVVVAVFPKPLITRFVRIKPATWETGISMRFEVYGCGG |
| | GGSGGGGSCMEALGMESGEIHSDQITASSQYSTNWSAERSRLNYPENGWTPGEDSYREWIQVDLGLLRFVTAVGTQGA |
| | ISKETKKKYYVKTYKIDVSSNGEDWITIKEGNKPVLFQGNTNPTDVVVAVFPKPLITRFVRIKPATWETGISMRFEVY |
| | GCKITDYPCMEALGMESGEIHSDQITASSQYSTNWSAERSRLNYPENGWTPGEDSYREWIQVDLGLLRFVTAVGTQGA |
| | ISKETKKKYYVKTYKIDVSSNGEDWITIKEGNKPVLFQGNTNPTDVVVAVFPKPLITRFVRIKPATWETGISMRFEVY |
| | GCGGGGSGGGGSEPKSCDKTHTCPPCPAPELLGGPSVFLFPPKPKDTLMISRTPEVTCVVVDVSHEDPEVKFNWYVDG |
| | VEVHNAKTKPREEQYNSTYRVVSVLAVLHQDWLNGKEYKCKVSNKALPAPIEKTISKAKGQPREPQVYTLPPSRDELT |
| | KNQVSLTCLVKGFYPSDIAVAWESNGQPENNYKTTPPVLDSDGSFFLYSKLTVDKSRWQQGNVFSCSVMHEALHAHYT |
| | QKSLSLSPGK |
| 122 | EDFKCMEALGMESGEIHSDQITASSQYSTNWSAERSRLNYPENGWTPGEDSYREWIQVDLGLLRFVTAVGTQGAISKE |
| | TKKKYYVKTYKIDVSSNGEDWITIKEGNKPVLFQGNTNPTDVVVAVFPKPLITRFVRIKPATWETGISMRFEVYGCKI |
| | TDYPCSGMLGMVSGLISDSQITSSNQGDRNWMPENIRLVTSRSGWALPPAPHSYINEWLQIDLGEEKIVRGIIIQGGK |
| | HRENKVFMRKFKIGYSNNGSDWKMIMDDSKRKAKSFEGNNNYDTPELRTFPALSTRFIRIYPERATHGGLGLRMELLG |
| | CGGGGSGGGGSEPKSCDKTHTCPPCPAPELLGGPSVFLFPPKPKDTLMISRTPEVTCVVVDVSHEDPEVKFNWYVDGV |
| | EVHNAKTKPREEQYNSTYRVVSVLTVLHQDWLNGKEYKCKVSNKALPAPIEKTISKAKGQPREPQVYTLPPSRDELTK |
| | NQVSLTCLVKGFYPSDIAVEWESNGQPENNYKTTPPVLDSDGSFFLYSKLTVDKSRWQQGNVFSCSVMHEALHNHYTQ |
| | KSLSLSPGK |
| 123 | EDFKCMEALGMESGEIHSDQITASSQYSTNWSAERSRLNYPENGWTPGADSYREWIQVDLGLLRFVTAVGTQGAISKE |
| | TKKKYYVKTYKIDVSSNGEDWITIKEGNKPVLFQGNTNPTDVVVAVFPKPLITRFVRIKPATWETGISMRFEVYGCKI |
| | TDYPCSGMLGMVSGLISDSQITSSNQGDRNWMPENIRLVTSRSGWALPPAPHSYINEWLQIDLGEEKIVRGIIIQGGK |
| | HRENKVFMRKFKIGYSNNGSDWKMIMDDSKRKAKSFEGNNNYDTPELRTFPALSTRFIRIYPERATHGGLGLRMELLG |
| | CGGGGSGGGGSERKCCVECPPCPAPPVAGPSVFLFPPKPKDQLMISRTPEVTCVVVDVSHEDPEVQFNWYVDGVEVHN |
| | AKTKPREEQFNSTFRVVSVLTVVHQDWLNGKEYKCKVSNKGLPAPIEKTISKTKGQPREPQVYTLPPSREEMTKNQVS |
| | LTCLVKGFYPSDIAVEWESNGQPENNYKTTPPMLDSDGSFFLYSKLTVDKSRWQQGNVFSCSVLHEALHNHYTQKSLS |
| | LSPGK |
| 124 | EDFKCMEALGMESGEIHSDQITASSQYSTNWSAERSRLNYPENGWTPGEDSYREWIQVDLGLLRFVTAVGTQGAISKE |
| | TKAKYYVKTYKIDVSSNGEDWITIKEGNKPVLFQGNTNPTDVVVAVFPKPLITRFVRIKPATWETGISMRFEVYGCKI |
| | TDYPCSGMLGMVSGLISDSQITSSNQGDRNWMPENIRLVTSRSGWALPPAPHSYINEWLQIDLGEEKIVRGIIIQGGK |
| | HRENKVFMRKFKIGYSNNGSDWKMIMDDSKRKAKSFEGNNNYDTPELRTFPALSTRFIRIYPERATHGGLGLRMELLG |
| | CGGGGSGGGGSERKCCVECPPCPAPPVAGPSVFLFPPKPKDQLMISRTPEVTCVVVDVSHEDPEVQFNWYVDGVEVHN |
| | AKTKPREEQFNSTFRVVSVLTVVHQDWLNGKEYKCKVSNKGLPAPIEKTISKTKGQPREPQVYTLPPSREEMTKNQVS |
| | LTCLVKGFYPSDIAVEWESNGQPENNYKTTPPMLDSDGSFFLYSKLTVDKSRWQQGNVFSCSVLHEALHNHYTQKSLS |
| | LSPGK |
| 125 | EDFKCMEALGMESGEIHSDQITASSQASTNWSAERSRLNYPENGWTPGEDSYREWIQVDLGLLRFVTAVGTQGAIAKE |
| | TKKKAYVKTYKIDVSSNGEDWITIKEGNKPVLFQGNTNPTDVVVAVFPKPLITRFVRIKPATWETGISMRFEVYGCKI |
| | TDYPCSGMLGMVSGLISDSQITSSNQGDRNWMPENIRLVTSRSGWALPPAPHSYINEWLQIDLGEEKIVRGIIIQGGK |
| | HRENKVFMRKFKIGYSNNGSDWKMIMDDSKRKAKSFEGNNNYDTPELRTFPALSTRFIRIYPERATHGGLGLRMELLG |
| | CGGGGSGGGGSERKCCVECPPCPAPPVAGPSVFLFPPKPKDQLMISRTPEVTCVVVDVSHEDPEVQFNWYVDGVEVHN |
| | AKTKPREEQFNSTFRVVSVLTVVHQDWLNGKEYKCKVSNKGLPAPIEKTISKTKGQPREPQVYTLPPSREEMTKNQVS |
| | LTCLVKGFYPSDIAVEWESNGQPENNYKTTPPMLDSDGSFFLYSKLTVDKSRWQQGNVFSCSVLHEALHNHYTQKSLS |
| | LSPGK |
| 126 | EDFKCMEALGMESGEIHSDQITASSQYSTNWSAERSRLNYPENGWTPGEDSYREWIQVDLGLLRFVTAVGTQGAISKE |
| | TKKKYYVKTYKIDVSSNGEDWITIKEGNKPVLFQGNTNPTDVVVAVFPKPLITRFVRIKPATWETGISMRFEVYGCKI |
| | TDYPCSGMLGMVSGLISDSQITSSNQGDRNWMPENIRLVTSRSGWALPPAPHSYINEWLQIDLGEEKIVRGIIIQGGK |
| | HRENKVFMEEFEIGYSNNGSDWKMIMDDSKRKAKSFEGNNNYDTPELRTFPALSTRFIRIYPERATHGGLGLRMELLG |
| | CEVEAGAPAPAGAPAPATCPPCPAPELLGGPSVFLFPPKPKDTLMISRTPEVTCVVVDVSHEDPEVKFNWYVDGVEVH |
| | NAKTKPREEQYNSTYRVVSVLTVLHQDWLNGKEYKCKVSNKALPAPIEKTISKAKGQPREPQVYTLPPSRDELTKNQV |
| | SLTCLVKGFYPSDIAVEWESNGQPENNYKTTPPVLDSDGSFFLYSKLTVDKSRWQQGNVFSCSVMHEALHNHYTQKSL |
| | SLSPGK |
| 127 | EDFKCMEALGMESGEIHSDQITASSQYSTNWSAERSRLNYPENGWTPGEDSYREWIQVDLGLLRFVTAVGTQGAISKE |
| | TKKKYYVKTYKIDVSSNGEDWITIKEGNKPVLFQGNTNPTDVVVAVFPKPLITRFVRIKPATWETGISMRFEVYGCKI |
| | TDYPCSGMLGMVSGLISDSQITSSNQGDRNWMPENIRLVTSRSGWALPPAPHSYINEWLQIDLGEEKIVRGIIIQGGK |
| | HRENKVFMAAFAIGYSNNGSDWKMIMDDSKRKAKSFEGNNNYDTPELRTFPALSTRFIRIYPERATHGGLGLRMELLG |
| | CEVEAGAPAPAGAPAPATCPPCPAPELLGGPSVFLFPPKPKDTLMISRTPEVTCVVVDVSHEDPEVKFNWYVDGVEVH |
| | NAKTKPREEQYNSTYRVVSVLTVLHQDWLNGKEYKCKVSNKALPAPIEKTISKAKGQPREPQVYTLPPSRDELTKNQV |
| | SLTCLVKGFYPSDIAVEWESNGQPENNYKTTPPVLDSDGSFFLYSKLTVDKSRWQQGNVFSCSVMHEALHNHYTQKSL |
| | SLSPGK |
| 128 | EDFKCMEALGMESGEIHSDQITASSQYSTNWSAERSRLNYPENGWTPGEDSYREWIQVDLGLLRFVTAVGTQGAISKE |
| | TKKKYYVKTYEIDVSSNGEDWITIEEGNKPVLFQGNTNPTDVVVAVFPKPLITREVRIKPATWETGISMRFEVYGCKI |
| | TDYPCSGMLGMVSGLISDSQITSSNQGDRNWMPENIRLVTSRSGWALPPAPHSYINEWLQIDLGEEKIVRGIIIQGGK |
| | HRENKVFMEEFEIGYSNNGSDWKMIMDDSKRKAKSFEGNNNYDTPELRTFPALSTRFIRIYPERATHGGLGLRMELLG |
| | CGGGGSGGGGSEPKSCDKTHTCPPCPAPELLGGPSVFLFPPKPKDTLMISRTPEVTCVVVDVSHEDPEVKFNWYVDGV |
| | EVHNAKTKPREEQYNSTYRVVSVLTVLHQDWLNGKEYKCKVSNKALPAPIEKTISKAKGQPREPQVYTLPPSRDELTK |
| | NQVSLTCLVKGFYPSDIAVEWESNGQPENNYKTTPPVLDSDGSFFLYSKLTVDKSRWQQGNVFSCSVMHEALHNHYTQ |
| | KSLSLSPGK |
| 129 | EDFKCMEALGMESGEIHSDQITASSQASTNWSAERSRLNYPENGWTPGEDSYREWIQVDLGLLRFVTAVGTQGAIAKE |
| | TKKKAYVKTYEIDVSSNGEDWITIEEGNKPVLFQGNTNPTDVVVAVFPKPLITRFVRIKPATWETGISMRFEVYGCKI |
| | TDYPCSGMLGMVSGLISDSQITSSNQGDRNWMPENIRLVTSRSGWALPPAPHSYINEWLQIDLGEEKIVRGIIIQGGK |
| | HRENKVFMEEFEIGYSNNGSDWKMIMDDSKRKAKSFEGNNNYDTPELRTFPALSTRFIRIYPERATHGGLGLRMELLG |
| | CGGGGSGGGGSEPKSCDKTHTCPPCPAPELLGGPSVFLFPPKPKDTLMISRTPEVTCVVVDVSHEDPEVKFNWYVDGV |
| | EVHNAKTKPREEQYNSTYRVVSVLTVLHQDWLNGKEYKCKVSNKALPAPIEKTISKAKGQPREPQVYTLPPSRDELTK |
| | NQVSLTCLVKGFYPSDIAVEWESNGQPENNYKTTPPVLDSDGSFFLYSKLTVDKSRWQQGNVFSCSVMHEALHNHYTQ |
| | KSLSLSPGK |
| 130 | TCPPCPAPEFLGGPSVFLFPPKPKDTLMISRTPEVTCVVVDVSQEDPEVQFNWYVDGVEVHNAKTKPREEQFNSTYRV |
| | VSVLTVLHQDWLNGKEYKCKVSNKGLPSSIEKTISKAKGQPREPQVYTLPPSQEEMTENQVSLTCLVKGFYPSDIAVE |
| | WESNGQPENNYKTTPPVLDSDGSFFLYSWLTVDKSRWQEGNVFSCSVMHEALHNHYTQKSLSLSLGKGAPAPAGAPAP |
| | AEDFKCMEALGMESGEIHSDQITASSQYSTNWSAERSRLNYPENGWTPGEDSYREWIQVDLGLLRFVTAVGTQGAISK |
| | ETKKKYYVKTYKIDVSSNGEDWITIKEGNKPVLFQGNTNPTDVVVAVFPKPLITRFVRIKPATWETGISMRFEVYGCK |
| | ITDYPCSGMLGMVSGLISDSQITSSNQGDRNWMPENIRLVTSRSGWALPPAPHSYINEWLQIDLGEEKIVRGIIIQGG |
| | KHRENKVFMRKFKIGYSNNGSDWKMIMDDSKRKAKSFEGNNNYDTPELRTFPALSTRFIRIYPERATHGGLGLRMELL |
| | GCEVEA |
| 131 | TCPPCPAPEFLGGPSVFLFPPKPKDTLMISRTPEVTCVVVDVSQEDPEVQFNWYVDGVEVHNAKTKPREEQFNSTYRV |
| | VSVLTVLHQDWLNGKEYKCKVSNKGLPSSIEKTISKAKGQPREPQVYTLPPSQEEMTENQVSLTCLVKGFYPSDIAVE |
| | WESNGQPENNYKTTPPVLDSDGSFFLYSWLTVDKSRWQEGNVFSCSVMHEALHNHYTQKSLSLSLGKGAPAPAGAPAP |
| | AEDFKCMEALGMESGEIHSDQITASSQASTNWSAERSRLNYPENGWAPGEASYREWIQVDLGLLRFVTAVGTQGAISK |
| | ETKKKYYVKTYKIDVSSNGEDWITIKEGNKPVLFQGNTNPTDVVVAVFPKPLITRFVRIKPATWETGISMRFEVYGCK |
| | ITDYPCSGMLGMVSGLISDSQITSSNQGDRNWMPENIRLVTSRSGWALPPAPHSYINEWLQIDLGEEKIVRGIIIQGG |
| | KHRENKVFMAAFAIGYSNNGSDWKMIMDDSKRKAKSFEGNNNYDTPELRTFPALSTRFIRIYPERATHGGLGLRMELL |
| | GCEVEA |
| 132 | TCPPCPAPEFLGGPSVFLFPPKPKDTLMISRTPEVTCVVVDVSQEDPEVQFNWYVDGVEVHNAKTKPREEQFNSTYRV |
| | VSVLTVLHQDWLNGKEYKCKVSNKGLPSSIEKTISKAKGQPREPQVYTLPPSQEEMTENQVSLTCLVKGFYPSDIAVE |
| | WESNGQPENNYKTTPPVLDSDGSFFLYSWLTVDKSRWQEGNVFSCSVMHEALHNHYTQKSLSLSLGKGAPAPAGAPAP |
| | AEDFKCSGMLGMVSGLISDSQITSSNQGDRNWMPENIRLVTSRSGWALPPAPHSYINEWLQIDLGEEKIVRGIIIQGG |
| | KHRENKVFMAAFAIGYSNNGSDWKMIMDDSKRKAKSFEGNNNYDTPELRTFPALSTRFIRIYPERATHGGLGLRMELL |
| | GCKITDYPCMEALGMESGEIHSDQITASSQASTNWSAERSRLNYPENGWAPGEASYREWIQVDLGLLRFVTAVGTQGA |
| | ISKETKKKYYVKTYKIDVSSNGEDWITIKEGNKPVLFQGNTNPTDVVVAVFPKPLITRFVRIKPATWETGISMRFEVY |
| | GCEVEA |
| 133 | EDFKCMEALGMESGEIHSDQITASSQYSTNWSAERSRLNYPENGWTPGEDSYREWIQVDLGLLRFVTAVGTQGAISKE |
| | TKKKYYVKTYKIDVSSNGEDWITIEEGNKPVLFQGNTNPTDVVVAVFPKPLITRFVRIKPATWETGISMRFEVYGCGG |
| | GGSGGGGSGGGGSGGGGSEPKSCDKTHTCPPCPAPELLGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALPAPIEKTISKAKGQPREPQVYTLPP |
| | SRDELTKNQVSLTCLVKGFYPSDIAVEWESNGQPENNYKTTPPVLDSDGSFFLYSKLTVDKSRWQQGNVFSCSVMHEA |
| | LHNHYTQKSLSLSPGKGGGGSGGGGSGGGGSGGGGSCMEALGMESGEIHSDQITASSQYSTNWSAERSRLNYPENGWT |
| | PGEDSYREWIQVDLGLLRFVTAVGTQGAISKETKKKYYVKTYKIDVSSNGEDWITIEEGNKPVLFQGNTNPTDVVVAV |
| | FPKPLITREVRIKPATWETGISMRFEVYGCEVEA |
| 134 | EDFKCMEALGMESGEIHSDQITASSQYSTNWSAERSRLNYPENGWTPGEDSYREWIQVDLGLLRFVTAVGTQGAISKE |
| | TKKKYYVKTYKIDVSSNGEDWITIEEGNKPVLFQGNTNPTDVVVAVFPKPLITRFVRIKPATWETGISMRFEVYGCGG |
| | GGSGGGGSGGGGSGGGGSCMEALGMESGEIHSDQITASSQYSTNWSAERSRLNYPENGWTPGEDSYREWIQVDLGLLR |
| | FVTAVGTQGAISKETKKKYYVKTYKIDVSSNGEDWITIEEGNKPVLFQGNTNPTDVVVAVFPKPLITRFVRIKPATWE |
| | TGISMRFEVYGCGGGGSGGGGSGGGGSGGGGSEPKSCDKTHTCPPCPAPELLGGPSVFLFPPKPKDTLMISRTPEVTC |
| | VVVDVSHEDPEVKFNWYVDGVEVHNAKTKPREEQYNSTYRVVSVLTVLHQDWLNGKEYKCKVSNKALPAPIEKTISKA |
| | KGQPREPQVYTLPPSRDELTKNQVSLTCLVKGFYPSDIAVEWESNGQPENNYKTTPPVLDSDGSFFLYSKLTVDKSRW |
| | QQGNVFSCSVMHEALHNHYTQKSLSLSPGKGGGGSGGGGSGGGGSGGGGSCMEALGMESGEIHSDQITASSQYSTNWS |
| | AERSRLNYPENGWTPGEDSYREWIQVDLGLLRFVTAVGTQGAISKETKKKYYVKTYKIDVSSNGEDWITIEEGNKPVL |
| | FQGNTNPTDVVVAVFPKPLITRFVRIKPATWETGISMRFEVYGCEVEA |
| 135 | EDFKCMEALGMESGEIHSDQITASSQYSTNWSAERSRLNYPENGWTPGEDSYREWIQVDLGLLRFVTAVGTQGAISKE |
| | TKKKYYVKTYKIDVSSNGEDWITIKEGNKPVLFQGNTNPTDVVVAVFPKPLITRFVRIKPATWETGISMRFEVYGCGG |
| | GGSGGGGSGGGGSGGGGSCMEALGMESGEIHSDQITASSQYSTNWSAERSRLNYPENGWTPGEDSYREWIQVDLGLLR |
| | FVTAVGTQGAISKETKKKYYVKTYKIDVSSNGEDWITIKEGNKPVLFQGNTNPTDVVVAVFPKPLITRFVRIKPATWE |
| | TGISMRFEVYGCGGGGSGGGGSGGGGSGGGGSEPKSCDKTHTCPPCPAPELLGGPSVFLFPPKPKDTLMISRTPEVTC |
| | VVVDVSHEDPEVKFNWYVDGVEVHNAKTKPREEQYNSTYRVVSVLTVLHQDWLNGKEYKCKVSNKALPAPIEKTISKA |
| | KGQPREPQVYTLPPSRDELTKNQVSLTCLVKGFYPSDIAVEWESNGQPENNYKTTPPVLDSDGSFFLYSKLTVDKSRW |
| | QQGNVFSCSVMHEALHNHYTQKSLSLSPGK |
| 136 | EDFKCMEALGMESGEIHSDQITASSQYSTNWSAERSRLNYPENGWTPGEDSYREWIQVDLGLLRFVTAVGTQGAISKE |
| | TKKKYYVKTYKIDVSSNGEDWITIKEGNKPVLFQGNTNPTDVVVAVFPKPLITRFVRIKPATWETGISMRFEVYGCGG |
| | GGSGGGGSGGGGSGGGGSCMEALGMESGEIHSDQITASSQYSTNWSAERSRLNYPENGWTPGEDSYREWIQVDLGLLR |
| | FVTAVGTQGAISKETKKKYYVKTYKIDVSSNGEDWITIKEGNKPVLFQGNTNPTDVVVAVFPKPLITRFVRIKPATWE |
| | TGISMRFEVYGCGGGGSGGGGSGGGGSGGGGSCMEALGMESGEIHSDQITASSQYSTNWSAERSRLNYPENGWTPGED |
| | SYREWIQVDLGLLRFVTAVGTQGAISKETKKKYYVKTYKIDVSSNGEDWITIKEGNKPVLFQGNTNPTDVVVAVFPKP |
| | LITRFVRIKPATWETGISMRFEVYGCGGGGSGGGGSGGGGSGGGGSEPKSCDKTHTCPPCPAPELLGGPSVFLFPPKP |
| | KDTLMISRTPEVTCVVVDVSHEDPEVKFNWYVDGVEVHNAKTKPREEQYNSTYRVVSVLTVLHQDWLNGKEYKCKVSN |
| | KALPAPIEKTISKAKGQPREPQVYTLPPSRDELTKNQVSLTCLVKGFYPSDIAVEWESNGQPENNYKTTPPVLDSDGS |
| | FFLYSKLTVDKSRWQQGNVFSCSVMHEALHNHYTQKSLSLSPGK |
| 137 | EDFKCMEALGMESGEIHSDQITASSQYSTNWSAERSRLNYPENGWTPGEDSYREWIQVDLGLLRFVTAVGTQGAISKE |
| | TKKKYYVKTYKIDVSSNGEDWITIKEGNKPVLFQGNTNPTDVVVAVFPKPLITRFVRIKPATWETGISMRFEVYGCGG |
| | GGSGGGGSGGGGSGGGGSEPKSCDKTHTCPPCPAPELLGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALPAPIEKTISKAKGQPREPQVYTLPP |
| | SRDELTKNQVSLTCLVKGFYPSDIAVEWESNGQPENNYKTTPPVLDSDGSFFLYSKLTVDKSRWQQGNVFSCSVMHEA |
| | LHNHYTQKSLSLSPGKGGGGSGGGGSGGGGSGGGGSCMEALGMESGEIHSDQITASSQYSTNWSAERSRLNYPENGWT |
| | PGEDSYREWIQVDLGLLRFVTAVGTQGAISKETKKKYYVKTYKIDVSSNGEDWITIKEGNKPVLFQGNTNPTDVVVAV |
| | FPKPLITRFVRIKPATWETGISMRFEVYGCEVEA |
| 138 | EDFKCMEALGMESGEIHSDQITASSQYSTNWSAERSRLNYPENGWTPGEDSYREWIQVDLGLLRFVTAVGTQGAISKE |
| | TKKKYYVKTYKIDVSSNGEDWITIKEGNKPVLFQGNTNPTDVVVAVFPKPLITRFVRIKPATWETGISMRFEVYGCKI |
| | TDYPCSGMLGMVSGLISDSQITSSNQGDRNWMPENIRLVTSRSGWALPPAPHSYINEWLQIDLGEEKIVRGIIIQGGK |
| | HRENKVFMRKFKIGYSNNGSDWKMIMDDSKRKAKSFEGNNNYDTPELRTFPALSTRFIRIYPERATHGGLGLRMELLG |
| | CGGGGSGGGGSCMEALGMESGEIHSDQITASSQYSTNWSAERSRLNYPENGWTPGEDSYREWIQVDLGLLRFVTAVGT |
| | QGAISKETKKKYYVKTYKIDVSSNGEDWITIKEGNKPVLFQGNTNPTDVVVAVFPKPLITRFVRIKPATWETGISMRF |
| | EVYGCKITDYPCSGMLGMVSGLISDSQITSSNQGDRNWMPENIRLVTSRSGWALPPAPHSYINEWLQIDLGEEKIVRG |
| | IIIQGGKHRENKVFMRKFKIGYSNNGSDWKMIMDDSKRKAKSFEGNNNYDTPELRTFPALSTRFIRIYPERATHGGLG |
| | LRMELLGCGGGGSGGGGSEPKSCDKTHTCPPCPAPELLGGPSVFLFPPKPKDTLMISRTPEVTCVVVDVSHEDPEVKF |
| | NWYVDGVEVHNAKTKPREEQYNSTYRVVSVLAVLHQDWLNGKEYKCKVSNKALPAPIEKTISKAKGQPREPQVYTLPP |
| | SRDELTKNQVSLTCLVKGFYPSDIAVAWESNGQPENNYKTTPPVLDSDGSFFLYSKLTVDKSRWQQGNVFSCSVMHEA |
| | LHAHYTQKSLSLSPGK |
| 139 | EDFKCMEALGMESGEIHSDQITASSQYSTNWSAERSRLNYPENGWTPGADSYREWIQVDLGLLRFVTAVGTQGAISKE |
| | TKKKYYVKTYKIDVSSNGEDWITIKEGNKPVLFQGNTNPTDVVVAVFPKPLITRFVRIKPATWETGISMRFEVYGCKI |
| | TDYPCSGMLGMVSGLISDSQITSSNQGDRNWMPENIRLVTSRSGWALPPAPHSYINEWLQIDLGEEKIVRGIIIQGGK |
| | HRENKVFMRKFKIGYSNNGSDWKMIMDDSKRKAKSFEGNNNYDTPELRTFPALSTRFIRIYPERATHGGLGLRMELLG |
| | CGGGGSGGGGSCMEALGMESGEIHSDQITASSQYSTNWSAERSRLNYPENGWTPGADSYREWIQVDLGLLRFVTAVGT |
| | QGAISKETKKKYYVKTYKIDVSSNGEDWITIKEGNKPVLFQGNTNPTDVVVAVFPKPLITRFVRIKPATWETGISMRF |
| | EVYGCKITDYPCSGMLGMVSGLISDSQITSSNQGDRNWMPENIRLVTSRSGWALPPAPHSYINEWLQIDLGEEKIVRG |
| | IIIQGGKHRENKVFMRKFKIGYSNNGSDWKMIMDDSKRKAKSFEGNNNYDTPELRTFPALSTRFIRIYPERATHGGLG |
| | LRMELLGCGGGGSGGGGSEPKSCDKTHTCPPCPAPELLGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALPAPIEKTISKAKGQPREPQVYTLPP |
| | SRDELTKNQVSLTCLVKGFYPSDIAVEWESNGQPENNYKTTPPVLDSDGSFFLYSKLTVDKSRWQQGNVFSCSVMHEA |
| | LHNHYTQKSLSLSPGK |
| 140 | EDFKCMEALGMESGEIHSDQITASSQYSTNWSAERSRLNYPENGWTPGEDSYREWIQVDLGLLRFVTAVGTQGAISKE |
| | TKAKYYVKTYKIDVSSNGEDWITIKEGNKPVLFQGNTNPTDVVVAVFPKPLITRFVRIKPATWETGISMRFEVYGCKI |
| | TDYPCSGMLGMVSGLISDSQITSSNQGDRNWMPENIRLVTSRSGWALPPAPHSYINEWLQIDLGEEKIVRGIIIQGGK |
| | HRENKVFMRKFKIGYSNNGSDWKMIMDDSKRKAKSFEGNNNYDTPELRTFPALSTRFIRIYPERATHGGLGLRMELLG |
| | CGGGGSGGGGSCMEALGMESGEIHSDQITASSQYSTNWSAERSRLNYPENGWTPGEDSYREWIQVDLGLLRFVTAVGT |
| | QGAISKETKAKYYVKTYKIDVSSNGEDWITIKEGNKPVLFQGNTNPTDVVVAVFPKPLITRFVRIKPATWETGISMRF |
| | EVYGCKITDYPCSGMLGMVSGLISDSQITSSNQGDRNWMPENIRLVTSRSGWALPPAPHSYINEWLQIDLGEEKIVRG |
| | IIIQGGKHRENKVFMRKFKIGYSNNGSDWKMIMDDSKRKAKSFEGNNNYDTPELRTFPALSTRFIRIYPERATHGGLG |
| | LRMELLGCGGGGSGGGGSEPKSCDKTHTCPPCPAPELLGGPSVFLFPPKPKDTLMISRTPEVTCVVVDVSHEDPEVKF |
| | NWYVDGVEVHNAKTKPREEQYASTYRVVSVLAVLHQDWLNGKEYKCKVSNKALPAPIEKTISKAKGQPREPQVYTLPP |
| | SRDELTKNQVSLTCLVKGFYPSDIAVAWESNGQPENNYKTTPPVLDSDGSFFLYSKLTVDKSRWQQGNVFSCSVMHEA |
| | LHAHYTQKSLSLSPGK |
| 141 | EDFKCMEALGMESGEIHSDQITASSQASTNWSAERSRLNYPENGWTPGEDSYREWIQVDLGLLRFVTAVGTQGAIAKE |
| | TKKKAYVKTYKIDVSSNGEDWITIKEGNKPVLFQGNTNPTDVVVAVFPKPLITRFVRIKPATWETGISMRFEVYGCKI |
| | TDYPCSGMLGMVSGLISDSQITSSNQGDRNWMPENIRLVTSRSGWALPPAPHSYINEWLQIDLGEEKIVRGIIIQGGK |
| | HRENKVFMRKFKIGYSNNGSDWKMIMDDSKRKAKSFEGNNNYDTPELRTFPALSTRFIRIYPERATHGGLGLRMELLG |
| | CGGGGSGGGGSCMEALGMESGEIHSDQITASSQASTNWSAERSRLNYPENGWTPGEDSYREWIQVDLGLLRFVTAVGT |
| | QGAIAKETKKKAYVKTYKIDVSSNGEDWITIKEGNKPVLFQGNTNPTDVVVAVFPKPLITRFVRIKPATWETGISMRF |
| | EVYGCKITDYPCSGMLGMVSGLISDSQITSSNQGDRNWMPENIRLVTSRSGWALPPAPHSYINEWLQIDLGEEKIVRG |
| | IIIQGGKHRENKVFMRKFKIGYSNNGSDWKMIMDDSKRKAKSFEGNNNYDTPELRTFPALSTRFIRIYPERATHGGLG |
| | LRMELLGCGGGGSGGGGSEPKSCDKTHTCPPCPAPELLGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALPAPIEKTISKAKGQPREPQVYTLPP |
| | SRDELTKNQVSLTCLVKGFYPSDIAVEWESNGQPENNYKTTPPVLDSDGSFFLYSKLTVDKSRWQQGNVFSCSVMHEA |
| | LHNHYTQKSLSLSPGK |
| 142 | EDFKCMEALGMESGEIHSDQITASSQYSTNWSAERSRLNYPENGWTPGEDSYREWIQVDLGLLRFVTAVGTQGAISKE |
| | TKKKYYVKTYKIDVSSNGEDWITIKEGNKPVLFQGNTNPTDVVVAVFPKPLITRFVRIKPATWETGISMRFEVYGCKI |
| | TDYPCSGMLGMVSGLISDSQITSSNQGDRNWMPENIRLVTSRSGWALPPAPHSYINEWLQIDLGEEKIVRGIIIQGGK |
| | HRENKVFMRKFKIGYSNNGSDWKMIMDDSKRKAKSFEGNNNYDTPELRTFPALSTRFIRIYPERATHGGLGLRMELLG |
| | CGGGGSGGGGSEPKSCDKTHTCPPCPAPELLGGPSVFLFPPKPKDTLMISRTPEVTCVVVDVSHEDPEVKFNWYVDGV |
| | EVHNAKTKPREEQYNSTYRVVSVLTVLHQDWLNGKEYKCKVSNKALPAPIEKTISKAKGQPREPQVYTLPPSRDELTK |
| | NQVSLTCLVKGFYPSDIAVEWESNGQPENNYKTTPPVLDSDGSFFLYSKLTVDKSRWQQGNVFSCSVMHEALHNHYTQ |
| | KSLSLSPGKGGGGSGGGGSCMEALGMESGEIHSDQITASSQYSTNWSAERSRLNYPENGWTPGEDSYREWIQVDLGLL |
| | RFVTAVGTQGAISKETKKKYYVKTYKIDVSSNGEDWITIKEGNKPVLFQGNTNPTDVVVAVFPKPLITRFVRIKPATW |
| | ETGISMRFEVYGCKITDYPCSGMLGMVSGLISDSQITSSNQGDRNWMPENIRLVTSRSGWALPPAPHSYINEWLQIDL |
| | GEEKIVRGIIIQGGKHRENKVFMRKFKIGYSNNGSDWKMIMDDSKRKAKSFEGNNNYDTPELRTFPALSTRFIRIYPE |
| | RATHGGLGLRMELLGCEVEA |
| 143 | EDFKCMEALGMESGEIHSDQITASSQYSTNWSAERSRLNYPENGWTPGADSYREWIQVDLGLLRFVTAVGTQGAISKE |
| | TKKKYYVKTYKIDVSSNGEDWITIKEGNKPVLFQGNTNPTDVVVAVFPKPLITRFVRIKPATWETGISMRFEVYGCKI |
| | TDYPCSGMLGMVSGLISDSQITSSNQGDRNWMPENIRLVTSRSGWALPPAPHSYINEWLQIDLGEEKIVRGIIIQGGK |
| | HRENKVFMRKFKIGYSNNGSDWKMIMDDSKRKAKSFEGNNNYDTPELRTFPALSTRFIRIYPERATHGGLGLRMELLG |
| | CGGGGSGGGGSEPKSCDKTHTCPPCPAPELLGGPSVFLFPPKPKDTLMISRTPEVTCVVVDVSHEDPEVKFNWYVDGV |
| | EVHNAKTKPREEQYNSTYRVVSVLAVLHQDWLNGKEYKCKVSNKALPAPIEKTISKAKGQPREPQVYTLPPSRDELTK |
| | NQVSLTCLVKGFYPSDIAVAWESNGQPENNYKTTPPVLDSDGSFFLYSKLTVDKSRWQQGNVFSCSVMHEALHAHYTQ |
| | KSLSLSPGKGGGGSGGGGSCMEALGMESGEIHSDQITASSQYSTNWSAERSRLNYPENGWTPGADSYREWIQVDLGLL |
| | RFVTAVGTQGAISKETKKKYYVKTYKIDVSSNGEDWITIKEGNKPVLFQGNTNPTDVVVAVFPKPLITRFVRIKPATW |
| | ETGISMRFEVYGCKITDYPCSGMLGMVSGLISDSQITSSNQGDRNWMPENIRLVTSRSGWALPPAPHSYINEWLQIDL |
| | GEEKIVRGIIIQGGKHRENKVFMRKFKIGYSNNGSDWKMIMDDSKRKAKSFEGNNNYDTPELRTFPALSTRFIRIYPE |
| | RATHGGLGLRMELLGCEVEA |
| 144 | ENFQCNVPLGMESGRIANEQISASSTYSDGRWTPQQSRLHGDDNGWTPNLDSNKEYLQVDLRFLTMLTAIATQGAISR |
| | ETQNGYYVKSYKLEVSTNGEDWMVYRHGKNHKVFQANNDATEVVLNKLHAPLLTRFVRIRPQTWHSGIALRLELFGCG |
| | GGGSGGGGSGGGGSGGGGSEPKSCDKTHTCPPCPAPELLGGPSVFLFPPKPKDTLMISRTPEVTCVVVDVSHEDPEVK |
| | FNWYVDGVEVHNAKTKPREEQYNSTYRVVSVLTVLHQDWLNGKEYKCKVSNKALPAPIEKTISKAKGQPREPQVYTLP |
| | PSRDELTKNQVSLTCLVKGFYPSDIAVEWESNGQPENNYKTTPPVLDSDGSFFLYSKLTVDKSRWQQGNVFSCSVMHE |
| | ALHNHYTQKSLSLSPGK |
| 145 | ENFQCNVPLGMESGRIANEQISASSTYSDGRWTPQQSRLHGDDNGWTPNLDSNKEYLQVDLRFLTMLTAIATQGAISR |
| | ETQNGYYVKSYKLEVSTNGEDWMVYRHGKNHKVFQANNDATEVVLNKLHAPLLTRFVRIRPQTWHSGIALRLELFGCG |
| | GGGSGGGGSGGGGSGGGGSCNVPLGMESGRIANEQISASSTYSDGRWTPQQSRLHGDDNGWTPNLDSNKEYLQVDLRF |
| | LTMLTAIATQGAISRETQNGYYVKSYKLEVSTNGEDWMVYRHGKNHKVFQANNDATEVVLNKLHAPLLTRFVRIRPQT |
| | WHSGIALRLELFGCGGGGSGGGGSGGGGSGGGGSEPKSCDKTHTCPPCPAPELLGGPSVFLFPPKPKDTLMISRTPEV |
| | TCVVVDVSHEDPEVKFNWYVDGVEVHNAKTKPREEQYNSTYRVVSVLTVLHQDWLNGKEYKCKVSNKALPAPIEKTIS |
| | KAKGQPREPQVYTLPPSRDELTKNQVSLTCLVKGFYPSDIAVEWESNGQPENNYKTTPPVLDSDGSFFLYSKLTVDKS |
| | RWQQGNVFSCSVMHEALHNHYTQKSLSLSPGK |
| 146 | ENFQCNVPLGMESGRIANEQISASSTYSDGRWTPQQSRLHGDDNGWTPNLDSNKEYLQVDLRFLTMLTAIATQGAISR |
| | ETQNGYYVKSYKLEVSTNGEDWMVYRHGKNHKVFQANNDATEVVLNKLHAPLLTRFVRIRPQTWHSGIALRLELFGCG |
| | GGGSGGGGSGGGGSGGGGSCNVPLGMESGRIANEQISASSTYSDGRWTPQQSRLHGDDNGWTPNLDSNKEYLQVDLRF |
| | LTMLTAIATQGAISRETQNGYYVKSYKLEVSTNGEDWMVYRHGKNHKVFQANNDATEVVLNKLHAPLLTRFVRIRPQT |
| | WHSGIALRLELFGCGGGGSGGGGSGGGGSGGGGSCNVPLGMESGRIANEQISASSTYSDGRWTPQQSRLHGDDNGWTP |
| | NLDSNKEYLQVDLRFLTMLTAIATQGAISRETQNGYYVKSYKLEVSTNGEDWMVYRHGKNHKVFQANNDATEVVLNKL |
| | HAPLLTREVRIRPQTWHSGIALRLELFGCGGGGSGGGGSGGGGSGGGGSEPKSCDKTHTCPPCPAPELLGGPSVFLFP |
| | PKPKDTLMISRTPEVTCVVVDVSHEDPEVKFNWYVDGVEVHNAKTKPREEQYNSTYRVVSVLTVLHQDWLNGKEYKCK |
| | VSNKALPAPIEKTISKAKGQPREPQVYTLPPSRDELTKNQVSLTCLVKGFYPSDIAVEWESNGQPENNYKTTPPVLDS |
| | DGSFFLYSKLTVDKSRWQQGNVFSCSVMHEALHNHYTQKSLSLSPGK |
| 147 | ENFQCNVPLGMESGRIANEQISASSTYSDGRWTPQQSRLHGDDNGWTPNLDSNKEYLQVDLRFLTMLTAIATQGAISR |
| | ETQNGYYVKSYKLEVSTNGEDWMVYEHGKNHKVFQANNDATEVVLNKLHAPLLTRFVRIRPQTWHSGIALRLELFGCG |
| | GGGSGGGGSGGGGSGGGGSEPKSCDKTHTCPPCPAPELLGGPSVFLFPPKPKDTLMISRTPEVTCVVVDVSHEDPEVK |
| | FNWYVDGVEVHNAKTKPREEQYNSTYRVVSVLTVLHQDWLNGKEYKCKVSNKALPAPIEKTISKAKGQPREPQVYTLP |
| | PSRDELTKNQVSLTCLVKGFYPSDIAVEWESNGQPENNYKTTPPVLDSDGSFFLYSKLTVDKSRWQQGNVFSCSVMHE |
| | ALHNHYTQKSLSLSPGK |
| 148 | ENFQCNVPLGMESGRIANEQISASSTYSDGRWTPQQSRLHGDDNGWTPNLDSNKEYLQVDLRFLTMLTAIATQGAISR |
| | ETQNGYYVKSYKLEVSTNGEDWMVYEHGKNHKVFQANNDATEVVLNKLHAPLLTRFVRIRPQTWHSGIALRLELFGCG |
| | GGGSGGGGSGGGGSGGGGSCNVPLGMESGRIANEQISASSTYSDGRWTPQQSRLHGDDNGWTPNLDSNKEYLQVDLRF |
| | LTMLTAIATQGAISRETQNGYYVKSYKLEVSTNGEDWMVYEHGKNHKVFQANNDATEVVLNKLHAPLLTREVRIRPQT |
| | WHSGIALRLELFGCGGGGSGGGGSGGGGSGGGGSEPKSCDKTHTCPPCPAPELLGGPSVFLFPPKPKDTLMISRTPEV |
| | TCVVVDVSHEDPEVKFNWYVDGVEVHNAKTKPREEQYNSTYRVVSVLTVLHQDWLNGKEYKCKVSNKALPAPIEKTIS |
| | KAKGQPREPQVYTLPPSRDELTKNQVSLTCLVKGFYPSDIAVEWESNGQPENNYKTTPPVLDSDGSFFLYSKLTVDKS |
| | RWQQGNVFSCSVMHEALHNHYTQKSLSLSPGK |
| 149 | ENFQCNVPLGMESGRIANEQISASSTYSDGRWTPQQSRLHGDDNGWTPNLDSNKEYLQVDLRFLTMLTAIATQGAISR |
| | ETQNGYYVKSYKLEVSTNGEDWMVYEHGKNHKVFQANNDATEVVLNKLHAPLLTRFVRIRPQTWHSGIALRLELFGCG |
| | GGGSGGGGSGGGGSGGGGSCNVPLGMESGRIANEQISASSTYSDGRWTPQQSRLHGDDNGWTPNLDSNKEYLQVDLRF |
| | LTMLTAIATQGAISRETQNGYYVKSYKLEVSTNGEDWMVYEHGKNHKVFQANNDATEVVLNKLHAPLLTRFVRIRPQT |
| | WHSGIALRLELFGCGGGGSGGGGSGGGGSGGGGSCNVPLGMESGRIANEQISASSTYSDGRWTPQQSRLHGDDNGWTP |
| | NLDSNKEYLQVDLRFLTMLTAIATQGAISRETQNGYYVKSYKLEVSTNGEDWMVYEHGKNHKVFQANNDATEVVLNKL |
| | HAPLLTREVRIRPQTWHSGIALRLELFGCGGGGSGGGGSGGGGSGGGGSEPKSCDKTHTCPPCPAPELLGGPSVFLFP |
| | PKPKDTLMISRTPEVTCVVVDVSHEDPEVKFNWYVDGVEVHNAKTKPREEQYNSTYRVVSVLTVLHQDWLNGKEYKCK |
| | VSNKALPAPIEKTISKAKGQPREPQVYTLPPSRDELTKNQVSLTCLVKGFYPSDIAVEWESNGQPENNYKTTPPVLDS |
| | DGSFFLYSKLTVDKSRWQQGNVFSCSVMHEALHNHYTQKSLSLSPGK |
| 150 | ENFQCNVPLGMESGRIANEQISASSTASDGRWTPQQSRLHGDDNGWTPNLDSNKEYLQVDLRFLTMLTAIATQGAIAR |
| | ETQNGAYVKSYKLEVSTNGEDWMVYEHGKNHKVFQANNDATEVVLNKLHAPLLTRFVRIRPQTWHSGIALRLELFGCG |
| | GGGSGGGGSGGGGSGGGGSEPKSCDKTHTCPPCPAPELLGGPSVFLFPPKPKDTLMISRTPEVTCVVVDVSHEDPEVK |
| | FNWYVDGVEVHNAKTKPREEQYNSTYRVVSVLTVLHQDWLNGKEYKCKVSNKALPAPIEKTISKAKGQPREPQVYTLP |
| | PSRDELTKNQVSLTCLVKGFYPSDIAVEWESNGQPENNYKTTPPVLDSDGSFFLYSKLTVDKSRWQQGNVFSCSVMHE |
| | ALHNHYTQKSLSLSPGK |
| 151 | ENFQCNVPLGMESGRIANEQISASSTASDGRWTPQQSRLHGDDNGWTPNLDSNKEYLQVDLRFLTMLTAIATQGAIAR |
| | ETQNGAYVKSYKLEVSTNGEDWMVYEHGKNHKVFQANNDATEVVLNKLHAPLLTRFVRIRPQTWHSGIALRLELFGCG |
| | GGGSGGGGSGGGGSGGGGSCNVPLGMESGRIANEQISASSTASDGRWTPQQSRLHGDDNGWTPNLDSNKEYLQVDLRF |
| | LTMLTAIATQGAIARETQNGAYVKSYKLEVSTNGEDWMVYEHGKNHKVFQANNDATEVVLNKLHAPLLTRFVRIRPQT |
| | WHSGIALRLELFGCGGGGSGGGGSGGGGSGGGGSCNVPLGMESGRIANEQISASSTASDGRWTPQQSRLHGDDNGWTP |
| | NLDSNKEYLQVDLRFLTMLTAIATQGAIARETQNGAYVKSYKLEVSTNGEDWMVYEHGKNHKVFQANNDATEVVLNKL |
| | HAPLLTREVRIRPQTWHSGIALRLELFGCGGGGSGGGGSGGGGSGGGGSEPKSCDKTHTCPPCPAPELLGGPSVFLFP |
| | PKPKDTLMISRTPEVTCVVVDVSHEDPEVKFNWYVDGVEVHNAKTKPREEQYNSTYRVVSVLTVLHQDWLNGKEYKCK |
| | VSNKALPAPIEKTISKAKGQPREPQVYTLPPSRDELTKNQVSLTCLVKGFYPSDIAVEWESNGQPENNYKTTPPVLDS |
| | DGSFFLYSKLTVDKSRWQQGNVFSCSVMHEALHNHYTQKSLSLSPGK |
| 152 | ENFQCNVPLGMESGRIANEQISASSTYSDGRWTPQQSRLHGDDNGWTPNLDSNKEYLQVDLRFLTMLTAIATQGAISR |
| | ETQNGYYVKSYELEVSTNGEDWMVYEHGKNHKVFQANNDATEVVLNKLHAPLLTRFVRIRPQTWHSGIALRLELFGCG |
| | GGGSGGGGSGGGGSGGGGSEPKSCDKTHTCPPCPAPELLGGPSVFLFPPKPKDTLMISRTPEVTCVVVDVSHEDPEVK |
| | FNWYVDGVEVHNAKTKPREEQYNSTYRVVSVLTVLHQDWLNGKEYKCKVSNKALPAPIEKTISKAKGQPREPQVYTLP |
| | PSRDELTKNQVSLTCLVKGFYPSDIAVEWESNGQPENNYKTTPPVLDSDGSFFLYSKLTVDKSRWQQGNVFSCSVMHE |
| | ALHNHYTQKSLSLSPGK |
| 153 | ENFQCNVPLGMESGRIANEQISASSTYSDGRWTPQQSRLHGDDNGWTPNLDSNKEYLQVDLRFLTMLTAIATQGAISR |
| | ETQNGYYVKSYELEVSTNGEDWMVYEHGKNHKVFQANNDATEVVLNKLHAPLLTRFVRIRPQTWHSGIALRLELFGCG |
| | GGGSGGGGSGGGGSGGGGSCNVPLGMESGRIANEQISASSTYSDGRWTPQQSRLHGDDNGWTPNLDSNKEYLQVDLRF |
| | LTMLTAIATQGAISRETQNGYYVKSYELEVSTNGEDWMVYEHGKNHKVFQANNDATEVVLNKLHAPLLTRFVRIRPQT |
| | WHSGIALRLELFGCGGGGSGGGGSGGGGSGGGGSCNVPLGMESGRIANEQISASSTYSDGRWTPQQSRLHGDDNGWTP |
| | NLDSNKEYLQVDLRFLTMLTAIATQGAISRETQNGYYVKSYELEVSTNGEDWMVYEHGKNHKVFQANNDATEVVLNKL |
| | HAPLLTREVRIRPQTWHSGIALRLELFGCGGGGSGGGGSGGGGSGGGGSEPKSCDKTHTCPPCPAPELLGGPSVFLFP |
| | PKPKDTLMISRTPEVTCVVVDVSHEDPEVKFNWYVDGVEVHNAKTKPREEQYNSTYRVVSVLTVLHQDWLNGKEYKCK |
| | VSNKALPAPIEKTISKAKGQPREPQVYTLPPSRDELTKNQVSLTCLVKGFYPSDIAVEWESNGQPENNYKTTPPVLDS |
| | DGSFFLYSKLTVDKSRWQQGNVFSCSVMHEALHNHYTQKSLSLSPGK |
| 154 | ENFQCNVPLGMESGRIANEQISASSTYSDGRWTPQQSRLHGDDNGWTPNLDSNKEYLQVDLRFLTMLTAIATQGAISR |
| | ETQNGYYVKSYKLEVSTNGEDWMVYRHGKNHKVFQANNDATEVVLNKLHAPLLTRFVRIRPQTWHSGIALRLELFGCR |
| | VTDAPCSNMLGMLSGLIADSQISASSTQEYLWSPSAARLVSSRSGWFPRIPQAQPGEEWLQVDLGTPKTVKGVIIQGA |
| | RGGDSITAVEARAFVRKFKVSYSLNGKDWEYIQDPRTQQPKLFEGNMHYDTPDIRRFDPIPAQYVRVYPERWSPAGIG |
| | MRLEVLGCDWTDGAPAPAGAPAPATCPPCPAPEFLGGPSVFLFPPKPKDTLMISRTPEVTCVVVDVSQEDPEVQFNWY |
| | VDGVEVHNAKTKPREEQFNSTYRVVSVLTVLHQDWLNGKEYKCKVSNKGLPSSIEKTISKAKGQPREPQVYTLPPSQE |
| | EMTENQVSLTCLVKGFYPSDIAVEWESNGQPENNYKTTPPVLDSDGSFFLYSWLTVDKSRWQEGNVFSCSVMHEALHN |
| | HYTQKSLSLSLGK |
| 155 | ENFQCNVPLGMESGRIANEQISASSTYSDGRWTPQQSRLHGDDNGWTPNLDSNKEYLQVDLRFLTMLTAIATQGAISR |
| | ETQNGYYVKSYKLEVSTNGEDWMVYRHGKNHKVFQANNDATEVVLNKLHAPLLTRFVRIRPQTWHSGIALRLELFGCR |
| | VTDAPCSNMLGMLSGLIADSQISASSTQEYLWSPSAARLVSSRSGWFPRIPQAQPGEEWLQVDLGTPKTVKGVIIQGA |
| | RGGDSITAVEARAFVAAFAVSYSLNGKDWEYIQDPRTQQPKLFEGNMHYDTPDIRRFDPIPAQYVRVYPERWSPAGIG |
| | MRLEVLGCDWTDGAPAPAGAPAPATCPPCPAPELLGGPSVFLFPPKPKDTLMISRTPEVTCVVVDVSHEDPEVKFNWY |
| | VDGVEVHNAKTKPREEQYNSTYRVVSVLTVLHQDWLNGKEYKCKVSNKALPAPIEKTISKAKGQPREPQVYTLPPSRD |
| | ELTKNQVSLTCLVKGFYPSDIAVEWESNGQPENNYKTTPPVLDSDGSFFLYSKLTVDKSRWQQGNVFSCSVMHEALHN |
| | HYTQKSLSLSPGK |
| 156 | ENFQCNVPLGMESGRIANEQISASSTYSDGRWTPQQSRLHGDDNGWTPNLDSNKEYLQVDLRFLTMLTAIATQGAISR |
| | ETQNGYYVKSYKLEVSTNGEDWMVYRHGKNHKVFQANNDATEVVLNKLHAPLLTRFVRIRPQTWHSGIALRLELFGCR |
| | VTDAPCSNMLGMLSGLIADSQISASSTQEYLWSPSAARLVSSRSGWFPRIPQAQPGEEWLQVDLGTPKTVKGVIIQGA |
| | RGGDSITAVEARAFVEEFEVSYSLNGKDWEYIQDPRTQQPKLFEGNMHYDTPDIRRFDPIPAQYVRVYPERWSPAGIG |
| | MRLEVLGCDWTDGAPAPAGAPAPATCPPCPAPELLGGPSVFLFPPKPKDTLMISRTPEVTCVVVDVSHEDPEVKFNWY |
| | VDGVEVHNAKTKPREEQYNSTYRVVSVLTVLHQDWLNGKEYKCKVSNKALPAPIEKTISKAKGQPREPQVYTLPPSRD |
| | ELTKNQVSLTCLVKGFYPSDIAVEWESNGQPENNYKTTPPVLDSDGSFFLYSKLTVDKSRWQQGNVFSCSVMHEALHN |
| | HYTQKSLSLSPGK |
| 157 | TCPPCPAPEFLGGPSVFLFPPKPKDTLMISRTPEVTCVVVDVSQEDPEVQFNWYVDGVEVHNAKTKPREEQFNSTYRV |
| | VSVLTVLHQDWLNGKEYKCKVSNKGLPSSIEKTISKAKGQPREPQVYTLPPSQEEMTENQVSLTCLVKGFYPSDIAVE |
| | WESNGQPENNYKTTPPVLDSDGSFFLYSWLTVDKSRWQEGNVFSCSVMHEALHNHYTQKSLSLSLGKGAPAPAGAPAP |
| | AENFQCNVPLGMESGRIANEQISASSTYSDGRWTPQQSRLHGDDNGWTPNLDSNKEYLQVDLRFLTMLTAIATQGAIS |
| | RETQNGYYVKSYKLEVSTNGEDWMVYRHGKNHKVFQANNDATEVVLNKLHAPLLTRFVRIRPQTWHSGIALRLELFGC |
| | RVTDAPCSNMLGMLSGLIADSQISASSTQEYLWSPSAARLVSSRSGWFPRIPQAQPGEEWLQVDLGTPKTVKGVIIQG |
| | ARGGDSITAVEARAFVRKFKVSYSLNGKDWEYIQDPRTQQPKLFEGNMHYDTPDIRRFDPIPAQYVRVYPERWSPAGI |
| | GMRLEVLGCDWTD |
| 158 | TCPPCPAPEFLGGPSVFLFPPKPKDTLMISRTPEVTCVVVDVSQEDPEVQFNWYVDGVEVHNAKTKPREEQFNSTYRV |
| | VSVLTVLHQDWLNGKEYKCKVSNKGLPSSIEKTISKAKGQPREPQVYTLPPSQEEMTENQVSLTCLVKGFYPSDIAVE |
| | WESNGQPENNYKTTPPVLDSDGSFFLYSWLTVDKSRWQEGNVFSCSVMHEALHNHYTQKSLSLSLGKGAPAPAGAPAP |
| | ACNVPLGMESGRIANEQISASSTASDGRWTPQQSRLHGDDNGWAPNLASNKEYLQVDLRFLTMLTAIATQGAISRETQ |
| | NGYYVKSYKLEVSTNGEDWMVYRHGKNHKVFQANNDATEVVLNKLHAPLLTRFVRIRPQTWHSGIALRLELFGCRVTD |
| | APCSNMLGMLSGLIADSQISASSTQEYLWSPSAARLVSSRSGWFPRIPQAQPGEEWLQVDLGTPKTVKGVIIQGARGG |
| | DSITAVEARAFVEEFEVSYSLNGKDWEYIQDPRTQQPKLFEGNMHYDTPDIRRFDPIPAQYVRVYPERWSPAGIGMRL |
| | EVLGCDWTD |
| 159 | TCPPCPAPEFLGGPSVFLFPPKPKDTLMISRTPEVTCVVVDVSQEDPEVQFNWYVDGVEVHNAKTKPREEQFNSTYRV |
| | VSVLTVLHQDWLNGKEYKCKVSNKGLPSSIEKTISKAKGQPREPQVYTLPPSQEEMTENQVSLTCLVKGFYPSDIAVE |
| | WESNGQPENNYKTTPPVLDSDGSFFLYSWLTVDKSRWQEGNVFSCSVMHEALHNHYTQKSLSLSLGKGAPAPAGAPAP |
| | ACNVPLGMESGRIANEQISASSTASDGRWTPQQSRLHGDDNGWAPNLASNKEYLQVDLRFLTMLTAIATQGAISRETQ |
| | NGYYVKSYKLEVSTNGEDWMVYRHGKNHKVFQANNDATEVVLNKLHAPLLTRFVRIRPQTWHSGIALRLELFGCRVTD |
| | APCSNMLGMLSGLIADSQISASSTQEYLWSPSAARLVSSRSGWFPRIPQAQPGEEWLQVDLGTPKTVKGVIIQGARGG |
| | DSITAVEARAFVAAFAVSYSLNGKDWEYIQDPRTQQPKLFEGNMHYDTPDIRRFDPIPAQYVRVYPERWSPAGIGMRL |
| | EVLGCDWTD |
| 160 | ENFQCNVPLGMESGRIANEQISASSTYSDGRWTPQQSRLHGDDNGWTPNLDSNKEYLQVDLRFLTMLTAIATQGAISR |
| | ETQNGYYVKSYKLEVSTNGEDWMVYRHGKNHKVFQANNDATEVVLNKLHAPLLTRFVRIRPQTWHSGIALRLELFGCG |
| | GGGSGGGGSGGGGSGGGGSEPKSCDKTHTCPPCPAPELLGGPSVFLFPPKPKDTLMISRTPEVTCVVVDVSHEDPEVK |
| | FNWYVDGVEVHNAKTKPREEQYNSTYRVVSVLTVLHQDWLNGKEYKCKVSNKALPAPIEKTISKAKGQPREPQVYTLP |
| | PSRDELTKNQVSLTCLVKGFYPSDIAVEWESNGQPENNYKTTPPVLDSDGSFFLYSKLTVDKSRWQQGNVFSCSVMHE |
| | ALHNHYTQKSLSLSPGKGGGGSGGGGSGGGGSGGGGSCNVPLGMESGRIANEQISASSTYSDGRWTPQQSRLHGDDNG |
| | WTPNLDSNKEYLQVDLRFLTMLTAIATQGAISRETQNGYYVKSYKLEVSTNGEDWMVYRHGKNHKVFQANNDATEVVL |
| | NKLHAPLLTRFVRIRPQTWHSGIALRLELFGCDWTD |
| 161 | ENFQCNVPLGMESGRIANEQISASSTYSDGRWTPQQSRLHGDDNGWTPNLDSNKEYLQVDLRFLTMLTAIATQGAISR |
| | ETQNGYYVKSYKLEVSTNGEDWMVYRHGKNHKVFQANNDATEVVLNKLHAPLLTRFVRIRPQTWHSGIALRLELFGCG |
| | GGGSGGGGSGGGGSGGGGSCNVPLGMESGRIANEQISASSTYSDGRWTPQQSRLHGDDNGWTPNLDSNKEYLQVDLRF |
| | LTMLTAIATQGAISRETQNGYYVKSYKLEVSTNGEDWMVYRHGKNHKVFQANNDATEVVLNKLHAPLLTRFVRIRPQT |
| | WHSGIALRLELFGCGGGGSGGGGSGGGGSGGGGSEPKSCDKTHTCPPCPAPELLGGPSVFLFPPKPKDTLMISRTPEV |
| | TCVVVDVSHEDPEVKFNWYVDGVEVHNAKTKPREEQYNSTYRVVSVLTVLHQDWLNGKEYKCKVSNKALPAPIEKTIS |
| | KAKGQPREPQVYTLPPSRDELTKNQVSLTCLVKGFYPSDIAVEWESNGQPENNYKTTPPVLDSDGSFFLYSKLTVDKS |
| | RWQQGNVFSCSVMHEALHNHYTQKSLSLSPGKGGGGSGGGGSGGGGSGGGGSCNVPLGMESGRIANEQISASSTYSDG |
| | RWTPQQSRLHGDDNGWTPNLDSNKEYLQVDLRFLTMLTAIATQGAISRETQNGYYVKSYKLEVSTNGEDWMVYRHGKN |
| | HKVFQANNDATEVVLNKLHAPLLTRFVRIRPQTWHSGIALRLELFGCDWTD |
| 162 | ENFQCNVPLGMESGRIANEQISASSTYSDGRWTPQQSRLHGDDNGWTPNLDSNKEYLQVDLRFLTMLTAIATQGAISR |
| | ETQNGYYVKSYKLEVSTNGEDWMVYEHGKNHKVFQANNDATEVVLNKLHAPLLTRFVRIRPQTWHSGIALRLELFGCG |
| | GGGSGGGGSGGGGSGGGGSEPKSCDKTHTCPPCPAPELLGGPSVFLFPPKPKDTLMISRTPEVTCVVVDVSHEDPEVK |
| | FNWYVDGVEVHNAKTKPREEQYNSTYRVVSVLTVLHQDWLNGKEYKCKVSNKALPAPIEKTISKAKGQPREPQVYTLP |
| | PSRDELTKNQVSLTCLVKGFYPSDIAVEWESNGQPENNYKTTPPVLDSDGSFFLYSKLTVDKSRWQQGNVFSCSVMHE |
| | ALHNHYTQKSLSLSPGKGGGGSGGGGSGGGGSGGGGSCNVPLGMESGRIANEQISASSTYSDGRWTPQQSRLHGDDNG |
| | WTPNLDSNKEYLQVDLRFLTMLTAIATQGAISRETQNGYYVKSYKLEVSTNGEDWMVYEHGKNHKVFQANNDATEVVL |
| | NKLHAPLLTREVRIRPQTWHSGIALRLELFGCDWTD |
| 163 | ENFQCNVPLGMESGRIANEQISASSTYSDGRWTPQQSRLHGDDNGWTPNLDSNKEYLQVDLRFLTMLTAIATQGAISR |
| | ETQNGYYVKSYKLEVSTNGEDWMVYEHGKNHKVFQANNDATEVVLNKLHAPLLTRFVRIRPQTWHSGIALRLELFGCG |
| | GGGSGGGGSGGGGSGGGGSCNVPLGMESGRIANEQISASSTYSDGRWTPQQSRLHGDDNGWTPNLDSNKEYLQVDLRF |
| | LTMLTAIATQGAISRETQNGYYVKSYKLEVSTNGEDWMVYEHGKNHKVFQANNDATEVVLNKLHAPLLTRFVRIRPQT |
| | WHSGIALRLELFGCGGGGSGGGGSGGGGSGGGGSEPKSCDKTHTCPPCPAPELLGGPSVFLFPPKPKDTLMISRTPEV |
| | TCVVVDVSHEDPEVKFNWYVDGVEVHNAKTKPREEQYNSTYRVVSVLTVLHQDWLNGKEYKCKVSNKALPAPIEKTIS |
| | KAKGQPREPQVYTLPPSRDELTKNQVSLTCLVKGFYPSDIAVEWESNGQPENNYKTTPPVLDSDGSFFLYSKLTVDKS |
| | RWQQGNVFSCSVMHEALHNHYTQKSLSLSPGKGGGGSGGGGSGGGGSGGGGSCNVPLGMESGRIANEQISASSTYSDG |
| | RWTPQQSRLHGDDNGWTPNLDSNKEYLQVDLRFLTMLTAIATQGAISRETQNGYYVKSYKLEVSTNGEDWMVYEHGKN |
| | HKVFQANNDATEVVLNKLHAPLLTRFVRIRPQTWHSGIALRLELFGCDWTD |
| 164 | EDFKCMEALGMESGEIHSDQITASSQYSTNWSAERSRLNYPENGWTPGEDSYREWIQVDLGLLRFVTAVGTQGAISKE |
| | TKKKYYVKTYKIDVSSNGEDWITIKEGNKPVLFQGNTNPTDVVVAVFPKPLITRFVRIKPATWETGISMRFEVYGCGA |
| | PAPAGAPAPACSNMLGMLSGLIADSQISASSTQEYLWSPSAARLVSSRSGWFPRIPQAQPGEEWLQVDLGTPKTVKGV |
| | IIQGARGGDSITAVEARAFVRKFKVSYSLNGKDWEYIQDPRTQQPKLFEGNMHYDTPDIRRFDPIPAQYVRVYPERWS |
| | PAGIGMRLEVLGCGAPAPAGAPAPAEPKSCDKTHTCPPCPAPELLGGPSVFLFPPKPKDTLMISRTPEVTCVVVDVSH |
| | EDPEVKFNWYVDGVEVHNAKTKPREEQYNSTYRVVSVLTVLHQDWLNGKEYKCKVSNKALPAPIEKTISKAKGQPREP |
| | QVYTLPPSRDELTKNQVSLTCLVKGFYPSDIAVEWESNGQPENNYKTTPPVLDSDGSFFLYSKLTVDKSRWQQGNVFS |
| | CSVMHEALHNHYTQKSLSLSPGK |
| 165 | ENFQCNVPLGMESGRIANEQISASSTYSDGRWTPQQSRLHGDDNGWTPNLDSNKEYLQVDLRFLTMLTAIATQGAISR |
| | ETQNGYYVKSYKLEVSTNGEDWMVYRHGKNHKVFQANNDATEVVLNKLHAPLLTRFVRIRPQTWHSGIALRLELFGCG |
| | APAPAGAPAPACSGMLGMVSGLISDSQITSSNQGDRNWMPENIRLVTSRSGWALPPAPHSYINEWLQIDLGEEKIVRG |
| | IIIQGGKHRENKVFMRKFKIGYSNNGSDWKMIMDDSKRKAKSFEGNNNYDTPELRTFPALSTRFIRIYPERATHGGLG |
| | LRMELLGCGAPAPAGAPAPAEPKSCDKTHTCPPCPAPELLGGPSVFLFPPKPKDTLMISRTPEVTCVVVDVSHEDPEV |
| | KFNWYVDGVEVHNAKTKPREEQYNSTYRVVSVLTVLHQDWLNGKEYKCKVSNKALPAPIEKTISKAKGQPREPQVYTL |
| | PPSRDELTKNQVSLTCLVKGFYPSDIAVEWESNGQPENNYKTTPPVLDSDGSFFLYSKLTVDKSRWQQGNVFSCSVMH |
| | EALHNHYTQKSLSLSPGK |
| 166 | EDFKCMEALGMESGEIHSDQITASSQASTNWSAERSRLNYPENGWTPGEDSYREWIQVDLGLLRFVTAVGTQGAIAKE |
| | TKKKAYVKTYKIDVSSNGEDWITIKEGNKPVLFQGNTNPTDVVVAVFPKPLITRFVRIKPATWETGISMRFEVYGCGA |
| | PAPAGAPAPACSNMLGMLSGLIADSQISASSTQEYLWSPSAARLVSSRSGWFPRIPQAQPGEEWLQVDLGTPKTVKGV |
| | IIQGARGGDSITAVEARAFVRKFKVSYSLNGKDWEYIQDPRTQQPKLFEGNMHYDTPDIRRFDPIPAQYVRVYPERWS |
| | PAGIGMRLEVLGCGAPAPAGAPAPAEPKSCDKTHTCPPCPAPELLGGPSVFLFPPKPKDTLMISRTPEVTCVVVDVSH |
| | EDPEVKFNWYVDGVEVHNAKTKPREEQYNSTYRVVSVLTVLHQDWLNGKEYKCKVSNKALPAPIEKTISKAKGQPREP |
| | QVYTLPPSRDELTKNQVSLTCLVKGFYPSDIAVEWESNGQPENNYKTTPPVLDSDGSFFLYSKLTVDKSRWQQGNVFS |
| | CSVMHEALHNHYTQKSLSLSPGK |
| 167 | FRNDKCGDTIKIESPGYLTSPGYPHSYHPSEKCEWLIQAPDPYQRIMINFNPHFDLEDRDCKYDYVEVFDGENENGHF |
| | RGKFCGKIAPPPVVSSGPFLFIKFVSDYETHGAGFSIRYEIFKRGPECSQNYTTPSGVIKSPGFPEKYPNSLECTYIV |
| | FVPKMSEIILEFESFDLEPDSNPPGGMFCRYDRLEIWDGFPDVGPHIGRYCGQKTPGRIRSSSGILSMVFYTDSAIAK |
| | EGFSANYSVLQSSVSEDFKCMEALGMESGEIHSDQITASSQYSTNWSAERSRLNYPENGWTPGEDSYREWIQVDLGLL |
| | RFVTAVGTQGAISKETKKKYYVKTYKIDVSSNGEDWITIKEGNKPVLFQGNTNPTDVVVAVFPKPLITRFVRIKPATW |
| | ETGISMRFEVYGCKITDYPCSGMLGMVSGLISDSQITSSNQGDRNWMPENIRLVTSRSGWALPPAPHSYINEWLQIDL |
| | GEEKIVRGIIIQGGKHRENKVFMRKFKIGYSNNGSDWKMIMDDSKRKAKSFEGNNNYDTPELRTFPALSTRFIRIYPE |
| | RATHGGLGLRMELLGCEVEAECPPCPAPPVAGPSVFLFPPKPKDTLMISRTPEVTCVVVDVSHEDPEVQFNWYVDGVE |
| | VHNAKTKPREEQFNSTFRVVSVLTVVHQDWLNGKEYKCKVSNKGLPAPIEKTISKTKGQPREPQVYTLPPSREEMTKN |
| | QVSLTCLVKGFYPSDISVEWESNGQPENNYKTTPPMLDSDGSFFLYSKLTVDKSRWQQGNVFSCSVMHEALHNHYTQK |
| | SLSLSPGK |
| 168 | FRGKFCGKIAPPPVVSSGPFLFIKFVSDYETHGAGFSIRYEIFKRGPECSQNYTTPSGVIKSPGFPEKYPNSLECTYI |
| | VFVPKMSEIILEFESFDLEPDSNPPGGMFCRYDRLEIWDGFPDVGPHIGRYCGQKTPGRIRSSSGILSMVFYTDSAIA |
| | KEGFSANYSVLQSSVSEDFKCMEALGMESGEIHSDQITASSQYSTNWSAERSRLNYPENGWTPGEDSYREWIQVDLGL |
| | LRFVTAVGTQGAISKETKKKYYVKTYKIDVSSNGEDWITIKEGNKPVLFQGNTNPTDVVVAVFPKPLITRFVRIKPAT |
| | WETGISMRFEVYGCKITDYPEPRGPTIKPCPPCKCPAPNLLGGPSVFIFPPKIKDVLMISLSPIVTCVVVDVSEDDPD |
| | VQISWFVNNVEVHTAQTQTHREDYNSTLRVVSALPIQHQDWMSGKEFKCKVNNKDLPAPIERTISKPKGSVRAPQVYV |
| | LPPPEEEMTKKQVTLTCMVTDFMPEDIYVEWTNNGKTELNYKNTEPVLDSDGSYFMYSKLRVEKKNWVERNSYSCSVV |
| | HEGLHNHHTTKSFSRTPGK |
| 191 | EDFKCMEALGMESGEIHSDQITASSQYSTNWSAERSRLNYPENGWTPGEDSYREWIQVDLGLLRFVTAVGTQGAISKE |
| | TKKKYYVKTYKIDVSSNGEDWITIKEGNKPVLFQGNTNPTDVVVAVFPKPLITRFVRIKPATWETGISMRFEVYGCKI |
| | TDYPCSGMLGMVSGLISDSQITSSNQGDRNWMPENIRLVTSRSGWALPPAPHSYINEWLQIDLGEEKIVRGIIIQGGK |
| | HRENKVFMRKFKIGYSNNGSDWKMIMDDSKRKAKSFEGNNNYDTPELRTFPALSTRFIRIYPERATHGGLGLRMELLG |
| | CGGGGSGGGGSEPKSCDKTHTCPPCPAPELLGGPSVFLFPPKPKDTLMISRTPEVTCVVVDVSHEDPEVKFNWYVDGV |
| | EVHNAKTKPREEQYNSTYRVVSVLAVLHQDWLNGKEYKCKVSNKALPAPIEKTISKAKGQPREPQVYTLPPSRDELTK |
| | NQVSLTCLVKGFYPSDIAVAWESNGQPENNYKTTPPVLDSDGSFFLYSKLTVDKSRWQQGNVFSCSVMHEALHAHYTQ |
| | KSLSLSPGK |
| 192 | EDFKCMEALGMESGEIHSDQITASSQYSTNWSAERSRLNYPENGWTPGEDSYREWIQVDLGLLRFVTAVGTQGAISKE |
| | TKKKYYVKTYKIDVSSNGEDWITIKEGNKPVLFQGNTNPTDVVVAVFPKPLITRFVRIKPATWETGISMRFEVYGCKI |
| | TDYPCSGMLGMVSGLISDSQITSSNQGDRNWMPENIRLVTSRSGWALPPAPHSYINEWLQIDLGEEKIVRGIIIQGGK |
| | HRENKVFMRKFKIGYSNNGSDWKMIMDDSKRKAKSFEGNNNYDTPELRTFPALSTRFIRIYPERATHGGLGLRMELLG |
| | CGGGGSGGGGSEPKSCDKTHTCPPCPAPELLGGPSVFLFPPKPKDTLMISRTPEVTCVVVDVSHEDPEVKFNWYVDGV |
| | EVHNAKTKPREEQYNSTYRVVSVLTVLHQDWLNGKEYKCKVSNKALPAPIEKTISKAKGQPREPQVYTLPPSRDELTK |
| | NQVSLTCLVKGFYPSDIAVEWESNGQPENNYKTTPPVLDSDGSFFLYSKLTVDKSRWQQGNVFSCSVMHEALHNHYTQ |
| | KSLSLSPGKGGGGSGGGGSGGGGSEEQAKTFLDKFNHEAEDLFYQSSGLGKGDFR |
| 193 | EDFKCMEALGMESGEIHSDQITASSQASTNWSAERSRLNYPENGWTPGEDSYREWIQVDLGLLRFVTAVGTQGAIAKE |
| | TKKKAYVKTYKIDVSSNGEDWITIKEGNKPVLFQGNTNPTDVVVAVFPKPLITRFVRIKPATWETGISMRFEVYGCKI |
| | TDYPCSGMLGMVSGLISDSQITSSNQGDRNWMPENIRLVTSRSGWALPPAPHSYINEWLQIDLGEEKIVRGIIIQGGK |
| | HRENKVFMRKFKIGYSNNGSDWKMIMDDSKRKAKSFEGNNNYDTPELRTFPALSTRFIRIYPERATHGGLGLRMELLG |
| | CGGGGSGGGGSEPKSCDKTHTCPPCPAPELLGGPSVFLFPPKPKDTLMISRTPEVTCVVVDVSHEDPEVKFNWYVDGV |
| | EVHNAKTKPREEQYNSTYRVVSVLTVLHQDWLNGKEYKCKVSNKALPAPIEKTISKAKGQPREPQVYTLPPSRDELTK |
| | NQVSLTCLVKGFYPSDIAVEWESNGQPENNYKTTPPVLDSDGSFFLYSKLTVDKSRWQQGNVFSCSVMHEALHNHYTQ |
| | KSLSLSPGK |
| 194 | ENFQCNVPLGMESGRIANEQISASSTYSDGRWTPQQSRLHGDDNGWTPNLDSNKEYLQVDLRFLTMLTAIATQGAISR |
| | ETQNGYYVKSYKLEVSTNGEDWMVYRHGKNHKVFQANNDATEVVLNKLHAPLLTRFVRIRPQTWHSGIALRLELFGCR |
| | VTDAPCSNMLGMLSGLIADSQISASSTQEYLWSPSAARLVSSRSGWFPRIPQAQPGEEWLQVDLGTPKTVKGVIIQGA |
| | RGGDSITAVEARAFVRKFKVSYSLNGKDWEYIQDPRTQQPKLFEGNMHYDTPDIRRFDPIPAQYVRVYPERWSPAGIG |
| | MRLEVLGCGAPAPAGAPAPAEPKSCDKTHTCPPCPAPELLGGPSVFLFPPKPKDTLMISRTPEVTCVVVDVSHEDPEV |
| | KFNWYVDGVEVHNAKTKPREEQYNSTYRVVSVLTVLHQDWLNGKEYKCKVSNKALPAPIEKTISKAKGQPREPQVYTL |
| | PPSRDELTKNQVSLTCLVKGFYPSDIAVEWESNGQPENNYKTTPPVLDSDGSFFLYSKLTVDKSRWQQGNVFSCSVMH |
| | EALHNHYTQKSLSLSPGK |
| 195 | EDFKCMEALGMESGEIHSDQITASSQYSTNWSAERSRLNYPENGWTPGEDSYREWIQVDLGLLRFVTAVGTQGAISKE |
| | TKKKYYVKTYKIDVSSNGEDWITIKEGNKPVLFQGNTNPTDVVVAVFPKPLITRFVRIKPATWETGISMRFEVYGCGG |
| | GGSGGGGSGGGGSGGGGSEPKSCDKTHTCPPCPAPELLGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALPAPIEKTISKAKGQPREPQVYTLPP |
| | SRDELTKNQVSLTCLVKGFYPSDIAVEWESNGQPENNYKTTPPVLDSDGSFFLYSKLTVDKSRWQQGNVFSCSVMHEA |
| | LHNHYTQKSLSLSPGK |
| 196 | EDFKCMEALGMESGEIHSDQITASSQYSTNWSAERSRLNYPENGWTPGEDSYREWIQVDLGLLRFVTAVGTQGAISKE |
| | TKKKYYVKTYKIDVSSNGEDWITIEEGNKPVLFQGNTNPTDVVVAVFPKPLITRFVRIKPATWETGISMRFEVYGCGA |
| | PAPAGAPAPAGAPAPAGAPAPACMEALGMESGEIHSDQITASSQYSTNWSAERSRLNYPENGWTPGEDSYREWIQVDL |
| | GLLRFVTAVGTQGAISKETKKKYYVKTYKIDVSSNGEDWITIEEGNKPVLFQGNTNPTDVVVAVFPKPLITRFVRIKP |
| | ATWETGISMRFEVYGCGAPAPAGAPAPAGAPAPAGAPAPAEPKSCDKTHTCPPCPAPELLGGPSVFLFPPKPKDTLMI |
| | SRTPEVTCVVVDVSHEDPEVKFNWYVDGVEVHNAKTKPREEQYNSTYRVVSVLTVLHQDWLNGKEYKCKVSNKALPAP |
| | IEKTISKAKGQPREPQVYTLPPSRDELTKNQVSLTCLVKGFYPSDIAVEWESNGQPENNYKTTPPVLDSDGSFFLYSK |
| | LTVDKSRWQQGNVFSCSVMHEALHNHYTQKSLSLSPGK |
| 197 | EDFKCMEALGMESGEIHSDQITASSQYSTNWSAERSRLNYPENGWTPGEDSYREWIQVDLGLLRFVTAVGTQGAISKE |
| | TKKKYYVKTYKIDVSSNGEDWITIEEGNKPVLFQGNTNPTDVVVAVFPKPLITRFVRIKPATWETGISMRFEVYGCGA |
| | PAPAGAPAPAGAPAPAGAPAPACMEALGMESGEIHSDQITASSQYSTNWSAERSRLNYPENGWTPGEDSYREWIQVDL |
| | GLLRFVTAVGTQGAISKETKKKYYVKTYKIDVSSNGEDWITIEEGNKPVLFQGNTNPTDVVVAVFPKPLITRFVRIKP |
| | ATWETGISMRFEVYGCGAPAPAGAPAPAGAPAPAGAPAPACMEALGMESGEIHSDQITASSQYSTNWSAERSRLNYPE |
| | NGWTPGEDSYREWIQVDLGLLRFVTAVGTQGAISKETKKKYYVKTYKIDVSSNGEDWITIEEGNKPVLFQGNTNPTDV |
| | VVAVFPKPLITRFVRIKPATWETGISMRFEVYGCGAPAPAGAPAPAGAPAPAGAPAPAEPKSCDKTHTCPPCPAPELL |
| | GGPSVFLFPPKPKDTLMISRTPEVTCVVVDVSHEDPEVKFNWYVDGVEVHNAKTKPREEQYNSTYRVVSVLTVLHQDW |
| | LNGKEYKCKVSNKALPAPIEKTISKAKGQPREPQVYTLPPSRDELTKNQVSLTCLVKGFYPSDIAVEWESNGQPENNY |
| | KTTPPVLDSDGSFFLYSKLTVDKSRWQQGNVFSCSVMHEALHNHYTQKSLSLSPGK |
| 198 | EDFKCMEALGMESGEIHSDQITASSQYSTNWSAERSRLNYPENGWTPGEDSYREWIQVDLGLLRFVTAVGTQGAISKE |
| | TKKKYYVKTYKIDVSSNGEDWITIEEGNKPVLFQGNTNPTDVVVAVFPKPLITRFVRIKPATWETGISMRFEVYGCGA |
| | PAPAGAPAPAGAPAPAGAPAPAEPKSCDKTHTCPPCPAPELLGGPSVFLFPPKPKDTLMISRTPEVTCVVVDVSHEDP |
| | EVKFNWYVDGVEVHNAKTKPREEQYNSTYRVVSVLTVLHQDWLNGKEYKCKVSNKALPAPIEKTISKAKGQPREPQVY |
| | TLPPSRDELTKNQVSLTCLVKGFYPSDIAVEWESNGQPENNYKTTPPVLDSDGSFFLYSKLTVDKSRWQQGNVFSCSV |
| | MHEALHNHYTQKSLSLSPGKGAPAPAGAPAPAGAPAPAGAPAPACMEALGMESGEIHSDQITASSQYSTNWSAERSRL |
| | NYPENGWTPGEDSYREWIQVDLGLLRFVTAVGTQGAISKETKKKYYVKTYKIDVSSNGEDWITIEEGNKPVLFQGNTN |
| | PTDVVVAVFPKPLITREVRIKPATWETGISMRFEVYGCEVEA |
| 199 | ENFQCNVPLGMESGRIANEQISASSTYSDGRWTPQQSRLHGDDNGWTPNLDSNKEYLQVDLRFLTMLTAIATQGAISR |
| | ETQNGYYVKSYKLEVSTNGEDWMVYEHGKNHKVFQANNDATEVVLNKLHAPLLTRFVRIRPQTWHSGIALRLELFGCG |
| | APAPAGAPAPAGAPAPAGAPAPACNVPLGMESGRIANEQISASSTYSDGRWTPQQSRLHGDDNGWTPNLDSNKEYLQV |
| | DLRFLTMLTAIATQGAISRETQNGYYVKSYKLEVSTNGEDWMVYEHGKNHKVFQANNDATEVVLNKLHAPLLTRFVRI |
| | RPQTWHSGIALRLELFGCGAPAPAGAPAPAGAPAPAGAPAPAEPKSCDKTHTCPPCPAPELLGGPSVFLFPPKPKDTL |
| | MISRTPEVTCVVVDVSHEDPEVKFNWYVDGVEVHNAKTKPREEQYNSTYRVVSVLTVLHQDWLNGKEYKCKVSNKALP |
| | APIEKTISKAKGQPREPQVYTLPPSRDELTKNQVSLTCLVKGFYPSDIAVEWESNGQPENNYKTTPPVLDSDGSFFLY |
| | SKLTVDKSRWQQGNVFSCSVMHEALHNHYTQKSLSLSPGK |
| 200 | ENFQCNVPLGMESGRIANEQISASSTYSDGRWTPQQSRLHGDDNGWTPNLDSNKEYLQVDLRFLTMLTAIATQGAISR |
| | ETQNGYYVKSYKLEVSTNGEDWMVYEHGKNHKVFQANNDATEVVLNKLHAPLLTRFVRIRPQTWHSGIALRLELFGCG |
| | APAPAGAPAPAGAPAPAGAPAPACNVPLGMESGRIANEQISASSTYSDGRWTPQQSRLHGDDNGWTPNLDSNKEYLQV |
| | DLRFLTMLTAIATQGAISRETQNGYYVKSYKLEVSTNGEDWMVYEHGKNHKVFQANNDATEVVLNKLHAPLLTRFVRI |
| | RPQTWHSGIALRLELFGCGAPAPAGAPAPAGAPAPAGAPAPACNVPLGMESGRIANEQISASSTYSDGRWTPQQSRLH |
| | GDDNGWTPNLDSNKEYLQVDLRFLTMLTAIATQGAISRETQNGYYVKSYKLEVSTNGEDWMVYEHGKNHKVFQANNDA |
| | TEVVLNKLHAPLLTREVRIRPQTWHSGIALRLELFGCGAPAPAGAPAPAGAPAPAGAPAPAEPKSCDKTHTCPPCPAP |
| | ELLGGPSVFLFPPKPKDTLMISRTPEVTCVVVDVSHEDPEVKFNWYVDGVEVHNAKTKPREEQYNSTYRVVSVLTVLH |
| | QDWLNGKEYKCKVSNKALPAPIEKTISKAKGQPREPQVYTLPPSRDELTKNQVSLTCLVKGFYPSDIAVEWESNGQPE |
| | NNYKTTPPVLDSDGSFFLYSKLTVDKSRWQQGNVFSCSVMHEALHNHYTQKSLSLSPGK |
| 201 | ENFQCNVPLGMESGRIANEQISASSTYSDGRWTPQQSRLHGDDNGWTPNLDSNKEYLQVDLRFLTMLTAIATQGAISRETQNGYY |
| | VKSYKLEVSTNGEDWMVYEHGKNHKVFQANNDATEVVLNKLHAPLLTRFVRIRPQTWHSGIALRLELFGCGAPAPAGAPAPAGAP |
| | APAGAPAPAEPKSCDKTHTCPPCPAPELLGGPSVFLFPPKPKDTLMISRTPEVTCVVVDVSHEDPEVKFNWYVDGVEVHNAKTKP |
| | REEQYNSTYRVVSVLTVLHQDWLNGKEYKCKVSNKALPAPIEKTISKAKGQPREPQVYTLPPSRDELTKNQVSLTCLVKGFYPSD |
| | IAVEWESNGQPENNYKTTPPVLDSDGSFFLYSKLTVDKSRWQQGNVFSCSVMHEALHNHYTQKSLSLSPGKGAPAPAGAPAPAGA |
| | PAPAGAPAPACNVPLGMESGRIANEQISASSTYSDGRWTPQQSRLHGDDNGWTPNLDSNKEYLQVDLRFLTMLTAIATQGAISRE |
| | TQNGYYVKSYKLEVSTNGEDWMVYEHGKNHKVFQANNDATEVVLNKLHAPLLTRFVRIRPQTWHSGIALRLELFGCDWTD |
| 202 | FRNDKCGDTIKIESPGYLTSPGYPHSYHPSEKCEWLIQAPDPYQRIMINFNPHFDLEDRDCKYDYVEVFDGENENGHFRGKFCGK |
| | IAPPPVVSSGPFLFIKFVSDYETHGAGFSIRYEIFKRGPECSQNYTTPSGVIKSPGFPEKYPNSLECTYIVFVPKMSEIILEFES |
| | FDLEPDSNPPGGMFCRYDRLEIWDGFPDVGPHIGRYCGQKTPGRIRSSSGILSMVFYTDSAIAKEGFSANYSVLQSSVSEDFKCM |
| | EALGMESGEIHSDQITASSQYSTNWSAERSRLNYPENGWTPGEDSYREWIQVDLGLLRFVTAVGTQGAISKETKKKYYVKTYKID |
| | VSSNGEDWITIKEGNKPVLFQGNTNPTDVVVAVFPKPLITRFVRIKPATWETGISMRFEVYGCKITDYPCSGMLGMVSGLISDSQ |
| | ITSSNQGDRNWMPENIRLVTSRSGWALPPAPHSYINEWLQIDLGEEKIVRGIIIQGGKHRENKVFMRKFKIGYSNNGSDWKMIMD |
| | DSKRKAKSFEGNNNYDTPELRTFPALSTRFIRIYPERATHGGLGLRMELLGCEVEAPTAGPTTPNGNLVDECDDDQANCHSGTGD |
| | DFQLTGGTTVLATEKPTVIDSTIQSEFPTYGFNCEFGWGSHKTFCHWEHDNHVQLKWSVLTSKTGPIQDHTGDGNFTYSQADENQ |
| | KGKVARLVSPVVYSQNSAHCMTFWYHMSGSHVGTLRVKLRYQKPEEYDQLVWMAIGHQGDHWKEGRVLLHKSLKLYQVIFEGEIG |
| | KGNLGGIAVDDISINNHISQEDCAKPADLDKKNPEIKIDETGSTPGYEGEGEGDKNISRKPGNVLKTLDPECPPCPAPPVAGPSV |
| | FLFPPKPKDTLMISRTPEVTCVVVDVSHEDPEVQFNWYVDGVEVHNAKTKPREEQFNSTFRVVSVLTVVHQDWLNGKEYKCKVSN |
| | KGLPAPIEKTISKTKGQPREPQVYTLPPSREEMTKNQVSLTCLVKGFYPSDISVEWESNGQPENNYKTTPPMLDSDGSFFLYSKL |
| | TVDKSRWQQGNVFSCSVMHEALHNHYTQKSLSLSPGK |
| 203 | EDFKCMEALGMESGEIHSDQITASSQYSTNWSAERSRLNYPENGWTPGEDSYREWIQVDLGLLRFVTAVGTQGAISKETKKKYYV |
| | KTYKIDVSSNGEDWITIKEGNKPVLFQGNTNPTDVVVAVFPKPLITRFVRIKPATWETGISMRFEVYGCKITDYPCSGMLGMVSG |
| | LISDSQITSSNQGDRNWMPENIRLVTSRSGWALPPAPHSYINEWLQIDLGEEKIVRGIIIQGGKHRENKVFMRKFKIGYSNNGSD |
| | WKMIMDDSKRKAKSFEGNNNYDTPELRTFPALSTRFIRIYPERATHGGLGLRMELLGCGGGGSGGGGSEPKSCDKTHTCPPCPAP |
| | ELLGGPSVFLFPPKPKDTLMISRTPEVTCVVVDVSHEDPEVKFNWYVDGVEVHNAKTKPREEQYASTYRVVSVLAVLHQDWLNGK |
| | EYKCKVSNKALPAPIEKTISKAKGQPREPQVYTLPPSRDELTKNQVSLTCLVKGFYPSDIAVAWESNGQPENNYKTTPPVLDSDG |
| | SFFLYSKLTVDKSRWQQGNVFSCSVMHEALHAHYTQKSLSLSPGKGGGGSGGGGSEEQAKTFLDKFNHEAEDLFYQSSGLGKGDF |
| | R |
| 204 | EDFKCMEALGMESGEIHSDQITASSQYSTNWSAERSRLNYPENGWTPGEDSYREWIQVDLGLLRFVTAVGTQGAISKETKKKYYV |
| | KTYEIDVSSNGEDWITIEEGNKPVLFQGNTNPTDVVVAVFPKPLITRFVRIKPATWETGISMRFEVYGCKITDYPCSGMLGMVSG |
| | LISDSQITSSNQGDRNWMPENIRLVTSRSGWALPPAPHSYINEWLQIDLGEEKIVRGIIIQGGKHRENKVFMEEFEIGYSNNGSD |
| | WKMIMDDSKRKAKSFEGNNNYDTPELRTFPALSTRFIRIYPERATHGGLGLRMELLGCGGGGSGGGGSEPKSCDKTHTCPPCPAP |
| | ELLGGPSVFLFPPKPKDTLMISRTPEVTCVVVDVSHEDPEVKFNWYVDGVEVHNAKTKPREEQYNSTYRVVSVLTVLHQDWLNGK |
| | EYKCKVSNKALPAPIEKTISKAKGQPREPQVYTLPPSRDELTKNQVSLTCLVKGFYPSDIAVEWESNGQPENNYKTTPPVLDSDG |
| | SFFLYSKLTVDKSRWQQGNVFSCSVMHEALHNHYTQKSLSLSPGKGGGGSGGGGSEEQAKTFLDKFNHEAEDLFYQSSGLGKGDF |
| | R |
| 205 | EDFKCMEALGMESGEIHSDQITASSQYSTNWSAERSRLNYPENGWTPGEDSYREWIQVDLGLLRFVTAVGTQGAISKETKKKYYV |
| | KTYKIDVSSNGEDWITIKEGNKPVLFQGNTNPTDVVVAVFPKPLITRFVRIKPATWETGISMRFEVYGCKITDYPCSGMLGMVSG |
| | LISDSQITSSNQGDRNWMPENIRLVTSRSGWALPPAPHSYINEWLQIDLGEEKIVRGIIIQGGKHRENKVFMRKFKIGYSNNGSD |
| | WKMIMDDSKRKAKSFEGNNNYDTPELRTFPALSTRFIRIYPERATHGGLGLRMELLGCGGGGSGGGGSEPKSCDKTHTCPPCPAP |
| | ELLGGPSVFLFPPKPKDTLMISRTPEVTCVVVDVSHEDPEVKFNWYVDGVEVHNAKTKPREEQYNSTYRVVSVLTVLHQDWLNGK |
| | EYKCKVSNKALPAPIEKTISKAKGQPREPQVYTLPPSRDELTKNQVSLTCLVKGFYPSDIAVEWESNGQPENNYKTTPPVLDSDG |
| | SFFLYSKLTVDKSRWQQGNVFSCSVMHEALHNHYTQKSLSLSPGKGGGGSGGGGSGGGGSEEQAKTFLDKFNHEAEDLFYQSSGL |
| | GKGDFRGGGGSGGGGSGGGGSEEQAKTFLDKFNHEAEDLFYQSSGLGKGDFR |
| 206 | EDFKCMEALGMESGEIHSDQITASSQYSTNWSAERSRLNYPENGWTPGEDSYREWIQVDLGLLRFVTAVGTQGAISKETKKKYYV |
| | KTYKIDVSSNGEDWITIKEGNKPVLFQGNTNPTDVVVAVFPKPLITRFVRIKPATWETGISMRFEVYGDKITDYPCSNMLGMLSG |
| | LIADSQISASSTQEYLWSPSAARLVSSRSGWFPRIPQAQPGEEWLQVDLGTPKTVKGVIIQGARGGDSITAVEARAFVRKFKVSY |
| | SLNGKDWEYIQDPRTQQPKLFEGNMHYDTPDIRRFDPIPAQYVRVYPERWSPAGIGMRLEVLGCGGGGSGGGGSEPKSCDKTHTC |
| | PPCPAPELLGGPSVFLFPPKPKDTLMISRTPEVTCVVVDVSHEDPEVKFNWYVDGVEVHNAKTKPREEQYNSTYRVVSVLTVLHQ |
| | DWLNGKEYKCKVSNKALPAPIEKTISKAKGQPREPQVYTLPPSRDELTKNQVSLTCLVKGFYPSDIAVEWESNGQPENNYKTTPP |
| | VLDSDGSFFLYSKLTVDKSRWQQGNVFSCSVMHEALHNHYTQKSLSLSPGK |
| 207 | ENFQCNVPLGMESGRIANEQISASSTYSDGRWTPQQSRLHGDDNGWTPNLDSNKEYLQVDLRFLTMLTAIATQGAISRETQNGYY |
| | VKSYKLEVSTNGEDWMVYRHGKNHKVFQANNDATEVVLNKLHAPLLTRFVRIRPQTWHSGIALRLELFGCRVTDAPCSGMLGMVS |
| | GLISDSQITSSNQGDRNWMPENIRLVTSRSGWALPPAPHSYINEWLQIDLGEEKIVRGIIIQGGKHRENKVFMRKFKIGYSNNGS |
| | DWKMIMDDSKRKAKSFEGNNNYDTPELRTFPALSTRFIRIYPERATHGGLGLRMELLGCGGGGSGGGGSEPKSCDKTHTCPPCPA |
| | PELLGGPSVFLFPPKPKDTLMISRTPEVTCVVVDVSHEDPEVKFNWYVDGVEVHNAKTKPREEQYNSTYRVVSVLTVLHQDWLNG |
| | KEYKCKVSNKALPAPIEKTISKAKGQPREPQVYTLPPSRDELTKNQVSLTCLVKGFYPSDIAVEWESNGQPENNYKTTPPVLDSD |
| | GSFFLYSKLTVDKSRWQQGNVFSCSVMHEALHNHYTQKSLSLSPGK |
| 208 | EDFKCMEALGMESGEIHSDQITASSQASTNWSAERSRLNYPENGWTPGEDSYREWIQVDLGLLRFVTAVGTQGAIAKETKKKAYV |
| | KTYKIDVSSNGEDWITIKEGNKPVLFQGNTNPTDVVVAVFPKPLITRFVRIKPATWETGISMRFEVYGCKITDYPCSNMLGMLSG |
| | LIADSQISASSTQEYLWSPSAARLVSSRSGWFPRIPQAQPGEEWLQVDLGTPKTVKGVIIQGARGGDSITAVEARAFVRKFKVSY |
| | SLNGKDWEYIQDPRTQQPKLFEGNMHYDTPDIRRFDPIPAQYVRVYPERWSPAGIGMRLEVLGCGGGGSGGGGSEPKSCDKTHTC |
| | PPCPAPELLGGPSVFLFPPKPKDTLMISRTPEVTCVVVDVSHEDPEVKFNWYVDGVEVHNAKTKPREEQYNSTYRVVSVLTVLHQ |
| | DWLNGKEYKCKVSNKALPAPIEKTISKAKGQPREPQVYTLPPSRDELTKNQVSLTCLVKGFYPSDIAVEWESNGQPENNYKTTPP |
| | VLDSDGSFFLYSKLTVDKSRWQQGNVFSCSVMHEALHNHYTQKSLSLSPGK |
| 209 | ENFQCNVPLGMESGRIANEQISASSTYSDGRWTPQQSRLHGDDNGWTPNLDSNKEYLQVDLRFLTMLTAIATQGAISRETQNGYY |
| | VKSYKLEVSTNGEDWMVYRHGKNHKVFQANNDATEVVLNKLHAPLLTRFVRIRPQTWHSGIALRLELFGCRVTDAPCSNMLGMLS |
| | GLIADSQISASSTQEYLWSPSAARLVSSRSGWFPRIPQAQPGEEWLQVDLGTPKTVKGVIIQGARGGDSITAVEARAFVRKFKVS |
| | YSLNGKDWEYIQDPRTQQPKLFEGNMHYDTPDIRRFDPIPAQYVRVYPERWSPAGIGMRLEVLGCGGGGSGGGGSERKCCVECPP |
| | CPAPPVAGPSVFLFPPKPKDTLMISRTPEVTCVVVDVSHEDPEVQFNWYVDGVEVHNAKTKPREEQFNSTFRVVSVLTVVHQDWL |
| | NGKEYKCKVSNKGLPAPIEKTISKTKGQPREPQVYTLPPSREEMTKNQVSLTCLVKGFYPSDIAVEWESNGQPENNYKTTPPMLD |
| | SDGSFFLYSKLTVDKSRWQQGNVFSCSVMHEALHNHYTQKSLSLSPGK |

TABLE 22
Binding assay strategy results.

| | | | | | | Reduced | | | | | Heparin |
|---|---|---|---|---|---|---|---|---|---|---|---|
| SEQ ID | | | | NRP | NRP | heparin | VEGF | b1 | b1b2 | Length | affinity |
| NO. | Structure | Mutation | Strategy | 1 | 2 | mutation | binding | No. | No. | (monomer) | column |
| 113 | b1-Fc | WT | single b1 | Yes | No | No | Unmodified | 1 | | 396 | no binding |
| 114 | b1-Fc | K373E, | single b1, heparin binding | Yes | No | Yes | Unmodified | 1 | | 406 | no binding |
| | | | ↓(E) in b1 | | | | | | | | |
| 115 | b1b1-Fc | K373E, | double b1, heparin binding | Yes | No | Yes | Unmodified | 2 | | 576 | no binding |
| | | | ↓ (E) in b1 | | | | | | | | |
| 116 | b1b1b1-Fc | K373E, | triple b1, heparin binding ↓ | Yes | No | Yes | Unmodified | 3 | | 746 | no binding |
| | | | (E) in b1 | | | | | | | | |
| 117 | b1-Fc | Y297A, | single b1, no VEGF | Yes | No | Yes | Removed | 1 | | 406 | |
| | | S345A, | binding | | | | | | | | |
| | | Y353A, | | | | | | | | | |
| | | K373E | | | | | | | | | |
| 118 | b1b1b1-Fc | Y297A, | triple b1, no VEGF binding | Yes | No | Yes | Removed | 3 | | 746 | |
| | | S345A, | | | | | | | | | |
| | | Y353A, | | | | | | | | | |
| | | K373E | | | | | | | | | |
| 119 | b1-Fc | K358E, | single b1, heparin binding ↓ | Yes | No | Yes | Unmodified | 1 | | 406 | |
| | | K373E | in b1 (two site) | | | | | | | | |
| 120 | b1b1b1-Fc | K358E, | triple b1, heparin binding ↓ | Yes | No | Yes | Unmodified | 3 | | 746 | |
| | | K373E | in b1 (two site) | | | | | | | | |
| 121 | b1b1-b1b1-Fc | WT | quadruple b1 | Yes | No | No | Unmodified | 4 | | 868 | binding to |
| | | | | | | | | | | | heparin |
| 122 | b1b2-Fc | WT | single b1b2 | Yes | No | No | Unmodified | | 1 | 555 | binding to |
| | | | | | | | | | | | heparin |
| 123 | b1b2-Fc | E319A | single b1b2 (VEGF binding | Yes | No | No | Increased | | 1 | 551 | |
| | | | ↑) | | | | | | | | |
| 124 | b1b2-Fc | K351A | single b1b2 (VEGF binding | Yes | No | No | Decreased | | 1 | 551 | binding to |
| | | | ↓) | | | | | | | | heparin |
| 125 | b1b2-Fc | Y297A, | single b1b2, no VEGF | Yes | No | No | Removed | | 1 | 551 | binding to |
| | | S345A, | binding | | | | | | | | heparin |
| | | Y353A | | | | | | | | | |
| 126 | b1b2-Fc | R513E, | single b1b2, heparin | Yes | No | Yes | Unmodified | | 1 | 552 | |
| | | K514E, | binding↓ (E) in b2 | | | | | | | | |
| | | K516E | | | | | | | | | |
| 127 | b1b2-Fc | R513A, | single b1b2, heparin | Yes | No | Yes | Unmodified | | 1 | 552 | |
| | | K514A, | binding↓ (A) in b2 | | | | | | | | |
| | | K516A | | | | | | | | | |
| 128 | b1b2-Fc | K358E, | single b1b2, heparin | Yes | No | Yes | Unmodified | | 1 | 555 | no binding |
| | | K373E, | binding ↓ in b1b2 | | | | | | | | (60%), |
| | | R513E, | | | | | | | | | binding |
| | | K514E, | | | | | | | | | (40%) |
| | | K516E | | | | | | | | | |
| 129 | b1b2-Fc | Y297A, | single b1b2, no VEGF | Yes | No | Yes | Removed | | 1 | 555 | no binding |
| | | S345A, | binding, heparin binding ↓ | | | | | | | | (60%), |
| | | Y353A, | in b1b2 | | | | | | | | binding |
| | | K358E, | | | | | | | | | (40%) |
| | | K373E, | | | | | | | | | |
| | | R513E, | | | | | | | | | |
| | | K514E, | | | | | | | | | |
| | | K516E | | | | | | | | | |
| 130 | Fc-b1b2 | WT | b1b2 in C-term, single b1b2 | Yes | No | No | Unmodified | | 1 | 552 | |
| 131 | Fc-b1b2 | Y297A, | b1b2 in C-term, single | Yes | No | Yes | Unmodified | | 1 | 552 | |
| | | S345A, | b1b2, heparin binding↓ (E) | | | | | | | | |
| | | Y353A, | in b2 | | | | | | | | |
| | | R513E, | | | | | | | | | |
| | | K514E, | | | | | | | | | |
| | | K516E | | | | | | | | | |
| 132 | Fc-b1b2 | Y297A, | b1b2 in C-term, single | Yes | No | Yes | Unmodified | | 1 | 552 | |
| | | S345A, | b1b2, heparin binding↓ (A) | | | | | | | | |
| | | Y353A, | in b2 | | | | | | | | |
| | | R513A, | | | | | | | | | |
| | | K514A, | | | | | | | | | |
| | | K516A | | | | | | | | | |
| 133 | b1-Fc-b1 | K373E, | single b1 in N-term and C- | Yes | No | Yes | Unmodified | 1 | | 580 | |
| | | | term, heparin binding (E) ↓ | | | | | | | | |
| | | | in b1 | | | | | | | | |
| 134 | b1b1-Fc-b | K373E, | double b1 in N-term and | Yes | No | Yes | Unmodified | 2 | | 750 | |
| | | | single b1 in C-term, heparin | | | | | | | | |
| | | | binding (E) ↓ in b1 | | | | | | | | |
| 135 | b1b1-Fc | WT | single b1 in N-term and C- | Yes | No | No | Unmodified | 1 | | 576 | binding to |
| | | | term | | | | | | | | heparin |
| 136 | b1b1b1-Fc | WT | single b1 in N-term and C- | Yes | No | No | Unmodified | 1 | | 746 | binding to |
| | | | term | | | | | | | | heparin |
| 137 | b1-Fc-b1 | WT | single b1 in N-term and C- | Yes | No | No | Unmodified | 1 | | 580 | binding to |
| | | | term | | | | | | | | heparin |
| 138 | b1b2-b1b2-Fc | WT | double b1b2 | Yes | No | No | Unmodified | | 2 | 874 | binding to |
| | | | | | | | | | | | heparin |
| 139 | b1b2-b1b2-Fc | E319A | double b1b2 (VEGF | Yes | No | No | Increased | | 2 | 874 | |
| | | | binding ↑) | | | | | | | | |
| 140 | b1b2-b1b2-Fc | K351A | double b1b2 (VEGF | Yes | No | No | Decreased | | 2 | 874 | |
| | | | binding ↓) | | | | | | | | |
| 141 | b1b2-b1b2-Fc | Y297A, | double b1b2, no VEGF | Yes | No | No | Removed | | 2 | 874 | |
| | | S345A, | binding | | | | | | | | |
| | | Y353A | | | | | | | | | |
| 142 | b1b2-Fc-b1b2 | WT | single b1b2 in N-term and | Yes | No | No | Unmodified | | 1 | 878 | |
| | | | C-term | | | | | | | | |
| 143 | b1b2-Fc-b1b2 | E319A | double b1 in N-term and | Yes | No | No | Increased | 2 | | 878 | |
| | | | single b1 in C-term, | | | | | | | | |
| | | | increased VEGF binding | | | | | | | | |
| 144 | b1-Fc | WT | single b1 | No | Yes | No | Unmodified | 1 | | 407 | |
| 145 | b1b1-Fc | WT | double b1 | No | Yes | No | Unmodified | 2 | | 578 | |
| 146 | b1b1b1-Fc | WT | triple b1 | No | Yes | No | Unmodified | 3 | | 749 | |
| 147 | b1-Fc | K373E, | single b1, heparin binding ↓ | No | Yes | Yes | Unmodified | 1 | | 407 | |
| | | | (E) in b1 | | | | | | | | |
| 148 | b1b1-Fc | K373E, | double b1, heparin binding | No | Yes | Yes | Unmodified | 2 | | 578 | |
| | | | ↓ (E) in b1 | | | | | | | | |
| 149 | b1b1b1-Fc | K373E, | triple b1, heparin binding ↓ | No | Yes | Yes | Unmodified | 3 | | 749 | |
| | | | (E) in b1 | | | | | | | | |
| 150 | b1-Fc | Y297A, | single b1, No VEGF | No | Yes | Yes | Decreased | 1 | | 407 | |
| | | S345A, | binding, heparin binding ↓ | | | | | | | | |
| | | Y353A, | (E) in b1 | | | | | | | | |
| | | K373E | | | | | | | | | |
| 151 | b1b1b1-Fc | Y297A, | triple b1, No VEGF | No | Yes | Yes | Decreased | 3 | | 749 | |
| | | S345A, | binding, heparin binding ↓ | | | | | | | | |
| | | Y353A, | (E) in b1 | | | | | | | | |
| | | K373E | | | | | | | | | |
| 152 | b1-Fc | K358E, | single b1, heparin binding ↓ | No | Yes | Yes | Unmodified | 1 | | 407 | |
| | | K373E | (E) in b1 (two sites) | | | | | | | | |
| 153 | b1b1b1-Fc | K358E, | triple b1, heparin binding ↓ | No | Yes | Yes | Unmodified | 3 | | 749 | |
| | | K373E | (E) in b1 (two sites) | | | | | | | | |
| 154 | b162-Fc | WT | single b1b2 | No | Yes | No | Unmodified | | 1 | 559 | binding to |
| | | | | | | | | | | | heparin |
| 155 | b1b2-Fc | R513A, | single b1b2, heparin | No | Yes | Yes | Unmodified | | 1 | 559 | |
| | | K514A, | binding ↓ (A) in b2 | | | | | | | | |
| | | K516A | | | | | | | | | |
| 156 | b1b2-Fc | R513E, | single b1b2, heparin | No | Yes | Yes | Unmodified | | 1 | 559 | |
| | | K514E, | binding ↓ (E) in b2 | | | | | | | | |
| | | K516E | | | | | | | | | |
| 157 | Fc-b1b2 | WT | single b1b2 in C-term | No | Yes | No | Unmodified | | 1 | 559 | |
| 158 | Fc-b1b2 | Y297A, | single b1b2 in C-term, | No | Yes | Yes | Unmodified | | 1 | 555 | |
| | | S345A, | heparin binding ↓ (A) in b2 | | | | | | | | |
| | | Y353A, | | | | | | | | | |
| | | R513E, | | | | | | | | | |
| | | K514E, | | | | | | | | | |
| | | K516E | | | | | | | | | |
| 159 | Fc-b1b2 | Y297A, | single b1b2 in C-term, | No | Yes | Yes | Unmodified | | 1 | 555 | |
| | | S345A, | heparin binding ↓ (E) in b2 | | | | | | | | |
| | | Y353A, | | | | | | | | | |
| | | R513A, | | | | | | | | | |
| | | K514A, | | | | | | | | | |
| | | K516A | | | | | | | | | |
| 160 | b1-Fc-b1 | WT | single b1 in N-term and C- | No | Yes | No | Unmodified | 1 | | 582 | |
| | | | term | | | | | | | | |
| 161 | b1b1-Fc-b1 | WT | double b1 in N-term and | No | Yes | No | Unmodified | 3 | | 753 | |
| | | | single b1 in C-term | | | | | | | | |
| 162 | b1-Fc-b1 | K373E, | single b1 in N-term and C- | No | Yes | Yes | Unmodified | 1 | | 582 | |
| | | | term, heparin binding (E) ↓ | | | | | | | | |
| | | | in b1 | | | | | | | | |
| 163 | b1b1-Fc-b1 | K373E, | double b1 in N-term and | No | Yes | Yes | Unmodified | 3 | | 753 | |
| | | | single b1 in C-term, heparin | | | | | | | | |
| | | | binding (E) ↓ in b1 | | | | | | | | |
| 164 | N1(b1)/ | WT | N1(b1), N2(b2) | Yes | Yes | No | Unmodified | | 1 | 569 | |
| | N2(b2)-Fc | | | | | | | | | | |
| 165 | N2(b1)/ | WT | N2(b1), N1(b2) | Yes | Yes | No | Unmodified | | 1 | 564 | |
| | N1(b2)-Fc | | | | | | | | | | |
| 166 | N1(b1)/ | Y297A, | N1(b1), N2(b2), no binding | Yes | Yes | No | Removed | | 1 | 569 | |
| | N2(b2)-Fc | S345A, | VEGF | | | | | | | | |
| | | Y353A | | | | | | | | | |
| 202 | N1(a1a2b1b2c)- | | ECD domain of N1 | Yes | No | No | Unmodified | 1 | | 1057 | |
| | Fc | | | | | | | | | | |
| 167 | N1(a1a2b1b2)- | | a1a2 and b1b2 domain of | Yes | No | No | Unmodified | | 1 | 788 | |
| | Fc | | N1 | | | | | | | | |
| 168 | N1(a2b1)-Fc | | a2 and b1 domain of N1 | Yes | No | No | Unmodified | 1 | | 565 | |
| 191 | b1b2-Fc | WT | single b1 | Yes | No | No | Unmodified | 1 | | 555 | |
| 192 | b1b2-Fc- | | single b1 in N-term and | Yes | No | No | Unmodified | | 1 | 601 | |
| | ACE2-1 | | alpha domain of ACE2 in | | | | | | | | |
| | | | C-term | | | | | | | | |
| 193 | b1b2-Fc | Y297A, | single b1b2 in N-term , no | Yes | No | No | Removed | | 1 | 555 | |
| | | S345A, | VEGF binding | | | | | | | | |
| | | Y353A | | | | | | | | | |
| 194 | b1b2-Fc | WT | single b1b2-Fc | No | Yes | No | Unmodified | | 1 | 564 | |
| 195 | b1-Fc | WT | single b1 | Yes | No | No | Unmodified | 1 | | 406 | |
| 196 | b1-b1-Fc | K373E, | double b1 in N-term, | Yes | No | Yes | Unmodified | 2 | | 584 | |
| | | | heparin binding ↓(E) in b1 | | | | | | | | |
| 197 | b1-b1-b1-Fc | K373E, | triple b1 in N-term, heparin | Yes | No | Yes | Unmodified | 3 | | 758 | |
| | | | binding ↓(E) in b1 | | | | | | | | |
| 198 | b1-Fc-b1 | K373E, | single b1 in N-term and | Yes | No | Yes | Unmodified | 2 | | 588 | |
| | | | single b1 in C-term, | | | | | | | | |
| | | | heparin binding ↓(E) in b1 | | | | | | | | |
| 199 | b1-b1-Fc | K358E | double b1, heparin binding | No | Yes | Yes | Unmodified | 2 | | 586 | |
| | | | ↓ (E) in b1 | | | | | | | | |
| 200 | b1-b1-b1-Fc | K358E | triple b1, heparin binding ↓ | No | Yes | Yes | Unmodified | 3 | | 761 | |
| | | | (E) in b1 | | | | | | | | |
| 201 | b1-Fc-b1 | K358E | single b1 in N-term and | No | Yes | Yes | Unmodified | 2 | | 590 | |
| | | | single b1 in C-term, heparin | | | | | | | | |
| | | | binding ↓ (E) in b1 | | | | | | | | |
| 203 | b1b2-Fc-ACE2 | WT | single b1b2, IgG2Fc, ACE2 | Yes | No | No | Unmodified | | 1 | 596 | |
| | | | alpha domain | | | | | | | | |
| 204 | b1b2-Fc-ACE2 | K358E, | single b1b2, IgG2Fc, | Yes | No | Yes | Unmodified | | 1 | 596 | |
| | | K373E, | repeated ACE2 alpha | | | | | | | | |
| | | R513E, | domain, heparin binding | | | | | | | | |
| | | K514E, | mutation | | | | | | | | |
| | | K516E | | | | | | | | | |
| 205 | b1b2-Fc-ACE2 | WT | single b1b2, IgG2Fc, | Yes | No | No | Unmodified | | 1 | 647 | |
| | | | repeated ACE2 alpha | | | | | | | | |
| | | | domain | | | | | | | | |
| 206 | Nrp1(b1)/ | WT | b1 of NRP1 and b2 of | Yes | Yes | No | Unmodified | | 1 | 561 | |
| | Nrp2(b2)-Fc | | NRP2 | | | | | | | | |
| 207 | Nrp2(b1)/ | WT | b1 of NRP2 and b2 of | Yes | Yes | No | Unmodified | | 1 | 556 | |
| | Nrp1(b2)-Fc | | NRP1 | | | | | | | | |
| 208 | Nrp1(b1) | Y297A, | b1 of NRP1 and b2 of | Yes | Yes | No | No binding | | 1 | 561 | |
| | Nrp2(b2)-Fc | S345A, | NRP2, No VEGF binding | | | | | | | | |
| | | Y353A | | | | | | | | | |
| 209 | b1b2-Fc | WT | Single b1b2 domain | No | Yes | No | Unmodified | | 1 | 558 | |

The column labeled “Structure” shows the overall design and orientation of motifs comprising a given construct (as described in FIG. 4).
The column labeled “Strategy” shows the corresponding strategy and/or anticipated effect; here, a downward pinging arrow (“↓”) means decreased. “E” means that the amino acid was substituted with a glutamic acid residue. “A” means that the amino acid was substituted to an alanine residue. N1 = NRP1; N2 = NRP2.
The column labeled “Reduced heparin mutation” identifies whether the corresponding construct possess a mutation that reduces heparin binding.
The column labeled “VEGF binding” indicates whether a given construct's VEGF binding has been increased, decreased, or is unmodified.
The column labeled “b1 No.” and “b1b2 No.” indicates the b1 and b1b2 subunits used, respectively.
The column labeled “Heparin affinity column” shows the results of heparin binding.

Results of Heparin Binding

The results of heparin affinity were summarized in the table below. Some of the constructs tested did not bind to heparin column. For example, SEQ ID NOs: 113, 128, 129, 114, 115, 116 and 133 showed no heparin binding. Among the foregoing constructs, SEQ ID NO: 113 has wild type of b1 domain, whereas the other SEQ ID NOs: 128, 129, 114, 115, 116, and 133 include at least one mutation to the putative heparin binding sites.

Except SEQ ID NO: 133, all of the constructs that have a wild type b (domain only, or a b1b2 domain, were shown to bind to heparin. The construct molecules SEQ ID NOs: 122, 124 and 125, which have a wild type b1b2 domain, showed stronger heparin binding than the constructs having only a b1 domain. And, a single b1 domain did not result in heparin binding, however, increasing the number of b1 domains resulted in heparin binding: thus indicating that increased avidity of b1 domain could interact with heparin.

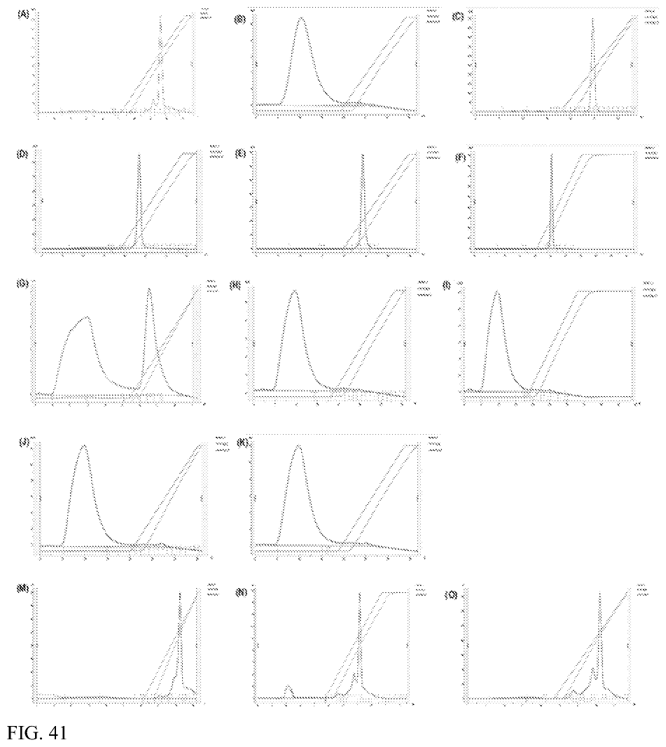

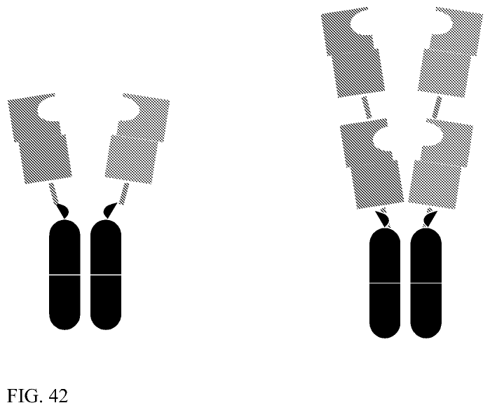

TABLE 23
Heparin affinity results of construct molecules. Here, the letter in the column entitled
“Heparin binding affinity profile” corresponds to the letters in FIG. 41.

| | Heparin | | | | Heparin HP |
|---|---|---|---|---|---|
| | binding | | VEGF | Heparin | elution |
| | affinity | | binding | binding site | conductivity |
| SEQ ID NO. | profile | Structure | site | mutation | (mS/cm) |
| 113 | A | N1(b1)-Fc | WT | WT | No binding |
| 121 | B | N1(b1b1)- | WT | WT | 30 |
| | | N1(b1b1)-Fc | | | |
| 122 | C | N1(b1b2)-Fc | WT | WT | 49 |
| 125 | N | N1(b1b2)-Fc | Y297A, | WT | 49 |
| | | | S345A, | | |
| | | | Y353A | | |
| 124 | O | N1(b1b2)-Fc | K351A | WT | 49 |
| 128 | G | N1(b1b2)-Fc | WT | K358E, K373E, | No binding |
| | | | | R513E, K514E, | (60%), 16 |
| | | | | K516E | (40%) |
| 129 | L | N1(b1b2)-Fc | Y297A, | K358E, K373E, | No binding |
| | | | S345A, | R513E, K514E, | (60%), 16 |
| | | | Y353A | K516E | (40%) |
| 154 | M | N2(b1b2)-Fc | WT | WT | 49 |
| 135 | D | N1(b1)-N1(b1)-Fc | WT | WT | 16 |
| 136 | E | N1(b1)-N1(b1)- | WT | WT | 16 |
| | | N1(b1)-Fc | | | |
| 137 | F | N1(b1)-Fc-N1(b1) | WT | WT | 18 |
| 114 | H | N1(b1)-Fc | WT | K373E | No binding |
| 115 | I | N1(b1)-N1(b1)-Fc | WT | K373E | No binding |
| 116 | J | N1(b1)-N1(b1)- | WT | K373E | No binding |
| | | N1(b1)-Fc | | | |
| 133 | K | N1(b1)-Fc-N1(b1) | WT | K373E | No binding |

### Example 14. CendR Decoy Sequences

Overview

Receptor binding domain CendR motifs were evaluated in order to display and characterize neuropilin-1 b1-domain-antibody-fusion constructs binding to CendR motifs, and the binding of b1 to viral proteins and/or CendR peptides.

The constructs tested in the present example comprised either one or multiple recognition binding domain targeting neuropilin-1 b1 domains in tandem expressed as recombinant fusion molecules on the n-terminus of the human IgG Fc. FIG. 42.

Without wishing to be bound by theory, it is hypothesized that the constructs of the present invention bind the CendR motifs of glycoproteins of SARS-CoV-2, Respiratory Syncytial Virus (RSV), and other viral pathogens through neuropilin-1 b1 domains. And, Viral Recognition Binding Domain (RBD) targeting is primarily mediated by binding of neuropilin b1 domain to the RBD cleaved CendR motif (e.g., RXXROH) of viral constructs expressed on spike proteins on virus particle membranes. Neuropilin-1 is a cell surface receptor involved in multiple developmental process including axon guidance, angiogenesis, and heterophilic cell adhesion. Neuropilin-1 b1 domain forms part of the tandem coagulation factor domains along with the b2 domain and both domains belong to the domain family referred to as F5/8 type C, or the discoidin domain family. Neuropiln-1 b1 domain has been identified as the primary driver of binding to the S1 domain of virus spike proteins and may aid in viral infectivity. The recombinant fusion of the RBD binding b1 domain with human IgG Fc activates the adaptive immune system to engage and prevent infectivity of viral pathogens. Neuropilin-1 b1 domain in conjunction with other neuropilin ectodomains mediates binding of ligands including semaphorins, coagulation factors V and VIII, and VEGF in axon guidance cue and angiogenic factor binding as part of the developmental process and other physiological activities.

Binding analyses were established using Bio-Layer Interferometry (BLI) on an Octet® Red 96 system (ForteBio). The specific objectives of the present example were as follows: (1) Establish construct molecules binding to RBD CendR motif of virus particles; and (2) Characterize construct molecules binding parameters to RBD CendR motif targets recombinant virus proteins

Binding of Construct Molecules Recombinant Viral Proteins

To assess the binding of the construct molecules to viral proteins, BLI binding studies using the Octet red 96 system were conducted. Briefly, constructs were immobilized on Anti-Human Fc Capture (AHC) biosensors and interrogated for binding to recombinant viral proteins that had been commercially sourced. Using BLI technology, the binding to constructs was evaluated across various viruses and both kinetics and binding affinity (equilibrium binding constant, K_{D}) were assessed in the monovalent format. Additionally, CendR peptide sequences from various viruses were immobilized on streptavidin (SA) biosensors These studies were performed to determine whether construct molecules exhibit broad spectrum activity, and with what affinity the construct molecules have for the target proteins and peptide.

Construct molecules tested are described in the table below.

TABLE 24
Constructs evaluated in CendR studies.

| SEQ ID NO. | Sequence |
|---|---|
| 113 | EDFKCMEALGMESGEIHSDQITASSQYSTNWSAERSRLNYPENGWTPGEDSYREWIQ |
| | VDLGLLRFVTAVGTQGAISKETKKKYYVKTYKIDVSSNGEDWITIKEGNKPVLFQGN |
| | TNPTDVVVAVFPKPLITRFVRIKPATWETGISMRFEVYGCGGGGSGGGGSEPKSCDK |
| | THTCPPCPAPELLGGPSVFLFPPKPKDTLMISRTPEVTCVVVDVSHEDPEVKFNWYV |
| | DGVEVHNAKTKPREEQYNSTYRVVSVLAVLHQDWLNGKEYKCKVSNKALPAPIEKTI |
| | SKAKGQPREPQVYTLPPSRDELTKNQVSLTCLVKGFYPSDIAVAWESNGQPENNYKT |
| | TPPVLDSDGSFFLYSKLTVDKSRWQQGNVFSCSVMHEALHAHYTQKSLSLSPGK |
| 121 | EDFKCMEALGMESGEIHSDQITASSQYSTNWSAERSRLNYPENGWTPGEDSYREWIQ |
| | VDLGLLRFVTAVGTQGAISKETKKKYYVKTYKIDVSSNGEDWITIKEGNKPVLFQGN |
| | TNPTDVVVAVFPKPLITRFVRIKPATWETGISMRFEVYGCKITDYPCMEALGMESGE |
| | IHSDQITASSQYSTNWSAERSRLNYPENGWTPGEDSYREWIQVDLGLLRFVTAVGTQ |
| | GAISKETKKKYYVKTYKIDVSSNGEDWITIKEGNKPVLFQGNTNPTDVVVAVFPKPL |
| | ITRFVRIKPATWETGISMRFEVYGCGGGGSGGGGSCMEALGMESGEIHSDQITASSQ |
| | YSTNWSAERSRLNYPENGWTPGEDSYREWIQVDLGLLRFVTAVGTQGAISKETKKKY |
| | YVKTYKIDVSSNGEDWITIKEGNKPVLFQGNTNPTDVVVAVFPKPLITRFVRIKPAT |
| | WETGISMRFEVYGCKITDYPCMEALGMESGEIHSDQITASSQYSTNWSAERSRLNYP |
| | ENGWTPGEDSYREWIQVDLGLLRFVTAVGTQGAISKETKKKYYVKTYKIDVSSNGED |
| | WITIKEGNKPVLFQGNTNPTDVVVAVFPKPLITRFVRIKPATWETGISMRFEVYGCG |
| | GGGSGGGGSEPKSCDKTHTCPPCPAPELLGGPSVFLFPPKPKDTLMISRTPEVTCVV |
| | VDVSHEDPEVKFNWYVDGVEVHNAKTKPREEQYNSTYRVVSVLAVLHQDWLNGKEYK |
| | CKVSNKALPAPIEKTISKAKGQPREPQVYTLPPSRDELTKNQVSLTCLVKGFYPSDI |
| | AVAWESNGQPENNYKTTPPVLDSDGSFFLYSKLTVDKSRWQQGNVFSCSVMHEALHA |
| | HYTQKSLSLSPGK |
| 122 | EDFKCMEALGMESGEIHSDQITASSQYSTNWSAERSRLNYPENGWTPGEDSYREWIQ |
| | VDLGLLRFVTAVGTQGAISKETKKKYYVKTYKIDVSSNGEDWITIKEGNKPVLFQGN |
| | TNPTDVVVAVFPKPLITRFVRIKPATWETGISMRFEVYGCKITDYPCSGMLGMVSGL |
| | ISDSQITSSNQGDRNWMPENIRLVTSRSGWALPPAPHSYINEWLQIDLGEEKIVRGI |
| | IIQGGKHRENKVFMRKFKIGYSNNGSDWKMIMDDSKRKAKSFEGNNNYDTPELRTFP |
| | ALSTRFIRIYPERATHGGLGLRMELLGCGGGGSGGGGSEPKSCDKTHTCPPCPAPEL |
| | LGGPSVFLFPPKPKDTLMISRTPEVTCVVVDVSHEDPEVKFNWYVDGVEVHNAKTKP |
| | REEQYNSTYRVVSVLTVLHQDWLNGKEYKCKVSNKALPAPIEKTISKAKGQPREPQV |
| | YTLPPSRDELTKNQVSLTCLVKGFYPSDIAVEWESNGQPENNYKTTPPVLDSDGSFF |
| | LYSKLTVDKSRWQQGNVFSCSVMHEALHNHYTQKSLSLSPGK |
| 154 | ENFQCNVPLGMESGRIANEQISASSTYSDGRWTPQQSRLHGDDNGWTPNLDSNKEYL |
| | QVDLRFLTMLTAIATQGAISRETQNGYYVKSYKLEVSTNGEDWMVYRHGKNHKVFQA |
| | NNDATEVVLNKLHAPLLTRFVRIRPQTWHSGIALRLELFGCRVTDAPCSNMLGMLSG |
| | LIADSQISASSTQEYLWSPSAARLVSSRSGWFPRIPQAQPGEEWLQVDLGTPKTVKG |
| | VIIQGARGGDSITAVEARAFVRKFKVSYSLNGKDWEYIQDPRTQQPKLFEGNMHYDT |
| | PDIRRFDPIPAQYVRVYPERWSPAGIGMRLEVLGCGGGGSGGGGSEPKSCDKTHTCP |
| | PCPAPELLGGPSVFLFPPKPKDTLMISRTPEVTCVVVDVSHEDPEVKFNWYVDGVEV |
| | HNAKTKPREEQYNSTYRVVSVLTVLHQDWLNGKEYKCKVSNKALPAPIEKTISKAKG |
| | QPREPQVYTLPPSRDELTKNQVSLTCLVKGFYPSDIAVEWESNGQPENNYKTTPPVL |
| | DSDGSFFLYSKLTVDKSRWQQGNVFSCSVMHEALHNHYTQKSLSLSPGK |
| 193 | EDFKCMEALGMESGEIHSDQITASSQASTNWSAERSRLNYPENGWTPGEDSYREWIQ |
| | VDLGLLRFVTAVGTQGAIAKETKKKAYVKTYKIDVSSNGEDWITIKEGNKPVLFQGN |
| | TNPTDVVVAVFPKPLITRFVRIKPATWETGISMRFEVYGCKITDYPCSGMLGMVSGL |
| | ISDSQITSSNQGDRNWMPENIRLVTSRSGWALPPAPHSYINEWLQIDLGEEKIVRGI |
| | IIQGGKHRENKVFMRKFKIGYSNNGSDWKMIMDDSKRKAKSFEGNNNYDTPELRTFP |
| | ALSTRFIRIYPERATHGGLGLRMELLGCGGGGSGGGGSEPKSCDKTHTCPPCPAPEL |
| | LGGPSVFLFPPKPKDTLMISRTPEVTCVVVDVSHEDPEVKFNWYVDGVEVHNAKTKP |
| | REEQYNSTYRVVSVLTVLHQDWLNGKEYKCKVSNKALPAPIEKTISKAKGQPREPQV |
| | YTLPPSRDELTKNQVSLTCLVKGFYPSDIAVEWESNGQPENNYKTTPPVLDSDGSFF |
| | LYSKLTVDKSRWQQGNVFSCSVMHEALHNHYTQKSLSLSPGK |
| 194 | ENFQCNVPLGMESGRIANEQISASSTYSDGRWTPQQSRLHGDDNGWTPNLDSNKEYL |
| | QVDLRFLTMLTAIATQGAISRETQNGYYVKSYKLEVSTNGEDWMVYRHGKNHKVFQA |
| | NNDATEVVLNKLHAPLLTRFVRIRPQTWHSGIALRLELFGCRVTDAPCSNMLGMLSG |
| | LIADSQISASSTQEYLWSPSAARLVSSRSGWFPRIPQAQPGEEWLQVDLGTPKTVKG |
| | VIIQGARGGDSITAVEARAFVRKFKVSYSLNGKDWEYIQDPRTQQPKLFEGNMHYDT |
| | PDIRRFDPIPAQYVRVYPERWSPAGIGMRLEVLGCGAPAPAGAPAPAEPKSCDKTHT |
| | CPPCPAPELLGGPSVFLFPPKPKDTLMISRTPEVTCVVVDVSHEDPEVKFNWYVDGV |
| | EVHNAKTKPREEQYNSTYRVVSVLTVLHQDWLNGKEYKCKVSNKALPAPIEKTISKA |
| | KGQPREPQVYTLPPSRDELTKNQVSLTCLVKGFYPSDIAVEWESNGQPENNYKTTPP |
| | VLDSDGSFFLYSKLTVDKSRWQQGNVFSCSVMHEALHNHYTQKSLSLSPGK |
| 195 | EDFKCMEALGMESGEIHSDQITASSQYSTNWSAERSRLNYPENGWTPGEDSYREWIQ |
| | VDLGLLRFVTAVGTQGAISKETKKKYYVKTYKIDVSSNGEDWITIKEGNKPVLFQGN |
| | TNPTDVVVAVFPKPLITRFVRIKPATWETGISMRFEVYGCGGGGSGGGGSGGGGSGG |
| | GGSEPKSCDKTHTCPPCPAPELLGGPSVFLFPPKPKDTLMISRTPEVTCVVVDVSHE |
| | DPEVKFNWYVDGVEVHNAKTKPREEQYNSTYRVVSVLTVLHQDWLNGKEYKCKVSNK |
| | ALPAPIEKTISKAKGQPREPQVYTLPPSRDELTKNQVSLTCLVKGFYPSDIAVEWES |
| | NGQPENNYKTTPPVLDSDGSFFLYSKLTVDKSRWQQGNVFSCSVMHEALHNHYTQKS |
| | LSLSPGK |
| 135 | EDFKCMEALGMESGEIHSDQITASSQYSTNWSAERSRLNYPENGWTPGEDSYREWIQ |
| | VDLGLLRFVTAVGTQGAISKETKKKYYVKTYKIDVSSNGEDWITIKEGNKPVLFQGN |
| | TNPTDVVVAVFPKPLITRFVRIKPATWETGISMRFEVYGCGGGGSGGGGSGGGGSGG |
| | GGSCMEALGMESGEIHSDQITASSQYSTNWSAERSRLNYPENGWTPGEDSYREWIQV |
| | DLGLLRFVTAVGTQGAISKETKKKYYVKTYKIDVSSNGEDWITIKEGNKPVLFQGNT |
| | NPTDVVVAVFPKPLITRFVRIKPATWETGISMRFEVYGCGGGGSGGGGSGGGGSGGG |
| | GSEPKSCDKTHTCPPCPAPELLGGPSVFLFPPKPKDTLMISRTPEVTCVVVDVSHED |
| | PEVKFNWYVDGVEVHNAKTKPREEQYNSTYRVVSVLTVLHQDWLNGKEYKCKVSNKA |
| | LPAPIEKTISKAKGQPREPQVYTLPPSRDELTKNQVSLTCLVKGFYPSDIAVEWESN |
| | GQPENNYKTTPPVLDSDGSFFLYSKLTVDKSRWQQGNVFSCSVMHEALHNHYTQKSL |
| | SLSPGK |
| 136 | EDFKCMEALGMESGEIHSDQITASSQYSTNWSAERSRLNYPENGWTPGEDSYREWIQ |
| | VDLGLLRFVTAVGTQGAISKETKKKYYVKTYKIDVSSNGEDWITIKEGNKPVLFQGN |
| | TNPTDVVVAVFPKPLITRFVRIKPATWETGISMRFEVYGCGGGGSGGGGSGGGGSGG |
| | GGSCMEALGMESGEIHSDQITASSQYSTNWSAERSRLNYPENGWTPGEDSYREWIQV |
| | DLGLLRFVTAVGTQGAISKETKKKYYVKTYKIDVSSNGEDWITIKEGNKPVLFQGNT |
| | NPTDVVVAVFPKPLITRFVRIKPATWETGISMRFEVYGCGGGGSGGGGSGGGGSGGG |
| | GSCMEALGMESGEIHSDQITASSQYSTNWSAERSRLNYPENGWTPGEDSYREWIQVD |
| | LGLLRFVTAVGTQGAISKETKKKYYVKTYKIDVSSNGEDWITIKEGNKPVLFQGNTN |
| | PTDVVVAVFPKPLITRFVRIKPATWETGISMRFEVYGCGGGGSGGGGSGGGGSGGGG |
| | SEPKSCDKTHTCPPCPAPELLGGPSVFLFPPKPKDTLMISRTPEVTCVVVDVSHEDP |
| | EVKFNWYVDGVEVHNAKTKPREEQYNSTYRVVSVLTVLHQDWLNGKEYKCKVSNKAL |
| | PAPIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVMHEALHNHYTQKSLS |
| | LSPGK |
| 137 | EDFKCMEALGMESGEIHSDQITASSQYSTNWSAERSRLNYPENGWTPGEDSYREWIQ |
| | VDLGLLRFVTAVGTQGAISKETKKKYYVKTYKIDVSSNGEDWITIKEGNKPVLFQGN |
| | TNPTDVVVAVFPKPLITRFVRIKPATWETGISMRFEVYGCGGGGSGGGGSGGGGSGG |
| | GGSEPKSCDKTHTCPPCPAPELLGGPSVFLFPPKPKDTLMISRTPEVTCVVVDVSHE |
| | DPEVKFNWYVDGVEVHNAKTKPREEQYNSTYRVVSVLTVLHQDWLNGKEYKCKVSNK |
| | ALPAPIEKTISKAKGQPREPQVYTLPPSRDELTKNQVSLTCLVKGFYPSDIAVEWES |
| | NGQPENNYKTTPPVLDSDGSFFLYSKLTVDKSRWQQGNVFSCSVMHEALHNHYTQKS |
| | LSLSPGKGGGGSGGGGSGGGGSGGGGSCMEALGMESGEIHSDQITASSQYSTNWSAE |
| | RSRLNYPENGWTPGEDSYREWIQVDLGLLRFVTAVGTQGAISKETKKKYYVKTYKID |
| | VSSNGEDWITIKEGNKPVLFQGNTNPTDVVVAVFPKPLITRFVRIKPATWETGISMR |
| | FEVYGCEVEA |
| 114 | EDFKCMEALGMESGEIHSDQITASSQYSTNWSAERSRLNYPENGWTPGEDSYREWIQ |
| | VDLGLLRFVTAVGTQGAISKETKKKYYVKTYKIDVSSNGEDWITIEEGNKPVLFQGN |
| | TNPTDVVVAVFPKPLITRFVRIKPATWETGISMRFEVYGCGGGGSGGGGSGGGGSGG |
| | GGSEPKSCDKTHTCPPCPAPELLGGPSVFLFPPKPKDTLMISRTPEVTCVVVDVSHE |
| | DPEVKFNWYVDGVEVHNAKTKPREEQYNSTYRVVSVLTVLHQDWLNGKEYKCKVSNK |
| | ALPAPIEKTISKAKGQPREPQVYTLPPSRDELTKNQVSLTCLVKGFYPSDIAVEWES |
| | NGQPENNYKTTPPVLDSDGSFFLYSKLTVDKSRWQQGNVFSCSVMHEALHNHYTQKS |
| | LSLSPGK |
| 115 | EDFKCMEALGMESGEIHSDQITASSQYSTNWSAERSRLNYPENGWTPGEDSYREWIQ |
| | VDLGLLRFVTAVGTQGAISKETKKKYYVKTYKIDVSSNGEDWITIEEGNKPVLFQGN |
| | TNPTDVVVAVFPKPLITRFVRIKPATWETGISMRFEVYGCGGGGSGGGGSGGGGSGG |
| | GGSCMEALGMESGEIHSDQITASSQYSTNWSAERSRLNYPENGWTPGEDSYREWIQV |
| | DLGLLRFVTAVGTQGAISKETKKKYYVKTYKIDVSSNGEDWITIEEGNKPVLFQGNT |
| | NPTDVVVAVFPKPLITRFVRIKPATWETGISMRFEVYGCGGGGSGGGGSGGGGSGGG |
| | GSEPKSCDKTHTCPPCPAPELLGGPSVFLFPPKPKDTLMISRTPEVTCVVVDVSHED |
| | PEVKFNWYVDGVEVHNAKTKPREEQYNSTYRVVSVLTVLHQDWLNGKEYKCKVSNKA |
| | LPAPIEKTISKAKGQPREPQVYTLPPSRDELTKNQVSLTCLVKGFYPSDIAVEWESN |
| | GQPENNYKTTPPVLDSDGSFFLYSKLTVDKSRWQQGNVFSCSVMHEALHNHYTQKSL |
| | SLSPGK |
| 116 | EDFKCMEALGMESGEIHSDQITASSQYSTNWSAERSRLNYPENGWTPGEDSYREWIQ |
| | VDLGLLRFVTAVGTQGAISKETKKKYYVKTYKIDVSSNGEDWITIEEGNKPVLFQGN |
| | TNPTDVVVAVFPKPLITRFVRIKPATWETGISMRFEVYGCGGGGSGGGGSGGGGSGG |
| | GGSCMEALGMESGEIHSDQITASSQYSTNWSAERSRLNYPENGWTPGEDSYREWIQV |
| | DLGLLRFVTAVGTQGAISKETKKKYYVKTYKIDVSSNGEDWITIEEGNKPVLFQGNT |
| | NPTDVVVAVFPKPLITRFVRIKPATWETGISMRFEVYGCGGGGSGGGGSGGGGSGGG |
| | GSCMEALGMESGEIHSDQITASSQYSTNWSAERSRLNYPENGWTPGEDSYREWIQVD |
| | LGLLRFVTAVGTQGAISKETKKKYYVKTYKIDVSSNGEDWITIEEGNKPVLFQGNTN |
| | PTDVVVAVFPKPLITRFVRIKPATWETGISMRFEVYGCGGGGSGGGGSGGGGSGGGG |
| | SEPKSCDKTHTCPPCPAPELLGGPSVFLFPPKPKDTLMISRTPEVTCVVVDVSHEDP |
| | EVKFNWYVDGVEVHNAKTKPREEQYNSTYRVVSVLTVLHQDWLNGKEYKCKVSNKAL |
| | PAPIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVMHEALHNHYTQKSLS |
| | LSPGK |
| 133 | EDFKCMEALGMESGEIHSDQITASSQYSTNWSAERSRLNYPENGWTPGEDSYREWIQ |
| | VDLGLLRFVTAVGTQGAISKETKKKYYVKTYKIDVSSNGEDWITIEEGNKPVLFQGN |
| | TNPTDVVVAVFPKPLITRFVRIKPATWETGISMRFEVYGCGGGGSGGGGSGGGGSGG |
| | GGSEPKSCDKTHTCPPCPAPELLGGPSVFLFPPKPKDTLMISRTPEVTCVVVDVSHE |
| | DPEVKFNWYVDGVEVHNAKTKPREEQYNSTYRVVSVLTVLHQDWLNGKEYKCKVSNK |
| | ALPAPIEKTISKAKGQPREPQVYTLPPSRDELTKNQVSLTCLVKGFYPSDIAVEWES |
| | NGQPENNYKTTPPVLDSDGSFFLYSKLTVDKSRWQQGNVFSCSVMHEALHNHYTQKS |
| | LSLSPGKGGGGSGGGGSGGGGSGGGGSCMEALGMESGEIHSDQITASSQYSTNWSAE |
| | RSRLNYPENGWTPGEDSYREWIQVDLGLLRFVTAVGTQGAISKETKKKYYVKTYKID |
| | VSSNGEDWITIEEGNKPVLFQGNTNPTDVVVAVFPKPLITRFVRIKPATWETGISMR |
| | FEVYGCEVEA |
| 148 | ENFQCNVPLGMESGRIANEQISASSTYSDGRWTPQQSRLHGDDNGWTPNLDSNKEYL |
| | QVDLRFLTMLTAIATQGAISRETQNGYYVKSYKLEVSTNGEDWMVYEHGKNHKVFQA |
| | NNDATEVVLNKLHAPLLTRFVRIRPQTWHSGIALRLELFGCGGGGSGGGGSGGGGSG |
| | GGGSCNVPLGMESGRIANEQISASSTYSDGRWTPQQSRLHGDDNGWTPNLDSNKEYL |
| | QVDLRFLTMLTAIATQGAISRETQNGYYVKSYKLEVSTNGEDWMVYEHGKNHKVFQA |
| | NNDATEVVLNKLHAPLLTRFVRIRPQTWHSGIALRLELFGCGGGGSGGGGSGGGGSG |
| | GGGSEPKSCDKTHTCPPCPAPELLGGPSVFLFPPKPKDTLMISRTPEVTCVVVDVSH |
| | EDPEVKFNWYVDGVEVHNAKTKPREEQYNSTYRVVSVLTVLHQDWLNGKEYKCKVSN |
| | KALPAPIEKTISKAKGQPREPQVYTLPPSRDELTKNQVSLTCLVKGFYPSDIAVEWE |
| | SNGQPENNYKTTPPVLDSDGSFFLYSKLTVDKSRWQQGNVFSCSVMHEALHNHYTQK |
| | SLSLSPGK |
| 149 | ENFQCNVPLGMESGRIANEQISASSTYSDGRWTPQQSRLHGDDNGWTPNLDSNKEYL |
| | QVDLRFLTMLTAIATQGAISRETQNGYYVKSYKLEVSTNGEDWMVYEHGKNHKVFQA |
| | NNDATEVVLNKLHAPLLTRFVRIRPQTWHSGIALRLELFGCGGGGSGGGGSGGGGSG |
| | GGGSCNVPLGMESGRIANEQISASSTYSDGRWTPQQSRLHGDDNGWTPNLDSNKEYL |
| | QVDLRFLTMLTAIATQGAISRETQNGYYVKSYKLEVSTNGEDWMVYEHGKNHKVFQA |
| | NNDATEVVLNKLHAPLLTRFVRIRPQTWHSGIALRLELFGCGGGGSGGGGSGGGGSG |
| | GGGSCNVPLGMESGRIANEQISASSTYSDGRWTPQQSRLHGDDNGWTPNLDSNKEYL |
| | QVDLRFLTMLTAIATQGAISRETQNGYYVKSYKLEVSTNGEDWMVYEHGKNHKVFQA |
| | NNDATEVVLNKLHAPLLTRFVRIRPQTWHSGIALRLELFGCGGGGSGGGGSGGGGSG |
| | GGGSEPKSCDKTHTCPPCPAPELLGGPSVFLFPPKPKDTLMISRTPEVTCVVVDVSH |
| | EDPEVKFNWYVDGVEVHNAKTKPREEQYNSTYRVVSVLTVLHQDWLNGKEYKCKVSN |
| | KALPAPIEKTISKAKGQPREPQVYTLPPSRDELTKNQVSLTCLVKGFYPSDIAVEWE |
| | SNGQPENNYKTTPPVLDSDGSFFLYSKLTVDKSRWQQGNVFSCSVMHEALHNHYTQK |
| | SLSLSPGK |
| 162 | ENFQCNVPLGMESGRIANEQISASSTYSDGRWTPQQSRLHGDDNGWTPNLDSNKEYL |
| | QVDLRFLTMLTAIATQGAISRETQNGYYVKSYKLEVSTNGEDWMVYEHGKNHKVFQA |
| | NNDATEVVLNKLHAPLLTRFVRIRPQTWHSGIALRLELFGCGGGGSGGGGSGGGGSG |
| | GGGSEPKSCDKTHTCPPCPAPELLGGPSVFLFPPKPKDTLMISRTPEVTCVVVDVSH |
| | EDPEVKFNWYVDGVEVHNAKTKPREEQYNSTYRVVSVLTVLHQDWLNGKEYKCKVSN |
| | KALPAPIEKTISKAKGQPREPQVYTLPPSRDELTKNQVSLTCLVKGFYPSDIAVEWE |
| | SNGQPENNYKTTPPVLDSDGSFFLYSKLTVDKSRWQQGNVFSCSVMHEALHNHYTQK |
| | SLSLSPGKGGGGSGGGGSGGGGSGGGGSCNVPLGMESGRIANEQISASSTYSDGRWT |
| | PQQSRLHGDDNGWTPNLDSNKEYLQVDLRFLTMLTAIATQGAISRETQNGYYVKSYK |
| | LEVSTNGEDWMVYEHGKNHKVFQANNDATEVVLNKLHAPLLTRFVRIRPQTWHSGIA |
| | LRLELFGCDWTD |
| 196 | EDFKCMEALGMESGEIHSDQITASSQYSTNWSAERSRLNYPENGWTPGEDSYREWIQ |
| | VDLGLLRFVTAVGTQGAISKETKKKYYVKTYKIDVSSNGEDWITIEEGNKPVLFQGN |
| | TNPTDVVVAVFPKPLITRFVRIKPATWETGISMRFEVYGCGAPAPAGAPAPAGAPAP |
| | AGAPAPACMEALGMESGEIHSDQITASSQYSTNWSAERSRLNYPENGWTPGEDSYRE |
| | WIQVDLGLLRFVTAVGTQGAISKETKKKYYVKTYKIDVSSNGEDWITIEEGNKPVLF |
| | QGNTNPTDVVVAVFPKPLITRFVRIKPATWETGISMRFEVYGCGAPAPAGAPAPAGA |
| | PAPAGAPAPAEPKSCDKTHTCPPCPAPELLGGPSVFLFPPKPKDTLMISRTPEVTCV |
| | VVDVSHEDPEVKFNWYVDGVEVHNAKTKPREEQYNSTYRVVSVLTVLHQDWLNGKEY |
| | KCKVSNKALPAPIEKTISKAKGQPREPQVYTLPPSRDELTKNQVSLTCLVKGFYPSD |
| | IAVEWESNGQPENNYKTTPPVLDSDGSFFLYSKLTVDKSRWQQGNVFSCSVMHEALH |
| | NHYTQKSLSLSPGK |
| 197 | EDFKCMEALGMESGEIHSDQITASSQYSTNWSAERSRLNYPENGWTPGEDSYREWIQ |
| | VDLGLLRFVTAVGTQGAISKETKKKYYVKTYKIDVSSNGEDWITIEEGNKPVLFQGN |
| | TNPTDVVVAVFPKPLITRFVRIKPATWETGISMRFEVYGCGAPAPAGAPAPAGAPAP |
| | AGAPAPACMEALGMESGEIHSDQITASSQYSTNWSAERSRLNYPENGWTPGEDSYRE |
| | WIQVDLGLLRFVTAVGTQGAISKETKKKYYVKTYKIDVSSNGEDWITIEEGNKPVLF |
| | QGNTNPTDVVVAVFPKPLITRFVRIKPATWETGISMRFEVYGCGAPAPAGAPAPAGA |
| | PAPAGAPAPACMEALGMESGEIHSDQITASSQYSTNWSAERSRLNYPENGWTPGEDS |
| | YREWIQVDLGLLRFVTAVGTQGAISKETKKKYYVKTYKIDVSSNGEDWITIEEGNKP |
| | VLFQGNTNPTDVVVAVFPKPLITRFVRIKPATWETGISMRFEVYGCGAPAPAGAPAP |
| | AGAPAPAGAPAPAEPKSCDKTHTCPPCPAPELLGGPSVFLFPPKPKDTLMISRTPEV |
| | TCVVVDVSHEDPEVKFNWYVDGVEVHNAKTKPREEQYNSTYRVVSVLTVLHQDWLNG |
| | KEYKCKVSNKALPAPIEKTISKAKGQPREPQVYTLPPSRDELTKNQVSLTCLVKGFY |
| | PSDIAVEWESNGQPENNYKTTPPVLDSDGSFFLYSKLTVDKSRWQQGNVFSCSVMHE |
| | ALHNHYTQKSLSLSPGK |
| 198 | EDFKCMEALGMESGEIHSDQITASSQYSTNWSAERSRLNYPENGWTPGEDSYREWIQ |
| | VDLGLLRFVTAVGTQGAISKETKKKYYVKTYKIDVSSNGEDWITIEEGNKPVLFQGN |
| | TNPTDVVVAVFPKPLITRFVRIKPATWETGISMRFEVYGCGAPAPAGAPAPAGAPAP |
| | AGAPAPAEPKSCDKTHTCPPCPAPELLGGPSVFLFPPKPKDTLMISRTPEVTCVVVD |
| | VSHEDPEVKFNWYVDGVEVHNAKTKPREEQYNSTYRVVSVLTVLHQDWLNGKEYKCK |
| | VSNKALPAPIEKTISKAKGQPREPQVYTLPPSRDELTKNQVSLTCLVKGFYPSDIAV |
| | EWESNGQPENNYKTTPPVLDSDGSFFLYSKLTVDKSRWQQGNVFSCSVMHEALHNHY |
| | TQKSLSLSPGKGAPAPAGAPAPAGAPAPAGAPAPACMEALGMESGEIHSDQITASSQ |
| | YSTNWSAERSRLNYPENGWTPGEDSYREWIQVDLGLLRFVTAVGTQGAISKETKKKY |
| | YVKTYKIDVSSNGEDWITIEEGNKPVLFQGNTNPTDVVVAVFPKPLITRFVRIKPAT |
| | WETGISMRFEVYGCEVEA |
| 199 | ENFQCNVPLGMESGRIANEQISASSTYSDGRWTPQQSRLHGDDNGWTPNLDSNKEYL |
| | QVDLRFLTMLTAIATQGAISRETQNGYYVKSYKLEVSTNGEDWMVYEHGKNHKVFQA |
| | NNDATEVVLNKLHAPLLTRFVRIRPQTWHSGIALRLELFGCGAPAPAGAPAPAGAPA |
| | PAGAPAPACNVPLGMESGRIANEQISASSTYSDGRWTPQQSRLHGDDNGWTPNLDSN |
| | KEYLQVDLRFLTMLTAIATQGAISRETQNGYYVKSYKLEVSTNGEDWMVYEHGKNHK |
| | VFQANNDATEVVLNKLHAPLLTRFVRIRPQTWHSGIALRLELFGCGAPAPAGAPAPA |
| | GAPAPAGAPAPAEPKSCDKTHTCPPCPAPELLGGPSVFLFPPKPKDTLMISRTPEVT |
| | CVVVDVSHEDPEVKFNWYVDGVEVHNAKTKPREEQYNSTYRVVSVLTVLHQDWLNGK |
| | EYKCKVSNKALPAPIEKTISKAKGQPREPQVYTLPPSRDELTKNQVSLTCLVKGFYP |
| | SDIAVEWESNGQPENNYKTTPPVLDSDGSFFLYSKLTVDKSRWQQGNVFSCSVMHEA |
| | LHNHYTQKSLSLSPGK |
| 200 | ENFQCNVPLGMESGRIANEQISASSTYSDGRWTPQQSRLHGDDNGWTPNLDSNKEYL |
| | QVDLRFLTMLTAIATQGAISRETQNGYYVKSYKLEVSTNGEDWMVYEHGKNHKVFQA |
| | NNDATEVVLNKLHAPLLTRFVRIRPQTWHSGIALRLELFGCGAPAPAGAPAPAGAPA |
| | PAGAPAPACNVPLGMESGRIANEQISASSTYSDGRWTPQQSRLHGDDNGWTPNLDSN |
| | KEYLQVDLRFLTMLTAIATQGAISRETQNGYYVKSYKLEVSTNGEDWMVYEHGKNHK |
| | VFQANNDATEVVLNKLHAPLLTRFVRIRPQTWHSGIALRLELFGCGAPAPAGAPAPA |
| | GAPAPAGAPAPACNVPLGMESGRIANEQISASSTYSDGRWTPQQSRLHGDDNGWTPN |
| | LDSNKEYLQVDLRFLTMLTAIATQGAISRETQNGYYVKSYKLEVSTNGEDWMVYEHG |
| | KNHKVFQANNDATEVVLNKLHAPLLTRFVRIRPQTWHSGIALRLELFGCGAPAPAGA |
| | PAPAGAPAPAGAPAPAEPKSCDKTHTCPPCPAPELLGGPSVFLFPPKPKDTLMISRT |
| | PEVTCVVVDVSHEDPEVKFNWYVDGVEVHNAKTKPREEQYNSTYRVVSVLTVLHQDW |
| | LNGKEYKCKVSNKALPAPIEKTISKAKGQPREPQVYTLPPSRDELTKNQVSLTCLVK |
| | GFYPSDIAVEWESNGQPENNYKTTPPVLDSDGSFFLYSKLTVDKSRWQQGNVFSCSV |
| | MHEALHNHYTQKSLSLSPGK |
| 201 | ENFQCNVPLGMESGRIANEQISASSTYSDGRWTPQQSRLHGDDNGWTPNLDSNKEYL |
| | QVDLRFLTMLTAIATQGAISRETQNGYYVKSYKLEVSTNGEDWMVYEHGKNHKVFQA |
| | NNDATEVVLNKLHAPLLTRFVRIRPQTWHSGIALRLELFGCGAPAPAGAPAPAGAPA |
| | PAGAPAPAEPKSCDKTHTCPPCPAPELLGGPSVFLFPPKPKDTLMISRTPEVTCVVV |
| | DVSHEDPEVKFNWYVDGVEVHNAKTKPREEQYNSTYRVVSVLTVLHQDWLNGKEYKC |
| | KVSNKALPAPIEKTISKAKGQPREPQVYTLPPSRDELTKNQVSLTCLVKGFYPSDIA |
| | VEWESNGQPENNYKTTPPVLDSDGSFFLYSKLTVDKSRWQQGNVFSCSVMHEALHNH |
| | YTQKSLSLSPGKGAPAPAGAPAPAGAPAPAGAPAPACNVPLGMESGRIANEQISASS |
| | TYSDGRWTPQQSRLHGDDNGWTPNLDSNKEYLQVDLRFLTMLTAIATQGAISRETQN |
| | GYYVKSYKLEVSTNGEDWMVYEHGKNHKVFQANNDATEVVLNKLHAPLLTRFVRIRP |
| | QTWHSGIALRLELFGCDWTD |

Materials

Materials used in the present example are provided in the table below.

TABLE 25
Materials used in the CendR studies.

| Material | Source | Catalog No. | Lot No. |
|---|---|---|---|
| Recombinant RSV Protein | Antibodies Online | ABIN2006856 | |
| | Sino Biological | 11049-V08B | |
| Recombinant SARS-Cov-2 | Sino Biological | | |
| Protein | | | |
| NHS-Ester biotinylation Kit | ThermoFisher | A39256 | N/A |
| | Scientific | | |
| Anti-hIgG Fc Capture | Sartorius | 18-5060 | N/A |
| (AHC) Biosensors | | | |
| Streptavidin Biosensors | Sartorius | 18-5020 | |
| 1X Kinetic Buffer | Sartorius | 18-1105 | 910031 |
| Running buffer: HBST, pH: | Pinetree | | |
| 7.3 | therapeutics | | |
| Pierce Antibody | ThermoFisher | 90407 | |
| Biotinylation | Scientific | | |
| Kit for IP | | | |

CendR Peptides

The CendR peptides evaluated are provided in the table below.

TABLE 26
CendR peptides evaluated.

| Virus | Protein | Sequence | SEQ ID NO. | Catalog No. |
|---|---|---|---|---|
| Dengue | polyprotein | GTCTQSGERRREKR | 169 | 122453-1 |
| RSV | F | KNTNVTLSKKRKRR | 170 | 122453-2 |
| Hantavirus | glycoprotein | LTHKMIEESHRLRR | 171 | 122453-3 |
| EBV | EBNA3C | VSFKPPPPPSRRRR | 172 | 122453-4 |
| EBV (uncleaved) | EBNA3C | VSFKPPPPPSRRRRGACVVY | 173 | 122453-14 |
| SARS-COV-2 Wuhan | Spike | CASYQTQTNSPRRAR | 174 | 112551-7 |
| SARS-COV-2 Wuhan | Spike | CASYQTQTNSPRRARSVASQSIIAYTMSLG | 175 | 112551-5 |
| (uncleaved) | | | | |
| SARS-COV-2 UK | Spike | ASYQTQTNSHRRAR | 176 | 122453-5 |
| SARS-COV-2 India | Spike | ASYQTQTNSRRRAR | 177 | 122453-6 |
| SARS-COV-2 India | Spike | ASYQTQTNSRRRARSVASQSIIAY | 178 | 122453-15 |
| (uncleaved) | | | | |
| HCoV-OC43 | Spike | GSGYCVDYSKNRRSR | 179 | 122453-7 |
| MERS-COV | Spike | LLEPVSISTGSRSAR | 180 | 122453-8 |
| MERS-COV | Spike | LLEPVSISTGSRSARSAIEDLLFDK | 181 | 122453-16 |
| (uncleaved) | | | | |
| HSV-1 | VP22 | ERPRAPARSASRPRR | 182 | 122453-9 |
| HSV-1 | VP22 | ERPRAPARSASRPRRPV | 183 | 122453-17 |
| (uncleaved) | | | | |
| IAV H5N1 | Hemagglutinin | VLATGLRNVPQRKKR | 184 | 122453-10 |
| HPV | L1 | PTTSSTSTTAKRKKR | 185 | 122453-11 |
| Human | attachment | IDMLKARVKNRVAR | 186 | 122453-12 |
| Metapneumovirus | glycoprotein | | | |
| HIV | GP-160/GP-120 | AKRRVVQREKR | 187 | 122453-13 |
| HIV (uncleaved) | GP-160 | AKRRVVQREKRAVGIGALFLG | 188 | 122453-18 |

Construct molecules binding viral proteins and CendR peptide as well as the K_{D} determination were performed using the Octet Red 96 system, as described below.

Construct Molecules Binding to Viral Proteins: Methods

Construct molecules were immobilized at varying concentrations (0.55-1.56 μg/mL) of in 1×Kinetic buffer (1×PBS pH 7.4, 0.1% w/v BSA, 0.002% v/v Tween-20) onto Anti-hIgG Fc biosensors with a loading time of 180 seconds for each. Baseline post-loading was performed for 60 seconds. Next, 25 nM, 50 nM, and 100 nM of recombinant viral protein/s were prepared in 1×Kinetic Buffer. Reference sensors were generated by applying Viral proteins over a blank Anti-hIgG Fc biosensors (no construct molecules). The association (Kₐ) and dissociation (K_{d}) steps were 300 seconds each. Data was analyzed using the Octet analysis software with 1:1 or 2:1 model fit applied which reports a dissociation constant K_{D} (M).

Construct Molecules Binding to Biotinylated CendR Peptide: Methods

Constructs having the amino acid sequences set forth in SEQ ID NOs: 113, 122, 154, and 193 were evaluated. CendR peptides were immobilized at varying concentrations (0.05-0.65 μg/mL) of in 1×Kinetic buffer (described above) onto streptavidin (SA) biosensors with a loading time of 180 seconds for each. Baseline post-loading was performed for 60 seconds. Next, 100 nM of the construct molecules having an amino acid sequence set forth in SEQ ID NOs: SEQ ID NOs: 113, 122, 154, and 193 molecules were prepared in 1×Kinetic Buffer. Reference sensors were generated by applying construct molecules over a blank streptavidin biosensors surface (no CendR peptide). The association (Kₐ) and dissociation (K_{d}) steps were 300 seconds each. The data was analyzed using the Octet analysis software with 1:1 model fit applied which reports a dissociation constant K_{D} (M).

Results

Binding of Construct Molecules to Recombinant RSV F Protein

The impact of constructs binding RSV-F proteins with high affinity increases the likelihood of the platform being able to provide a therapeutic mechanism to fight RSV (see table below).

TABLE 27
Monovalent Binding kinetics of Vi-TRAP
Against Various Viral Proteins.

| SEQ ID NO. | Target | K_{D} |
|---|---|---|
| 113 | RSV F protein | <1 nM |
| 121 | RSV F protein | <1 nM |
| 122 | RSV F protein | <1 nM |
| 135 | RSV F protein | <1 nM |
| 136 | RSV F protein | <1 nM |
| 137 | RSV F protein | <1 nM |
| 115 | RSV F protein | <1 nM |
| 116 | RSV F protein | <1 nM |
| 133 | RSV F protein | <1 nM |

Binding of Construct Molecules to the CendR Peptide

The table below shows the results of the study evaluating construct molecule binding to CendR peptides.

TABLE 28
Monovalent binding kinetics of construct molecules against
various viral proteins. Here, “+++” means
strongest (top tier) binding signal; “++”
means tier two binding; “+” means lowest tier
binding signal; and “NB” means none binding.

| | CendR | Construct SEQ ID NO. | | | |
|---|---|---|---|---|---|
| CendR Sequence Species | SEQ ID NO. | 113 | 122 | 154 | 193 |
| Dengue | 169 | ++ | +++ | + | NB |
| RSV | 170 | + | +++ | ++ | NB |
| Hantavirus | 171 | ++ | +++ | + | NB |
| EBV | 172 | ++ | +++ | + | NB |
| EBV (uncleaved) | 173 | NB | NB | + | NB |
| SARS-CoV-2 Wuhan | 174 | ++ | +++ | + | NB |
| SARS-CoV-2 Wuhan | 175 | NB | NB | + | NB |
| (uncleaved) | | | | | |
| HCoV-OC43 | 176 | ++ | +++ | + | NB |
| MERS-CoV | 177 | ++ | +++ | + | NB |
| MERS-CoV (uncleaved) | 178 | NB | NB | + | NB |
| HSV-1 | 179 | ++ | +++ | + | NB |
| HSV-1 (uncleaved) | 180 | NB | NB | + | NB |
| IAV H5N1 | 181 | ++ | +++ | + | NB |
| HPV | 182 | ++ | +++ | + | NB |
| Human Metapneumovirus | 183 | ++ | +++ | + | NB |
| HIV | 184 | ++ | +++ | + | NB |
| HIV (uncleaved) | 185 | NB | NB | NB | NB |

Conclusions

The goals of the studies described herein were to establish binding characteristics and determine K_{D} of construct molecules to recombinant RBD viral protein and the CendR peptide Based on the data presented, the following conclusions can be made: (1) the construct molecules bind RSV F protein with high affinity in the monovalent format; and (2) Constructs having an amino acid sequence set forth in SEQ ID NOs: 113, 122, and 154 exhibit broad spectrum of viral CendR motifs in the octet assay format. Taken together, the data presented here suggests that the biological effects of the construct molecules of the present invention bind and may prevent infectivity.

### Example 15. ELISA Testing of Respiratory Syncytial Virus (RSV)

Construct molecules were evaluated testing binding affinity of constructs of the present invention to respiratory syncytial virus (RSV) F glycoprotein via ELISA.

Reagents and Materials

Reagents used were as follows: 1×DPBS (Corning, Corning, NY; Catalog No. 21-031-CM); Tween-20 (100%) (Boston BioProducts, Ashland, MA; Catalog No. P-934); Washing buffer: PBST (0.02% Tween-20 in 1×DPBS); Dry Milk Powder (Research Products International, Mt. Prospect, IL; Catalog No. M17200-1000.0); RSV-F (amino acids 1-529), (Extracellular Domain) protein (His tag), ABIN2006856, (Antibodies-online, Inc, Limerick, PA); Anti-Human IgG-HRP Conjugate (Abcam, Cambridge, United Kingdom; Catalog No. ab6759); TMB ELISA Substrate (High Sensitivity) (Abcam, Cambridge, United Kingdom; Catalog No. ab171523); ELISA STOP Solution (Invitrogen by ThermoFisher Scientific, Waltham, MA; Catalog No. SS04); and Pierce™ 96-well high binding ELISA plate (ThermoFisher Scientific, Waltham, MA; Catalog No. 15041).

Equipment

The ELISA testing was performed with the following equipment: 450 nm 96 well SpectraMax M2e Microplate Reader (Molecular Devices, San Jose, CA); Wellwash Versa Microplate Washer (ThermoFisher Scientific, Waltham, MA); P20, P200, P1000, and Multi-channel pipets (Eppendorf).

Procedure

The ELISA testing was performed according to the following steps:

Step 1: Plate 100 μL of 3 μg/mL of RSV F protein in PBS in all the wells of a 96-well high protein bind plate using a multichannel pipettor; Step 2: Incubate plate overnight at 2-8C; Step 3: Wash plates 3× with 1×PBS with 0.05% Tween-20 (PBST); Step 4: Add 200 μL per well on all plates with 5% Dry Milk in 1×PBS; Step 5: Incubate plates 1 hour at room temperature; Step 6: Wash plate 3× with 1×PBS with 0.05% Tween-20 (PBST); Step 7: Add 100 μL of PBS in columns 8-12; Step 8: Add 200 μL (1 μg/mL in PBS) of each construct in wells A1 to H1; Step 9: Using a multichannel pipettor serially transfer 100 μL of each construct from Col1 to Col 11. Discard the extra 100 μL from Col 11. Leave Col 12 with PBS only; Step 10: Incubate 1 hour at RT; Step 11: Wash plate 3× with 1×PBS with 0.05% Tween-20 (PBST); Step 12: Add 200 μL of Rabbit anti-human IgG HRP at a 1:20,000 dilution in PBS to all wells on all plates; Step 13: Incubate 1 hour at RT; Step 14: Wash plate 3× with 1×PBS with 0.05% Tween-20 (PBST); Step 15: Add 100 μL of TMB reagent to all wells; Step 16: Incubate at room temperature for 15 minutes; Step 17: Add 100 μL of stop reagent to each well; Step 18: Read Absorption at ABS450.

Results of the ELISA assay are presented in the tables below.

TABLE 29
ELISA results. Half maximal effective concentration
(EC₅₀) amounts are shown in nM.

| SEQ ID NO. | EC₅₀ (nM) |
|---|---|
| 122 | 23.3 |
| 154 | 185.2 |
| 203 | 69.71 |
| 192 | 53.86 |
| 128 | 42.47 |
| 129 | 592.6 |
| 204 | 91.47 |
| 205 | 39.19 |
| 206 | 22.72 |
| 193 | 749.2 |
| 207 | 17.07 |
| 208 | 1570 |

TABLE 30
Half maximal effective concentration (EC₅₀) amounts
for a given construct as determined by a Varioskan
Plate reader (ThermoFisher) and SpectroMax.

EC50 (nM)

| SEQ ID NO. | Varioskan | SpectroMax |
|---|---|---|
| 113 | 372.10 | 323.10 |
| 191 | 91.00 | 95.65 |
| 121 | 5.65 | 5.90 |

### Example 16. Binding Analysis of Respiratory Syncytial Virus (RSV)

Construct molecules were evaluated testing binding affinity of constructs of the present invention to respiratory syncytial virus (RSV) F glycoprotein via an Octet® Red 96 system (ForteBio).

TABLE 31
Binding results of constructs and RSV.

| | | | | Reduced | | |
|---|---|---|---|---|---|---|
| SEQ ID | | | | Heparin | VEGF | |
| NO. | Structure | Mutation | Strategy | binding | binding | Results |
| 113 | b1-Fc | WT | single b1 | No | Unmodified | + |
| 114 | b1-Fc | K373E, | single b1, heparin | Yes | Unmodified | No |
| | | | binding ↓(E) in | | | binding |
| | | | b1 | | | |
| 121 | b1b1- | WT | quadruple b1 | No | Unmodified | ++ |
| | b1b1-Fc | | | | | |
| 135 | b1b1-Fc | WT | single b1 in N- | No | Unmodified | ++ |
| | | | term and C-term | | | |
| 137 | b1-Fc-b1 | WT | single b1 in N- | No | Unmodified | + |
| | | | term and C-term | | | |

Here, “+++” means strongest (top tier) binding signal; “++” means tier two binding; “+” means lowest tier binding signal; and “NB” means none binding. “↓” means decreased. “(E)” means the amino acid is substituted with a glutamic acid residue.

### Example 17. ELISA Testing of SARS-CoV-2

Construct molecules were evaluated testing binding affinity of constructs of the present invention to SARS-CoV-2 via ELISA. The ELISA was performed according the methods described in Example 15 above.

TABLE 32
ELISA results.

| SEQ | | | | Reduced | | |
|---|---|---|---|---|---|---|
| ID | | Mu- | | Heparin | VEGF | EC₅₀ |
| NO. | Structure | tation | Strategy | binding | binding | (nM) |
| 122 | b1b2-Fc | WT | single b1b2 | No | Un- | 3.09 |
| | | | | | modified | |
| 138 | b1b2- | WT | double b1b2 | No | Un- | 109.8 |
| | b1b2-Fc | | | | modified | |
| 139 | b1b2- | E319A | double b1b2 | No | Increased | 81.72 |
| | b1b2-Fc | | (VEGF binding | | | |
| 142 | b1b2-Fc- | WT | single b1b2 in | No | Un- | Un- |
| | b1b2 | | N-term and C- | | modified | stable |
| | | | term | | | |

Half maximal effective concentration (EC₅₀) amounts are shown in nM.
N-term and C-term means N-terminus and C-terminus, respectively.
“↓” means increased.

### Example 18. Binding Analysis of Influenza A H5N1 Virus (IAV H5N1)

Construct molecules were evaluated testing binding affinity of constructs of the present invention to IAV H5NT via an Octet® Red 96 system (ForteBio).

TABLE 33

| | | | | Reduced | | |
|---|---|---|---|---|---|---|
| SEQ ID | | | | Heparin | VEGF | |
| NO. | Structure | Mutation | Strategy | binding | binding | Results |
| 113 | b1-Fc | WT | single b1 | No | Unmodified | 18.95 |
| | | | | | | nM |
| 114 | b1-Fc | K373E, | single b1, heparin | Yes | Unmodified | ND |
| | | | binding ↓(E) in b1 | | | |
| 115 | b1b1-Fc | K373E, | double b1, heparin | Yes | Unmodified | ND |
| | | | binding ↓ (E) in b1 | | | |
| 116 | b1b1b1-Fc | K373E, | triple b1, heparin | Yes | Unmodified | ND |
| | | | binding ↓ (E) in b1 | | | |
| 121 | b1b1- | WT | quadruple b1 | No | Unmodified | 7.817 |
| | b1b1-Fc | | | | | pM |
| 122 | b1b2-Fc | WT | single b1b2 | No | Unmodified | 17.46 |
| | | | | | | nM |
| 155 | b1b2-Fc | R513A, | single b1b2, | Yes | Unmodified | ND |
| | | K514A, | heparin binding ↓ | | | |
| | | K516A | (A) in b2 | | | |
| 128 | b1b2-Fc | K358E, | single b1b2, | Yes | Unmodified | ND |
| | | K373E, | heparin binding ↓ | | | |
| | | R513E, | in b1b2 | | | |
| | | K514E, | | | | |
| | | K516E | | | | |
| 129 | b1b2-Fc | Y297A, | single b1b2, no | Yes | Removed | ND |
| | | S345A, | VEGF binding, | | | |
| | | Y353A, | heparin binding ↓ | | | |
| | | K358E, | in b1b2 | | | |
| | | K373E, | | | | |
| | | R513E, | | | | |
| | | K514E, | | | | |
| | | K516E | | | | |
| 133 | b1-Fc-b1 | K373E, | single b1 in N-term | Yes | Unmodified | ND |
| | | | and C-term, | | | |
| | | | heparin binding (E) | | | |
| | | | ↓ in b1 | | | |
| 135 | b1b1-Fc | WT | single b1 in N-term | No | Unmodified | ND |
| | | | and C-term | | | |
| 136 | b1b1b1-Fc | WT | single b1 in N-term | No | Unmodified | ND |
| | | | and C-term | | | |
| 137 | b1-Fc-b1 | WT | single b1 in N-term | No | Unmodified | ND |
| | | | and C-term | | | |

ND = Not determined.
N-term and C-term means N-terminus and C-terminus, respectively.
“↓” means decreased “(A)” means the amino acid is substituted with an alanine residue. “(E)” means the amino acid is substituted with a glutamic acid residue.

### Example 19. Bioinformatic Approaches to Identifying CendR

Predicting Furin Cleavage Sites

Furin is a conserved protease that cleaves a “R-X-[KR]-R” motif resulting in CendR peptides. Furin is an ancient gene family with orthologs in all vertebrates and paralogs dating back to the divergence of animals and fungi.

In order to identify furin cleavage sites in human, viral, and bacterial proteins—and the subsequent formation of CendR peptides for targeting by constructs of the present invention—a bioinformatic evaluation was performed to identified extended consensus sequences.

To identify furin cleavage sites, a RefSeq peptide database was evaluated, comprising the following records: Human: 114,963 records; Viruses: 477,258 records; and Bacteria: 161,430,766 records. In addition, two prediction software programs were used: ProP prediction software, and the PiTou Furin cleavage site computational prediction tool.

The ProP tool is a neural network with 94.7% sensitivity and 83.7% specificity. An exemplary description of ProP is provided in Duckert et al., Prediction of proprotein convertase cleavage sites Protein Eng Des Sel. 2004 Jan. 17(1):107-12, the disclosure of which is incorporated herein by reference in its entirety.

PiTou Furin cleavage site computational prediction tool (hereinafter “PiTou”) is a knowledge-based tool, with 96.9% sensitivity and 97.3% specificity. PiTou is based on 131 known furin cleavage sites; and 4265 arginine sites where furin does not cleave. Log(odds) scores are provided based on a profile Hmm of furin binding sites and physical properties such as volume, charge, and hydrophobicity.

An exemplary description of the PiTou Furin cleavage site computational prediction tool is provided in Tian et al., Computational prediction of furin cleavage sites by a hybrid method and understanding mechanism underlying diseases. Sci Rep. 2012; 2: 261, the disclosure of which is incorporated herein by reference in its entirety.

The RefSeq peptide results are shown in the table below.

TABLE 34
RefSeq peptide results

| | No. of sites | No. of distinct | |
|---|---|---|---|
| Database | (Score >0) | peptides | Database size |
| Human | 101,892 | 50,046 | 115,140 |
| Viruses | 117,993 | 80,359 | 477,258 |
| Bacteria | 60,546,480 | 42,054,923 | 161,430,766 |

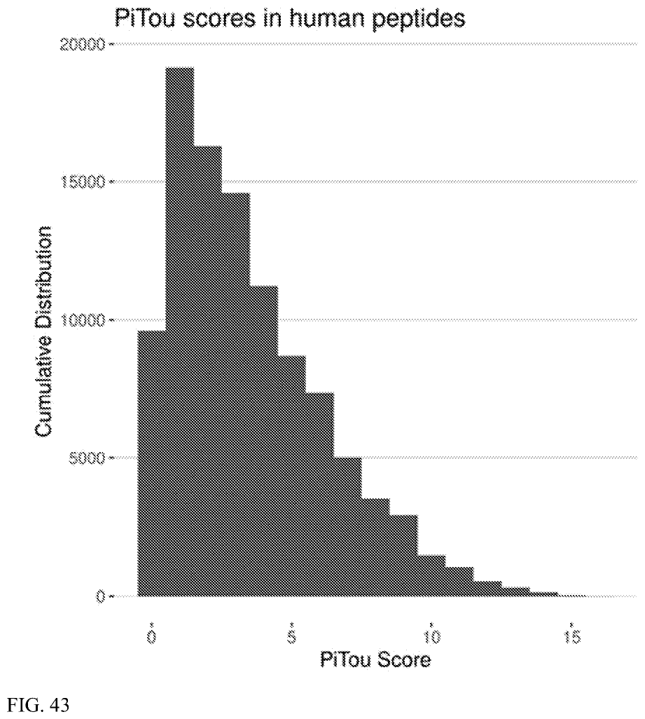

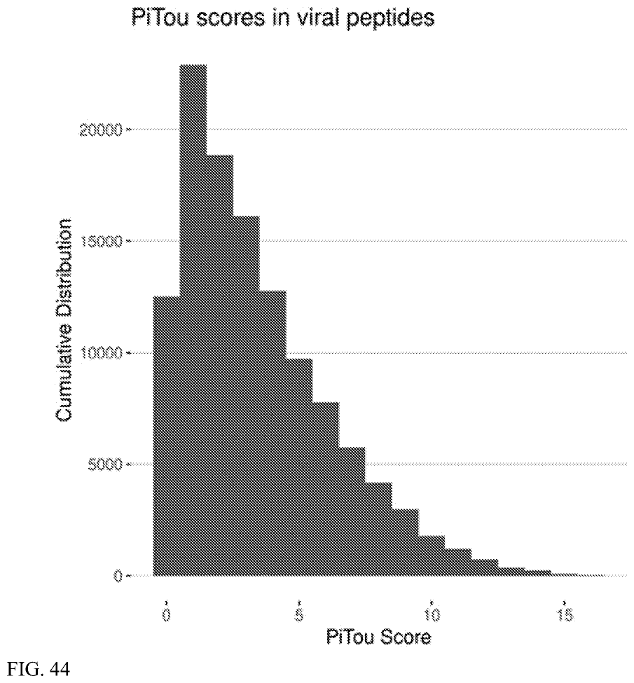

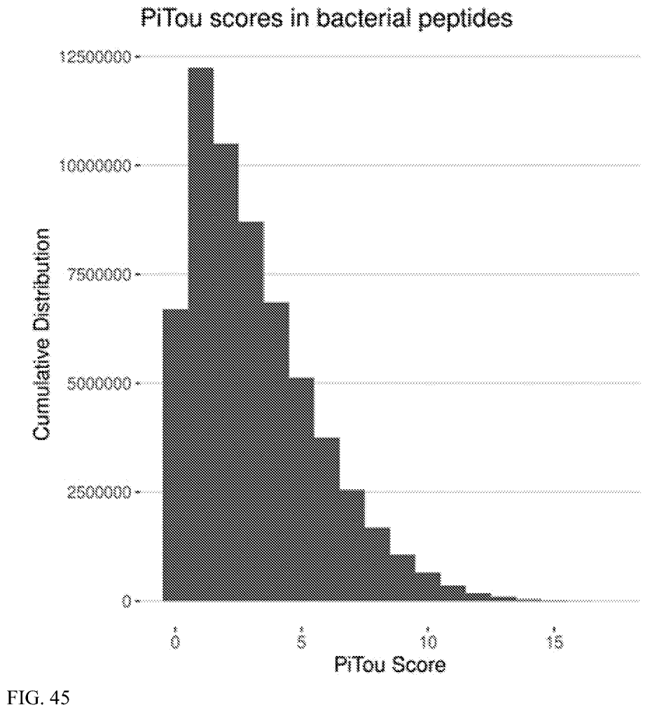

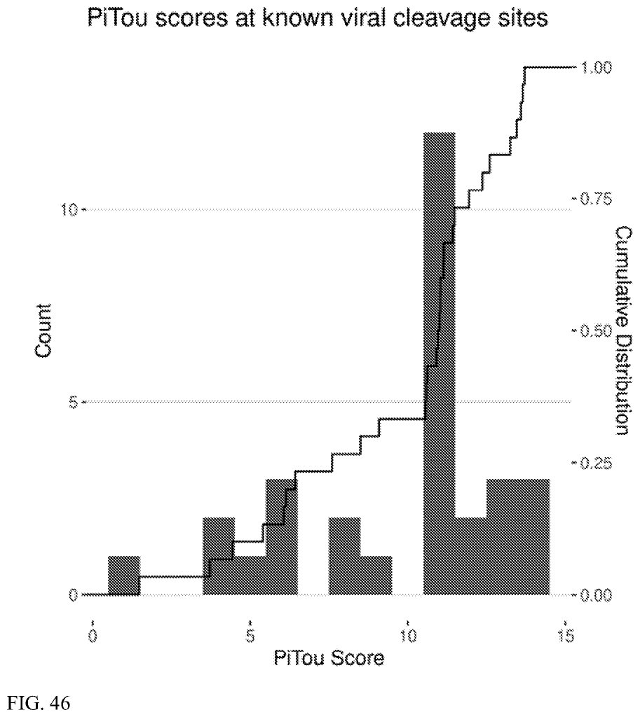

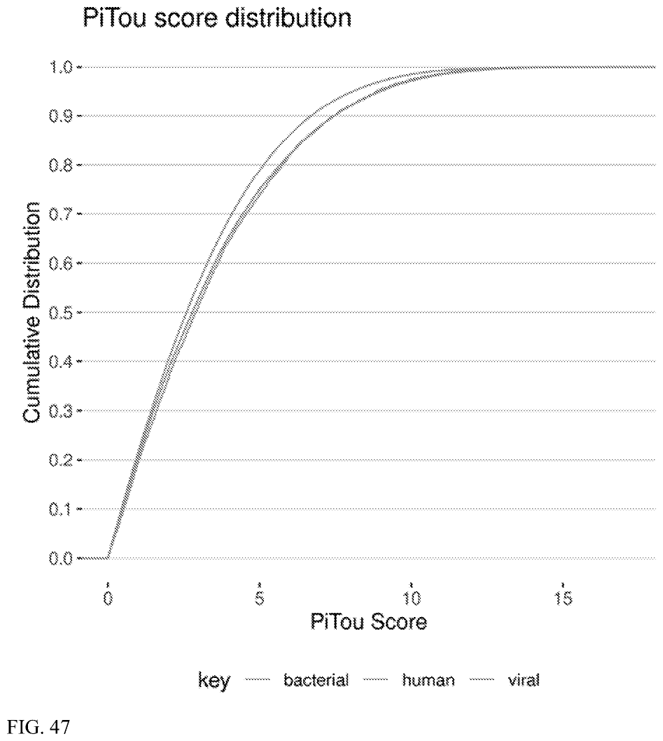

FIGS. 43-45 show the cumulative distribution of PiTou scores in human, viral, and bacterial peptides, respectively. FIG. 46 shows the PiTou scores at known viral cleavage sites. Known sites were based off of 30 viruses, and the PiTou scores were as follows: 90%>5 (99%); 75%>7 (99.9%); and 50%>11 (99.99%). FIG. 47 shows a prioritized PiTou score distribution.

Novel predicted targets were prioritized based on known diseases, PiTou score, secreted peptides, and conservation. Human known diseases were identified based on OMIM, ClinVar, GnomAD, and MGI. Bacteria and viruses were identified based on WHO, RKI, the Bode Science Center, and Major Infectious Diseases 3^{rd} Ed.

TABLE 35
PiTou score In(odds).

| PiTou Score | Odds | Probability |
|---|---|---|
| 0 | 1:1 | 50% |
| 2.3 | 10:1 | 90% |
| 4.6 | 100:1 | 99% |
| 6.9 | 1000:1 | 99.9% |
| 9.2 | 10000:1 | 99.99% |
| 11.5 | 100000:1 | 99.999% |
| 13.8 | 1000000:1 | 99.9999% |

Secreted peptides were prioritized based on DeepSig, a neural network with separate models for eukaryotes and bacterial; Cell-Ploc 2.0 (a pre-computed subcellular localization of different proteins); and Spdb 5.1 (a database of signal peptides in Swiss-Prot & EMBL entries; see Choo, et al. 2005).

An exemplary description of DeepSig is provided in Savojardo et al., DeepSig: deep learning improves signal peptide detection in proteins. Bioinformatics. 2018 May 15; 34(10):1690-1696, the disclosure of which is incorporated herein by reference in its entirety.

An exemplary description of Cell-Ploc 2.0 is provided in Chou and Shen, Cell-PLoc: a package of Web servers for predicting subcellular localization of proteins in various organisms, Nat Protoc. 2008; 3(2):153-62, the disclosure of which is incorporated herein by reference in its entirety.

An exemplary description of Spdb 5.1 is provided in Choo et al., 2005, the disclosure of which is incorporated herein by reference in its entirety.

TABLE 36
Top scoring viral proteins with predicted furin cleavage sites. Here,
the column entitled “Protein” shows the NCBI Accession No. of the predicted protein. The
column entitled “Score” shows the PiTou score.

| Protein | Description | R_position | Motif-P14-P1 | Score |
|---|---|---|---|---|
| NP_041724.2 | polyprotein [West Nile | 215 | GRCTKTRHSRRSRR | 15.3974 |
| | virus] | | | |
| YP_001129354.1 | ORF8 [Human | 440 | ASPMTTSASRRKRR | 15.2278 |
| | gammaherpesvirus 8] | | | |
| YP_009273009.1 | nucleocapsid protein | 433 | ENDKPKSQRRRKKR | 14.6575 |
| | [Rousettus bat | | | |
| | coronavirus] | | | |
| YP_173238.1 | spike glycoprotein | 760 | YNSPSSSSSRRKRR | 14.6348 |
| | [Human coronavirus | | | |
| | HKU1] | | | |
| YP_009337505.1 | Cy89 [Cynomolgus | 432 | EHANNTNATRRRKR | 14.6088 |
| | cytomegalovirus] | | | |
| NP_044011.1 | MC60R [Molluscum | 364 | ALPAARPRRSRTRR | 13.8656 |
| | contagiosum virus subtype | | | |
| | 1] | | | |
| NP_044078.1 | MC127R [Molluscum | 143 | PALRGGSYRARKRR | 13.8432 |
| | contagiosum virus subtype | | | |
| | 1] | | | |
| YP_209233.1 | spike glycoprotein (S) | 769 | GLCVDYSKSRRARR | 13.7021 |
| | [Murine hepatitis virus | | | |
| | strain JHM] | | | |
| NP_043135.1 | polyprotein [Tick-borne | 205 | YGRCGKQEGSRTRR | 13.647 |
| | encephalitis virus] | | | |
| YP_308669.1 | hemagglutinin [Influenza | 346 | LRNTPQRERRRKKR | 13.5885 |
| | A virus | | | |
| | (A/goose/Guangdong/1/ | | | |
| | 1996(H5N1))] | | | |
| YP_009137477.1 | UL55 [Papio ursinus | 453 | ANMTNASTSSRTKR | 13.3161 |
| | cytomegalovirus] | | | |
| NP_041726.1 | polyprotein precursor | 210 | GKCDSAGRSRRSRR | 13.2778 |
| | [Yellow fever virus] | | | |
| NP_073286.1 | polyprotein [Dengue virus | 204 | GTCTQSGERRREKR | 13.2573 |
| | 4] | | | |
| YP_004940082.1 | envelope glycoprotein B | 451 | SVVNATSKSSRRRR | 13.165 |
| | [Aotine betaherpesvirus 1] | | | |
| NP_040831.1 | spike protein [Infectious | 690 | ISLLLTNPSSRRKR | 12.7323 |
| | bronchitis virus] | | | |
| NP_044095.1 | MC144R [Molluscum | 213 | RAPPLRALLRRLRR | 12.3666 |
| | contagiosum virus subtype | | | |
| | 1] | | | |
| YP_073779.1 | envelope glycoprotein B | 398 | LENFSNASRKRRKR | 12.3138 |
| | [Human betaherpesvirus | | | |
| | 7] | | | |
| NP_044005.1 | MC054L [Molluscum | 162 | PENADAAGARRRRR | 12.2349 |
| | contagiosum virus subtype | | | |
| | 1] | | | |
| NP_042932.1 | glycoprotein B [Human | 399 | MFLEQGSEKIRRRR | 12.0529 |
| | betaherpesvirus 6A] | | | |
| YP_001129508.1 | BALF4 [Human | 432 | RGSTSAAVLRRRRR | 12.0276 |
| | herpesvirus 4 type 2] | | | |
| YP_401713.1 | envelope glycoprotein B | 432 | RGSTPAAVLRRRRR | 11.9466 |
| | [Human | | | |
| | gammaherpesvirus 4] | | | |
| YP_009094054.1 | capsid [Rabbit astrovirus | 21 | VPSRGQRARRRRAR | 11.8699 |
| | TN/2208/2010] | | | |
| YP_009010983.1 | ORF2 [Porcine astrovirus | 30 | TTVSNRRGRRRRRR | 11.7415 |
| | 4] | | | |
| YP_009362094.1 | glycoprotein precursor | 124 | LTHKMIEESHRLRR | 11.5376 |
| | [Sangassou | | | |
| | orthohantavirus] | | | |
| NP_044596.1 | Fusion protein (F) | 136 | KNTNVTLSKKRKRR | 11.4852 |
| | [Respiratory syncytial | | | |
| | virus] | | | |
| NP_056863.1 | fusion glycoprotein | 136 | KNLNVSISKKRKRR | 11.4368 |
| | [Human | | | |
| | orthopneumovirus] | | | |
| NP_056922.1 | fusion protein [Measles | 112 | RPVQSVASSRRHKR | 11.4277 |
| | morbillivirus] | | | |
| YP_009505809.1 | capsid protein precursor | 69 | GQGPRNRRRSRLRR | 11.3216 |
| | [Bat astrovirus | | | |
| | Hp/Guangxi/LC03/2007] | | | |
| YP_009256201.1 | nucleocapsid protein | 29 | PSQKRGRSRSRSRR | 11.2968 |
| | [Ferret coronavirus] | | | |
| NP_042099.1 | putative DNA-binding | 137 | DISDVKVLAARLKR | 11.2105 |
| | virion core protein | | | |
| | [Variola virus] | | | |
| YP_001129465.1 | EBNA-3C [Human | 501 | VSFKPPPPPSRRRR | 11.1623 |
| | herpesvirus 4 type 2] | | | |
| NP_043984.1 | MC033L [Molluscum | 578 | RSTRTARRARRARR | 11.1578 |
| | contagiosum virus subtype | | | |
| | 1] | | | |
| NP_054711.1 | fusion protein [Mumps | 102 | NIASPSSGSRRHKR | 11.1454 |
| | rubulavirus] | | | |
| NP_057863.1 | p27 [Human T-cell | 10 | MPKTRRRPRR | 11.0752 |
| | leukemia virus type I] | | | |
| YP_001469634.1 | polyprotein [Hepatitis C | 2197 | SHITAETASRRLKR | 11.0478 |
| | virus genotype 6] | | | |
| YP_081514.1 | envelope glycoprotein B | 460 | NRSSLNLTHNRTKR | 11.0306 |
| | [Human betaherpesvirus | | | |
| | 5] | | | |
| NP_056776.2 | polyprotein [Dengue virus | 205 | GTCTTMGEHRREKR | 11.0097 |
| | 2] | | | |
| NP_043979.1 | MC028L [Molluscum | 19 | RRKSKYQPLRRQRR | 11.0076 |
| | contagiosum virus subtype | | | |
| | 1] | | | |
| NP_057865.1 | gp46 SU [Human T-cell | 312 | SLSPVPTLGSRSRR | 10.9953 |
| | leukemia virus type I] | | | |
| NP_049560.1 | reverse | 1041 | SLSPVPTLGSRSRR | 10.9953 |
| | transcriptase/envelope | | | |
| | protein [Simian T- | | | |
| | lymphotropic virus 1] | | | |
| NP_690589.2 | structural polyprotein | 325 | LQASLTCSPHRQRR | 10.9598 |
| | [Chikungunya virus] | | | |
| NP_042023.1 | glycoprotein [Borna | 249 | YTSSCRPRLKRRRR | 10.9553 |
| | disease virus 1] | | | |
| YP_009555245.1 | nucleocapsid protein | 274 | EVROKILNKPRQKR | 10.8307 |
| | [Human coronavirus | | | |
| | OC43] | | | |
| NP_043993.1 | MC042L [Molluscum | 343 | LVGDTRRPPARPRR | 10.7428 |
| | contagiosum virus subtype | | | |
| | 1] | | | |
| YP_009824998.1 | spike glycoprotein | 537 | FYIKITNGTRRFRR | 10.7175 |
| | [Infectious bronchitis | | | |
| | virus] | | | |
| YP_007188578.1 | ORF1a polyprotein | 1106 | QSVTVKPKRLRKKR | 10.6809 |
| | [Betacoronavirus England | | | |
| | 1] | | | |
| YP_009047202.1 | 1AB polyprotein [Middle | 1106 | QNVTVKPKRLRKKR | 10.6809 |
| | East respiratory syndrome- | | | |
| | related coronavirus] | | | |
| YP_009755897.1 | spike protein [Canada | 558 | QYYQLFSNNTRRKR | 10.6322 |
| | goose coronavirus] | | | |
| YP_009361855.1 | ORFla protein [Bat | 1102 | QIVVSKPKRLRKKR | 10.5947 |
| | coronavirus] | | | |
| NP_040154.2 | envelope glycoprotein B | 494 | SPQKHPTRNTRSRR | 10.5828 |
| | [Human alphaherpesvirus | | | |
| | 3] | | | |
| YP_009163895.1 | L2 [Human | 9 | MYRTKRVKR | 10.5448 |
| | papillomavirus] | | | |
| YP_009664789.1 | capsid protein precursor | 48 | STTTAPRAPRRRRR | 10.5338 |
| | [Mamastrovirus 18] | | | |
| YP_009380532.1 | capsid protein | 45 | RAQQARRRRNRTRR | 10.5301 |
| | [Mamastrovirus 2] | | | |
| YP_009272536.1 | polyprotein [Hepatitis C | 2196 | SHITAETAARRLRR | 10.4354 |
| | virus genotype 7] | | | |
| NP_056788.1 | capsid protein [Hepatitis E | 333 | VSRYSSTARHRLRR | 10.2448 |
| | virus] | | | |
| NP_598404.1 | fusion protein [Human | 106 | KISTVTDTKTRQKR | 9.72278 |
| | rubulavirus 2] | | | |
| YP_009268921.1 | glycoprotein [Borna | 249 | YTSSCRPRLVRRRR | 9.65791 |
| | disease virus 2] | | | |
| NP_041006.1 | env propeptide, (putative; | 308 | SLAPVPPPATRRRR | 9.40027 |
| | first expressed exon); | | | |
| | putative [Human T- | | | |
| | lymphotropic virus 2] | | | |
| YP_009724390.1 | surface glycoprotein | 685 | ASYQTQTNSPRRAR | 9.19633 |
| | [Severe acute respiratory | | | |
| | syndrome coronavirus 2] | | | |
| YP_009141014.1 | glycoprotein precursor | 1060 | WILRVVLRRSRIRR | 8.95328 |
| | [Bhanja virus] | | | |
| YP_002455789.1 | envelope glycoprotein | 307 | LSPLPGAPLTRRRR | 8.92879 |
| | [Human T-lymphotropic | | | |
| | virus 4] | | | |
| YP 009047204.1 | spike protein [Middle East | 887 | LEPVSISTGSRSAR | 8.55386 |
| | respiratory syndrome- | | | |
| | related coronavirus] | | | |
| YP 007188579.1 | spike protein | 887 | LEPVSISTGSRSAR | 8.55386 |
| | [Betacoronavirus England | | | |
| | 1] | | | |
| YP 142353.1 | transmembrane | 490 | RVCCKRVRRRRSGR | 8.3646 |
| | glycoprotein G [Mokola | | | |
| | lyssavirus] | | | |
| YP 009137152.1 | envelope glycoprotein L | 159 | RAGCVNFDYSRTRR | 8.32858 |
| | [Human alphaherpesvirus | | | |
| | 2] | | | |
| YP_009137096.1 | envelope glycoprotein H | 545 | AGVPSAVQRERARR | 7.98401 |
| | [Human alphaherpesvirus | | | |
| | 1] | | | |
| NP_950235.1 | glycoprotein precursor | 247 | VQDTHPSPTNRSKR | 7.60291 |
| | [Crimean-Congo | | | |
| | hemorrhagic fever | | | |
| | orthonairovirus] | | | |
| YP_009254001.1 | G [Lonestar tick chuvirus | 618 | VNPEWTPFRERLRR | 7.57886 |
| | 1] | | | |
| NP_050220.1 | Glycoprotein B [Human | 399 | MVLEQGSVNLRRRR | 7.3109 |
| | betaherpesvirus 6B] | | | |
| YP_009055225.1 | glycoprotein [Marburg | 435 | SPTTRPPIYFRKKR | 7.06092 |
| | marburgvirus] | | | |
| NP_056796.1 | transmembrane | 218 | GMSCDIFTNSRGKR | 6.6124 |
| | glycoprotein G [Rabies | | | |
| | lyssavirus] | | | |
| NP_982303.1 | polyprotein [Oropouche | 707 | ASKLLDINLGRSTR | 6.2933 |
| | virus] | | | |
| YP_001531156.1 | glycoprotein [Marburg | 435 | SSTAQHLVYFRRKR | 6.13352 |
| | marburgvirus] | | | |
| YP_009227121.1 | glycoprotein precursor | 516 | LLFVCKAVKGIKRR | 5.42962 |
| | [Tofla virus] | | | |
| YP_003848705.1 | glycoprotein [Rift Valley | 466 | KPLCVGYERVVVKR | 5.24779 |
| | fever virus] | | | |
| YP_009555241.1 | spike surface glycoprotein | 757 | SGYCVDYSKNRRSR | 4.64604 |
| | [Human coronavirus | | | |
| | OC43] | | | |
| YP_009362075.1 | glycoprotein precursor | 291 | GAKFETSDRIRLHR | 4.63 |
| | [Kaeng Khoi virus] | | | |
| NP_067151.1 | fusion protein [Human | 109 | NQESNENTNPRTKR | 4.43207 |
| | respirovirus 3] | | | |
| YP_009177714.1 | glycoprotein [Tacheng | 267 | HLGPNKEYMRVLRR | 4.22628 |
| | Tick Virus 4] | | | |
| YP_009512922.1 | polyprotein, partial [Tete | 728 | NYWAHLENTORVQR | 4.1588 |
| | orthobunyavirus] | | | |
| YP_009118475.1 | hemagglutinin [Influenza | 463 | ADSEMDKLYERVKR | 3.97254 |
| | A virus | | | |
| | (A/Shanghai/02/2013 | | | |
| | (H7N9))] | | | |

TABLE 37
Top 62 highest scoring viral proteins with predicted furin cleavage
sites. Here, the column entitled “Protein” shows the NCBI Accession No. of the predicted
protein. The column entitled “Score” shows the PiTou score.

| Protein | Description | R_position | Motif-P14-P1 | Score |
|---|---|---|---|---|
| NP_041724.2 | polyprotein [West Nile | 215 | GRCTKTRHSRRSRR | 15.3974 |
| | virus] | | | |
| YP_001129354.1 | ORF8 [Human | 440 | ASPMTTSASRRKRR | 15.2278 |
| | gammaherpesvirus 8] | | | |
| YP_173238.1 | spike glycoprotein | 760 | YNSPSSSSSRRKRR | 14.6348 |
| | [Human coronavirus | | | |
| | HKU1] | | | |
| NP_044011.1 | MC60R [Molluscum | 364 | ALPAARPRRSRTRR | 13.8656 |
| | contagiosum virus subtype | | | |
| | 1] | | | |
| NP_043135.1 | polyprotein [Tick-borne | 205 | YGRCGKQEGSRTRR | 13.647 |
| | encephalitis virus] | | | |
| YP_308669.1 | hemagglutinin [Influenza | 346 | LRNTPQRERRRKKR | 13.5885 |
| | A virus | | | |
| | (A/goose/Guangdong/1/ | | | |
| | 1996(H5N1))] | | | |
| NP_041726.1 | polyprotein precursor | 210 | GKCDSAGRSRRSRR | 13.2778 |
| | [Yellow fever virus] | | | |
| NP_073286.1 | polyprotein [Dengue virus | 204 | GTCTQSGERRREKR | 13.2573 |
| | 4]] | | | |
| NP_040831.1 | spike protein [Infectious | 690 | ISLLLTNPSSRRKR | 12.7323 |
| | bronchitis virus] | | | |
| YP_073779.1 | envelope glycoprotein B | 398 | LENFSNASRKRRKR | 12.3138 |
| | [Human betaherpesvirus | | | |
| | 7] | | | |
| NP_042932.1 | glycoprotein B [Human | 399 | MFLEQGSEKIRRRR | 12.0529 |
| | betaherpesvirus 6A] | | | |
| YP_001129508.1 | BALF4 [Human | 432 | RGSTSAAVLRRRRR | 12.0276 |
| | herpesvirus 4 type 2] | | | |
| YP_401713.1 | envelope glycoprotein B | 432 | RGSTPAAVLRRRRR | 11.9466 |
| | [Human | | | |
| | gammaherpesvirus 4] | | | |
| YP_009362094.1 | glycoprotein precursor | 124 | LTHKMIEESHRLRR | 11.5376 |
| | [Sangassou | | | |
| | orthohantavirus] | | | |
| NP_044596.1 | Fusion protein (F) | 136 | KNTNVTLSKKRKRR | 11.4852 |
| | [Respiratory syncytial | | | |
| | virus] | | | |
| NP__056863.1 | fusion glycoprotein | 136 | KNLNVSISKKRKRR | 11.4368 |
| | [Human | | | |
| | orthopneumovirus] | | | |
| NP_056922.1 | fusion protein [Measles | 112 | RPVQSVASSRRHKR | 11.4277 |
| | morbillivirus] | | | |
| YP_001129465.1 | EBNA-3C [Human | 501 | VSFKPPPPPSRRRR | 11.1623 |
| | herpesvirus 4 type 2] | | | |
| NP_054711.1 | fusion protein [Mumps | 102 | NIASPSSGSRRHKR | 11.1454 |
| | rubulavirus] | | | |
| NP_057863.1 | p27 [Human T-cell | 10 | MPKTRRRPRR | 11.0752 |
| | leukemia virus type I] | | | |
| YP_001469634.1 | polyprotein [Hepatitis C | 2197 | SHITAETASRRLKR | 11.0478 |
| | virus genotype 6] | | | |
| YP_081514.1 | envelope glycoprotein B | 460 | NRSSLNLTHNRTKR | 11.0306 |
| | [Human betaherpesvirus] | | | |
| NP_056776.2 | polyprotein [Dengue virus | 205 | GTCTTMGEHRREKR | 11.0097 |
| | 2] | | | |
| NP_057865.1 | gp46 SU [Human T-cell | 312 | SLSPVPTLGSRSRR | 10.9953 |
| | leukemia virus type I] | | | |
| NP_690589.2 | structural polyprotein | 325 | LQASLTCSPHRQRR | 10.9598 |
| | [Chikungunya virus] | | | |
| NP_042023.1 | glycoprotein [Borna | 249 | YTSSCRPRLKRRRR | 10.9553 |
| | disease virus 1] | | | |
| YP_009555245.1 | nucleocapsid protein | 274 | EVRQKILNKPRQKR | 10.8307 |
| | [Human coronavirus | | | |
| | OC43] | | | |
| YP_009824998.1 | spike glycoprotein | 537 | FYIKITNGTRRFRR | 10.7175 |
| | [Infectious bronchitis | | | |
| | virus] | | | |
| YP_007188578.1 | ORF1a polyprotein | 1106 | QSVTVKPKRLRKKR | 10.6809 |
| | [Betacoronavirus England | | | |
| | 1] | | | |
| YP_009047202.1 | 1AB polyprotein [Middle | 1106 | QNVTVKPKRLRKKR | 10.6809 |
| | East respiratory syndrome- | | | |
| | related coronavirus] | | | |
| NP_040154.2 | envelope glycoprotein B | 494 | SPQKHPTRNTRSRR | 10.5828 |
| | [Human alphaherpesvirus | | | |
| | 3] | | | |
| YP_009163895.1 | L2 [Human | 9 | MYRTKRVKR | 10.5448 |
| | papillomavirus] | | | |
| YP_009664789.1 | capsid protein precursor | 48 | STTTAPRAPRRRRR | 10.5338 |
| | [Mamastrovirus 18] | | | |
| YP_009380532.1 | capsid protein | 45 | RAQQARRRRNRTRR | 10.5301 |
| | [Mamastrovirus 2] | | | |
| YP_009272536.1 | polyprotein [Hepatitis C | 2196 | SHITAETAARRLRR | 10.4354 |
| | virus genotype 7] | | | |
| NP_056788.1 | capsid protein [Hepatitis E | 333 | VSRYSSTARHRLRR | 10.2448 |
| | virus] | | | |
| NP_598404.1 | fusion protein [Human | 106 | KISTVTDTKTRQKR | 9.72278 |
| | rubulavirus 2] | | | |
| YP_009268921.1 | glycoprotein [Borna | 249 | YTSSCRPRLVRRRR | 9.65791 |
| | disease virus 2] | | | |
| NP_041006.1 | env propeptide, (putative; | 308 | SLAPVPPPATRRRR | 9.40027 |
| | first expressed exon); | | | |
| | putative [Human T- | | | |
| | lymphotropic virus 2] | | | |
| YP_009724390.1 | surface glycoprotein | 685 | ASYQTQTNSPRRAR | 9.19633 |
| | [Severe acute respiratory | | | |
| | syndrome coronavirus 2] | | | |
| YP_009141014.1 | glycoprotein precursor | 1060 | WILRVVLRRSRIRR | 8.95328 |
| | [Bhanja virus] | | | |
| YP_002455789.1 | envelope glycoprotein | 307 | LSPLPGAPLTRRRR | 8.92879 |
| | [Human T-lymphotropic | | | |
| | virus 4] | | | |
| YP_009047204.1 | spike protein [Middle East | 887 | LEPVSISTGSRSAR | 8.55386 |
| | respiratory syndrome- | | | |
| | related coronavirus] | | | |
| YP_007188579.1 | spike protein | 887 | LEPVSISTGSRSAR | 8.55386 |
| | [Betacoronavirus England | | | |
| | 1] | | | |
| YP_142353.1 | transmembrane | 490 | RVCCKRVRRRRSGR | 8.3646 |
| | glycoprotein G [Mokola | | | |
| | lyssavirus] | | | |
| YP_009137152.1 | envelope glycoprotein L | 159 | RAGCVNFDYSRTRR | 8.32858 |
| | [Human alphaherpesvirus | | | |
| | 2] | | | |
| YP_009137096.1 | envelope glycoprotein H | 545 | AGVPSAVORERARR | 7.98401 |
| | [Human alphaherpesvirus] | | | |
| NP_950235.1 | glycoprotein precursor | 247 | VQDTHPSPTNRSKR | 7.60291 |
| | [Crimean-Congo | | | |
| | hemorrhagic fever | | | |
| | orthonairovirus] | | | |
| YP_009254001.1 | G [Lonestar tick chuvirus | 618 | VNPEWTPFRERLRR | 7.57886 |
| | 1] | | | |
| NP_050220.1 | Glycoprotein B [Human | 399 | MVLEQGSVNLRRRR | 7.3109 |
| | betaherpesvirus 6B] | | | |
| YP_009055225.1 | glycoprotein [Marburg | 435 | SPTTRPPIYFRKKR | 7.06092 |
| | marburgvirus] | | | |
| NP_056796.1 | transmembrane | 218 | GMSCDIFTNSRGKR | 6.6124 |
| | glycoprotein G [Rabies | | | |
| | lyssavirus] | | | |
| NP_982303.1 | polyprotein [Oropouche | 707 | ASKLLDINLGRSTR | 6.2933 |
| | virus] | | | |
| YP_001531156.1 | glycoprotein [Marburg | 435 | SSTAQHLVYFRRKR | 6.13352 |
| | marburgvirus] | | | |
| YP_009227121.1 | glycoprotein precursor | 516 | LLFVCKAVKGIKRR | 5.42962 |
| | [Tofla virus] | | | |
| YP_003848705.1 | glycoprotein [Rift Valley | 466 | KPLCVGYERVVVKR | 5.24779 |
| | fever virus] | | | |
| YP_009555241.1 | spike surface glycoprotein | 757 | SGYCVDYSKNRRSR | 4.64604 |
| | [Human coronavirus | | | |
| | OC43] | | | |
| YP_009362075.1 | glycoprotein precursor | 291 | GAKFETSDRIRLHR | 4.63 |
| | [Kaeng Khoi virus] | | | |
| NP_067151.1 | fusion protein [Human | 109 | NQESNENTNPRTKR | 4.43207 |
| | respirovirus 3] | | | |
| YP_009177714.1 | glycoprotein [Tacheng | 267 | HLGPNKEYMRVLRR | 4.22628 |
| | Tick Virus 4] | | | |
| YP_009512922.1 | polyprotein, partial [Tete | 728 | NYWAHLENTORVQR | 4.1588 |
| | orthobunyavirus] | | | |
| YP_009118475.1 | hemagglutinin [Influenza | 463 | ADSEMDKLYERVKR | 3.97254 |
| | A virus | | | |
| | (A/Shanghai/02/2013 | | | |
| | (H7N9))] | | | |

### Example 20. VEGF Binding Assay

To assess the binding of the constructs of the present invention molecules to VEGF₁₆₅: BLI binding studies using the Octet red 96 system were conducted. Briefly, constructs were immobilized on Anti-Human Fc Capture (AHC) biosensors and interrogated for binding to recombinant VEGF₁₆₅ that had been commercially sourced. Using BLI technology, the binding to VEGF₁₆₅ was interrogated using SEQ ID NOs: 122, 154, and 193 for binding affinity (equilibrium binding constant, K_{D}) in the monovalent format.

TABLE 38
Materials and equipment used in the VEGF binding assay.

| Name | Vendor | Catalog No. | Lot No. |
|---|---|---|---|
| 2X Kinetic Buffer | Sartorius | 18-1105 | 910031 |
| 1X PBS (Phosphate | Corning | 21-031-CM | 0421019 |
| buffered saline) | | | |
| HBS Buffer | | N/A | 20210510 |
| HEPES Buffer | Gibco | 15630-108 | 230487 |
| Solution | | | |
| VEGF₁₆₅ | Stem Cell | 78073 | 1000057286 |
| | Technologies | | |
| Red96e Forte Bio | Sartorius | | SN: FB-90470 |
| 96 Well Black Plate | Greiner Bio One | 655209 | N/A |
| AHC Biosensors | Sartorius | 18-5063 | 2104000311 |

Methods

Construct molecules of the present invention binding to VEGF₁₆₅ as well as the K_{D} determination of the interaction were performed using the Octet Red 96 system, while K_{D} determination. Briefly, molecules were immobilized at 1.5 μg/mL in 1× or 2×Kinetic buffer onto Anti-hIgG Fc biosensors with a loading times of 120 or 180 seconds. Baseline post-loading was performed for 60 seconds. Next, 50 nM, 25 nM, and 12.5 nM of recombinant viral protein/s were prepared in 1× or 2×Kinetic Buffer. Reference sensors were generated by applying VEGF₁₆₅ over a blank Anti-hIgG Fc biosensors (no molecules). The association and dissociation steps were 300 seconds each. The data were analyzed using the Octet analysis software with 1:1 or 2:1 model fit applied which reports a dissociation constant K_{D} (M).

To assess the binding of the molecules to VEGF₁₆s: BLI binding studies using the Octet red 96 system were conducted. Briefly, the constructs were immobilized on Anti-Human Fc Capture (AHC) biosensors and interrogated for binding to recombinant VEGF₁₆S that had been commercially sourced. Using BLI technology, the binding to VEGF₁₆₅ was interrogated using SEQ ID NOs: 122, 154, and 209 for binding affinity (equilibrium binding constant, K_{D}) in the monovalent format.

Results

The constructs binding VEGF₁₆₅ with single nanomolar to sub-nanomolar affinity displays the natural occurring interaction between the neuropilin domains and the natural ligand. See table below.

TABLE 39
Results of VEGF₁₆₅ binding assay.

| SEQ ID NO. | VEGF₁₆₅ Binding K_{D} (M) |
|---|---|
| 122 | 6.3E−10 |
| 154 | 3.0E−09 |
| 193 | 1.6E−09 |

The goals of the present example was to determine if the natural ligand VEGF₁₆₅ binds canonical neuropilin binding domains present in present invention's constructs. From the foregoing results, the following conclusions can be made: VEGF₁₆s binds to construct molecules containing neuropilin domains in-line with published literature on VEGF₁₆₅ and neuropilin interactions; the affinity (K_{D}) of construct molecules to VEGF₁₆S is strong, ranging from single digit nanomolar to sub-nanomolar; and, from the data presented here, the construct molecules maintain the established interaction of VEGF₁₆₅ and neuropilin domains and help establish the therapeutic potential for a platform that uses natural occurring interactions to fight viral diseases.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims. It is further to be understood that all values are approximate, and are provided for description.

We claim:

1. A polypeptide comprising:
(a) a b1 domain, or a derivative or fragment thereof, of a neuropilin; and
(b) an immunoglobulin domain;
wherein the b1 domain is capable of binding to a coat protein of a virus

2. The polypeptide of claim 1, further comprising an ACE2 domain, or a derivative or a fragment thereof, of an angiotensin converting enzyme 2.

3. A polypeptide comprising:
(a) an ACE2 domain, or a derivative or a fragment thereof, of an angiotensin converting enzyme 2; and
(b) an immunoglobulin domain;
wherein the ACE2 domain is capable of binding to a coat protein of a virus selected from the group consisting of herpesviridae, papillomaviridae, coronaviridae, flaviviridae, togaviridae, bornaviridae, bunyaviridae, filoviridae, orthomyxoviridae, paramyxoviridae, pneumoviridae, and retroviridae.

4. The polypeptide of claim 3, further comprising a b1 domain, or a derivative or fragment thereof, of a neuropilin.

5. A polypeptide comprising:
(a) a b1 domain, or a derivative or fragment thereof, of a neuropilin; and
(b) an ACE2 domain, or a derivative or fragment thereof, of angiotensin converting enzyme 2;
wherein the b1 domain and ACE2 domain are each capable of binding to a coat protein of a virus selected from the group consisting of herpesviridae, papillomaviridae, coronaviridae, flaviviridae, togaviridae, bornaviridae, bunyaviridae, filoviridae, orthomyxoviridae, paramyxoviridae, pneumoviridae, and retroviridae.

6. The polypeptide of claim 5, further comprising an immunoglobulin domain.

7. The polypeptide of any of claims 1-2 and 4-6, wherein the b1 domain, or derivative or fragment thereof, comprises the amino acid sequence of SEQ ID. NOS: SEQ ID No. 3 (NRP1 b1) or SEQ ID NO. 11 (NRP2 b1).

8. The polypeptide of any of claims 1-2 and 4-7, wherein the b1 domain, or derivative or fragment thereof, comprises the amino acid sequence of SEQ ID NO: 3.

9. The polypeptide of any of claims 1-8, wherein the polypeptide is capable of binding to a coat protein of a coronaviridae virus.

10. The polypeptide of claim 9, wherein the polypeptide is capable of binding to a coat protein of COVID-19.

11. The polypeptide of claim 10, wherein the coat protein is an S protein of COVID-19.

12. The polypeptide of any of claims 1-2 and 4-11, wherein the b1 domain, or derivative or fragment thereof, comprises a mutation that enhances the affinity for an S protein of COVID-19 when compared with the unmutated b1 domain.

13. The polypeptide of claim 12, wherein the b1 domain, or derivative or fragment thereof, comprises a mutation at a position selected from the group consisting of E319 and K351.

14. The polypeptide of claim 13, wherein the b1 domain comprises the amino acid sequence of any of SEQ ID. NOS: SEQ ID NO. 4 (NRP1 b1 E319A) and SEQ ID NO. 5 (NRP1 b1 K351A).

15. The polypeptide of any of claims 1-2 and 4-14, wherein the polypeptide contains a plurality of b1 domains, or derivatives or fragments thereof.

16. The polypeptide of any of claims 1-2 and 4-15, wherein the b1 domain, or derivative or fragment thereof, further comprises a linker, a b2 domain of neuropilin, or a combination thereof.

17. The polypeptide of claim 16, wherein the b1 domain, or derivative or fragment thereof, is selected from the group consisting of SEQ ID NOS: SEQ ID NO. 7-SEQ ID NO. 14.

18. The polypeptide of any of claim 2-17, wherein the ACE2 domain, or derivative or fragment thereof comprises a sequence selected from the group consisting of SEQ ID NOS: SEQ ID NO. 38-SEQ ID NO. 39.

19. The polypeptide of any of claim 2-18, wherein the polypeptide comprises a plurality of ACE2 domains, or derivatives or fragments thereof.

20. The polypeptide of any of claims 2-20, wherein the ACE2 domain contains a mutation at a position selected from the group consisting of F28, D30, and L79.

21. The polypeptide of claim 20, wherein the ACE2 domain, derivative or fragment thereof comprises the amino acid sequence of SEQ ID. NOs: SEQ ID NO. 40-SEQ ID NO. 43.

22. The polypeptide of any of claims 2-6 and 15-16, further comprising a linker between the b1 domain and ACE2 domain.

23. The polypeptide of claim 22, wherein the linker is selected from the group consisting of SEQ ID NOs: 44-50.

24. The polypeptide of any of claims 1-4 and 6-23, wherein the immunoglobulin domain comprises a Fc domain.

25. The polypeptide of any of claims 1-4 and 6-24, wherein the immunoglobulin domain consists essentially of a Fc domain.

26. The polypeptide of any of claims 24 and 25, wherein the Fc domain contains a mutation that reduces ADCC when compared with a wildtype Fc domain.

27. The polypeptide of claim 26, wherein the mutation is at position N297 as determined by KABAT numbering.

28. The polypeptide of any of claims 24 and 25, wherein the Fc domain contains one or more mutations that enhance affinity for a FcRn when compared with a wildtype Fc domain.

29. The polypeptide of claim 28, wherein the mutation is at a position selected from the group consisting of T307, E380, and N434 as determined by KABAT numbering, or combinations thereof.

30. The polypeptide of any of claims 24 and 25, wherein the Fc domain contains a mutation that reduces affinity for Fcγ receptor subtypes when compared with a wildtype Fc domain.

31. The polypeptide of claim 30, wherein the mutation is at a position selected from the group consisting of L324 and L325 as determined by KABAT numbering, or combinations thereof.

32. The polypeptide of any of claims 18-21, wherein the Fc domain is selected from the group consisting of human IgG1, human IgG2, human IgG3, human IgG4, and human IgA.

33. The polypeptide of claim 32, wherein the Fc domain comprises the amino acid sequence selected from the group consisting of SEQ ID. NOs: 23-31.

34. The polypeptide of claim 32, wherein the Fc domain sequence comprises the amino acid sequence of SEQ ID. NOS: 23, 30, or 31.

35. The polypeptide of any of claims 2, 4, and 6, wherein the polypeptide has a configuration selected from the group consisting of:
i. (b1), IgG1 WT, ACE2-1;
ii. (b1b2), IgG1 (T307A/E380A/N434A), ACE2-2;
iii. (b1b1)-(G4S)*2-(b1b1), IgG1 (N297A), ACE2-3;
iv. (b1b2)-(G4S)*2-(b1b2), IgG1 (L324A/L325A), ACE2-4;
v. (b1b2)-(G4S)*2-(b1b2) with b1 (E319A), IgG1 (N297A/T307A/E380A/N434A), ACE2-5; and
vi. (b1b2)-(G4S)*2-(b1b2) with b1 (K351A), IgG1 (L324A/L325A/T307A/E380A/N434A), ACE2-6.

36. The polypeptide of any of claims 2, 4, and 6, wherein the polypeptide comprises the amino acid sequence selected from the group consisting of SEQ ID. NOS: 88-110.

37. The polypeptide of any of claims 1-2, 4, and 6, wherein the b1 domain is attached to the C-terminus of the Fc domain.

38. The polypeptide of any of claims 1-2, 4, and 6, wherein the b1 domain is attached to the N-terminus of the Fc domain.

39. The polypeptide of any of claims 2, 3-4, and 6, wherein the ACE2 domain is attached to the C-terminus of the Fc domain.

40. The polypeptide of any of claims 2, 3-4, and 6, wherein the ACE2 domain is attached to the N-terminus of the Fc domain.

41. The polypeptide of any of claims 1-40, further comprising a signal peptide.

42. The polypeptide of claim 41, wherein the signal peptide comprises the SEQ ID NO. 51.

43. A method of producing the polypeptide of any of claims 1-42, the method comprising recombinantly expressing a nucleic acid vector encoding the polypeptide in a host cell.

44. A pharmaceutical composition comprising the polypeptide of any of claims 1-42 and a pharmaceutically acceptable excipient.

45. A method of reducing COVID infection, the method comprising the administration of the polypeptides of any of claims 1-42 to a subject in need thereof.

46. A method of treating a subject suffering from COVID infection, the method comprising the administration of the polypeptides of any of claims 1-42 to a subject in need thereof.

47. A method of preventing COVID infection, the method comprising the administration of the polypeptides of any of claims 1-42 to a subject in need thereof.

48. A method of reducing symptoms of a COVID infection, the method comprising the administration of the polypeptides of any of claims 1-42 to a subject in need thereof.

49. A method of reducing transmission of a COVID infection, the method comprising the administration of the polypeptides of any of claims 1-42 to a subject in need thereof.

50. A recombinant polypeptide comprising:
(a) one or more mutant neuropilin (NRP) domains, or fragments thereof, and
(b) an immunoglobulin domain;
wherein the one or more mutant NRP domains result in reduced binding of the recombinant polypeptide to heparin or heparan sulfate relative to a wild-type NRP domain.

51. The recombinant polypeptide of claim 50, wherein the one or more mutant NRP domains are derived from an NRP1 or an NRP2 protein.

52. The recombinant polypeptide of claim 50, wherein the one or more mutant NRP domains are one or more mutant b1 domains, or one or more mutant b2 domains.

53. The recombinant polypeptide of claim 50, wherein the one or more mutant NRP domains has one or more amino substitutions selected from groups consisting of: K373E, K351A, E319A, K358E, R513E, K514E, K516E, R513A, K514A, K516A, Y297A, S345A, and Y353A, relative to the wild-type amino acid sequence set forth in SEQ ID NO: 1.

54. The recombinant polypeptide of claim 50, wherein the one or more mutant NRP domains result in reduced binding of the recombinant polypeptide to heparin.

55. The recombinant polypeptide of claim 50, wherein the one or more mutant NRP domains result in reduced binding of the recombinant polypeptide to heparan sulfate.

56. The recombinant polypeptide of claim 50, wherein the immunoglobin domain is an Fc domain.

57. A recombinant polypeptide comprising:
(a) one or more mutant neuropilin (NRP) b1 domains, NRP b2 domains, or fragments thereof, and
(b) an Fc domain;
wherein the one or more mutant NRP b1 domains, NRP b2 domains, or fragments thereof are derived from an NRP1 or an NRP2 protein;
wherein the one or more mutant NRP b1 domains, NRP b2 domains, or fragments thereof
have one or more amino substitutions selected from groups consisting of: K373E, K351A, E319A, K358E, R513E, K514E, K516E, R513A, K514A, K516A, Y297A, S345A, and Y353A, relative to the wild-type amino acid sequence set forth in SEQ ID NO: 1; and
wherein the one or more one or more amino substitutions result in reduced binding of the recombinant polypeptide to heparin or heparan sulfate.

58. A recombinant polypeptide comprising:
(a) one or more mutant neuropilin (NRP) b1 domains (b1), NRP b2 domains (b2), or fragments thereof; and
(b) an Fc domain;
wherein (a) and (b) comprise a construct having an orientation of: b1-Fc; b1b1-Fc;
b1b1b1-Fc; b1-Fc; b1b1b1-Fc; b1b2-Fc; b1b2-Fc; b1b2-Fc; b1b2-Fc; Fc-b1b2; Fc-b1b2; b1-Fc-b1; b1b1-Fc-b1; b1-Fc; b1b1-Fc; b1b1b1-Fc; b1-Fc; b1b1b1-Fc; b1-Fc; b1b1b1-Fc; b1b2-Fc; b1b2-Fc; Fc-b1b2; Fc-b1b2; b1-Fc-b1; b1b1-Fc-b1;
wherein the one or more b1, b2, or fragments thereof, are derived from an NRP1 or an NRP2 protein;
wherein the one or more b1, b2, or fragments thereof have one or more amino substitutions selected from groups consisting of: K373E, K351A, E319A, K358E, R513E, K514E, K516E, R513A, K514A, K516A, Y297A, S345A, and Y353A, relative to the wild-type amino acid sequence set forth in SEQ ID NO: 1; and
wherein the one or more one or more amino substitutions result in reduced binding of the recombinant polypeptide to heparin or heparan sulfate.

59. A recombinant polypeptide comprising:
(a) one or more mutant neuropilin (NRP) domains, or fragments thereof, and
(b) an immunoglobulin domain;
wherein the recombinant polypeptide is operable to bind to virus having an —Z₁-X₁-X₂—Z₂- (CendR) motif, wherein Z₁ and Z₂ are arginine or lysine, and X₁ and X₂ are any amino acid.

60. The recombinant polypeptide of claim 59, wherein the one or more mutant NRP domains, or fragments thereof, are derived from an NRP1 or an NRP2 protein.

61. The recombinant polypeptide of claim 59, wherein the one or more mutant NRP domains, or fragments thereof, are one or more mutant b1 domains, or one or more mutant b2 domains.

62. The recombinant polypeptide of claim 59, wherein the virus is a virus belonging to the Realm: Duplodnaviria; Monodnaviria; Riboviria; or Varidnaviria.

63. The recombinant polypeptide of claim 59, wherein the virus is a virus belonging to the Kingdom: Bamfordvirae; Heunggongvirae; Orthornavirae; Pararnavirae; or Shotokuvirae.

64. The recombinant polypeptide of claim 59, wherein the virus is a virus belonging to the Phylum: Artverviricota; Cossaviricota; Kitrinoviricota; Negarnaviricota; Nucleocytoviricota; Peploviricota; or Pisuviricota.

65. The recombinant polypeptide of claim 59, wherein the virus is a virus belonging to the Class: Alsuviricetes; Ellioviricetes; Flasuviricetes; Herviviricetes; Insthoviricetes; Monjiviricetes; Papovaviricetes; Pisoniviricetes; Pokkesviricetes; Revtraviricetes; or Stelpaviricetes.

66. The recombinant polypeptide of claim 59, wherein the virus is a virus belonging to the Order: Amarillovirales; Articulavirales; Bunyavirales; Chitovirales; Hepelivirales; Herpesvirales; Jingchuvirales; Martellivirales; Mononegavirales; Nidovirales; Ortervirales; Stellavirales; or Zurhausenvirales.

67. The recombinant polypeptide of claim 59, wherein the virus is a virus belonging to the Family: Astroviridae; Bunyaviridae; Bornaviridae; Chuviridae; Coronaviridae; Flaviviridae; Filoviridae; Hantaviridae; Hepeviridae; Herpesviridae; Nairoviridae; Orthomyxoviridae; Papillomaviridae; Paramyxoviridae; Peribunyaviridae; Phenuiviridae; Pneumoviridae; Poxviridae; Retroviridae; Rhabdoviridae; or Togaviridae.

68. The recombinant polypeptide of claim 59, wherein the virus is selected from the group consisting of: Dengue; respiratory syncytial virus (RSV); Hantavirus; Epstein-Barr virus (EBV); EBV (uncleaved); SARS-CoV-2 Wuhan; SARS-CoV-2 Wuhan (uncleaved); SARS-CoV-2 UK; SARS-CoV-2 India; SARS-CoV-2 India (uncleaved); HCoV-OC43; MERS-CoV; MERS-CoV (uncleaved); Herpes simplex virus (HSV) 1; HSV 1 (uncleaved); influenza A H5N1 virus (IAV H5N1); human papillomavirus (HPV); Human Metapneumovirus; and human immunodeficiency virus (HIV).

69. The recombinant polypeptide of claim 59, wherein the virus has a CendR motif selected from the group consisting of:
GTCTQSGERRREKR;
KNTNVTLSKKRKRR;
LTHKMIEESHRLRR;
VSFKPPPPPSRRRR;
VSFKPPPPPSRRRRGACVVY;
CASYQTQTNSPRRAR;
CASYQTQTNSPRRARSVASQSIIAYTMSLG;
ASYQTQTNSHRRAR;
ASYQTQTNSRRRAR;
ASYQTQTNSRRRARSVASQSIIAY;
GSGYCVDYSKNRRSR;
LLEPVSISTGSRSAR;
LLEPVSISTGSRSARSAIEDLLFDK;
ERPRAPARSASRPRR;
ERPRAPARSASRPRRPV;
VLATGLRNVPQRKKR;
PTTSSTSTTAKRKKR;
IDMLKARVKNRVAR;
AKRRVVQREKR;
and
AKRRVVQREKRAVGIGALFLG.

70. A recombinant polypeptide comprising:
(a) one or more mutant neuropilin (NRP) b1 domains (b1), NRP b2 domains (b2), or fragments thereof; and
(b) an Fc domain;
wherein the one or more b1, b2, or fragments thereof, are derived from an NRP1 or an NRP2 protein;
wherein the recombinant polypeptide is operable to bind to virus having an —Z₁-X₁-X₂-Z₂- (CendR) motif, wherein Z₁ and Z₂ are arginine or lysine, and X₁ and X₂ are any amino acid; and
wherein the virus is selected from the group consisting of: Dengue; respiratory syncytial virus (RSV); Hantavirus; Epstein-Barr virus (EBV); EBV (uncleaved); SARS-CoV-2 Wuhan; SARS-CoV-2 Wuhan (uncleaved); SARS-CoV-2 UK; SARS-CoV-2 India; SARS-CoV-2 India (uncleaved); HCoV-OC43; MERS-CoV; MERS-CoV (uncleaved); Herpes simplex virus (HSV) 1; HSV 1 (uncleaved); influenza A H5N1 virus (IAV H5N1); human papillomavirus (HPV); Human Metapneumovirus; and human immunodeficiency virus (HIV).

71. A recombinant polypeptide comprising:
(a) one or more mutant neuropilin (NRP) b1 domains (b1), NRP b2 domains (b2), or fragments thereof; and
(b) an Fc domain;
wherein the one or more b1, b2, or fragments thereof, are derived from an NRP1 or an NRP2 protein;
wherein the recombinant polypeptide is operable to bind to virus having an —Z₁-X₁-X₂—Z₂- (CendR) motif, wherein Z₁ and Z₂ are arginine or lysine, and X₁ and X₂ are any amino acid; and
wherein the virus has a CendR motif selected from the group consisting of:
GTCTQSGERRREKR;
KNTNVTLSKKRKRR;
LTHKMIEESHRLRR;
VSFKPPPPPSRRRR;
VSFKPPPPPSRRRRGACVVY;
CASYQTQTNSPRRAR;
CASYQTQTNSPRRARSVASQSIIAYTMSLG;
ASYQTQTNSHRRAR;
ASYQTQTNSRRRAR;
ASYQTQTNSRRRARSVASQSIIAY;
GSGYCVDYSKNRRSR;
LLEPVSISTGSRSAR;
LLEPVSISTGSRSARSAIEDLLFDK;
ERPRAPARSASRPRR;
ERPRAPARSASRPRRPV;
VLATGLRNVPQRKKR;
PTTSSTSTTAKRKKR;
IDMLKARVKNRVAR;
AKRRVVQREKR;
and
AKRRVVQREKRAVGIGALFLG.

72. A recombinant polypeptide comprising an amino acid sequence that is at least 90% identical to an amino acid sequence set forth in any one of SEQ ID NOs: 113-116, 121-122, 133-137, 148-149, 154, 162, and 193-201, or a pharmaceutically acceptable salt thereof.

73. A recombinant polypeptide consisting of an amino acid sequence that is at least 90% identical to an amino acid sequence according to set forth in any one of SEQ ID NOs: 113-116, 121-122, 133-137, 148-149, 154, 162, and 193-201, or a pharmaceutically acceptable salt thereof.

74. A recombinant polypeptide consisting of an amino acid sequence set forth in any one of SEQ ID NOs: 113-116, 121-122, 133-137, 148-149, 154, 162, and 193-201, or a pharmaceutically acceptable salt thereof.

75. A method of limiting the occurrence of, reducing the risk of, reducing the severity of, or treating a viral infection, in a subject in need thereof, said method comprising administering to the subject a composition comprising a therapeutically effective amount of a recombinant polypeptide, or a pharmaceutically acceptable salt thereof, comprising
(a) one or more mutant neuropilin (NRP) domains, or fragments thereof, and
(b) an immunoglobulin domain;
wherein the recombinant polypeptide is operable to bind to virus having an —Z₁-X₁-X₂-Z₂- (CendR) motif, wherein Z₁ and Z₂ are arginine or lysine, and X₁ and X₂ are any amino acid.

76. The method of claim 75, wherein the one or more mutant NRP domains, or fragments thereof, are derived from an NRP1 or an NRP2 protein.

77. The method of claim 75, wherein the one or more mutant NRP domains, or fragments thereof, are one or more mutant b1 domains, or one or more mutant b2 domains.

78. The method of claim 75, wherein the virus is a virus belonging to the Realm: Duplodnaviria; Monodnaviria; Riboviria; or Varidnaviria.

79. The method of claim 75, wherein the virus is a virus belonging to the Kingdom: Bamfordvirae; Heunggongvirae; Orthornavirae; Pararnavirae; or Shotokuvirae.

80. The method of claim 75, wherein the virus is a virus belonging to the Phylum: Artverviricota; Cossaviricota; Kitrinoviricota; Negarnaviricota; Nucleocytoviricota; Peploviricota; or Pisuviricota.

81. The method of claim 75, wherein the virus is a virus belonging to the Class: Alsuviricetes; Ellioviricetes; Flasuviricetes; Herviviricetes; Insthoviricetes; Monjiviricetes; Papovaviricetes; Pisoniviricetes; Pokkesviricetes; Revtraviricetes; or Stelpaviricetes.

82. The method of claim 75, wherein the virus is a virus belonging to the Order: Amarillovirales; Articulavirales; Bunyavirales; Chitovirales; Hepelivirales; Herpesvirales; Jingchuvirales; Martellivirales; Mononegavirales; Nidovirales; Ortervirales; Stellavirales; or Zurhausenvirales.

83. The method of claim 75, wherein the virus is a virus belonging to the Family: Astroviridae; Bunyaviridae; Bornaviridae; Chuviridae; Coronaviridae; Flaviviridae; Filoviridae; Hantaviridae; Hepeviridae; Herpesviridae; Nairoviridae; Orthomyxoviridae; Papillomaviridae; Paramyxoviridae; Peribunyaviridae; Phenuiviridae; Pneumoviridae; Poxviridae; Retroviridae; Rhabdoviridae; or Togaviridae.

84. The method of claim 75, wherein the virus is selected from the group consisting of: Dengue; respiratory syncytial virus (RSV); Hantavirus; Epstein-Barr virus (EBV); EBV (uncleaved); SARS-CoV-2 Wuhan; SARS-CoV-2 Wuhan (uncleaved); SARS-CoV-2 UK; SARS-CoV-2 India; SARS-CoV-2 India (uncleaved); HCoV-OC43; MERS-CoV; MERS-CoV (uncleaved); Herpes simplex virus (HSV) 1; HSV 1 (uncleaved); influenza A H5N1 virus (IAV H5N1); human papillomavirus (HPV); Human Metapneumovirus; and human immunodeficiency virus (HIV).

85. The method of claim 75, wherein the virus has a CendR motif selected from the group consisting of:
GTCTQSGERRREKR;
KNTNVTLSKKRKRR;
LTHKMIEESHRLRR;
VSFKPPPPPSRRRR;
VSFKPPPPPSRRRRGACVVY;
CASYQTQTNSPRRAR;
CASYQTQTNSPRRARSVASQSIIAYTMSLG;
ASYQTQTNSHRRAR;
ASYQTQTNSRRRAR;
ASYQTQTNSRRRARSVASQSIIAY;
GSGYCVDYSKNRRSR;
LLEPVSISTGSRSAR;
LLEPVSISTGSRSARSAIEDLLFDK;
ERPRAPARSASRPRR;
ERPRAPARSASRPRRPV;
VLATGLRNVPQRKKR;
PTTSSTSTTAKRKKR;
IDMLKARVKNRVAR;
AKRRVVQREKR;
and
AKRRVVQREKRAVGIGALFLG.

86. A method of limiting the occurrence of, reducing the risk of, reducing the severity of, or treating a viral infection, in a subject in need thereof, said method comprising administering to the subject a composition comprising a therapeutically effective amount of a recombinant polypeptide, or a pharmaceutically acceptable salt thereof, comprising:
(a) one or more mutant neuropilin (NRP) b1 domains (b1), NRP b2 domains (b2), or fragments thereof; and
(b) an Fc domain;
wherein the one or more b1, b2, or fragments thereof, are derived from an NRP1 or an NRP2 protein;
wherein the recombinant polypeptide is operable to bind to virus having an —Z₁-X₁-X₂-Z₂- (CendR) motif, wherein Z₁ and Z₂ are arginine or lysine, and X₁ and X₂ are any amino acid; and
wherein the virus is selected from the group consisting of: Dengue; respiratory syncytial virus (RSV); Hantavirus; Epstein-Barr virus (EBV); EBV (uncleaved); SARS-CoV-2 Wuhan; SARS-CoV-2 Wuhan (uncleaved); SARS-CoV-2 UK; SARS-CoV-2 India; SARS-CoV-2 India (uncleaved); HCoV-OC43; MERS-CoV; MERS-CoV (uncleaved); Herpes simplex virus (HSV) 1; HSV 1 (uncleaved); influenza A H5N1 virus (IAV H5N1); human papillomavirus (HPV); Human Metapneumovirus; and human immunodeficiency virus (HIV).

87. A method of limiting the occurrence of, reducing the risk of, reducing the severity of, or treating a viral infection, in a subject in need thereof, said method comprising administering to the subject a composition comprising a therapeutically effective amount of a recombinant polypeptide, or a pharmaceutically acceptable salt thereof, comprising:
(a) one or more mutant neuropilin (NRP) b1 domains (b1), NRP b2 domains (b2), or fragments thereof; and
(b) an Fc domain;
wherein the one or more b1, b2, or fragments thereof, are derived from an NRP1 or an NRP2 protein;
wherein the recombinant polypeptide is operable to bind to virus having an —Z₁-X₁-X₂-Z₂- (CendR) motif, wherein Z₁ and Z₂ are arginine or lysine, and X₁ and X₂ are any amino acid; and
wherein the virus has a CendR motif selected from the group consisting of:
GTCTQSGERRREKR;
KNTNVTLSKKRKRR;
LTHKMIEESHRLRR;
VSFKPPPPPSRRRR;
VSFKPPPPPSRRRRGACVVY;
CASYQTQTNSPRRAR;
CASYQTQTNSPRRARSVASQSIIAYTMSLG;
ASYQTQTNSHRRAR;
ASYQTQTNSRRRAR;
ASYQTQTNSRRRARSVASQSIIAY;
GSGYCVDYSKNRRSR;
LLEPVSISTGSRSAR;
LLEPVSISTGSRSARSAIEDLLFDK;
ERPRAPARSASRPRR;
ERPRAPARSASRPRRPV;
VLATGLRNVPQRKKR;
PTTSSTSTTAKRKKR;
IDMLKARVKNRVAR;
AKRRVVQREKR;
and
AKRRVVQREKRAVGIGALFLG.

88. A method of limiting the occurrence of, reducing the risk of, reducing the severity of, or treating a viral infection, in a subject in need thereof, said method comprising administering to the subject a composition comprising a therapeutically effective amount of a recombinant polypeptide comprising an amino acid sequence that is at least 90% identical to an amino acid sequence set forth in any one of SEQ ID NOs: 113-116, 121-122, 133-137, 148-149, 154, 162, and 193-201, or a pharmaceutically acceptable salt thereof.

89. A method of limiting the occurrence of, reducing the risk of, reducing the severity of, or treating a viral infection, in a subject in need thereof, said method comprising administering to the subject a composition comprising a therapeutically effective amount of a recombinant polypeptide consisting of an amino acid sequence that is at least 90% identical to an amino acid sequence according to set forth in any one of SEQ ID NOs: 113-116, 121-122, 133-137, 148-149, 154, 162, and 193-201, or a pharmaceutically acceptable salt thereof.

90. A method of limiting the occurrence of, reducing the risk of, reducing the severity of, or treating a viral infection, in a subject in need thereof, said method comprising administering to the subject a composition comprising a therapeutically effective amount of a recombinant polypeptide consisting of an amino acid sequence set forth in any one of SEQ ID NOs: 113-116, 121-122, 133-137, 148-149, 154, 162, and 193-201, or a pharmaceutically acceptable salt thereof.

91. A recombinant polypeptide comprising an amino acid sequence that is at least 90% identical to an amino acid sequence set forth in any one of SEQ ID NOs: 113-116, 121-122, 133-137, 148-149, 154, 162, and 193-201, or a pharmaceutically acceptable salt thereof, and further comprising an excipient.

92. The method of claim 75, wherein the virus is a virus belonging to the Family: Astroviridae; Bunyaviridae; Bornaviridae; Chuviridae; Flaviviridae; Filoviridae; Hantaviridae; Hepeviridae; Herpesviridae; Nairoviridae; Orthomyxoviridae; Papillomaviridae; Paramyxoviridae; Peribunyaviridae; Phenuiviridae; Pneumoviridae; Poxviridae; Retroviridae; Rhabdoviridae; or Togaviridae.

96. The method of claim 75, wherein the virus is selected from the group consisting of Dengue; respiratory syncytial virus (RSV); Hantavirus; Epstein-Barr virus (EBV); EBV (uncleaved); HCoV-OC43; MERS-CoV; MERS-CoV (uncleaved); Herpes simplex virus (HSV) 1; HSV 1 (uncleaved); influenza A H5N1 virus (IAV H5N1); human papillomavirus (HPV); Human Metapneumovirus; and human immunodeficiency virus (HIV).

97. The method of claim 75, wherein the virus has a CendR motif selected from the group consisting of:
GTCTQSGERRREKR;
KNTNVTLSKKRKRR;
LTHKMIEESHRLRR;
VSFKPPPPPSRRRR;
VSFKPPPPPSRRRRGACVVY;
GSGYCVDYSKNRRSR;
LLEPVSISTGSRSAR;
LLEPVSISTGSRSARSAIEDLLFDK;
ERPRAPARSASRPRR;
ERPRAPARSASRPRRPV;
VLATGLRNVPQRKKR;
PTTSSTSTTAKRKKR;
IDMLKARVKNRVAR;
AKRRVVQREKR;
and
AKRRVVQREKRAVGIGALFLG.

98. A method of limiting the occurrence of, reducing the risk of, reducing the severity of, or treating a viral infection, in a subject in need thereof, said method comprising administering to the subject a composition comprising a therapeutically effective amount of a recombinant polypeptide, or a pharmaceutically acceptable salt thereof, comprising:
(a) one or more mutant neuropilin (NRP) b1 domains (b1), NRP b2 domains (b2), or fragments thereof; and
(b) an Fc domain;
wherein the one or more b1, b2, or fragments thereof, are derived from an NRP1 or an NRP2 protein;
wherein the recombinant polypeptide is operable to bind to virus having an —Z₁-X₁-X₂-Z₂- (CendR) motif, wherein Z₁ and Z₂ are arginine or lysine, and X₁ and X₂ are any amino acid; and
wherein the virus is selected from the group consisting of: Dengue; respiratory syncytial virus (RSV); Hantavirus; Epstein-Barr virus (EBV); EBV (uncleaved); HCoV-OC43; MERS-CoV; MERS-CoV (uncleaved); Herpes simplex virus (HSV) 1; HSV 1 (uncleaved); influenza A H5N1 virus (IAV H5N1); human papillomavirus (HPV); Human Metapneumovirus; and human immunodeficiency virus (HIV).

99. A method of limiting the occurrence of, reducing the risk of, reducing the severity of, or treating a viral infection, in a subject in need thereof, said method comprising administering to the subject a composition comprising a therapeutically effective amount of a recombinant polypeptide, or a pharmaceutically acceptable salt thereof, comprising:
(a) one or more mutant neuropilin (NRP) b1 domains (b1), NRP b2 domains (b2), or fragments thereof; and
(b) an Fc domain;
wherein the one or more b1, b2, or fragments thereof, are derived from an NRP1 or an NRP2 protein;
wherein the recombinant polypeptide is operable to bind to virus having an —Z₁-X₁-X₂-Z₂- (CendR) motif, wherein Z₁ and Z₂ are arginine or lysine, and X₁ and X₂ are any amino acid; and
wherein the virus has a CendR motif selected from the group consisting of:
GTCTQSGERRREKR;
KNTNVTLSKKRKRR;
LTHKMIEESHRLRR;
VSFKPPPPPSRRRR;
VSFKPPPPPSRRRRGACVVY;
GSGYCVDYSKNRRSR;
LLEPVSISTGSRSAR;
LLEPVSISTGSRSARSAIEDLLFDK;
ERPRAPARSASRPRR;
ERPRAPARSASRPRRPV;
VLATGLRNVPQRKKR;
PTTSSTSTTAKRKKR;
IDMLKARVKNRVAR;
AKRRVVQREKR;
and
AKRRVVQREKRAVGIGALFLG.

100. The method of any one of claims 92-99, wherein the virus is not SARS-CoV-2.

101. The method of any one of claims 92-99, wherein the virus is not SARS-CoV-2 Wuhan; SARS-CoV-2 Wuhan (uncleaved); SARS-CoV-2 UK; SARS-CoV-2 India; or SARS-CoV-2 India (uncleaved).
